(19) United States
(12) Patent Application Publication
(10) Pub. No.: US 2008/0194585 A1
(43) Pub. Date: Aug. 14, 2008

(54) Fungicidal Pyrazine Derivatives

(76) Inventors: Paula Louise Sharpe, Middletown, DE (US); Thomas Martin Stevenson, Newark, DE (US); James Francis Bereznak, Newtown Square, PA (US); Ritesh Sheth, Newark, DE (US); Andrew Edmund Taggi, Newark, DE (US); Chip-Ping Tseng, Wilmington, DE (US); Wenming Zhang, Newark, DE (US)

Correspondence Address: Heiser, David E.;E.I. Du Pont De Nemours and Company, Legal Patent Records Center, 4417 Lancaster Pike, Wilmington, DE, 19805, US

(21) Appl. No.: 11/883,659

(22) PCT Filed: Feb. 14, 2006
(86) PCT No.: PCT/US2006/005528

§ 371 Date: Apr. 14, 2008

Related U.S. Application Data
(60) Provisional application No. 60/653,190, filed on Feb. 15, 2005.

(51) Int. Cl. A01N 43/60; C07D 403/04; C07D 241/24; A01P 3/00

(52) U.S. Cl. 514/255.05; 544/405; 544/406

(57) ABSTRACT

Compounds of Formula 1, and their N-oxides and agriculturally suitable salts, are disclosed as useful as fungicides (INSERT FORMULA I HERE) wherein R¹ is NR⁴R⁵, N═CR¹⁹R²¹, OR⁶, G¹ or G²; or C₁-C₈ alkyl, C₂-C₈ alkenyl, each optionally substituted; A is O, S or NR⁷; R² is cyano, NR⁸ N═CR⁹R¹⁰, NC(═O)R³⁰; or a 5- or 6-membered heteroaromatic ring, each ring or ring system optionally substituted; R_{?}3? is H, halogen, cyano, C₁-C₆ alkyl; J is C₁-C₈ alkyl or phenyl, optionally substituted; and R⁴, R⁵, R⁶, R⁷, R⁸, R⁹, R¹⁰, R¹⁹, R²¹, R³⁰, G and G² are as defined in the disclosure. Also disclosed are compositions containing the compounds of Formula 1 and a method for controlling plant diseases caused by fungal plant pathogens which involves applying an effective amount of a compound of Formula 1.

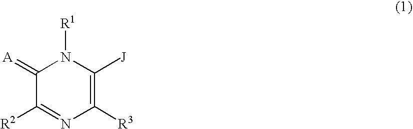

### FIELD OF THE INVENTION

This invention relates to certain pyrazine derivatives, their N-oxides, agriculturally suitable salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes.

WO 03/043993 discloses certain fungicidal 5-phenylpyrimidine compounds of Formula i

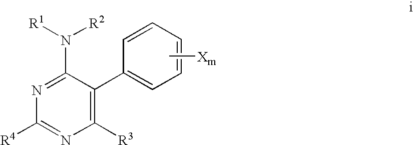

wherein, among others,
R¹ and R² are H, alkyl, haloalkyl, cycloalkyl or alkenyl;
R³ is H, halo, cyano, alkyl, haloalkyl or alkoxy;
R⁴ is H, halo, cyano, hydroxy, mercapto, azido, alkyl or alkenyl;
X is halo, alkyl, alkoxy or haloalkyl; and
m is a whole number from 1 to 5.

The need continues for new compounds which are more effective, less costly, less toxic, environmentally safer or have different modes of action.

### SUMMARY OF THE INVENTION

This invention is directed to compounds of Formula 1 including all geometric and stereoisomers, N-oxides, and agriculturally suitable salts thereof, agricultural compositions containing them and their use as fungicides:

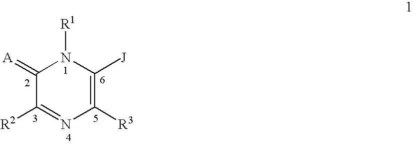

wherein
R¹ is NR⁴R⁵, N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G²;
A is O, S or NR⁷;
R⁷ is H, C₁-C₄ alkyl, C₁-C₄ haloalkyl, C₂-C₆ alkylcarbonyl or C₂-C₆ alkoxycarbonyl;
R² is cyano, NR⁸—N═CR⁹R¹⁰, O—N═CR⁹R¹⁰, NR⁸—NR¹¹R¹², O—NR¹¹R¹², CR¹³═NOR¹⁴, CR¹³═NNR¹¹R¹², C(W)NR²²R²³, NC(═O)R³⁰, NC(═O)NR³¹ or NC(═O)OR³²; or
R² is a 5- or 6-membered heteroaromatic ring or a 8-, 9- or 10-membered heteroaromatic bicyclic ring system, each ring or ring system optionally substituted with up to 5 substituents selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents selected from R²⁴;
W is O, S or ═NR²⁵;
R³ is H, halogen, cyano, C₁-C₆ alkyl, C₁-C₄ haloalkyl, C₃-C₆ cycloalkyl, C₃-C₆ halocycloalkyl, C₂-C₆ alkenyl, C₃-C₆ alkynyl, C₁-C₄ alkoxy, C₁-C₄ haloalkoxy, C₁-C₄ alkylthio, C₁-C₄ haloalkylthio, C₂-C₅ alkoxycarbonyl, hydroxycarbonyl, —SCN or —CHO;
each R⁴ and R⁵ are independently H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R⁴ and R⁵ are taken together as —(CH₂)₃—, —(CH₂)₄—, —(CH₂)₅—, —(CH₂)₆—, —CH₂CH₂OCH₂CH₂— or CH₂CH(CH₃)OCH(CH₃)CH₂—;
R⁶ is H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl;
R⁸ is H, C₁-C₄ alkyl or C₁-C₄ haloalkyl;
R⁹ is C₁-C₄ alkyl or C₁-C₄ haloalkyl;
R¹⁰ is H, C₁-C₄ alkyl or C₁-C₄ haloalkyl; or
R⁹ and R¹⁰ are taken together as —(CH₂)₃—, —(CH₂)₄—, —(CH₂)₅— or —(CH₂)₆—;
R¹¹ is H, C₁-C₄ alkyl or C₁-C₄ haloalkyl;
R¹² is H, C₁-C₄ alkyl, C₁-C₄ haloalkyl, C₂-C₃ alkylcarbonyl or C₂-C₃ alkoxycarbonyl; or
R¹¹ and R¹² are taken together as —(CH₂)₄—, —(CH₂)₅, —CH₂CH₂OCH₂CH₂— or —CH₂CH(CH₃)OCH(CH₃)CH₂—;
R¹³ is H, NH₂, C₁-C₄ alkyl or C₁-C₄ haloalkyl;
R¹⁴ is H, C₁-C₄ alkyl or C₁-C₄ haloalkyl;
J is C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents selected from the group consisting of halogen, cyano, nitro, hydroxy, C₁-C₄ alkoxy, C₁-C₄ haloalkoxy, C₁-C₄ alkylthio, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl and C₁-C₄ alkylamino; or
J is a phenyl, benzyl, naphthalene, 5- or 6-membered heteroaromatic ring or 8-, 9- or 10-membered heteroaromatic bicyclic ring system, each ring or ring system optionally substituted with up to 5 substituents selected from halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl and C₃-C₆ trialkylsilyl;
G¹ is a 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents selected from R¹⁷;
G² is a phenyl ring, 5- or 6-membered heteroaromatic ring, each ring or ring system optionally substituted with from 1 to 4 substituents independently selected from R¹⁸;
each R¹⁷ is independently C₁-C₂ alkyl, C₁-C₂ haloalkyl, halogen, cyano, nitro or C₁-C₂ alkoxy;
each R¹⁸ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, (C₁-C₄ alkyl)(C₃-C₆ cycloalkyl)amino, C₂₋C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R¹⁹ and R²¹ are independently H, C₁-C₄ alkyl, C₁-C₄ haloalkyl or C₃-C₈ cycloalkyl; or
R¹⁹ and R²¹ are taken together as —(CH₂)₄—, —(CH₂)₅, —CH₂CH₂OCH₂CH₂— or —CH₂CH(CH₃)OCH(CH₃)CH₂—;
each R²² and R²³ are independently H, C₁-C₄ alkyl, C₃-C₈ cycloalkyl, C₄-C₈ cycloalkylalkyl each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R²² and R²³ are taken together as —(CH₂)₄—, —(CH₂)₅, —CH₂CH₂OCH₂CH₂— or —CH₂CH(CH₃)OCH(CH₃)CH₂—;
each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ alkoxyalkyl, C₃-C₆ dialkoxyalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl; and
R²⁵ is H, C₁-C₄ alkyl or C₁-C₄ haloalkyl;
R³⁰ is H, C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, 5- or 6-membered heteroaromatic ring, each ring or ring system optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy and C₁-C₄ haloalkoxy; and
each R³¹ and R³² are independently C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, C₂-C₄ halogen, cyano, nitro, C₁-C₄ alkoxy and C₁-C₄ haloalkoxy.
   provided that when R² is NR⁸—N═CR⁹R¹⁰, O—N═CR⁹R¹⁰, NR⁸—NR¹¹R¹², O—NR¹¹R¹², CR¹³═NOR¹⁴ or CR¹³═NNR¹¹R¹², then J is phenyl substituted with at least one substituent selected from halogen and methyl.

This invention also relates to a fungicidal composition comprising a fungicidally effective amount of a compound of Formula 1 and at least one additional component selected from the group consisting of surfactants, solid diluents and liquid diluents.

This invention also relates to a fungicidal composition comprising a mixture of a compound of Formula 1 and at least one other fungicide (e.g. at least one additional fungicide having different mode of action).

This invention further relates to a method for controlling plant diseases caused by fungal plant pathogens comprising applying to the plant or portion thereof, or to the plant seed, a fungicidally effective amount of a compound of the invention (e.g. as a composition described herein).

### DETAILS OF THE INVENTION

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and Both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular. For example, a composition of the present invention comprises a biologically effective amount of “a” compound of Formula 1 which should be read that the composition includes one or at least one compound of Formula 1.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. “Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Dialkoxyalkyl” denotes dialkoxy substitution on alkyl. Examples of “dialkoxyalkyl” include (CH₃O)₂CH₂, (CH₃O)₂CH₂CH₂, (CH₃CH₂O)₂CH₂ and (CH₃CH₂O)₂CH₂CH₂. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O), CH₃CH₂S(O), CH₃CH₂CH₂S(O), (CH₃)₂CHS(O) and the different butylsulfinyl, pentylsulfinyl and hexylsulfinyl isomers. Examples of “alkylsulfonyl” include CH₃S(O)₂, CH₃CH₂S(O)₂, CH₃CH₂CH₂S(O)₂, (CH₃)₂CHS(O)₂ and the different butylsulfonyl, pentylsulfonyl and hexylsulfonyl isomers. “Alkylamino”, “dialkylamino”, and the like, are defined analogously to the above examples. “Alkylcycloalkylamino” denotes alkyl and cycloalkyl groups substituted with one amino group. Examples of “alkylcycloalkylamino” include methylcyclopropylamino and methylcyclohexylamino. “Cycloalkyl” includes, for example, cyclopropyl, cyclobutyl, cyclopentyl, and cyclohexyl. “Cycloalkenyl” includes groups such as cyclopentenyl and cyclohexenyl as well as groups with more than one double bond such as 1,3- and 1,4-cyclohexadienyl. Examples of “cycloalkylalkyl” include cyclopropylmethyl, cyclopentylethyl, and other cycloalkyl moieties bonded to straight-chain or branched alkyl groups. “Alkylcycloalkyl” denotes alkyl substitution on a cycloalkyl moiety. Examples include 4-methylcyclohexyl and 3-ethylcyclopentyl. The term “carbocyclic ring” denotes a ring wherein the atoms forming the ring backbone and selected only from carbon. The term “aromatic ring system” denotes fully unsaturated carbocycles and heterocycles in which the polycyclic ring system is aromatic. Aromatic indicates that each of ring atoms is essentially in the same plane and has a p-orbital perpendicular to the ring plane, and in which (4n+2) π electrons, when n is 0 or a positive integer, are associated with the ring to comply with Hückel's rule. The term “nonaromatic carbocyclic ring system” denotes fully saturated carbocycles as well as partially or fully unsaturated carbocycles wherein none of the rings in the ring system are aromatic. The term “nonaromatic heterocyclic ring system” denotes fully saturated heterocycles as well as partially or fully unsaturated heterocycles wherein none of the rings in the ring system are aromatic. The heterocyclic ring systems can be attached through any available carbon or nitrogen by replacement of a hydrogen on said carbon or nitrogen. The term “heteroaromatic ring” denotes a fully aromatic heterocyclic ring in which at least one ring atom is not carbon and which comprises 1 to 4 heteroatoms independently selected from the group consisting of nitrogen, oxygen and sulfur, provided that each heterocyclic ring includes no more than 4 nitrogens, no more than 2 oxygens and no more than 2 sulfurs. The term “heteroaromatic bicyclic ring system” denotes a bicyclic ring which contains at least one heteroatom and in which at least one ring of the bicyclic ring system is aromatic. The heteroaromatic rings or heterobicyclic ring systems can be attached through any available carbon or nitrogen by replacement of a hydrogen on said carbon or nitrogen. One skilled in the art will appreciate that not all nitrogen containing heterocycles can form N-oxides since the nitrogen requires an available lone pair of electrons for oxidation to the oxide; one skilled in the art will recognize those nitrogen containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethydioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “halocycloalkyl”, “haloalkoxy”, “haloalkylthio”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂. “Trialkylsilyl” includes 3 branched and/or straight-chain alkyl radicals attached to and linked through a silicon atom such as trimethylsilyl, triethylsilyl and t-butyldimethylsilyl.

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₄ cycloalkylalkyl designates cyclopropylmethyl; C₅ cycloalkylalkyl designates, for example, cyclopropylethyl or cyclobutylmethyl; and C₆ cycloalkylalkyl designates the various ring size of a cycloalkyl group substituted with an alkyl group containing a total of six carbon atoms, examples including cyclopentylmethyl, 1-cyclobutylethyl, 2-cyclobutylethyl and 2-cyclopropylpropyl. Examples of “alkylcarbonyl” include C(O)CH₃, C(O)CH₂CH₂CH₃ and C(O)CH(CH₃)₂. Examples of “alkoxycarbonyl” include CH₃OC(═O), CH₃CH₂OC(═O), CH₃CH₂CH₂OC(═O), (CH₃)₂CHOC(═O) and the different butoxy- or pentoxycarbonyl isomers. Examples of “alkylaminocarbonyl” include CH₃NHC(═O)—, CH₃CH₂NHC(═O)—, CH₃CH₂CH₂NHC(═O)—, (CH₃)₂CHNHC(═O)— and the different butylamino- or pentylaminocarbonyl isomers. Examples of “dialkylaminocarbonyl” include (CH₃)₂NC(═O)—, (CH₃CH₂)₂NC(═O)—, CH₃CH₂(CH₃)NC(═O)—, (CH₃)₂CHN(CH₃)C(═O)— and CH₃CH₂CH₂(CH₃)NC(′O)—. In the above recitations, when a compound of Formula 1 is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents is greater than 1, said substituents are independently selected from the group of defined substituents. Further, when the subscript indicates a range, e.g. (R)ᵢ₋ⱼ, then the number of substituents may be selected from the integers between i and j inclusive.

When a group contains a substituent which can be hydrogen, for example R³, R⁴, R⁵ or R⁷ then, when this substituent is taken as hydrogen, it is recognized that this is equivalent to said group being unsubstituted. When R² and R⁷ are taken together as —N═C(R¹⁶)—, the left-hand bond is connected as R² and the right-hand bond is connected as R⁷. The term “optionally substituted” in connection with groups listed for R¹, R², R⁴, R⁵, R⁶, R²², R²³, R³⁰, R³¹, R³², J, G¹ and G² refers to groups that are unsubstituted or have at least 1 non-hydrogen substituent. These groups may be substituted with as many optional substituents as can be accommodated by replacing a hydrogen atom with a non-hydrogen substituent on any available carbon or nitrogen atom. Commonly, the number of optional substituents (when present) ranges from 1 to 5. Examples of 5- or 6-membered heteroaromatic rings optionally substituted with from 1 to 5 substituents described for R² and J include the rings H-1 through H-24 illustrated in Exhibit 1 wherein each R²⁰ is independently halogen, C₁-C₆ alkyl, C₂-C₆ alkenyl, C₃-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl, and r is an integer from 0 to 5. Examples of 5- or 6-membered heteroaromatic rings optionally substituted with from 1 to 4 substituents described for R³⁰ and G² include the rings H-1 through H-24 illustrated in Exhibit 1 wherein R²⁰ is R¹⁸, and r is an integer from 0 to 4. Examples of 8-, 9- or 10-membered heteroaromatic bicyclic rings optionally substituted with from 1 to 5 substituents described for R² and J include the rings B-1 through B-39 illustrated in Exhibit 2 wherein each R²⁰ is independently halogen, C₁-C₆ alkyl, C₂-C₆ alkenyl, C₃-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl, and r is an integer from 0 to 5. Examples of 5- or 6-membered saturated or partially saturated heterocyclic rings, each optionally substituted with up to 5 substituents described for R² include the rings U-20 through U-68 illustrated in Exhibit 3 wherein each R²⁰ is independently halogen, C₁-C₆ alkyl, C₂-C₆ alkenyl, C₃-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl, and r is an integer from 0 to 5. Examples of 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents described for G¹ include the rings U-1 through U-77 illustrated in Exhibit 3 wherein R²⁰ is R¹⁷, and r is an integer from 0 to 4. Although R²⁰ groups are shown in the structures showed in Exhibit 1, Exhibit 2 and Exhibit 3, it is noted that they do not need to be present since they are optional substituents. The nitrogen atoms that require substitution to fill their valence are substituted with H or R²⁰. Note that some H groups in Exhibit 1 can only be substituted with less than 4 R²⁰ groups as described for G² (e.g. H-1 through H-24). Note that some B groups in Exhibit 2 can only be substituted with less than 5 R²⁰ groups (e.g. B-5 through B-9, B-21 through B-23, B-25 through B-27 and B-37 through B-39). Note that some U groups in Exhibit 3 can only be substituted with less than 5 R²⁰ groups (e.g. U-1, U-6, U-10, U-11, U-16 through U-19, U-24 through U-40, U-54, U-56 through U-60, U-62 through U-64 and U-66 through U-68). Note that when the attachment point between (R²⁰)ᵣ and the H, B or U group is illustrated as floating, (R²⁰)ᵣ can be attached to any available carbon atom or nitrogen atom of the H, B or U group. Note that when the attachment point of the H, B or U group is illustrated as floating, the H, B or U group can be attached to the remainder of Formula 1 through any available carbon atom or nitrogen atom of the H, B or U group by replacement of a hydrogen atom.

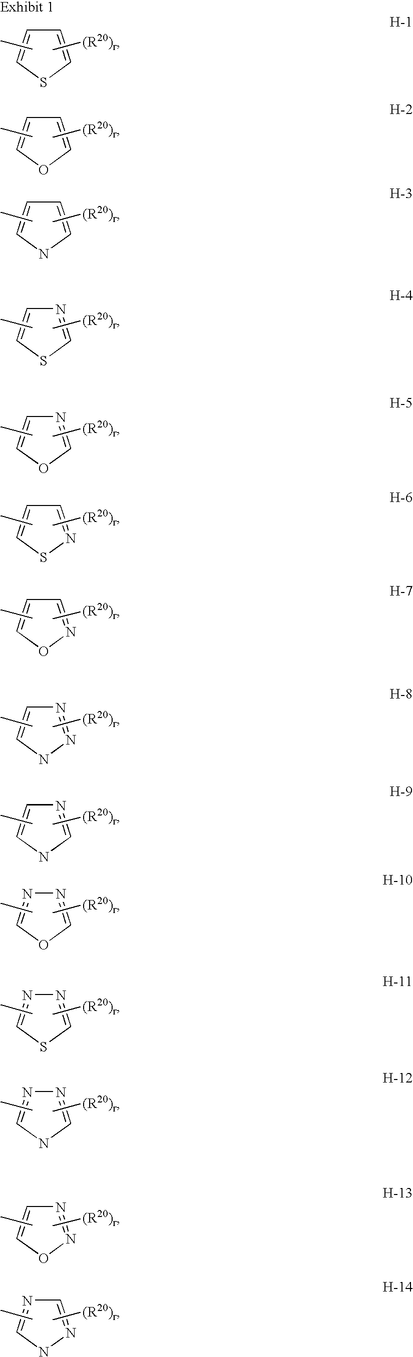

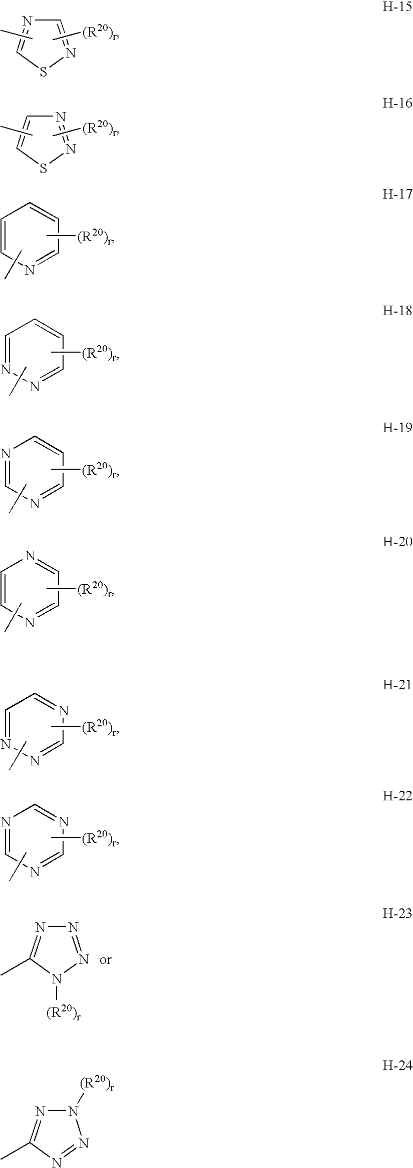

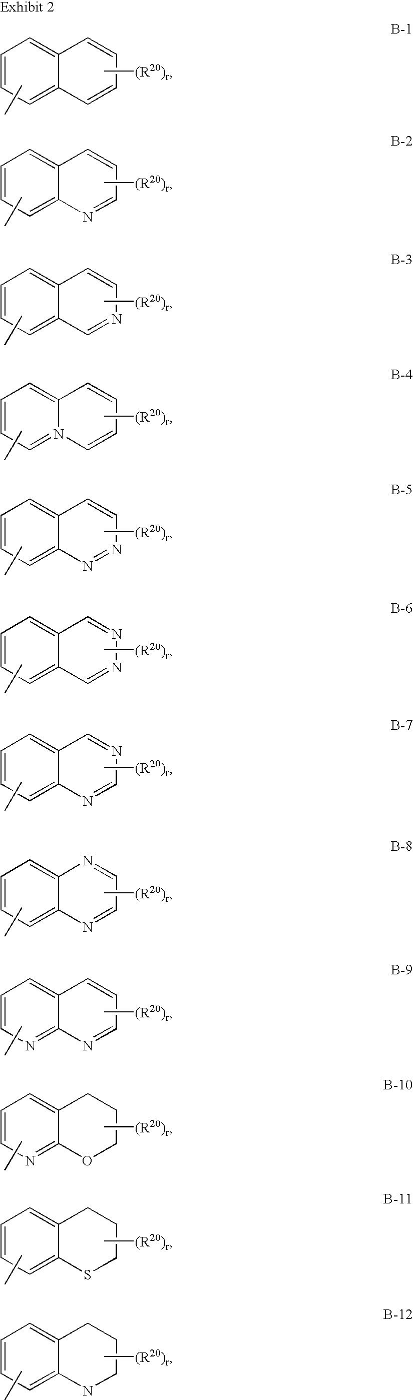

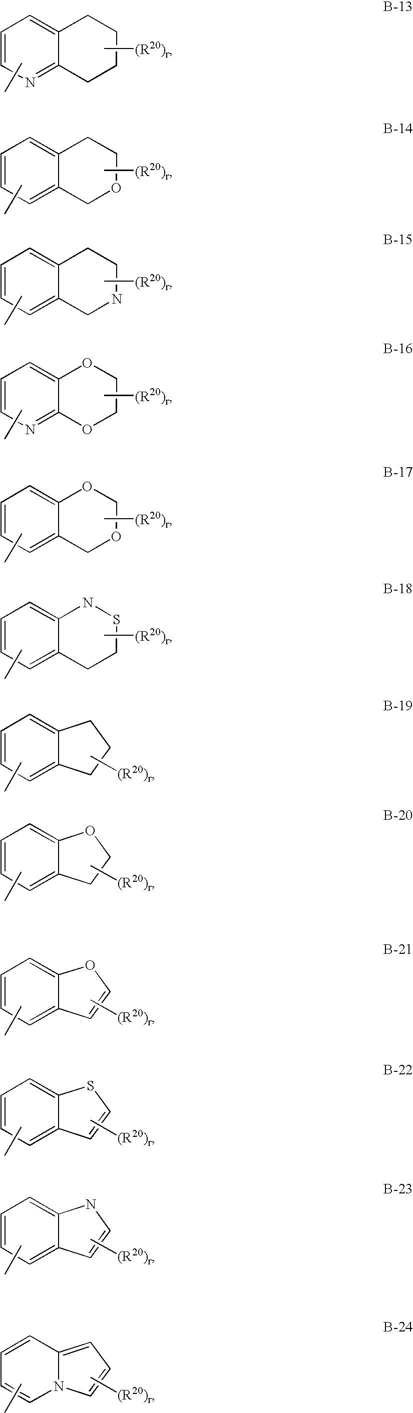

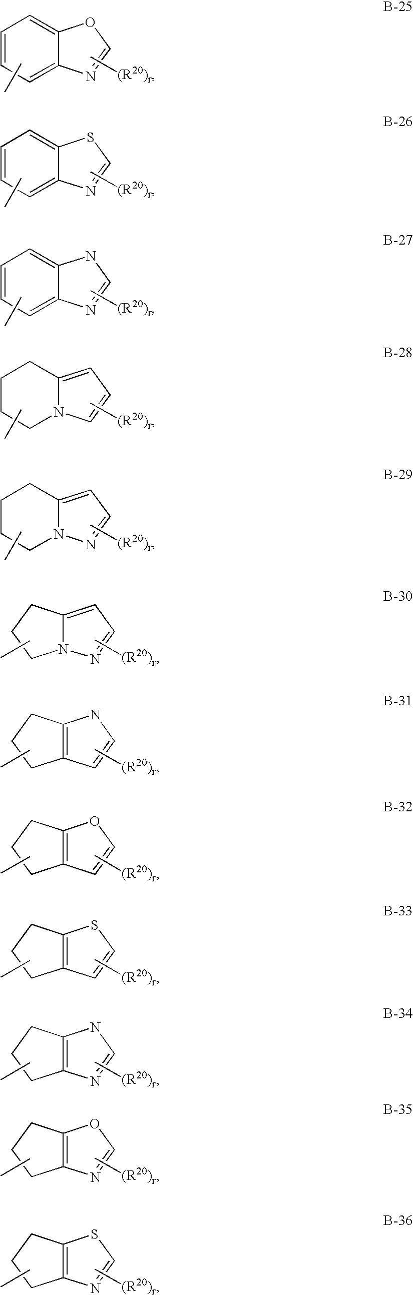

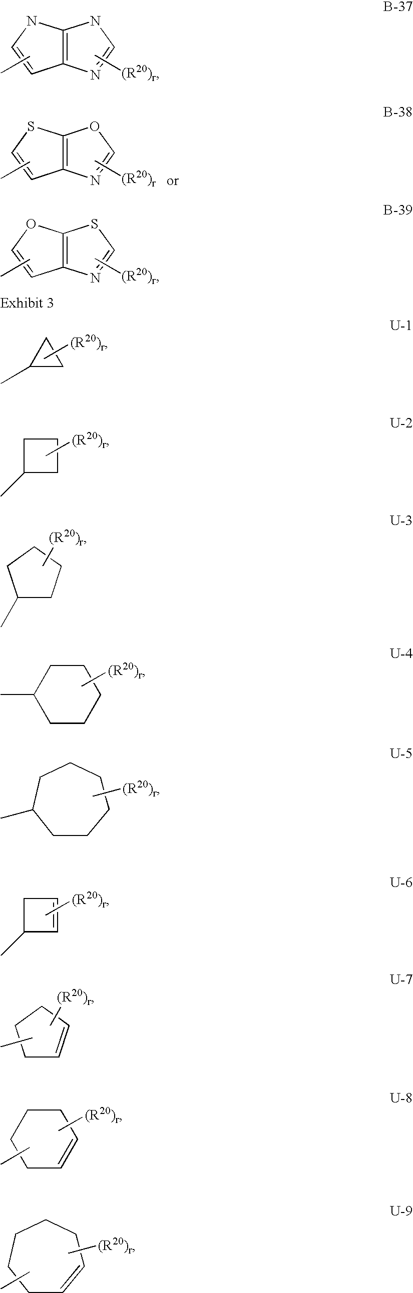

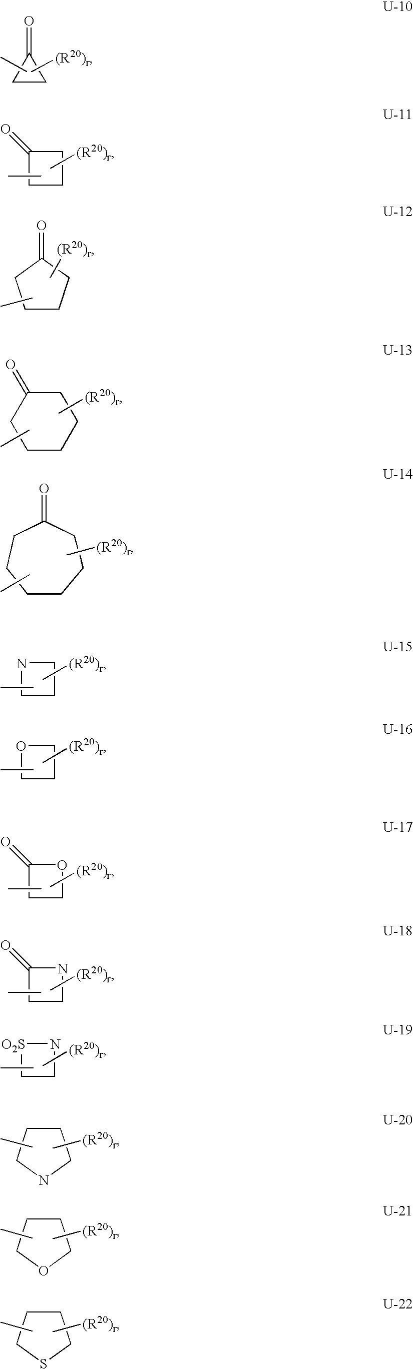

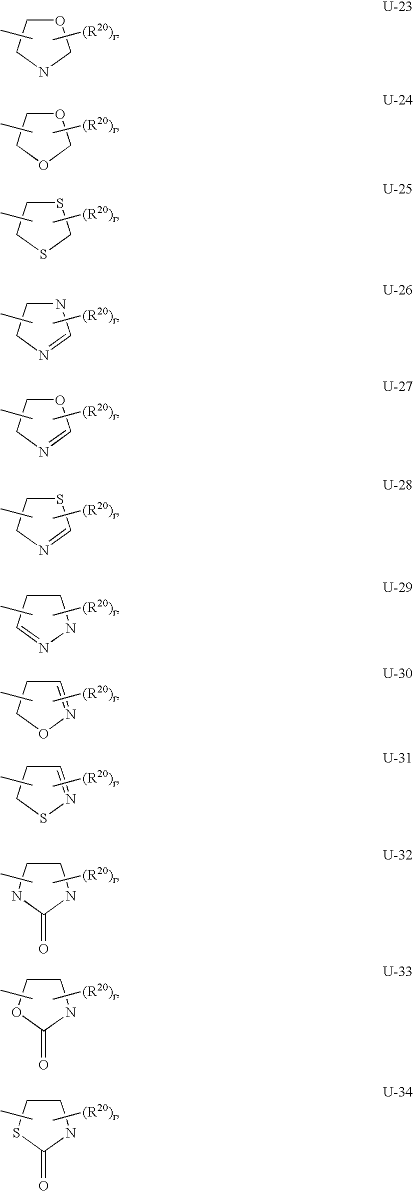

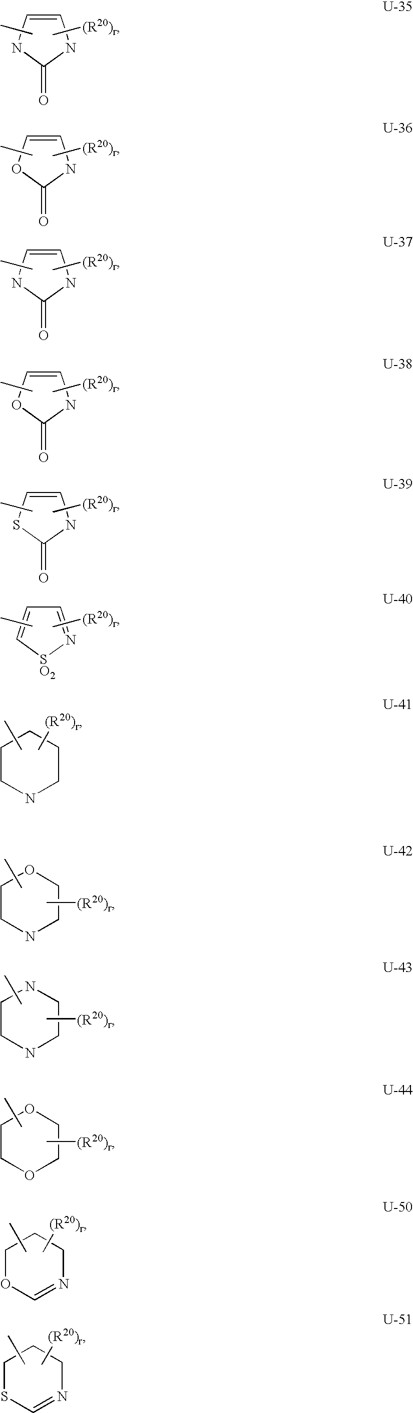

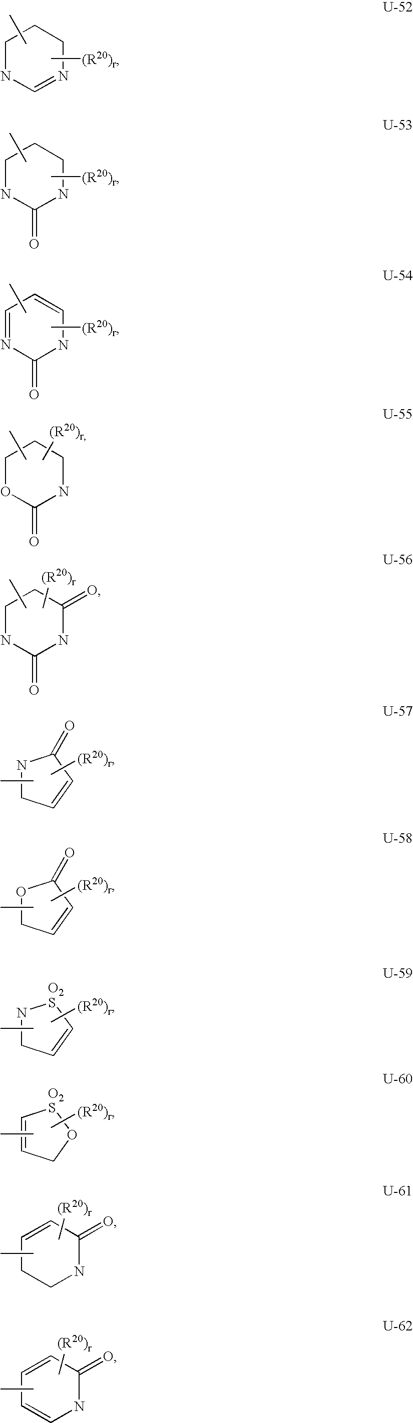

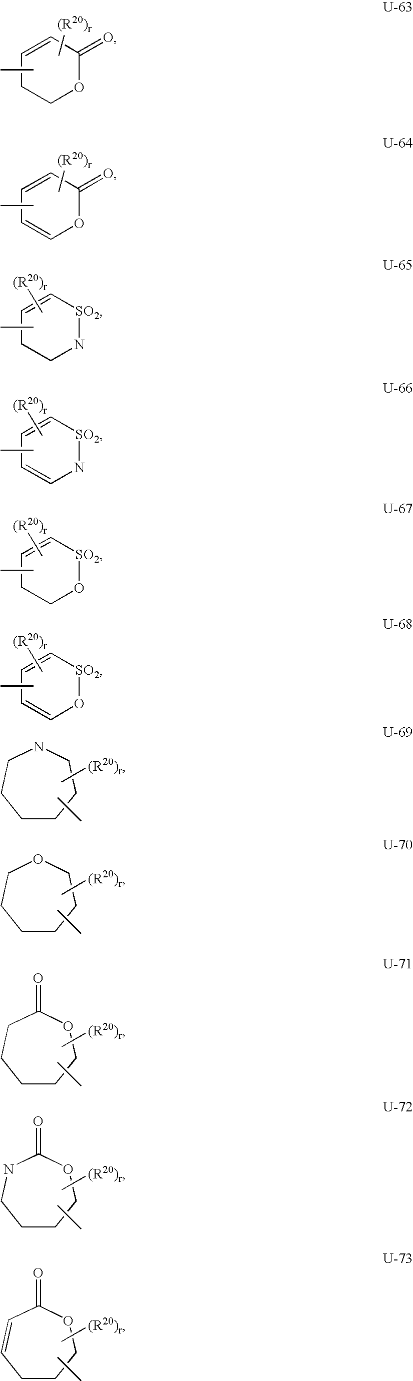

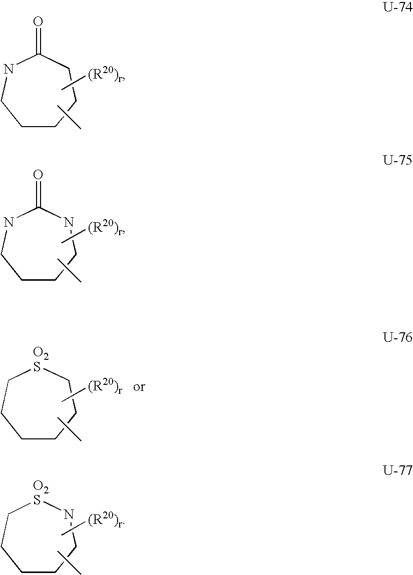

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. Accordingly, the present invention comprises compounds selected from Formula 1, N-oxides and agriculturally suitable salts thereof. The compounds of the invention may be present as a mixture of stereoisomers, individual stereoisomers, or as an optically active form. For example, when R¹ is 2-methylbutyl group, Formula 1 possesses a chiral center at the carbon atom identified with the asterisk (*). This invention comprises racemic mixtures, and also includes with compounds that are enriched compared to the racemic mixture with an enantiomer of Formula 1.

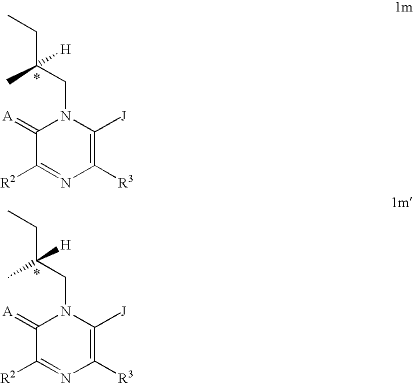

Included are the essentially pure enantiomers of compounds of Formula 1, for example, Formula 1m and Formula 1m′ (Formula 1 wherein R¹ is 2-methylbutyl group).

When a compound is enantiomerically enriched, one enantiomer is present in greater amounts than the other, and the extent of enrichment can be specified by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers).

For the compounds of Formula 1 where R¹ is 2-methylbutyl group, the more fungicidally active enantiomer is believed to be the enantiomer in which the hydrogen atom attached to the carbon atom identified with an asterisk (*) lies below the plane defined by the 3 non-hydrogen atoms attached to the carbon atom identified with the asterisk (*), as is shown in Formula 1m. The carbon atom identified with an asterisk (*) in Formula 1m has the S configuration.

Preferably the compositions of this invention have at least a 50% enantiomeric excess; more preferably at least a 75% enantiomeric excess; still more preferably at least a 90% enantiomeric excess; and most preferably at least a 94% enantiomeric excess of the more active isomer. Of particular note are enantiomerically pure embodiments of the more active isomer.

In particular, when J is a phenyl ring substituted with R²⁶ at the ortho position of the ring, or an analogous naphthalene, 5- or 6-membered heteroaromatic ring or 8-, 9- or 10-membered heteroaromatic bicyclic ring system, wherein R²⁶ is as described for J ring or ring system substituents in the Summary of the Invention, then Formula 1 possesses an axis of chirality differentiating two atropisomers (chiral rotational isomers). The atropisomers of Formula 1 can be separated because rotation about the single bond connecting J is prevented or greatly retarded. This invention comprises racemic mixtures of such rotomers. And also includes compounds that are enriched compared to the racemic mixture with an atropisomer of Formula 1n or 1n′.

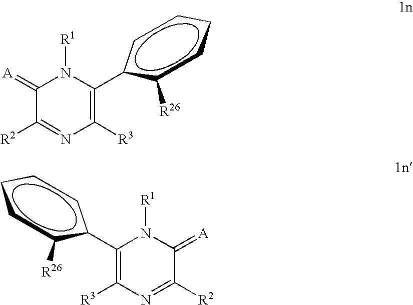

The salts of the compounds of the invention include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. The salts of the compounds of the invention also include those formed with organic bases (e.g., pyridine, ammonia, or triethylamine) or inorganic bases (e.g., hydrides, hydroxides, or carbonates of sodium, potassium, lithium, calcium, magnesium or barium) when the compound contains an acidic group such as a carboxylic acid or phenol.

Embodiments of the present invention include:
Embodiment 1. A compound of Formula 1 wherein R¹ is C₁-C₈ alkyl, C₄-C₆ alkylcycloalkyl, C₃-C₇ cycloalkyl, C₁-C₈ haloalkyl, NR⁴R⁵, N═CR¹⁹R²¹, C₁-C₄ alkylamino, G¹ or G².
Embodiment 2. A compound of Embodiment 1 wherein R¹ is C₂-C₆ alkyl, C₂-C₆ haloalkyl, C₃-C₈ cycloalkyl, C₄-C₈ alkylcycloalkyl, NR⁴R⁵, G¹ or G².
Embodiment 3. A compound of Embodiment 2 wherein R¹ is C₂-C₆ alkyl or C₂-C₆ haloalkyl.
Embodiment 4. A compound of Embodiment 3 wherein R¹ is C₄-C₅ alkyl or C₃-C₅ haloalkyl.
Embodiment 5. A compound of Embodiment 2 wherein R¹ is NR⁴R⁵.
Embodiment 6. A compound of Embodiment 2 wherein R¹ is G¹.
Embodiment 7. A compound of Embodiment 2 wherein R¹ is G².
Embodiment 8. A compound of Embodiment 5 wherein each R⁴ and R⁵ are independently H or C₁-C₈ alkyl.
Embodiment 9. A compound of Embodiment 8 wherein each R⁴ and R⁵ are independently H or C₁-C₄ alkyl.
Embodiment 10. A compound of Embodiment 6 wherein G¹ is a 5- to 6-membered nonaromatic carbocyclic or heterocyclic ring, optionally including 1 or 2 ring members selected from the group consisting of C(═O), C(═S), S(O) and S(O)₂.
Embodiment 11. A compound of Embodiment 10 wherein G¹ is a 5- to 6-membered nonaromatic carbocyclic or heterocyclic ring, optionally including 1 or 2 ring members selected from the group consisting of C(═O).
Embodiment 12. A compound of Embodiment 7 wherein G² is a phenyl ring, optionally substituted with from 1 to 4 substituents independently selected from R¹⁸.
Embodiment 13. A compound of Embodiment 7 wherein G2 is a phenyl optionally substituted by 1-3 halogens.
Embodiment 14. A compound of Embodiment 7 wherein G2 is Benzyl substituted by halogen or C₁-C₂ alkyl.
Embodiment 15. A compound of Embodiment 7 wherein G² is a 5- or 6-membered heteroaromatic ring, each ring or ring system optionally substituted with from 1 to 4 substituents independently selected from R¹⁸.
Embodiment 16. A compound of Formula 1 wherein A is O or S.
Embodiment 17. A compound of Embodiment 16 wherein A is O.
Embodiment 18. A compound of Formula 1 wherein A is NR⁷.
Embodiment 19. A compound of Embodiment 18 wherein R⁷ is H, C₁-C₄ alkyl or C₁-C₂ alkylcarbonyl.
Embodiment 20. A compound of Embodiment 19 wherein R⁷ is H or C₁-C₂ alkylcarbonyl.
Embodiment 21. A compound of Formula 1 wherein R² is cyano, NR⁸—N═CR⁹R¹⁰, O—N═CR⁹R¹⁰, NR⁸—NR¹¹R¹², O—NR¹¹R¹², CR¹³═NOR¹⁴, CR¹³═NNR¹¹R¹² C(W)NR²²R²³ or NC(═O)R³⁰.
Embodiment 22. A compound of Embodiment 21 wherein R² is cyano, NR⁸—N═CR⁹R¹⁰, CR¹³═NOR¹⁴, CR¹³═NNR¹¹R¹² or C(W)NR²²R²³.
Embodiment 23. A compound of Embodiment 22 wherein R² is cyano, NR⁸—N═CR⁹R¹⁰ or CONH₂.
Embodiment 24. A compound of Embodiment 23 wherein R² is cyano or CONH₂.
Embodiment 25. A compound of Embodiment 23 wherein W is O.
Embodiment 26. A compound of Embodiment 23 wherein each R²² and R²³ are independently H or C₁-C₄ alkyl.
Embodiment 27. A compound of Formula 1 wherein R² is a 5- or 6-membered heteroaromatic ring, each ring optionally substituted with up to 5 substituents selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents selected from R²⁴.
Embodiment 28. A compound of Embodiment 27 wherein R² is a 5- or 6-membered heteroaromatic ring, each ring optionally substituted with up to 4 substituents selected from R²⁴; or 5- or 6-membered saturated or partially saturated heterocyclic ring, optionally including 1-3 C(═O) groups as ring members, and optionally substituted with up to 5 substituents selected from R²⁴.
Embodiment 29. A compound of Embodiment 28 wherein R² is a 5- or 6-membered heteroaromatic ring, each ring optionally substituted with up to 3 substituents selected from R²⁴; or 5- or 6-membered saturated or partially saturated heterocyclic ring, optionally including 1-2 C(═O) groups as ring members, and optionally substituted with up to 3 substituents selected from R²⁴.
Embodiment 30. A compound of Embodiment 29 wherein R² is a 5- or 6-membered heteroaromatic ring, each ring optionally substituted with up to 3 substituents selected from R²⁴.
Embodiment 31. A compound of Embodiment 30 wherein R² is a 5-membered heteroaromatic ring, each ring optionally substituted with up to 3 substituents selected from R²⁴.
Embodiment 32. A compound of Embodiment 30 wherein R² is a 6-membered heteroaromatic ring, each ring optionally substituted with up to 3 substituents selected from R²⁴.
Embodiment 33. A compound of Embodiment 30 wherein R² is 1H-pyrazol-1-yl, 1H-1,2,4-triazol-1-yl or 2-pyridinyl.
Embodiment 34. A compound of Embodiment 33 wherein R² is 1H-pyrazol-1-yl or 1H-1,2,4-triazol-1-yl.
Embodiment 35. A compound of Embodiment 33 wherein R² is 2-pyridinyl.
Embodiment 36. A compound of Embodiment 27 wherein each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio or C₃-C₆ trialkylsilyl.
Embodiment 37. A compound of Embodiment 36 wherein each R²⁴ is independently halogen, C₁-C₆ alkyl, C₁-C₆ haloalkyl, cyano, nitro, C₁-C₆ alkoxy or C₁-C₆ haloalkoxy.
Embodiment 38. A compound of Embodiment 37 wherein each R²⁴ is independently halogen, C₁-C₆ alkyl, C₁-C₆ haloalkyl or cyano.
Embodiment 39. A compound of Embodiment 27 wherein each R²⁴ is independently halogen, C₁-C₄ alkyl, C₁-C₄ haloalkyl or cyano.
Embodiment 40. A compound of Embodiment 30 wherein each R²⁴ is independently halogen, C₁-C₆ alkyl, C₁-C₆ haloalkyl or cyano.
Embodiment 41. A compound of Embodiment 31 wherein each R²⁴ is independently halogen, C₁-C₆ alkyl, C₁-C₆ haloalkyl or cyano.
Embodiment 42. A compound of Embodiment 32 wherein each R²⁴ is independently halogen, C₁-C₆ alkyl, C₁-C₆ haloalkyl or cyano.
Embodiment 43. A compound of Embodiment 33 wherein R² is 1H-pyrazol-1-yl, 1H-1,2,4-triazol-1-yl or 2-pyridinyl, each optionally substituted with halogen, C₁-C₆ alkyl, C₁-C₆ haloalkyl or cyano.
Embodiment 44. A compound of Formula 1 wherein R³ is halogen, cyano, C₁-C₆ alkyl, C₁-C₄ haloalkyl, C₃-C₆ cycloalkyl, C₃-C₆ halocycloalkyl, or —CHO.
Embodiment 45. A compound of Embodiment 44 wherein R³ is halogen, cyano, C₁-C₆ alkyl or C₁-C₄ haloalkyl.
Embodiment 46. A compound of Embodiment 45 wherein R³ is halogen, cyano or C₁-C₆ alkyl.
Embodiment 47. A compound of Embodiment 46 wherein R³ is halogen, cyano or C₁-C₃ alkyl.
Embodiment 48. A compound of Embodiment 47 wherein R³ is chloro, bromo, fluoro or methyl.
Embodiment 49. A compound of Embodiment 48 wherein R³ is chloro.
Embodiment 50. A compound of Formula 1 wherein J is phenyl, benzyl, naphthalene, 5- or 6-membered heteroaromatic ring, each ring optionally substituted with up to 5 substituents selected from halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl and C₃-C₆ trialkylsilyl.
Embodiment 51. A compound of Embodiment 50 wherein J is phenyl, benzyl, naphthalene, 5- or 6-membered heteroaromatic ring, each ring optionally substituted with up to 3 substituents selected from halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl and C₃-C₆ trialkylsilyl.
Embodiment 52. A compound of Embodiment 51 wherein J is phenyl, benzyl, naphthalene, 5- or 6-membered heteroaromatic ring, each ring optionally substituted with up to 3 substituents selected from halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio and C₃-C₆ trialkylsilyl.
Embodiment 53. A compound of Embodiment 52 wherein J is phenyl, benzyl, naphthalene, 5- or 6-membered heteroaromatic ring, each ring optionally substituted with up to 3 substituents selected from halogen, C₁-C₆ alkyl, C₁-C₆ haloalkyl, C₂-C₆ haloalkenyl, cyano, nitro, C₁-C₆ alkoxy and C₁-C₆ haloalkoxy.
Embodiment 54. A compound of Embodiment 53 wherein J is phenyl or 5- or 6-membered heteroaromatic ring, each ring optionally substituted with up to 3 substituents selected from halogen, C₁-C₆ alkyl, C₁-C₆ haloalkyl, C₂-C₆ haloalkenyl, cyano, nitro, C₁-C₆ alkoxy and C₁-C₆ haloalkoxy.
Embodiment 55. A compound of Embodiment 54 wherein J is phenyl or 5- or 6-membered heteroaromatic ring, each ring optionally substituted with up to 3 substituents selected from halogen, C₁-C₆ alkyl, C₁-C₆ haloalkyl and C₁-C₆ alkoxy.
Embodiment 56. A compound of Embodiment 55 wherein J is phenyl, optionally substituted with up to 3 substituents selected from halogen, C₁-C₆ alkyl, C₁-C₆ haloalkyl and C₁-C₆ alkoxy.
Embodiment 57. A compound of Embodiment 56 wherein J is phenyl, optionally substituted with up to 3 substituents selected from halogen, C₁-C₆ alkyl and C₁-C₆ haloalkyl.
Embodiment 58. A compound of Embodiment 57 wherein J is phenyl, optionally substituted at the 2, 4 and 6 positions with substituents selected from halogen and C₁-C₆ alkyl.
Embodiment 59. A compound of Embodiment 58 wherein J is phenyl, optionally substituted at the 2, 4 and 6 positions with substituents selected from methyl, methoxy, chloro and fluoro.
Embodiment 60. A compound of Embodiment 59 wherein J is phenyl, optionally substituted at the 2, 4 and 6 positions with substituents selected from methyl, chloro and fluoro.
Embodiment 61. A compound of Embodiment 60 wherein J is 2,4-difluorophenyl, 2,6-difluorophenyl, 2,4,6-trifluorophenyl, 2,3,6-trifluorophenyl, 2-chloro-4-fluorophenyl or 2-chloro-6-fluorophenyl.
Embodiment 62. A compound of Formula 1 wherein J is C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents selected from the group consisting of halogen, cyano, nitro, hydroxy, C₁-C₄ alkoxy, C₁-C₄ haloalkoxy, C₁-C₄ alkylthio, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl and C₁-C₄ alkylamino.
Embodiment 63. A compound of Embodiment 62 wherein J is C₁-C₈ alkyl or C₃-C₈ cycloalkyl optionally substituted by one or more substituents selected from halogen and C₁-C₄ alkoxy.
Embodiment 64. A compound of Embodiment 63 wherein J is i-Pr, s-Bu or c-Pentyl. Combinations of Embodiments 1-64 are illustrated by:
Embodiment A. A compound of Formula 1 wherein
   A is O or S;
   R¹ is C₂-C₆ alkyl, C₂-C₆ haloalkyl, C₃-C₈ cycloalkyl, C₄-C₈ alkylcycloalkyl, NR⁴R⁵, G¹ or G²;
   R² is cyano or C(W)NR²²R²³; or a 5- or 6-membered heteroaromatic ring; or a 5- or 6-membered saturated or partially saturated heterocyclic ring, optionally including 1-3 C(═O) groups as ring members;
   R³ is halogen or C₁-C₆ alkyl;
   R⁴ and R⁵ are independently H, C₃-C₆ alkyl or C₃-C₆ haloalkyl; and
   J is phenyl optionally substituted at the 2, 3, 4 and 6 positions with substituents selected from methyl, methoxy, chloro and fluoro.
Of note are compounds of Embodiment A wherein
   R¹ is C₂-C₆ alkyl, C₂-C₆ haloalkyl or NR⁴R⁵;
   R² is 5- or 6-membered heteroaromatic ring or cyano;
   R³ is halogen or methyl; and
   J is phenyl optionally substituted at the 2, 4 and 6 positions with substituents selected from methyl, methoxy, chloro and fluoro.
Also of note are compounds of Embodiment A wherein
   R¹ is C₄-C₆ alkyl or C₄-C₆ haloalkyl;
   R² is 1H-pyrazol-1-yl, 1H-1,2,4-triazol-1-yl or 2-pyridinyl, each optionally substituted with halogen, cyano, C₁-C₆ alkyl or C₁-C₄ haloalkyl; and
   R³ is chloro, fluoro or methyl; and
   J is 2,4-difluorophenyl, 2,6-difluorophenyl, 2-fluoro-6-methylphenyl, 2,4,6-trifluorophenyl, 2,4-dimethylphenyl or 2-chloro-6-fluorophenyl.
Embodiment B. A compound of Embodiment A wherein
   A is O;
   R¹ is C₂-C₆ alkyl, C₂-C₆ haloalkyl, C₃-C₈ cycloalkyl, C₄-C₈ alkylcycloalkyl, G¹ or G²;
   R² is 5- or 6-membered heteroaromatic ring, cyano or CONH₂;
   R³ is halogen or methyl; and
   J is phenyl optionally substituted at the 2, 3, 4 and 6 positions with substituents selected from methyl, chloro and fluoro.
Embodiment C. A compound of Embodiment B wherein
   R¹ is C₂-C₆ alkyl, C₂-C₆ haloalkyl or phenyl, optionally substituted with from 1 to 4 substituents independently selected from R¹⁸;
   R² is 5- or 6-membered heteroaromatic ring or CONH₂; and
   R³ is bromo, chloro, fluoro or methyl.
Embodiment D. A compound of Embodiment C wherein
   R¹ is C₄-C₆ alkyl, C₄-C₆ haloalkyl or phenyl, optionally substituted with from 1 to 4 substituents independently selected from R¹⁸;
   R² is 1H-pyrazol-1-yl, 1H-1,2,4-triazol-1-yl or 2-pyridinyl, each optionally substituted with halogen, cyano, C₁-C₆ alkyl or C₁-C₄ haloalkyl; or CONH₂; and
   J is 2,4-difluorophenyl, 2,6-difluorophenyl, 2,4,6-trifluorophenyl, 2,3,6-trifluorophenyl, 2-chloro-4-fluorophenyl or 2-chloro-6-fluorophenyl.

Most preferred is the compound of Formula 1 selected from the group consisting of:
5-Chloro-6-(2,6-difluorophenyl)-1-(2-methylpropyl)-3-(1H-pyrazol-1-yl)-2(1H)-pyrazinone,
5-Chloro-1-[(2S)-2-methylbutyl)-3-(1H-pyrazol-1-yl)-6-(2,4,6-trifluorophenyl)-2(1H)-pyrazinone,
5-Chloro-6-(2,6-difluorophenyl)-1-phenyl-3-(1H-pyrazol-1-yl)-2(1H)-pyrazinone,
5-Chloro-6-(2-chloro-4-fluorophenyl)-1-(2-methylbutyl)-3-(3-methyl-1H-pyrazol-1-yl)-2(1H)-pyrazinone,
6-Chloro-5-(2,6-difluorophenyl)-3,4-dihydro-4-(2-methylbutyl)-3-oxopyrazinecarboxamide,
5-Chloro-6-(2-chloro-4-fluorophenyl)-1-(2-methylpropyl)-3-(1H-pyrazol-1-yl)-2(1H)-pyrazinone,
5-Chloro-1-(3-fluorophenyl)-3-(1H-pyrazol-1-yl)-6-(2,4,6-trifluorophenyl)-2(1H)-pyrazinone,
5-Chloro-1-phenyl-3-(1H-pyrazol-1-yl)-6-(2,3,6-trifluorophenyl)-2(1H)-pyrazinone.

This invention also relates to a fungicidal composition comprising a fungicidally effective amount of a compound of Formula 1 and at least one additional component selected from the group consisting of surfactants, solid diluents or liquid diluents. Noteworthy as embodiments are fungicidal compositions of the present invention are those comprising the compounds of embodiments described above.

This invention also relates to a fungicidal composition comprising a mixture of a compound of Formula 1 and at least one other fungicide having a different mode of action.

This invention also relates to a method for controlling plant diseases caused by fungal plant pathogens comprising applying to the plant or portion thereof, or to the plant seed or seedling, a fungicidally effective amount of a compound of the invention (i.e. as a composition described herein). Methods of use of note are those involving the compounds of embodiments described above.

The compounds of Formula 1 can be prepared by one or more of the following methods and variations as described in Schemes 1-14. The definitions of R¹, R², R³, R¹¹, R¹², R¹³, R¹⁴, R¹⁹, R²¹, R²², R²³, A and J in the compounds of Formulae 1-20 below are as defined above in the Summary of the Invention. Compounds of Formulae 1a-1l are various subsets of the compounds of Formula 1.

Compounds of Formula 1 wherein R² is a heterocycle linked through N can be made as shown in Scheme 1. Reaction of an heterocycle comprising NH of Formula 3 with a compound of Formula 2 wherein X¹ is halogen (e.g., Cl, Br, I), OS(O)₂CH₃ (methanesulfone), OS(O)₂CF₃, OS(O)₂Ph-p-CH₃ (p-toluenesulfone) and like as outlined in Scheme 1 in the presence of an acid acceptor gives the compounds of Formula 1 in which R² is a N-linked heterocycle. Suitable acid acceptors for the reaction include inorganic bases, such as alkali or alkaline earth metal (such as lithium, sodium, potassium, cesium) hydrides, alkoxides, carbonates, phosphates and hydroxides, and organic bases, such as triethylamine, N,N-diisopropylethylamine and 1,8-diazabicyclo[5.4.0]undec-7-ene. Preferred acid acceptors are potassium carbonate and potassium hydroxide. A wide variety of solvents are suitable for the reaction, including, for example but are not limited to N,N-dimethylformamide, N,N-dimethylacetamide, N-methylpyrrolidinone, acetonitrile and acetone, as well as mixtures of these solvents. This reaction can be conducted between about 0 and 200° C., and preferably between about 20 and 80° C.

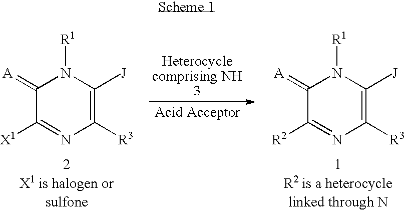

As shown in Scheme 2, compounds of Formula 1 in which R² is a hydrazone, oxime, hydrazine derivative or hydroxylamine derivative can be synthesized by a reaction of the appropriate nucleophile of Formula 4 with a compound of Formula 2 in the presence of an acid acceptor. Preferred solvents include N,N-dimethylformamide, N,N-dimethylacetamide, N-methylpyrrolidinone, acetonitrile and acetone. Acid acceptors such as tertiary amines, alkali carbonates, alkali hydroxides and alkali hydrides may be used in this reaction. Potassium carbonate and tertiary amines such as triethylamine are preferred acid acceptors for hydrazones and hydrazines. Alkali hydrides such as sodium hydride are preferred acid acceptors for the oximes and hydroxylamines.

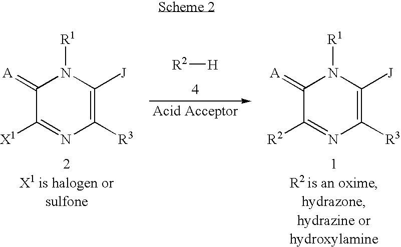

Compounds of Formula 1a and Formula 1b can be synthesized as shown in Scheme 3. Reaction of compounds of Formula 2 with a cyanide salt gives the products of Formula 1a. The reaction may be carried out in protic or aprotic solvents. Preferred solvents are N,N-dimethylformamide, lower alcohols and mixtures of these solvents with water. The reaction may be successfully carried out at temperatures from 0 to 200° C. with temperatures of 60-120° C. preferred. Compounds of Formula 1b may be obtained from the reaction of compounds of Formula 1a with hydrogen sulfide or other sulfide source. This reaction may be carried out in a variety of solvents and temperatures. Reaction in mixtures of lower alcohols and water is preferred. For a convenient procedure using ammonium as the sulfide source see Bagley et. al., Synlett, 2004, 2615-2617.

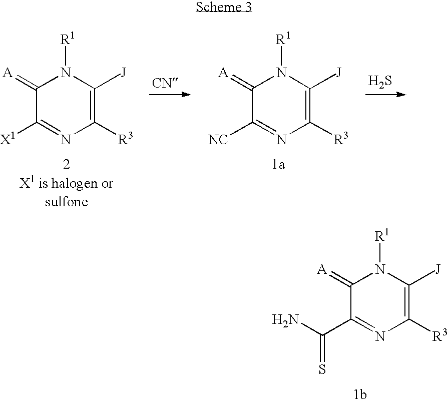

As shown in Scheme 4, compounds of Formula 1 wherein R² is a C-linked heterocycle can be obtained by transition metal catalyzed reactions of compounds of Formula 2 wherein X¹ is halogen with compounds of Formula 5. Transition metal catalyzed cross coupling reactions of halogenopyrazinones are known from the work of Hoornaert et. al., Tetrahedron, 1991, 47, 9259-9268 and Tetrahedron Letters, 2004, 45, 1885-1888. Reaction of various organometallic heterocycles of Formula 5 under palladium or nickel catalysis is possible. For synthesis of organometallic heterocycles suitable for use in this reaction see, Gribble and Li, “Palladium in Heterocyclic Chemistry”, Pergamon Press, Amsterdam, 2000, page 411. This book also describes a wide variety of catalysts and reaction conditions suitable for carrying out the cross coupling reactions described in Scheme 4. When the metal is magnesium, the coupling does not necessarily require added transition metal catalyst.

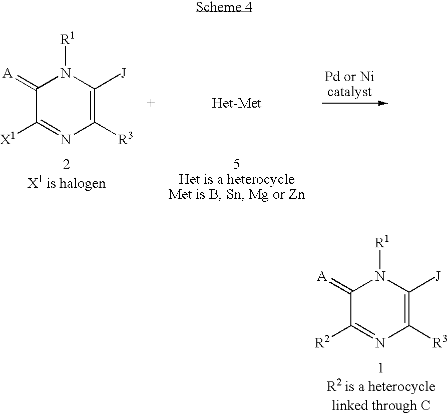

Compounds of Formula 1 wherein R² is a C-linked heterocycle can also be obtained by the conversion of a halogen substituted pyrazinone of Formula 2 into an organometallic derivative followed by a cross coupling reaction as shown in Scheme 5. Most preferably the organometallic pyrazinone is made by the reaction of a bimetallic reagent such as hexamethylditin with compounds of Formula 2 under palladium catalysis. Other reagents such as pinacolatodiborane may also be used. The resulting tin compound of Formula 6 can be transformed to compounds of Formula 1 by palladium catalyzed coupling with haloheterocycles of Formula 7. Examples of this reaction to make heterocyclic tin compounds may be found in Majeed et al., Tetrahedron, 1989, 45, 993-1006.

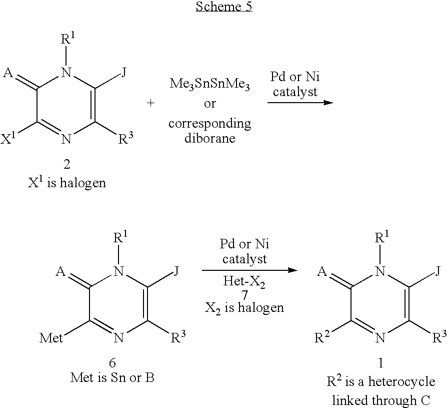

Compounds of Formula 1d (i.e. Formula 1 wherein R³ is alkoxy or thioalkyl or cyano) can be synthesized by the reaction of a halopyrazinone of Formula 1c with the appropriate nucleophile as shown in Scheme 6. The compound of Formula 1c is treated in an aprotic solvent with the appropriate nucleophile at temperatures between about 0 and 160° C. In the case of cyanide and thioalkyl nucleophiles the reaction is best carried out in solvents such as N,N-dimethylformamide and N-methypyrrolidinone. In the case of alkoxides, the reaction is best carried out in the alcohol from which the alkoxide is generated. Among appropriate acid acceptors are alkali metals such as sodium hydride. In the case of cyanide an acid acceptor is not necessary.

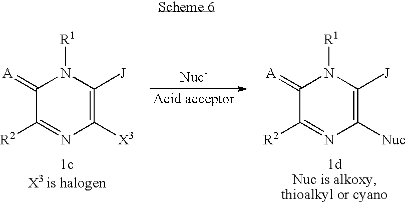

Compounds of Formula 1 wherein R³ is an alkyl, alkenyl, alkynyl or cycloalkyl group may be introduced by means of transition metal catalyzed reactions involving compounds of Formula 1c as shown in Scheme 7. The alkyl, alkenyl, alkynyl or cycloalkyl metal species may be derived from B, Sn, Si, Mg, Al or Zn. Conditions for the couplings are as described previously in Scheme 4 and description of conditions for these transformations is found in Gribble and Li (“Palladium in Heterocyclic Chemistry”, Pergamon Press, Amsterdam, 2000). Typical procedures for other palladium catalysed reactions of pyrazinones can be found in Tetrahedron, 2005, 61, 3953-3962. For alkynyl compounds the Sonogashira reaction is most useful. For alkenyl substrates the Heck and Stille reactions are most useful. For alkyl and cycloalkyl the Kumada and Suzuki couplings are very useful.

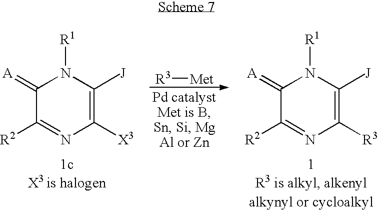

Compounds of Formula 9 (subset of compound of Formula 2 above) wherein X⁴ are halogens can be made by the reaction of cyanoamines of Formula 8 with oxalyl halides as shown in Scheme 8. The reaction is carried out with an excess of an oxalyl halide. The reaction is best carried out in an inert solvent such as 1,2-dichlorobenzene, toluene, chlorobenzene or xylenes at elevated temperatures between about 60 and 150° C. In some cases, the reaction can be carried out at lower temperatures from about 20 to about 60° C. if N,N-dimethylformamide is added to the mixture after the addition of the oxalyl halide. The addition of a halide source such as tetraalkylammonium halides or trialkylammonium halides can sometimes also result in higher yields of product and/or lower reaction temperatures. This type of cyclization can be found in J. Heterocyclic Chemistry, 1983, 20, 919-923, Bull Soc. Chim. Belg. 1994, 103, 583-589, J. Med. Chem., 2005, 48, 1910-1918, and Tetrahedron, 2004, 60, 11597-11612, and references cited therein.

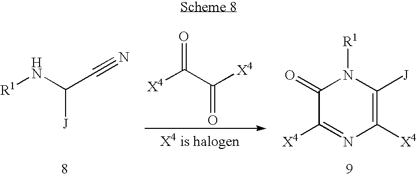

Scheme 9 shows how compounds of Formula 8 can be made by means of the Strecker reaction. This well known reaction involves the reaction of an aldehyde of Formula 10 and an amine of Formula 11 with a cyanide source. The free aldehyde of Formula 10 may be used or it can also be treated with sodium bisulfite prior to the addition to form a bisulfite adduct. The amine of Formula 11 may be in the form of a free base or as an acid addition salt. A variety of solvents and cyanide sources can be employed. For cases in which R¹ is aryl the presence of a Lewis acid such as indium(III) chloride can be advantageous. (For example, see, Ranu et. al., Tetrahedron, 2002, 58, 2529-2532 for typical conditions). This reaction has been the subject of a number of reviews. For conditions and variations of this reaction see the following reference and references cited therein: D. T. Mowry, Chemical Reviews, 1948, 42, 236, H. Groeger, Chemical Reviews, 2003, 103, 2795-2827, and M. North in “Comprehensive Organic Functional Group Transformations” A. R. Katritsky, O. Meth-Cohn and C. W. Rees Editors., Volume 3, 615-617; Pergamon, Oxford, 1995.

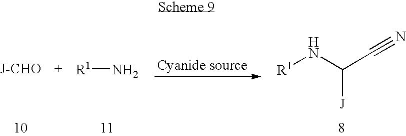

As seen in Scheme 10, compounds of Formula 1e can be made by reaction of compounds of Formula 1a with organometallic reagents of Formula 12 to form ketones of Formula 13, followed by reaction with hydroxylamines and hydrazines of Formula 14. The reaction of Formula 1a with organometallic reagents, preferably Grignard and lithium derivatives, can be carried out at temperatures from −100° C. to 25° C. Preferably the reaction is carried out in ether or tetrahydrofuran, beginning at −50 to −78° C. and then allowing the reaction mixture to warm to 20 to 25° C. The ketones of Formula 13 can be converted to the compounds of Formula 1e by reaction with the reagents of Formula 14 in a variety of solvents and temperatures. Preferred solvents for this transformation include lower alcohols, tetrahydrofuran and dioxane optionally mixed with water. Most preferred is the use of ethanol. The reaction can be carried out at temperatures from 0 to 120° C. and is most commonly done at the reflux temperature of the solvent used.

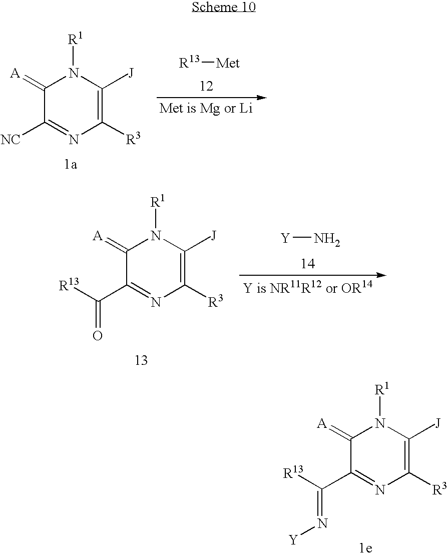

As shown in scheme 11, various amides of Formula 1f can be made by the reaction of compounds of Formula 2 with a compound of Formula 15 followed by reaction with an oxidizing agent and an amine of Formula 16. The compound of Formula 15 is treated with a strong base such as sodium hexamethyldisilazide, sodium hydride, or 1,8-diazabicyclo-[5.4.0]undec-7-ene and added to a compound of Formula 2. This mixture is further treated with an oxidant such as peracetic acid, t-butyl hydroperoxide, bleach, m-chloroperbenzoic acid, nickel peroxide or other oxidizing agent. Finally an amine of Formula 16 is added to give the compound of Formula 1f. Reaction temperatures of between −20 C and 80° C. are preferred with a temperature of 20 to 30° C. being most preferred. A variety of solvents may be employed with tetrahydrofuran being preferred. For a survey of the use of this amide formation technique with a variety of heterocyclic halides, see Zhang, Synlett, 2004, 2323-2326.

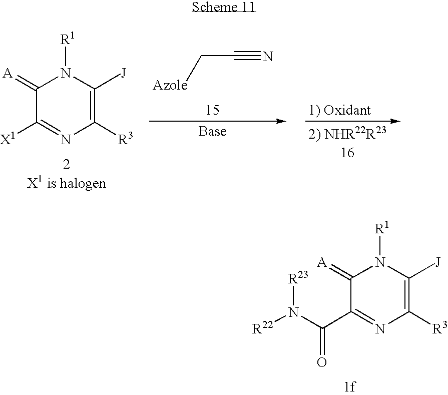

As shown in scheme 12, compounds of Formula 1g can be converted to a compound of Formula 1j by the following reactions. A compound of Formula 1g can be converted to a compound of Formula 17 by treatment with strong acid. A variety of acids may be successfully employed. Trifluoroacetic acid is a preferred acid for this transformation. The reaction is generally carried out at about 20 to 30° C. in an inert solvent such as dichloromethane. A variety of reagents can convert compounds of Formula 17 to compounds of Formula 1h. Many amination reagents are known in the literature and have been discussed in some detail in Vedejs, Org. Lett., 2003, 7, 4187-4190 and references cited within. A preferred reagent is O-di(p-methoxyphenyl)phosphinylhydroxylamine. The presence of a base such as sodium hydride is preferred. Reaction of compounds of Formula 1h with aldehydes and ketones of Formula 18 give compounds of Formula 1i. The reaction can be carried in the presence of an acid with or without a solvent. Appropriate solvents include tetrahydrofuran, dichloromethane or lower alcohols. Compounds of Formula 1i can be reduced to compounds of Formula 1j by standard reduction techniques. Generally these reactions are conducted by reaction of a boron-based reducing agent such as sodium borohydride or sodium triacetoxyborohydride with the compound of Formula 1i in a solvent such as lower alcohols or tetrahydrofuran. Other reduction techniques known to those skilled in the art may also be employed. A compendium of methods and techniques of reduction of imine type bonds can be found in Organic Reactions, (New York) 2002, 59, 1-714.

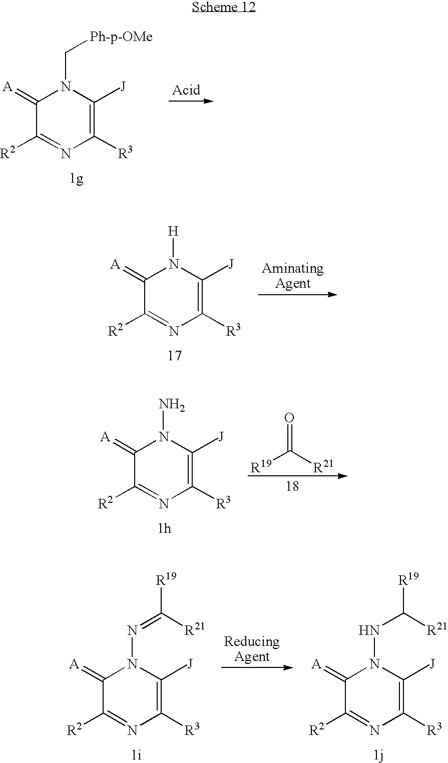

Compounds of Formula 1k in wherein A is NH and R² is a nitrile can be synthesized from compounds of enamines of Formula 19 by a two-step procedure as shown in Scheme 13. The enamines are reacted with [[[(4-methylphenyl)sulfonyl]oxy]imino]propanedinitrile in the presence of a base such as pyridine or triethylamine in a variety of solvents to afford compounds of Formula 20. Preferred solvents include chloroform, dichloromethane and N,N-dimethylformamide. In a second step the compounds of Formula 20 are reacted with an amine of Formula 11 to afford the desired compounds of Formula 1k. Examples of these procedures can be found in Lang et al., Helv. Chem. Acta., 1986, 69, 1025-1033.

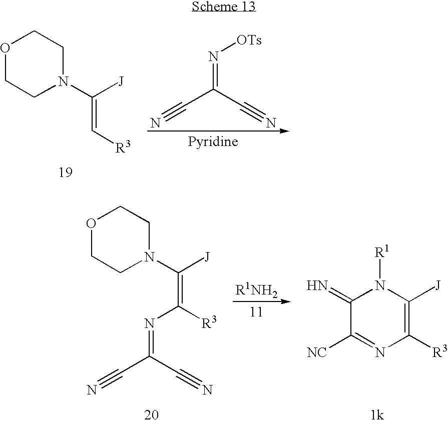

The synthesis of enamines of Formula 19 is well known in the art. For a review of preparative methods see for example Hickmott, et al., Tetrahedron, 1982, 38,1975-2050 and Tetrahedron, 1982, 38, 3363-3446.

Compounds of Formula 1I in wherein A is NH and R² is CONH₂ can be synthesized from compounds of Formula 1k in wherein A is NH and R² is a nitrile by acidic hydrolysis as shown in Scheme 14. Reagents such as trifluoroacetic acid and trifluoroacetic acid/sulfuric acid mixtures can be employed. This reaction can be conducted between about 0 and 200° C., and preferably between about 20 and 80° C.

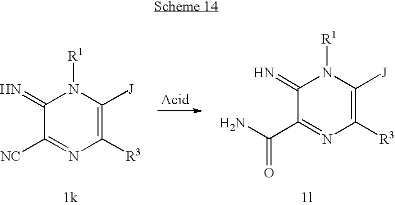

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. MPLC means medium pressure chromatography on silica gel. HPLC means high performance liquid chromatography. ¹H NMR spectra are reported in ppm downfield from tetramethylsilane; “s” means singlet, “d” means doublet, “t” means triplet, “m” means multiplet, “dd” means doublet of doublets, “ddd” means doublet of doublet of doublets, “br s” means broad singlet.

### EXAMPLE 1

### Preparation of 5-Chloro-6-(2,6-difluorophenyl)-1-(2-methylpropyl)-3-(1H-pyrazol-1-yl)-2(1H)-pyrazinone (Compound 1)

### Step A: Preparation of 2,6-Difluoro-α-[(2-methylpropyl)amino]benzeneacetonitrile

To a solution of isobutylamine (2.92 g, 40 mmol) and sodium cyanide (1.94 g, 40 mmol) in water (40 mL) was added a solution of 2,6-difluorobenzaldehyde (5.7 g, 40 mmol) in methanol (40 mL). The addition was done at such a rate so that the temperature remained below 35° C. The reaction mixture was stirred at room temperature for 18 h. The mixture was partitioned between water (150 mL) and dichloromethane (150 mL). The organic layer was washed with water (2×50 mL). The organic layer was dried (MgSO₄) and evaporated under reduced pressure to give an oil. Flash chromatographic purification on silica gel with hexanes as eluant and pooling of appropriate fractions gave 4.92 g of the title compound as an oil.

¹H NMR (CDCl₃) δ 8.4 (br s, 1H), 7.3-7.2 (m, 1H), 6.9 (m, 2H), 3.5 (m, 2H), 2.0 (m, 1H), 0.9 (m, 6H).

### Step B: Preparation of 3,5-Dichloro-6-(2,6-difluorophenyl)-1-(2-methylpropyl)-2(1H)-pyrazinone

A solution of oxalyl chloride (3.34 g, 26 mmol) in chlorobenzene (35 mL) was stirred at 25° C. and 2.46 g (80% pure, 9 mmol) of 2,6-difluoro-α-[(2-methylpropyl)amino]-benzeneacetonitrile (i.e. the product of Example 1 step A) was added via an addition funnel. The resulting reaction mixture was heated at 70° C. for 18 h and at 90° C. for 24 h. The solvent was evaporated under reduced pressure to leave an oil. This residue was subjected to silica gel chromatographic purification using a gradient of ethyl acetate/hexanes (1:9 to 2:3), and the appropriate fractions were pooled to give 1.2 g of the title compound as an oil which solidified on standing. This product was of sufficient purity to use in subsequent reactions.

¹H NMR (CDCl₃) δ 7.6 (m, 1H), 7.1 (m, 1H), 7.0 (m, 1H), 3.7 (m, 2H), 1.9 (m, 1H), 0.9 (m, 3H), 0.7 (d, 3H).

### Step C: Preparation of 5-Chloro-6-(2,6-difluorophenyl)-1-(2-methylpropyl)-3-(1H-pyrazol-1-yl)-2(1H)-pyrazinone (Compound 1)

A mixture of 3,5-dichloro-6-(2,6-difluorophenyl)-1-(2-methylpropyl)-2(1H)-pyrazinone (i.e. the product of Example 1 step B) (200 mg, 0.6 mmol), pyrazole (45 mg, 0.66 mmol) and potassium carbonate (166 mg, 1.2 mmol) dissolved in N,N-dimethylformamide (2 mL) was heated at 60° C. for 18 h. The mixture was partitioned between ethyl acetate (20 mL) and water (10 mL). The organic layer was washed with water (3×10 mL). The residue after evaporation was subjected to silica gel chromatographic purification using a gradient of hexanes/ethyl acetate (1:9 to 2:3) as eluant to give 60 mg of the title product, a compound of the present invention as an oil which later solidified, melting at 118-119° C.

¹H NMR (CDCl₃) δ 9.1 (m, 1H), 7.9 (m, 1H), 7.5 (m, 1H), 7.1 (m, 2H), 6.5 (m, 1H), 3.8 (d, 2H), 2.0 (m, 1H), 0.8 (d, 6H).

### EXAMPLE 2

### Preparation of 5-Chloro-6-(2,6-difluorophenyl)-1-(2-methylpropyl)-3-(2-pyridinyl)-2(1H)-pyrazinone (Compound 2)

A mixture of 3,5-dichloro-6-(2,6-difluorophenyl)-1-(2-methylpropyl)-2(1H)-pyrazinone (i.e. the product of Example 1 step B) (200 mg, 0.6 mmol), tributylstannylpyridine (Lancaster Synthesis, 240 mg, 0.63 mmol) and bis(triphenylphoshino)palladium(II) chloride (20 mg, 0.03 mmol) was heated in toluene at 110° C. for 18 h. The mixture was filtered through a pad of Celite®, diatomaceous filter aid, and rinsed with ethyl acetate. The solvent was evaporated under reduced pressure. The residue after evaporation was subjected to silica gel chromatographic purification using a gradient of ethyl acetate/hexanes (1:9 to 2:3), and the appropriate fractions were pooled to give 56 mg of the title product, a compound of the present invention as an oil.

¹H NMR (CDCl₃) δ 8.86 (m, 1H), 8.43 (m, 1H), 7.83 (m, 1H), 7.59 (m, 1H), 7.38 (m, 1H), 7.12 (m, 2H), 3.79 (d, 2H), 2.00 (m, 1H), 0.79 (d, 6H).

### EXAMPLE 3

### Preparation of 6-(2,6-Difluorophenyl)-1-(2-methylpropyl)-3-(1H-pyrazol-1-yl)-2(1H)-pyrazinone (Compound 342)

A mixture of 5-chloro-6-(2,6-difluorophenyl)-1-(2-methylpropyl)-3-(1H-pyrazol-1-yl)-2(1H)-pyrazinone (i.e. the product of Example 1 step C) (0.70 g, 1.92 mmol), triethylamine (0.40 mL, 2.88 mmol) and 10% Palladium on carbon (50 mg, 0.471 mmol) in ethyl Acetate (10 mL) was shaked under 50 psi (345 kPa) pressure of hydrogen overnight. The reaction mixture was filtered through Celite® diatomaceous filter aid. The solvent was removed with a rotary evaporator. The residue was taken up in ethyl acetate and was washed with water. The organic layer was dried, and the solvent was removed with a rotary evaporator. The residue was purified by silica gel flash chromatography (1 to 33% ethyl acetate in hexanes as eluant) to give 110 mg of the title product, a compound of the present invention as an oil which later solidified, melting at 91-92° C.

¹H NMR (CDCl₃), δ 9.10 (s, 1 H), 7.86 (s, 1 H), 7.54 (m, 1 H), 7.31 (s, 1 H), 7.09 (m, 2 H), 6.50 (s, 1 H), 3.80 (d, 2 H), 2.04 (m, 1 H), 0.78 (d, 6 H).

### EXAMPLE 4

### Preparation of 5-Chloro-6-(2,6-difluorophenyl)-1-[(4-methoxyphenyl)methyl]-3-(1H-pyrazol-1-yl)-2(1H)-pyrazinone (Compound 271), 1-Amino-5-chloro-6-(2,6-difluorophenyl)-3-(1H-pyrazol-1-)-2(1H)-pyrazinone (Compound 400) and 5-Chloro-6-(2,6-difluorophenyl)-1-[(1-methylethylidene)amino]-3-(1H-pyrazol-1-yl)-2(1H)-pyrazinone (Compound 392)

### Step A: Preparation of 2,6-Difluoro-α-[[(4-methoxyphenyl)methyl]amino]benzene-acetonitrile

To a solution of sodium hydrogensulfite (19.9 g, 0.191 mol) in water (180 mL) and methanol (18 mL) was added 2,6-difluorobenzaldehyde (25.95 g, 0.182 mol). The reaction mixture was stirred at room temperature for 15 minutes. A mild exotherm was observed to 30° C. Then sodium cyanide (8.93 g, 0.182 mol) was added and the reaction mixture was stirred for 25 minutes. The reaction mixture was cooled to 10° C. and 4-methoxybenzylamine (24.99 g, 0.182 mol) was added dropwise. The reaction was heated to 65° C. for 5 h and allowed to cool to room temperature overnight. The reaction mixture was diluted with diethyl ether (200 mL) and washed with brine (2×100 mL). The aqueous layer was extracted with diethyl ether once. The organic layers were combined, dried (MgSO₄), filtered and concentrated under reduced pressure to give 51.26 g of the title compound as an oil.

¹H NMR (CDCl₃) δ 7.37-7.28 (m, 3H), 6.96 (t, 2H), 6.88 (d, 2H), 4.94 (s, 1H), 4.05 (d, 1H), 3.89 (d, 1H), 3.81 (s, 3H), 2.27 (s, 1H).

### Step B: Preparation of 3,5-Dichloro-6-(2,6-difluorophenyl)-1-[(4-methoxyphenyl)-methyl]-2(1H)-pyrazinone

To a solution of 2,6-difluoro-α-[[(4-methoxyphenyl)methyl]amino]benzene-acetonitrile (i.e. the product of Example 4 step A) (48.8 g, 0.169 mol) in chlorobenzene (550 mL) was added oxalyl chloride (64.45 g, 0.507 mol) dropwise keeping temperature below 15° C. The reaction mixture was then warmed to room temperature and stirred for 30 minutes. Then triethylamine hydrochloride (46.6 g, 0.338 mol) was added and reaction mixture was heated to 80° C. for 2 h. The reaction mixture was allowed to stir at room temperature overnight. The resulting mixture was then concentrated under reduced pressure, and purified by silica gel flash chromatography (25% ethyl acetate in hexanes as eluant) to afford 31.2 g of the title compound as an oil.

¹H NMR (CDCl₃) δ 7.55 (s, 1H), 7.02 (dd, 2H), 6.77-6.67 (m, 4H), 5.04 (s, 2H), 3.75 (s, 3H).

### Step C: Preparation of 5-Chloro-6-(2,6-difluorophenyl)-1-[(4-methoxyphenyl)-methyl]-3-(1H-pyrazol-1-yl)-2(1H)-pyrazinone (Compounds 271)

To a solution of 3,5-dichloro-6-(2,6-difluorophenyl)-1-[(4-methoxyphenyl)-methyl]-2(1H)-pyrazinone (i.e. the product of Example 4 step B) (20 g, 50.0 mmol) in acetonitrile (250 mL) was added pyrazole (3.43 g, 60.0 mmol) and potassium bicarbonate (20.74 g, 150 mmol), and stirred at 60° C. for 3 h. The reaction mixture was then cooled to room temperature and poured into ice water (500 mL). After stirring for 10 minutes, resulting precipitate was filtered, rinsed with cold water, and dried to afford 21.17 g of the title product, a compound of the present invention as an off-white solid.

hu 1H NMR (CDCl₃) δ 9.13 (d, 1H), 7.90 (d, 1H), 7.54 (s, 1H), 7.05-6.97 (m, 2H), 6.83-6.75 (m, 2H), 6.74-6.68 (m, 2H), 6.52 (dd, 1H), 5.13 (s, 2H), 3.75 (s, 3H).

### Step D: Preparation of 5-Chloro-6-(2,6-difluorophenyl)-3-(1H-pyrazol-1-yl)-2(1H)-pyrazinone

A solution of 5-chloro-6-(2,6-difluorophenyl)-1-[(4-methoxyphenyl)-methyl]-3-(1H-pyrazol-1-yl)-2(1H)-pyrazinone (i.e. the product of Example 4 step C) (21.17 g, 49.0 mmol) in trifluoroacetic acid (37 mL, 493 mmol) was stirred under reflux for 6 h and allowed to cool to room temperature overnight. The reaction mixture was concentrated under reduced pressure and the resulting crude oil was purified by silica gel flash chromatography using 100% dichloromethane as eluant. It was the recrystallized from methanol to give 6.07 g of the title compound as an oil.

¹H NMR (CDCl₃) δ 12.74 (s, 1H), 8.63 (d, 1H), 7.84 (s, 1H), 7.44 (ddd, 1H), 7.02 (t, 2H), 6.64 (s, 1H).

### Step E: Preparation of 1-Amino-5-chloro-6-(2,6-difluorophenyl)-3-(1H-pyrazol-1-)-2(1H)-pyrazinone (Compound 400)

To a slurry sodium hydride (55% of oil dispersion, 42.5 mg, 0.974 mmol) in tetrahydrofuran (8 mL) was added a solution of 1-amino-5-chloro-6-(2,6-difluorophenyl)-3-(1H-pyrazol-1-)-2(1H)-pyrazinone (i.e. the product of Example 4 step D) (250 mg, 0.812 mmol) in tetrahydrofuran (11 mL) at approximately −78° C. The reaction mixture was stirred at −78° C. for 15 minutes and then at 0° C. for 15 additional minutes. Then 1,1-dimethylethyl [[bis(4-methoxyphenyl)phosphinyl]oxy]carbamate (262 mg, 8.93 mmol) was added and the reaction mixture was allowed to warm to room temperature overnight. The reaction mixture was then concentrated under reduced pressure and purified by MPLC (0 to 100% ethyl acetate in hexanes as eluant) to afford 36 mg of the title product, a compound of the present invention as an oil.

¹H NMR (CDCl₃) δ 9.12-9.03 (m, 1H), 7.91 (s, 1H), 7.64-7.49 (m, 1H), 7.17-7.05 (m, 2H), 6.54 (s, 1H), 5.43 (s, 2H).

### Step F: Preparation of 5-Chloro-6-(2,6-difluorophenyl)-1-[(1-methylethylidene)-amino]-3-(1H-pyrazol-1-yl)-2(1H)-pyrazinone (Compounds 392)

To a solution of 1-amino-5-chloro-6-(2,6-difluorophenyl)-3-(1H-pyrazol-1-)-2(1H)-pyrazinone (i.e. the product of Example 4 step E) (36 mg, 0.111 mmol) in acetone (10 mL) was added a solution of 2 M hydrogen chloride in diethyl ether (2 mL) and 4 Å molecular sieves. The reaction mixture was then stirred at room temperature overnight. The resulting mixture was concentrated under reduced pressure to give 40 mg of the title product, a compound of the present invention.

¹H NMR (CDCl₃) δ 9.10 (s, 1H), 7.90 (s, 1H), 7.54-7.45 (m, 1H), 7.12-7.03 (m, 1H), 7.03-6.95 (m, 1H), 6.51 (s, 1H), 2.10 (s, 3H), 1.94 (s, 3H).

### EXAMPLE 5

### Preparation of 5-Chloro-6-(1-methylpropyl)-1-(2-methylpropyl)-3-(1H-pyrazol-1-yl)-2(1H)-pyrazinone (Compound 424)

### Step A: Preparation of 3-Methyl-2-[(2-methylpropyl)amino]pentanenitrile

To a solution of sodium hydrogensulfite (2.31 g, 22.2 mmol) in water (20 mL) and methanol (2 mL) was added 2-methylbutyraldehyde (1.82 g, 21.1 mmol) at room temperature. The reaction mixture was then stirred for 15 minutes, and sodium cyanide (1.09 g, 22.2 mmol) was added. The reaction mixture was stirred for an additional 20 minutes. The reaction mixture was then cooled in an ice water bath and a solution of isobutylamine (1.70 g, 23.2 mmol) in methanol (4 mL) was added over approximately 2 minutes period. The reaction mixture was stirred at 0° C. for 15 minutes and then heated to 35° C. for 2 h. The reaction mixture was then extracted with ethyl acetate (2×20 mL) and the combined organic layers were washed with brine, dried (MgSO₄), and concentrated to give 3.1 g of the title compound as a yellow oil.

¹H NMR (CDCl₃) δ 3.41-3.33 (m, 1H), 2.71-2.65 (m, 1H), 2.44-2.36 (m, 1H), 1.79-1.66 (m, 2H), 1.66-1.54 (m, 1H), 1.39-1.29 (m, 1H), 1.10-1.03 (m, 3H), 0.97-0.89 (m, 9H).

### Step B: Preparation of 3,5-Dichloro-6-(1-methylpropyl)-1-(2-methylpropyl)-2(1H)-pyrazinone

A solution of 3-methyl-2-[(2-methylpropyl)amino]pentanenitrile (i.e. the product of Example 5 step A) (3.1 g, 18.4 mmol) in chlorobenzene (12 mL) was added over 20 minutes to a solution of oxalyl chloride (11.7 g, 92.1 mmol) in chlorobenzene (43 mL) at room temperature. Then N,N-dimethylformamide (3 mL) was added dropwise. The reaction mixture was then heated to 95° C. overnight. The reaction mixture was concentrated under reduced pressure and the residue was purified by MPLC (0 to 100% gradient of ethyl acetate in hexanes as eluant) to afford 3.7 g of the title compound as a yield solid.

¹H NMR (CDCl₃) δ 4.22-4.08 (m, 1H), 4.02-3.92 (m, 1H), 3.02-2.88 (m, 1H), 2.09-1.98 (m, 2H), 1.97-1.87 (m, 1H), 1.45 (d, 3H), 1.02-0.91 (m, 9H).

### Step C: Preparation of 5-Chloro-6-(1-methylpropyl)-1-(2-methylpropyl)-3-(1H-pyrazol-1-yl)-2(1H)-pyrazinone (Compound 424)

A mixture of 3,5-dichloro-6-(1-methylpropyl)-1-(2-methylpropyl)-2(1H)-pyrazinone (i.e. the product of Example 5 step B) (0.30 g, 1.09 mmol), pyrazole (0.081 g, 1.20 mmol) and potassium carbonate (0.30 g, 2.17 mmol) in N,N-dimethylformamide (4 mL) was heated at 60° C. overnight. The reaction mixture was then concentrated under reduced pressure. The residue was purified by MPLC (0 to 100% gradient of ethyl acetate in hexanes as eluant) to give 0.22 g of the title product, a compound of the present invention.

¹H NMR (CDCl₃) δ 8.96 (br s, 1H), 7.83 (br s, 1H), 6.45 (br s, 1H), 4.40-4.15 (m, 1H), 4.16-3.97 (m, 1H), 3.12-2.92 (m, 1H), 2.16-2.01 (m, 2H), 2.02-1.88 (m, 1H), 1.49 (d, 3H), 1.05-0.98 (m, 6H), 0.98-0.92 (m, 3H).

### EXAMPLE 6

### Preparation of 5-Chloro-6-(2-chloro-4-fluorophenyl)-1-(2-methylpropyl)-3-(1H-pyrazol-1-yl)-2(1H)-pyrazinone (Compound 53)

### Step A: Preparation of 2-Chloro-4-fluoro-α-[(2-methylpropyl)amino]benzene-acetonitrile

To a solution of sodium hydrogensulfite (1.53 g, 14.8 mmol) in a mixture of deionized water (14 mL) and methanol (1.3 mL) was added 2-chloro-4-fluoro-benzaldehyde (2.23 g, 14.1 mmol) at room temperature. The reaction mixture was stirred for 15 minutes, and sodium cyanide (0.724 g, 14.8 mmol) was added. The reaction mixture was stirred for an additional 20 minutes. The reaction mixture was cooled in an ice water bath and a solution of isobutylamine (1.13 g, 15.5 mmol) in methanol (2.67 mL) was added over approximately 2 minutes. The reaction mixture was stirred at 0° C. for 15 minutes and then heated to 35° C. for 2 h. The resulting mixture was then extracted ethyl acetate (2×20 mL) and the combined organic layers were washed with brine, dried (MgSO₄) and concentrated to give 3.09 g of the title compound as a yellow oil.

¹H NMR (CDCl₃) δ 7.65-7.61 (m, 1H), 7.22-7.18 (m, 1H), 7.10-7.04 (m, 1H), 5.01 (s, 1H), 2.70-2.64 (m, 1H), 2.58-2.51 (m, 1H), 1.81-1.71 (m, 1H), 0.97-0.92 (m, 6H).

### Step B: Preparation of 3,5-Dichloro-6-(2-chloro-4-fluorophenyl)-1-(2-methylpropyl)-2(1H)-pyrazinone

A solution of 2-chloro-4-fluoro-α-[(2-methylpropyl)amino]benzeneacetonitrile (i.e. the product of Example 6 step A) (3.09 g, 12.8 mmol) was dissolved in chlorobenzene (8 mL) and added dropwise over 20 minutes to a solution of oxalyl chloride (8.15 g, 64.2 mmol) in chlorobenzene (30 mL) at room temperature. The reaction mixture was then heated to 100° C. overnight. The solvent was removed under reduced pressure and the residue was purified by MPLC (0 to 100% ethyl acetate in hexanes as eluant) to give 2.13 g of the title compound as a solid.

¹H NMR (CDCl₃) δ 7.38-7.31 (m, 2H), 7.23-7.17 (m, 1H), 4.02-3.95 (m, 1H), 3.38-3.30 (m, 1H), 2.01-1.90 (m, 1H), 0.82 (d, 3H), 0.72 (d, 3 H).

### Step C: Preparation of 5-Chloro-6-(2-chloro-4-fluorophenyl)-1-(2-methylpropyl)-3-(1H-pyrazol-1-yl)-2(1H)-pyrazinone (Compound 53)

A mixture of 3,5-dichloro-6-(2-chloro-4-fluorophenyl)-1-(2-methylpropyl)-2(1H)-pyrazinone (i.e. the product of Example 6 step B) (0.350 g, 1.00 mmol), pyrazole (0.075 g, 1.10 mmol) and potassium carbonate (0.276 g, 2.00 mmol) in N,N-dimethylformamide (4 mL) was heated to 60° C. overnight. The reaction mixture was concentrated under reduced pressure and the residue was purified by MPLC (0 to 100% ethyl acetate in hexanes as eluant) to give 0.256 g of the title product, a compound of the present invention as a solid melting at 137-139° C.

¹H NMR (CDCl₃) δ 9.10 (d, 1H), 7.89 (d, 1H), 7.48-7.38 (m, 1H) 7.37-7.30 (m, 1H), 7.27-7.14 (m, 1H), 6.56-6.46 (m, 1H), 4.16-4.03 (m, 1H), 3.48-3.36 (m, 1H), 2.08-1.91 (m, 1H), 0.84 (d, 3H), 0.75 (d, 3H).

### EXAMPLE 7

### Separation of the atropisomers of 5-Chloro-6-(2-chloro-4-fluorophenyl)-1-(2-methylpropyl)-3-(1H-pyrazol-1-yl)-2(1H)-pyrazinone: (Compound 302) and (Compound 303)

5-Chloro-6-(2-chloro-4-fluorophenyl)-1-(2-methylpropyl)-3-( 1H-pyrazol-1-yl)-2(1H)-pyrazinone (i.e. the product of Example 6 step C) (40 mg, 0.10 mmol) was purified on a ChiralCel® OJ, analytical HPLC column by Daicel Chemical Industries, LTD., (0.1% formic acid in a mixture of 49.9% methanol and 50% acetonitrile as eluant, 1 mL/min) to afford 16 mg of the title product, the Compound 303 of the present invention at the retention time of 18.9 minutes, and 16.5 mg of the title product, the Compound 302 of the present invention at the retention time of 22.6 minutes.

¹H NMR (CDCl₃) of 5-Chloro-6-(2-chloro-4-fluorophenyl)-1-(2-methylpropyl)-3-(1H-pyrazol-1-yl)-2(1H)-pyrazinone (Compound 302): δ 9.10 (br s, 1H), 7.89 (br s, 1H), 7.42-7.37 (m, 1H), 7.36-7.31 (m, 1H), 7.24-7.16 (m, 1H), 6.51 (br s, 1H), 4.17-4.04 (m, 1H), 3.46-3.34 (m, 1H), 2.09-1.93 (m, 1H), 0.85 (d, 3H), 0.75 (d, 3H).

¹H NMR (CDCl₃) of 5-Chloro-6-(2-chloro-4-fluorophenyl)-1-(2-methylpropyl)-3-(1H-pyrazol-1-yl)-2(1H)-pyrazinone (Compound 303): δ 9.09 (br s, 1H), 7.89 (br s, 1H), 7.42-7.36 (m, 1H), 7.36-7.31 (m, 1H), 7.23-7.17 (m, 1H), 6.52 (br s, 1H), 4.16-4.04 (m, 1H), 3.45-3.34 (m, 1H), 2.09-1.93 (m, 1H), 0.84 (d, 3H), 0.75 (d, 3H).

### EXAMPLE 8

### Preparation of 6-Chloro-4-(3-fluorophenyl)-3,4-dihydro-3-oxo-5-(2,4,6-trifluorophenyl)pyrazinecarboxamide (Compound 414)

### Step A: Preparation of 2,4,6-Trifluoro-α-[(3-fluorophenyl)amino]benzeneacetonitrile

To a solution of 2,4,6-trifluorobenzaldehyde (3.20 g, 20.0 mmol) in tetrahydrofuran (25 mL) was added 3-fluorophenylaniline (2.02 g, 18.2 mmol), potassium cyanide (4.74 g, 72.7 mmol) and indium(III) chloride (4.02 g, 18.2 mmol) in sequence at room temperature. Then the reaction mixture was stirred overnight. The reaction mixture was diluted with water and extracted with ethyl acetate (2×100 mL). The organic extracts were dried (MgSO₄), filtered, and concentrated to afford 5.33 g of the title compound as an oil.

¹H NMR (CDCl₃) δ 7.25 (m, 1H), 6.81 (m, 2H), 6.62 (m, 1H), 6.53 (m, 2H), 5.64 (d, 1H), 4.42 (d, 1H).

### Step B: Preparation of 3,5-Dichloro-1-(3-fluorophenyl)-6-(2,4,6-trifluorophenyl)-2(1H)-pyrazinone

A solution of 2,4,6-trifluoro-α-[(3-fluorophenyl)amino]benzeneacetonitrile (i.e. the product of Example 8 step A) (5.33 g, 19.0 mmol) in chlorobenzene (20 mL) was treated dropwise with oxalyl chloride (8.30 mL, 95.2 mmol) at room temperature. The resulting mixture was heated to 100° C. for 2.5 h. One drop of N,N-dimethylformamide was then added, and heating was continued overnight. The reaction mixture was cooled to room temperature, and concentrated under reduced pressure. The residue was purified by silica gel flash chromatography (15 to 30% ethyl acetate in hexanes as eluant) to afford 6.49 g of the title compound as an oil.

¹H NMR (CDCl₃) δ 7.35 (m, 1H), 6.94 (m, 2H), 6.64 (m, 2H).

### Step C: Preparation of 6-Chloro-4-(3-fluorophenyl)-3,4-dihydro-3-oxo-5-(2,4,6-trifluorophenyl)pyrazinecarboxamide (Compound 414)

To a solution of 3,5-dichloro-1-(3-fluorophenyl)-6-(2,4,6-trifluorophenyl)-2(1H)-pyrazinone (i.e. the product of Example 8 step B) (0.39 g, 1.00 mmol) in tetrahydrofuran (5 mL) was added 1H-benzotriazole-1-acetonitrile (0.24 g, 1.50 mmol) and lithium bis(trimethylsilyl)amide (1.0 M solution in tetrahydrofuran, 2.5 mL, 2.50 mmol). The reaction mixture was stirred at room temperature for 1.5 h. Then a solution of ammonia in dioxane (0.5 M, 6 mL, 3.0 mmol) was added and the reaction mixture was stirred an additional 10 minutes. Peracetic acid (32 wt. % solution in acetic acid, 0.84 mL) was added dropwise to the reaction mixture and the resulting mixture was stirred at room temperature for 3 h. Saturated aqueous sodium hydrogensulfite was then added (50 mL) and the reaction mixture was extracted with ethyl acetate (2×50 mL). The combined organic extracts were dried (MgSO₄), filtered, and concentrated under reduced pressure. The residue was purified by silica gel flash chromatography (50 to 80% ethyl acetate in hexanes as eluant) to afford 0.15 g of the title product, a compound of the present invention as an oil.

¹H NMR (CDCl₃) δ 8.98 (s, 2H), 7.63 (m, 2H), 7.40 (m, 1H), 7.12 (m, 2H), 6.24 (s, 1H).

### EXAMPLE 9

### Preparation of 5-Bromo-6-(2,6-difluorophenyl)-1-(2-methylpropyl)-3-(1H-pyrazol-1-yl)-2(1H)-pyrazinone (Compound 99) and 5-Methyl-6-(2,6-difluorophenyl)-1-(2-methylpropyl)-3-(1H-pyrazol-1-yl)-2(1H)-pyrazinone (Compound 149)

### Step A: Preparation of 3,5-Dibromo-6-(2,6-difluorophenyl)-1-(2-methylpropyl)-2(1H)-pyrazinone

To a solution of oxalyl bromide (8.66 g, 40.1 mmol) in chlorobenzene (40 mL) was added a solution of 2,6-difluoro-α-[(2-methylpropyl)amino]benzeneacetonitrile (i.e. the product of Example 1 step A) (3.0 g, 13.3 mmol) in chlorobenzene (20 mL) at a temperature below 30° C. The reaction mixture was stirred at room temperature for 45 minutes. Then a catalytic amount of N,N-dimethylformamide was added and then heated at 100° C. for 18 h. The solvent was removed with a rotary evaporator. The residue was purified by silica gel flash chromatography (5% ethyl acetate in hexanes as eluant) to afford 2 g of the title compound as a solid melting at 125-126° C.

¹H NMR (CDCl₃) δ 7.6 (m, 1H), 7.1 (m, 2H), 3.7 (d, 2H), 1.9 (m, 1H), 0.7 (d, 6H).

### Step B: Preparation of 5-Bromo-6-(2,6-difluorophenyl)-1-(2-methylpropyl)-3-(1H-pyrazol-1-yl)-2(1H)-pyrazinone (Compound 99)

A mixture of 3,5-dibromo-6-(2,6-difluorophenyl)-1-(2-methylpropyl)-2(1H)-pyrazinone (i.e. the product of Example 9 step A) (1.4 g, 3.3 mmol), pyrazole (248 mg, 3.6 mmol) and potassium carbonate (1.3 g, 9.9 mmol) in acetonitrile (10 mL) was heated at 80° C. for 2 h, then 60° C. overnight. Then additional pyrazole (100 mg) was added, and heated at 80° C. for 2 h. the reaction mixture was diluted with water and the resulting solid was filtered. The filtered solid was dissolved with dichloromethane, passed through ChemElute®, diatomaceous earth column by Varian, and concentrated under reduced pressure to an oil. The residue was triturated with a mixture of hexanes and diethyl ether to give 1.05 g of the title product, a compound of the present invention as a white solid melting at 111-112° C.

¹H NMR (CDCl₃) δ 9.0 (d, 1H), 7.8 (s, 1H), 7.6 (m, 1H), 7.1 (m, 2H), 6.5 (d, 1H), 3.8 (d, 2H), 1.9 (m, 1H), 0.7 (d, 6H).

### Step C: Preparation of 5-Methyl-6-(2,6-difluorophenyl)-1-(2-methylpropyl)-3-(1H-pyrazol-1-yl)-2(1H)-pyrazinone (Compound 149)

To a solution of 5-bromo-6-(2,6-difluorophenyl)-1-(2-methylpropyl)-3-(1H-pyrazol-1-yl)-2(1H)-pyrazinone (i.e. the product of Example 9 step B) (200 mg, 0.48 mmol) and tetrakis(triphenylphosphine)palladium (16 mg, 0.015 mmol) in dimethoxyethane (5 mL) under nitrogen atmosphere was added a solution of 2 M trimethylaluminum in hexanes (0.26 mL, 0.51 mmol) dropwise at a temperature below 10° C. The reaction mixture was warmed to room temperature and then heated at 80° C. for about 90 minutes. The resulting mixture was cooled with an ice-water bath and quenched with saturated ammonium chloride aqueous solution (10 mL). The reaction mixture was diluted with ethyl acetate, and the separated organic layer was washed with brine. The resulting organic layer was passed through ChemElute®, diatomaceous earth column by Varian, and concentrated under reduced pressure to give an oil. This residue was purified by silica gel flash chromatography (5 to 40% ethyl acetate in hexanes as eluant) to afford 44 mg of the title product, a compound of the present invention as a white solid melting at 105-106° C.

¹H NMR (CDCl₃) δ 9.12 (s, 1H), 7.86 (s, 1H), 7.58 (m, 1H), 7.10 (m, 2H), 6.48 (s, 1H), 3.77 (d, 2H), 2.17 (s, 3H), 1.95 (m, 1H), 0.75 (d, 6H).

### EXAMPLE 10

### Preparation of 6-Chloro-5-(2,6-difluorophenyl)-3,4-dihydro-4-(2-methylpropyl)-3-oxopyrazinecarbonitrile (Compound 5)

A mixture of 3,5-dichloro-6-(2,6-difluorophenyl)-1-(2-methylpropyl)-2(1H)-pyrazinone (i.e. the product of Example 1 step B) (200 mg, 0.6 mmol) and sodium cyanide (31 mg, 0.63 mmol) in N,N-dimethylformamide (2 mL) was heated at 60° C. overnight. The reaction mixture was diluted with water and extracted with diethyl ether. The organic layer was separated and washed with water, passed through ChemElute®, diatomaceous earth column by Varian, and concentrated under reduced pressure to give an oil. This residue was purified by silica gel flash chromatography (10 to 20% ethyl acetate in hexanes as eluant) to afford 70 mg of the title product, a compound of the present invention as a white solid melting at 100-102° C.

¹H NMR (CDCl₃) δ 7.6 (m, 1H), 7.1 (m, 2H), 3.7 (d, 2H), 1.9 (m, 1H), 0.7 (m, 6H).

### EXAMPLE 11

### Preparation of 5-Chloro-6-(2,6-difluorophenyl)-1-(2-methylpropyl)-3-(1-methyl-1H-imidazol-4-yl)-2(1H)-pyrazinone (Compound 85)

To a solution of 4-iodo-1-methyl-1H-imidazole (0.31 g, 1.50 mmol) in dichloromethane (5 mL) was added ethylmagnesium bromide (3.0 M solution in tetrahydrofuran, 0.50 mL, 1.50 mmol). The reaction mixture was stirred at room temperature for 15 minutes and a solution of 3,5-dichloro-6-(2,6-difluorophenyl)-1-(2-methylpropyl)-2(1H)-pyrazinone (i.e. the product of Example 10 step A) (0.50 g, 1.50 mmol) in dichloromethane (5 mL) was added. The reaction mixture was stirred at room temperature overnight, and then quenched with saturated aqueous ammonium chloride solution (1 mL). The resulting mixture was passed through ChemElute®, diatomaceous earth column by Varian, and concentrated under reduced pressure to give an oil. This residue was purified by silica gel flash chromatography (5% methanol in ethyl acetate as eluant) to afford 150 mg of the title product, a compound of the present invention.

¹H NMR (CDCl₃) δ 8.35 (s, 1H), 7.59 (s, 1H), 7.58-7.51 (m, 1H), 7.08 (t, 2H), 3.78-3.74 (m, 5H), 2.01-1.92 (m, 1H), 0.76 (d, 6H).

### EXAMPLE 12

### Preparation of 5-Chloro-6-(2,6-difluorophenyl)-1-(2-methylpropyl)-3-(5-methyl-2-pyridinyl)-2(1H)-pyrazinone (Compound 209)

### Step A: Preparation of 5-Chloro-6-(2,6-difluorophenyl)-3-iodo-1-(2-methylpropyl)-2(1H)-pyrazinone

To a solution of 3,5-dichloro-6-(2,6-difluorophenyl)-1-(2-methylpropyl)-2(1H)-pyrazinone (i.e. the product of Example 10 step A) (0.50 g, 1.50 mmol) in acetonitrile (10 mL) was added sodium iodide (0.34 g, 2.25 mmol), hydroiodic acid (10 drops), and acetone (1 mL). The resulting mixture was heated at reflux for 2 h and allowed to cool to room temperature. The reaction mixture was diluted with diethyl ether, filtered, and concentrated in vacuo. The residue was passed through ChemElute®, diatomaceous earth column by Varian, washed with dichloromethane, and concentrated under reduced pressure to give an oil. This residue was purified by Bond Elut® SI, silica gel column by Varian, using dichloromethane as eluant to afford 620 mg of the title compound.

¹H NMR (CDCl₃) δ 7.62-7.55 (m, 1H), 7.10 (t, 2H), 3.68 (d, 2H), 1.93 (s, 1H), 0.77-0.73 (m, 6H).

### Step B: Preparation of 5-Chloro-6-(2,6-difluorophenyl)-1-(2-methylpropyl)-3-(5-methyl-2-pyridinyl)-2(1H)-pyrazinone (Compound 209)

To a solution of 5-chloro-6-(2,6-difluorophenyl)-3-iodo-1-(2-methylpropyl)-2(1H)-pyrazinone (i.e. the product of Example 12 step A) (0.50 g, 1.18 mmol) in tetrahydrofuran (20 mL) was added tetrakis(triphenylphosphine)palladium(O) (0.13 g, 0.12 mmol) and 4-methyl-2-pyridinylzinc bromide (Aldrich, 0.5 M solution in tetrahydrofuran, 3.54 mL, 1.77 mmol). The resulting mixture was heated at 80° C. overnight and concentrated in vacuo. The residue was purified by silica gel flash chromatography (20% ethyl acetate in dichloromethane eluant) to give afford 380 mg of the title product, a compound of the present invention.

¹H NMR (CDCl₃) δ 8.68 (s, 1H), 8.40 (s, 1H), 7.65-7.57 (m, 2H), 7.12 (t, 2H), 3.79 (d, 2H), 2.44 (s, 3H), 2.04-1.99 (m, 1H), 0.78 (d, 6H).

### EXAMPLE 13

### Preparation of 5-Chloro-6-(2,6-difluorophenyl)-3-formamido-1-(2-methylpropyl)-2(1H)-pyrazinone (Compound 422)

To a solution of 3,5-dichloro-6-(2,6-difluorophenyl)-1-(2-methylpropyl)-2(1H)-pyrazinone (i.e. the product of Example 10 step A) (500 mg, 1.5 mmol) and 4 Å molecular sieves (8.0 g) in N,N-dimethylformamide (6 mL) was added sodium hydride (55% dispersion in mineral oil, 0.297 g, 3.75 mmol) at room temperature. The reaction mixture was stirred for 15 minutes and formamide (0.203 g, 4.5 mmol) was added. The reaction mixture was stirred for 3 h at 60° C. and then filtered through a sintered glass frit and concentrated under reduced pressure. The residue was purified by MPLC (20 to 100% ethyl acetate in hexanes as eluant) to afford 258 mg of the title product, a compound of the present invention as an oil.

¹H NMR (CDCl₃) δ 9.41 (d, 1H), 9.15-9.08 (m, 1H), 7.62-7.53 (m, 1H), 7.14-7.07 (m, 2H), 3.71 (d, 2H), 1.94-1.84 (m, 1H), 0.76 (d, 6H).

### EXAMPLE 14

### Preparation of 5-(2,4-Difluorophenyl)-3,4-dihydro-3-imino-6-methyl-4-(2-methylbutyl)pyrazinecarbonitrile (Compound 426) and N-[3-Cyano-6-(2,4-difluorophenyl)-5-methyl-1-(2-methylbutyl)-2(1H)-pyrazinylidene]acetamide (Compound 430)

### Step A: Preparation of 4-[1-(2,4-Difluorophenyl)-1-propenyl]morpholine

To a solution of 1-(2,4-difluorophenyl)-1-propanone (17 g, 100 mmol) and morpholine (35 mL, 400 mmol) in toluene (350 mL) was added dropwise a 1 M solution of titanium(IV) chloride in toluene (50 mL, 50 mmol) at such a rate to maintain a temperature below −10° C. After the addition was complete the reaction mixture was allowed to room temperature and stirred overnight. It was then filtered through Celite® diatomaceous filter aid. The solvent was removed with a rotary evaporator to afford 16 g of the title compound as an oil.

¹H NMR (CDCl₃) δ 7.28 (m, 1H), 6.89 (dd, 1H), 6.82 (dd, 1H), 4.82 (q, 1H), 3.68 (m, 4H), 2.72 (m, 4H), 1.46 (d, 3H).

### Step B: Preparation of [[2-(2,4-Difluorophenyl)-1-methyl-2-(4-morpholinyl)ethenyl]-imino]propanedinitrile

To a solution of 4-[1-(2,4-difluorophenyl)-1-propenyl]morpholine (i.e. the product of Example 14 Step A) (8.0 g, 34 mmol) and [[[(4-methylphenyl)sulfonyl]oxy]imino]-propanedinitrile (8.3 g, 34 mmol) in diethyl ether (250 mL) at 0° C. was added dropwise a solution of pyridine (3.0 mL, 37 mmol) in diethyl ether (50 mL). After the addition was complete the reaction mixture was allowed to room temperature and stirred for three days. The reaction mixture was diluted with hexanes and a solid was filtered off. The solvent was removed from the filtrate with a rotary evaporator. The residue was triturated with chlorobutane and then water. The solid obtained was dried in a vacuum oven to afford 7.1 g of the title compound.

¹H NMR (CDCl₃) δ 7.24 (m, 1H), 7.05 (dd, 1H), 6.99 (dd, 1H), 3.74 (m, 4H), 2.99 (m, 4H), 2.45 (s, 3H).

### Step C: Preparation of 5-(2,4-Difluorophenyl)-3,4-dihydro-3-imino-6-methyl-4-(2-methylbutyl)pyrazinecarbonitrile (Compound 426)

To a solution of [[2-(2,4-difluorophenyl)-1-methyl-2-(4-morpholinyl)ethenyl]imino]-propanedinitrile (i.e. the product of Example 14 Step B) (2.0 g, 6.3 mmol) in chloroform (20 mL) was added 2-methylbutylamine (0.87 mL, 7.6 mmol) at room temperature. The reaction mixture was allowed to stand overnight. The solvent was removed with a rotary evaporator. The residue was purified by MPLC (15→30% ethyl acetate in hexanes as eluant) to afford an impure sample of the title compound (0.87 g). This material was purified further by MPLC (20→30% ethyl acetate in hexanes as eluant) to afford 0.4 g of the title product, a compound of the present invention as a red oil.

¹H NMR (CDCl₃) δ 7.25 (m, 1H), 7.08 (dd, 1H), 7.02 (dd, 1H), 3.76 (br s, 1H), 3.60 (br s, 1H), 1.92 (m, 1H), 1.90 (s, 3H), 0.72 (m, 6H).

### Step D: Preparation of N-[3-Cyano-6-(2,4-difluorophenyl)-5-methyl-1-(2-methyl-butyl)-2(1H)-pyrazinylidene]acetamide (Compound 430)

5-(2,4-Difluorophenyl)-3,4-dihydro-3-imino-6-methyl-4-(2-methylbutyl)pyrazine-carbonitrile (i.e. the product of Example 14 Step C) (0.13 g, 0.41 mmol) was dissolved in acetic anhydride (2 mL). The reaction mixture was stirred at room temperature overnight. The reaction mixture was concentrated with a rotary evaporator. Diethyl ether was added and the organic layer was washed with 1 N sodium hydroxide aqueous solution. It was dried (NaSO₄), and concentrated with a rotary evaporator. The residue was purified by MPLC (30→50% ethyl acetate in hexanes as eluant) to afford 90 mg of the title product, a compound of the present invention as a viscous oil.

¹H NMR (CDCl₃) δ 7.25 (m, 1H), 7.14 (dd, 1H), 7.07 (dd, 1H), 3.96 (br s, 1H), 3.84 (br s, 1H), 2.31(s, 3H), 2.09 (s, 3H), 1.82 (m, 1H), 1.17 (m, 1H), 1.01 (m, 1H), 0.72 (m, 6H).

By the procedures described herein together with methods known in the art, the following compounds of Tables 1 to 6 can be prepared. The following abbreviations are used in the Tables which follow: t means tertiary, s means secondary, n means normal, i means iso, c means cyclo, Me means methyl, Et means ethyl, Pr means propyl, i-Pr means isopropyl, Bu means butyl, Hex means hexyl, Ph means phenyl, OMe means methoxy, OEt means ethoxy, SMe means methylthio, S(O) means sulfinyl, S(O)₂ means sulfonyl, CN means cyano, NO₂ means nitro, and 2-Cl-4-F means 2-chloro-4-fluoro, and other substituent abbreviations are defined analogously.

TABLE 1a

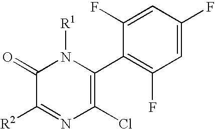

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3-dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH₂(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-Cl-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-1-yl |
| CH₂CH₂CH(Me)₂ | 1H-pyrazol-1-yl |
| CH₂-2-F-Ph | 1H-pyrazol-1-yl |
| CH₂-3-F-Ph | 1H-pyrazol-1-yl |
| CH₂-4-F-Ph | 1H-pyrazol-1-yl |
| CH₂-2-Me-Ph | 1H-pyrazol-1-yl |
| CH₂-3-Me-Ph | 1H-pyrazol-1-yl |
| CH₂-4-Me-Ph | 1H-pyrazol-1-yl |
| CH₂-2-OMe-Ph | 1H-pyrazol-1-yl |
| CH₂-3-OMe-Ph | 1H-pyrazol-1-yl |
| CH₂-4-OMe-Ph | 1H-pyrazol-1-yl |
| cis-2-Me-c-Hex | 1H-pyrazol-1-yl |
| trans-2-Me-c-Hex | 1H-pyrazol-1-yl |
| cis-3-Me-c-Hex | 1H-pyrazol-1-yl |
| trans-3-Me-c-Hex | 1H-pyrazol-1-yl |
| cis-4-Me-c-Hex | 1H-pyrazol-1-yl |
| trans-4-Me-c-Hex | 1H-pyrazol-1-yl |
| Me | 1H-1,2,4-triazol-1-yl |
| Et | 1H-1,2,4-triazol-1-yl |
| i-Pr | 1H-1,2,4-triazol-1-yl |
| n-Pr | 1H-1,2,4-triazol-1-yl |
| i-Bu | 1H-1,2,4-triazol-1-yl |
| n-Bu | 1H-1,2,4-triazol-1-yl |
| s-Bu | 1H-1,2,4-triazol-1-yl |
| 3-Me-Bu | 1H-1,2,4-triazol-1-yl |
| n-pentyl | 1H-1,2,4-triazol-1-yl |
| n-Hex | 1H-1,2,4-triazol-1-yl |
| 2-propenyl | 1H-1,2,4-triazol-1-yl |
| 2-Me-2-propenyl | 1H-1,2,4-triazol-1-yl |
| 3-butenyl | 1H-1,2,4-triazol-1-yl |
| 3-pentenyl | 1H-1,2,4-triazol-1-yl |
| 2-propynyl | 1H-1,2,4-triazol-1-yl |
| 3-butynyl | 1H-1,2,4-triazol-1-yl |
| 4-butynyl | 1H-1,2,4-triazol-1-yl |
| c-Pr | 1H-1,2,4-triazol-1-yl |
| c-pentyl | 1H-1,2,4-triazol-1-yl |
| c-Hex | 1H-1,2,4-triazol-1-yl |
| 2-cyclohexenyl | 1H-1,2,4-triazol-1-yl |
| 3-cyclohexenyl | 1H-1,2,4-triazol-1-yl |
| CH₂-c-Pr | 1H-1,2,4-triazol-1-yl |
| CH₂-c-Hex | 1H-1,2,4-triazol-1-yl |
| CH₂-2-cyclohexenyl | 1H-1,2,4-triazol-1-yl |
| 4-tetrahydropyranyl | 1H-1,2,4-triazol-1-yl |
| 3-tetrahydropyranyl | 1H-1,2,4-triazol-1-yl |
| 3-tetrahydrofuranyl | 1H-1,2,4-triazol-1-yl |
| Ph | 1H-1,2,4-triazol-1-yl |
| 2-Cl-phenyl | 1H-1,2,4-triazol-1-yl |
| 3-Cl-phenyl | 1H-1,2,4-triazol-1-yl |
| 4-Cl-phenyl | 1H-1,2,4-triazol-1-yl |
| 2-pyridinyl | 1H-1,2,4-triazol-1-yl |
| 2-pyrimidyl | 1H-1,2,4-triazol-1-yl |
| 2-pyrazinyl | 1H-1,2,4-triazol-1-yl |
| 2-thiazolyl | 1H-1,2,4-triazol-1-yl |
| 2-oxazolyl | 1H-1,2,4-triazol-1-yl |
| CF₃ | 1H-1,2,4-triazol-1-yl |
| CF₂CF₃ | 1H-1,2,4-triazol-1-yl |
| CH₂CF₃ | 1H-1,2,4-triazol-1-yl |
| CH(Me)CF₃ | 1H-1,2,4-triazol-1-yl |
| CH₂CH₂F | 1H-1,2,4-triazol-1-yl |
| CH₂CH₂CH₂F | 1H-1,2,4-triazol-1-yl |
| CH₂CF₂CF₃ | 1H-1,2,4-triazol-1-yl |
| CH₂CH₂CF₃ | 1H-1,2,4-triazol-1-yl |
| CH₂CH(Me)CF₃ | 1H-1,2,4-triazol-1-yl |
| (S)-CH₂CH(Me)CF₃ | 1H-1,2,4-triazol-1-yl |
| CH₂CH₂CH₂CH₂F | 1H-1,2,4-triazol-1-yl |
| 2-chloro-2-propenyl | 1H-1,2,4-triazol-1-yl |
| 3,3-dichloro-2-propenyl | 1H-1,2,4-triazol-1-yl |
| CH₂-2-tetrahydrofuranyl | 1H-1,2,4-triazol-1-yl |
| CH₂-2-tetrahydropyranyl | 1H-1,2,4-triazol-1-yl |
| CH₂CN | 1H-1,2,4-triazol-1-yl |
| CH₂NO₂ | 1H-1,2,4-triazol-1-yl |
| CH₂CH₂OH | 1H-1,2,4-triazol-1-yl |
| CH₂CH₂OMe | 1H-1,2,4-triazol-1-yl |
| CH₂CH(Me)OMe | 1H-1,2,4-triazol-1-yl |
| CH(Me)CH₂OMe | 1H-1,2,4-triazol-1-yl |
| CH(Me)CH(OMe)₂ | 1H-1,2,4-triazol-1-yl |
| CH₂-2-dioxolanyl | 1H-1,2,4-triazol-1-yl |
| CH₂CH₂OCF₃ | 1H-1,2,4-triazol-1-yl |
| CH₂CH₂SMe | 1H-1,2,4-triazol-1-yl |
| CH₂CH(Me)SMe | 1H-1,2,4-triazol-1-yl |
| CH₂CH₂S(O)Me | 1H-1,2,4-triazol-1-yl |
| CH₂CH₂S(O)₂Me | 1H-1,2,4-triazol-1-yl |
| CH₂CO₂Me | 1H-1,2,4-triazol-1-yl |
| CH₂CO₂-i-Pr | 1H-1,2,4-triazol-1-yl |
| CH(Me)CO₂Me | 1H-1,2,4-triazol-1-yl |
| CH₂C(O)Me | 1H-1,2,4-triazol-1-yl |
| CH₂CH₂C(O)Me | 1H-1,2,4-triazol-1-yl |
| CH₂SiMe₃ | 1H-1,2,4-triazol-1-yl |
| CH₂CH₂SiMe₃ | 1H-1,2,4-triazol-1-yl |
| CH₂OPh | 1H-1,2,4-triazol-1-yl |
| CH₂Ph | 1H-1,2,4-triazol-1-yl |
| CH₂CH₂Ph | 1H-1,2,4-triazol-1-yl |
| CH(Me)Ph | 1H-1,2,4-triazol-1-yl |
| CH₂-2-Cl-Ph | 1H-1,2,4-triazol-1-yl |
| CH₂-3-Cl-Ph | 1H-1,2,4-triazol-1-yl |
| CH₂-4-Cl-Ph | 1H-1,2,4-triazol-1-yl |
| CH₂-2-thienyl | 1H-1,2,4-triazol-1-yl |
| CH₂-2-pyridinyl | 1H-1,2,4-triazol-1-yl |
| CH₂-3-pyridinyl | 1H-1,2,4-triazol-1-yl |
| CH(Et)₂ | 1H-1,2,4-triazol-1-yl |
| CH₂CH(Et)₂ | 1H-1,2,4-triazol-1-yl |
| CH₂CH(n-Pr)Me | 1H-1,2,4-triazol-1-yl |
| CH(Me)Et | 1H-1,2,4-triazol-1-yl |
| CH(Me)-n-Pr | 1H-1,2,4-triazol-1-yl |
| CH(CF₃)Et | 1H-1,2,4-triazol-1-yl |
| CH(Et)-n-Pr | 1H-1,2,4-triazol-1-yl |
| CH(Me)-n-Bu | 1H-1,2,4-triazol-1-yl |
| 2,2-dimethylpropyl | 1H-1,2,4-triazol-1-yl |
| CH₂CH₂CH(Me)₂ | 1H-1,2,4-triazol-1-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3-dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-Cl-Ph | 2-pyridinyl |
| CH₂-3-Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| CH₂-2-F-Ph | 2-pyridinyl |
| CH₂-3-F-Ph | 2-pyridinyl |
| CH₂-4-F-Ph | 2-pyridinyl |
| CH₂-2-Me-Ph | 2-pyridinyl |
| CH₂-3-Me-Ph | 2-pyridinyl |
| CH₂-4-Me-Ph | 2-pyridinyl |
| CH₂-2-OMe-Ph | 2-pyridinyl |
| CH₂-3-OMe-Ph | 2-pyridinyl |
| CH₂-4-OMe-Ph | 2-pyridinyl |
| cis-2-Me-c-Hex | 2-pyridinyl |
| trans-2-Me-c-Hex | 2-pyridinyl |
| cis-3-Me-c-Hex | 2-pyridinyl |
| trans-3-Me-c-Hex | 2-pyridinyl |
| cis-4-Me-c-Hex | 2-pyridinyl |
| trans-4-Me-c-Hex | 2-pyridinyl |
| Me | CONH₂ |
| Et | CONH₂ |
| i-Pr | CONH₂ |
| n-Pr | CONH₂ |
| i-Bu | CONH₂ |
| n-Bu | CONH₂ |
| s-Bu | CONH₂ |
| 3-Me-Bu | CONH₂ |
| n-pentyl | CONH₂ |
| n-Hex | CONH₂ |
| 2-propenyl | CONH₂ |
| 2-Me-2-propenyl | CONH₂ |
| 3-butenyl | CONH₂ |
| 3-pentenyl | CONH₂ |
| 2-propynyl | CONH₂ |
| 3-butynyl | CONH₂ |
| 4-butynyl | CONH₂ |
| c-Pr | CONH₂ |
| c-pentyl | CONH₂ |
| c-Hex | CONH₂ |
| 2-cyclohexenyl | CONH₂ |
| 3-cyclohexenyl | CONH₂ |
| CH₂-c-Pr | CONH₂ |
| CH₂-c-Hex | CONH₂ |
| CH₂-2-cyclohexenyl | CONH₂ |
| 4-tetrahydropyranyl | CONH₂ |
| 3-tetrahydropyranyl | CONH₂ |
| 3-tetrahydrofuranyl | CONH₂ |
| Ph | CONH₂ |
| 2-Cl-phenyl | CONH₂ |
| 3-Cl-phenyl | CONH₂ |
| 4-Cl-phenyl | CONH₂ |
| 2-pyridinyl | CONH₂ |
| 2-pyrimidyl | CONH₂ |
| 2-pyrazinyl | CONH₂ |
| 2-thiazolyl | CONH₂ |
| 2-oxazolyl | CONH₂ |
| CF₃ | CONH₂ |
| CF₂CF₃ | CONH₂ |
| CH₂CF₃ | CONH₂ |
| CH(Me)CF₃ | CONH₂ |
| CH₂CH₂F | CONH₂ |
| CH₂CH₂CH₂F | CONH₂ |
| CH₂CF₂CF₃ | CONH₂ |
| CH₂CH₂CF₃ | CONH₂ |
| CH₂CH(Me)CF₃ | CONH₂ |
| (S)-CH₂CH(Me)CF₃ | CONH₂ |
| CH₂CH₂CH₂CH₂F | CONH₂ |
| 2-chloro-2-propenyl | CONH₂ |
| 3,3-dichloro-2-propenyl | CONH₂ |
| CH₂-2-tetrahydrofuranyl | CONH₂ |
| CH₂-2-tetrahydropyranyl | CONH₂ |
| CH₂CN | CONH₂ |
| CH₂NO₂ | CONH₂ |
| CH₂CH₂OH | CONH₂ |
| CH₂CH₂OMe | CONH₂ |
| CH₂CH(Me)OMe | CONH₂ |
| CH(Me)CH₂OMe | CONH₂ |
| CH(Me)CH(OMe)₂ | CONH₂ |
| CH₂-2-dioxolanyl | CONH₂ |
| CH₂CH₂OCF₃ | CONH₂ |
| CH₂CH₂SMe | CONH₂ |
| CH₂CH(Me)SMe | CONH₂ |
| CH₂CH₂S(O)Me | CONH₂ |
| CH₂CH₂S(O)₂Me | CONH₂ |
| CH₂CO₂Me | CONH₂ |
| CH₂CO₂-i-Pr | CONH₂ |
| CH(Me)CO₂Me | CONH₂ |
| CH₂C(O)Me | CONH₂ |
| CH₂CH₂C(O)Me | CONH₂ |
| CH₂SiMe₃ | CONH₂ |
| CH₂CH₂SiMe₃ | CONH₂ |
| CH₂OPh | CONH₂ |
| CH₂Ph | CONH₂ |
| CH₂CH₂Ph | CONH₂ |
| CH(Me)Ph | CONH₂ |
| CH₂-2-Cl-Ph | CONH₂ |
| CH₂-3-Cl-Ph | CONH₂ |
| CH₂-4-Cl-Ph | CONH₂ |
| CH₂-2-thienyl | CONH₂ |
| CH₂-2-pyridinyl | CONH₂ |
| CH₂-3-pyridinyl | CONH₂ |
| CH(Et)₂ | CONH₂ |
| CH₂CH(Et)₂ | CONH₂ |
| CH₂CH(n-Pr)Me | CONH₂ |
| CH(Me)Et | CONH₂ |
| CH(Me)-n-Pr | CONH₂ |
| CH(CF₃)Et | CONH₂ |
| CH(Et)-n-Pr | CONH₂ |
| CH(Me)-n-Bu | CONH₂ |
| 2,2-dimethylpropyl | CONH₂ |
| CH₂CH₂CH(Me)₂ | CONH₂ |

TABLE 1b

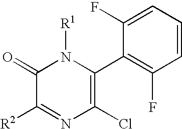

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| c-Hex | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| Ph | 1H-pyrazol-1-yl |
| 2-Cl-phenyl | 1H-pyrazol-1-yl |
| 3-Cl-phenyl | 1H-pyrazol-1-yl |
| 4-Cl-phenyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3-dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-Cl-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-1-yl |
| CH₂CH₂CH(Me)₂ | 1H-pyrazol-1-yl |
| Me | 1H-1,2,4-triazol-1-yl |
| Et | 1H-1,2,4-triazol-1-yl |
| i-Pr | 1H-1,2,4-triazol-1-yl |
| n-Pr | 1H-1,2,4-triazol-1-yl |
| i-Bu | 1H-1,2,4-triazol-1-yl |
| n-Bu | 1H-1,2,4-triazol-1-yl |
| s-Bu | 1H-1,2,4-triazol-1-yl |
| 3-Me-Bu | 1H-1,2,4-triazol-1-yl |
| n-pentyl | 1H-1,2,4-triazol-1-yl |
| n-Hex | 1H-1,2,4-triazol-1-yl |
| 2-propenyl | 1H-1,2,4-triazol-1-yl |
| 2-Me-2-propenyl | 1H-1,2,4-triazol-1-yl |
| 3-butenyl | 1H-1,2,4-triazol-1-yl |
| 3-pentenyl | 1H-1,2,4-triazol-1-yl |
| 2-propynyl | 1H-1,2,4-triazol-1-yl |
| 3-butynyl | 1H-1,2,4-triazol-1-yl |
| 4-butynyl | 1H-1,2,4-triazol-1-yl |
| c-Pr | 1H-1,2,4-triazol-1-yl |
| c-pentyl | 1H-1,2,4-triazol-1-yl |
| c-Hex | 1H-1,2,4-triazol-1-yl |
| 2-cyclohexenyl | 1H-1,2,4-triazol-1-yl |
| 3-cyclohexenyl | 1H-1,2,4-triazol-1-yl |
| CH₂-c-Pr | 1H-1,2,4-triazol-1-yl |
| CH₂-c-Hex | 1H-1,2,4-triazol-1-yl |
| CH₂-2-cyclohexenyl | 1H-1,2,4-triazol-1-yl |
| 4-tetrahydropyranyl | 1H-1,2,4-triazol-1-yl |
| 3-tetrahydropyranyl | 1H-1,2,4-triazol-1-yl |
| 3-tetrahydrofuranyl | 1H-1,2,4-triazol-1-yl |
| Ph | 1H-1,2,4-triazol-1-yl |
| 2-Cl-phenyl | 1H-1,2,4-triazol-1-yl |
| 3-Cl-phenyl | 1H-1,2,4-triazol-1-yl |
| 4-Cl-phenyl | 1H-1,2,4-triazol-1-yl |
| 2-pyridinyl | 1H-1,2,4-triazol-1-yl |
| 2-pyrimidyl | 1H-1,2,4-triazol-1-yl |
| 2-pyrazinyl | 1H-1,2,4-triazol-1-yl |
| 2-thiazolyl | 1H-1,2,4-triazol-1-yl |
| 2-oxazolyl | 1H-1,2,4-triazol-1-yl |
| CF₃ | 1H-1,2,4-triazol-1-yl |
| CF₂CF₃ | 1H-1,2,4-triazol-1-yl |
| CH₂CF₃ | 1H-1,2,4-triazol-1-yl |
| CH(Me)CF₃ | 1H-1,2,4-triazol-1-yl |
| CH₂CH₂F | 1H-1,2,4-triazol-1-yl |
| 2-chloro-2-propenyl | 1H-1,2,4-triazol-1-yl |
| 3,3-dichloro-2-propenyl | 1H-1,2,4-triazol-1-yl |
| CH₂-2-tetrahydrofuranyl | 1H-1,2,4-triazol-1-yl |
| CH₂-2-tetrahydropyranyl | 1H-1,2,4-triazol-1-yl |
| CH₂CN | 1H-1,2,4-triazol-1-yl |
| CH₂NO₂ | 1H-1,2,4-triazol-1-yl |
| CH₂CH₂OH | 1H-1,2,4-triazol-1-yl |
| CH₂CH₂OMe | 1H-1,2,4-triazol-1-yl |
| CH₂CH(Me)OMe | 1H-1,2,4-triazol-1-yl |
| CH(Me)CH₂OMe | 1H-1,2,4-triazol-1-yl |
| CH(Me)CH(OMe)₂ | 1H-1,2,4-triazol-1-yl |
| CH₂-2-dioxolanyl | 1H-1,2,4-triazol-1-yl |
| CH₂CH₂OCF₃ | 1H-1,2,4-triazol-1-yl |
| CH₂CH₂SMe | 1H-1,2,4-triazol-1-yl |
| CH₂CH(Me)SMe | 1H-1,2,4-triazol-1-yl |
| CH₂CH₂S(O)Me | 1H-1,2,4-triazol-1-yl |
| CH₂CH₂S(O)₂Me | 1H-1,2,4-triazol-1-yl |
| CH₂CO₂Me | 1H-1,2,4-triazol-1-yl |
| CH₂CO₂-i-Pr | 1H-1,2,4-triazol-1-yl |
| CH(Me)CO₂Me | 1H-1,2,4-triazol-1-yl |
| CH₂C(O)Me | 1H-1,2,4-triazol-1-yl |
| CH₂CH₂C(O)Me | 1H-1,2,4-triazol-1-yl |
| CH₂SiMe₃ | 1H-1,2,4-triazol-1-yl |
| CH₂CH₂SiMe₃ | 1H-1,2,4-triazol-1-yl |
| CH₂OPh | 1H-1,2,4-triazol-1-yl |
| CH₂Ph | 1H-1,2,4-triazol-1-yl |
| CH₂CH₂Ph | 1H-1,2,4-triazol-1-yl |
| CH(Me)Ph | 1H-1,2,4-triazol-1-yl |
| CH₂-2-Cl-Ph | 1H-1,2,4-triazol-1-yl |
| CH₂-3-Cl-Ph | 1H-1,2,4-triazol-1-yl |
| CH₂-4-Cl-Ph | 1H-1,2,4-triazol-1-yl |
| CH₂-2-thienyl | 1H-1,2,4-triazol-1-yl |
| CH₂-2-pyridinyl | 1H-1,2,4-triazol-1-yl |
| CH₂-3-pyridinyl | 1H-1,2,4-triazol-1-yl |
| CH(Et)₂ | 1H-1,2,4-triazol-1-yl |
| CH₂CH(Et)₂ | 1H-1,2,4-triazol-1-yl |
| CH₂CH(n-Pr)Me | 1H-1,2,4-triazol-1-yl |
| CH(Me)Et | 1H-1,2,4-triazol-1-yl |
| CH(Me)-n-Pr | 1H-1,2,4-triazol-1-yl |
| CH(CF₃)Et | 1H-1,2,4-triazol-1-yl |
| CH(Et)-n-Pr | 1H-1,2,4-triazol-1-yl |
| CH(Me)-n-Bu | 1H-1,2,4-triazol-1-yl |
| 2,2-dimethylpropyl | 1H-1,2,4-triazol-1-yl |
| CH₂CH₂CH(Me)₂ | 1H-1,2,4-triazol-1-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| c-Hex | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| Ph | 2-pyridinyl |
| 2-Cl-phenyl | 2-pyridinyl |
| 3-Cl-phenyl | 2-pyridinyl |
| 4-Cl-phenyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3-dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-Cl-Ph | 2-pyridinyl |
| CH₂-3-Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| Me | CONH₂ |
| Et | CONH₂ |
| i-Pr | CONH₂ |
| n-Pr | CONH₂ |
| i-Bu | CONH₂ |
| n-Bu | CONH₂ |
| s-Bu | CONH₂ |
| 3-Me-Bu | CONH₂ |
| n-pentyl | CONH₂ |
| n-Hex | CONH₂ |
| 2-propenyl | CONH₂ |
| 2-Me-2-propenyl | CONH₂ |
| 3-butenyl | CONH₂ |
| 3-pentenyl | CONH₂ |
| 2-propynyl | CONH₂ |
| 3-butynyl | CONH₂ |
| 4-butynyl | CONH₂ |
| c-Pr | CONH₂ |
| c-pentyl | CONH₂ |
| c-Hex | CONH₂ |
| 2-cyclohexenyl | CONH₂ |
| 3-cyclohexenyl | CONH₂ |
| CH₂-c-Pr | CONH₂ |
| CH₂-c-Hex | CONH₂ |
| CH₂-2-cyclohexenyl | CONH₂ |
| 4-tetrahydropyranyl | CONH₂ |
| 3-tetrahydropyranyl | CONH₂ |
| 3-tetrahydrofuranyl | CONH₂ |
| Ph | CONH₂ |
| 2-Cl-phenyl | CONH₂ |
| 3-Cl-phenyl | CONH₂ |
| 4-Cl-phenyl | CONH₂ |
| 2-pyridinyl | CONH₂ |
| 2-pyrimidyl | CONH₂ |
| 2-pyrazinyl | CONH₂ |
| 2-thiazolyl | CONH₂ |
| 2-oxazolyl | CONH₂ |
| CF₃ | CONH₂ |
| CF₂CF₃ | CONH₂ |
| CH₂CF₃ | CONH₂ |
| CH(Me)CF₃ | CONH₂ |
| CH₂CH₂F | CONH₂ |
| 2-chloro-2-propenyl | CONH₂ |
| 3,3-dichloro-2-propenyl | CONH₂ |
| CH₂-2-tetrahydrofuranyl | CONH₂ |
| CH₂-2-tetrahydropyranyl | CONH₂ |
| CH₂CN | CONH₂ |
| CH₂NO₂ | CONH₂ |
| CH₂CH₂OH | CONH₂ |
| CH₂CH₂OMe | CONH₂ |
| CH₂CH(Me)OMe | CONH₂ |
| CH(Me)CH₂OMe | CONH₂ |
| CH(Me)CH(OMe)₂ | CONH₂ |
| CH₂-2-dioxolanyl | CONH₂ |
| CH₂CH₂OCF₃ | CONH₂ |
| CH₂CH₂SMe | CONH₂ |
| CH₂CH(Me)SMe | CONH₂ |
| CH₂CH₂S(O)Me | CONH₂ |
| CH₂CH₂S(O)₂Me | CONH₂ |
| CH₂CO₂Me | CONH₂ |
| CH₂CO₂-i-Pr | CONH₂ |
| CH(Me)CO₂Me | CONH₂ |
| CH₂C(O)Me | CONH₂ |
| CH₂CH₂C(O)Me | CONH₂ |
| CH₂SiMe₃ | CONH₂ |
| CH₂CH₂SiMe₃ | CONH₂ |
| CH₂OPh | CONH₂ |
| CH₂Ph | CONH₂ |
| CH₂CH₂Ph | CONH₂ |
| CH(Me)Ph | CONH₂ |
| CH₂-2-Cl-Ph | CONH₂ |
| CH₂-3-Cl-Ph | CONH₂ |
| CH₂-4-Cl-Ph | CONH₂ |
| CH₂-2-thienyl | CONH₂ |
| CH₂-2-pyridinyl | CONH₂ |
| CH₂-3-pyridinyl | CONH₂ |
| CH(Et)₂ | CONH₂ |
| CH₂CH(Et)₂ | CONH₂ |
| CH₂CH(n-Pr)Me | CONH₂ |
| CH(Me)Et | CONH₂ |
| CH(Me)-n-Pr | CONH₂ |
| CH(CF₃)Et | CONH₂ |
| CH(Et)-n-Pr | CONH₂ |
| CH(Me)-n-Bu | CONH₂ |
| 2,2-dimethylpropyl | CONH₂ |
| CH₂CH₂CH(Me)₂ | CONH₂ |

TABLE 1c

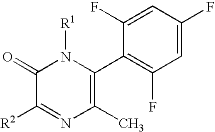

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| c-Hex | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| Ph | 1H-pyrazol-1-yl |
| 2-Cl-phenyl | 1H-pyrazol-1-yl |
| 3-Cl-phenyl | 1H-pyrazol-1-yl |
| 4-Cl-phenyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3-dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-Cl-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-1-yl |
| CH₂CH₂CH(Me)₂ | 1H-pyrazol-1-yl |
| CH₂-2-F-Ph | 1H-pyrazol-1-yl |
| CH₂-3-F-Ph | 1H-pyrazol-1-yl |
| CH₂-4-F-Ph | 1H-pyrazol-1-yl |
| CH₂-2-Me-Ph | 1H-pyrazol-1-yl |
| CH₂-3-Me-Ph | 1H-pyrazol-1-yl |
| CH₂-4-Me-Ph | 1H-pyrazol-1-yl |
| CH₂-2-OMe-Ph | 1H-pyrazol-1-yl |
| CH₂-3-OMe-Ph | 1H-pyrazol-1-yl |
| CH₂-4-OMe-Ph | 1H-pyrazol-1-yl |
| cis-2-Me-c-Hex | 1H-pyrazol-1-yl |
| trans-2-Me-c-Hex | 1H-pyrazol-1-yl |
| cis-3-Me-c-Hex | 1H-pyrazol-1-yl |
| trans-3-Me-c-Hex | 1H-pyrazol-1-yl |
| cis-4-Me-c-Hex | 1H-pyrazol-1-yl |
| trans-4-Me-c-Hex | 1H-pyrazol-1-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| c-Hex | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| Ph | 2-pyridinyl |
| 2-Cl-phenyl | 2-pyridinyl |
| 3-Cl-phenyl | 2-pyridinyl |
| 4-Cl-phenyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3-dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-Cl-Ph | 2-pyridinyl |
| CH₂-3-Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| CH₂-2-F-Ph | 2-pyridinyl |
| CH₂-3-F-Ph | 2-pyridinyl |
| CH₂-4-F-Ph | 2-pyridinyl |
| CH₂-2-Me-Ph | 2-pyridinyl |
| CH₂-3-Me-Ph | 2-pyridinyl |
| CH₂-4-Me-Ph | 2-pyridinyl |
| CH₂-2-OMe-Ph | 2-pyridinyl |
| CH₂-3-OMe-Ph | 2-pyridinyl |
| CH₂-4-OMe-Ph | 2-pyridinyl |
| cis-2-Me-c-Hex | 2-pyridinyl |
| trans-2-Me-c-Hex | 2-pyridinyl |
| cis-3-Me-c-Hex | 2-pyridinyl |
| trans-3-Me-c-Hex | 2-pyridinyl |
| cis-4-Me-c-Hex | 2-pyridinyl |
| trans-4-Me-c-Hex | 2-pyridinyl |

TABLE 1d

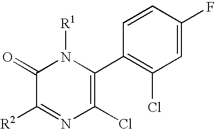

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| c-Hex | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| Ph | 1H-pyrazol-1-yl |
| 2-Cl-phenyl | 1H-pyrazol-1-yl |
| 3-Cl-phenyl | 1H-pyrazol-1-yl |
| 4-Cl-phenyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3-dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-Cl-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-1-yl |
| CH₂CH₂CH(Me)₂ | 1H-pyrazol-1-yl |
| CH₂-2-F-Ph | 1H-pyrazol-1-yl |
| CH₂-3-F-Ph | 1H-pyrazol-1-yl |
| CH₂-4-F-Ph | 1H-pyrazol-1-yl |
| CH₂-2-Me-Ph | 1H-pyrazol-1-yl |
| CH₂-3-Me-Ph | 1H-pyrazol-1-yl |
| CH₂-4-Me-Ph | 1H-pyrazol-1-yl |
| CH₂-2-OMe-Ph | 1H-pyrazol-1-yl |
| CH₂-3-OMe-Ph | 1H-pyrazol-1-yl |
| CH₂-4-OMe-Ph | 1H-pyrazol-1-yl |
| cis-2-Me-c-Hex | 1H-pyrazol-1-yl |
| trans-2-Me-c-Hex | 1H-pyrazol-1-yl |
| cis-3-Me-c-Hex | 1H-pyrazol-1-yl |
| trans-3-Me-c-Hex | 1H-pyrazol-1-yl |
| cis-4-Me-c-Hex | 1H-pyrazol-1-yl |
| trans-4-Me-c-Hex | 1H-pyrazol-1-yl |
| Me | 1H-1,2,4-triazol-1-yl |
| Et | 1H-1,2,4-triazol-1-yl |
| i-Pr | 1H-1,2,4-triazol-1-yl |
| n-Pr | 1H-1,2,4-triazol-1-yl |
| i-Bu | 1H-1,2,4-triazol-1-yl |
| n-Bu | 1H-1,2,4-triazol-1-yl |
| s-Bu | 1H-1,2,4-triazol-1-yl |
| 3-Me-Bu | 1H-1,2,4-triazol-1-yl |
| n-pentyl | 1H-1,2,4-triazol-1-yl |
| n-Hex | 1H-1,2,4-triazol-1-yl |
| 2-propenyl | 1H-1,2,4-triazol-1-yl |
| 2-Me-2-propenyl | 1H-1,2,4-triazol-1-yl |
| 3-butenyl | 1H-1,2,4-triazol-1-yl |
| 3-pentenyl | 1H-1,2,4-triazol-1-yl |
| 2-propynyl | 1H-1,2,4-triazol-1-yl |
| 3-butynyl | 1H-1,2,4-triazol-1-yl |
| 4-butynyl | 1H-1,2,4-triazol-1-yl |
| c-Pr | 1H-1,2,4-triazol-1-yl |
| c-pentyl | 1H-1,2,4-triazol-1-yl |
| c-Hex | 1H-1,2,4-triazol-1-yl |
| 2-cyclohexenyl | 1H-1,2,4-triazol-1-yl |
| 3-cyclohexenyl | 1H-1,2,4-triazol-1-yl |
| CH₂-c-Pr | 1H-1,2,4-triazol-1-yl |
| CH₂-c-Hex | 1H-1,2,4-triazol-1-yl |
| CH₂-2-cyclohexenyl | 1H-1,2,4-triazol-1-yl |
| 4-tetrahydropyranyl | 1H-1,2,4-triazol-1-yl |
| 3-tetrahydropyranyl | 1H-1,2,4-triazol-1-yl |
| 3-tetrahydrofuranyl | 1H-1,2,4-triazol-1-yl |
| Ph | 1H-1,2,4-triazol-1-yl |
| 2-Cl-phenyl | 1H-1,2,4-triazol-1-yl |
| 3-Cl-phenyl | 1H-1,2,4-triazol-1-yl |
| 4-Cl-phenyl | 1H-1,2,4-triazol-1-yl |
| 2-pyridinyl | 1H-1,2,4-triazol-1-yl |
| 2-pyrimidyl | 1H-1,2,4-triazol-1-yl |
| 2-pyrazinyl | 1H-1,2,4-triazol-1-yl |
| 2-thiazolyl | 1H-1,2,4-triazol-1-yl |
| 2-oxazolyl | 1H-1,2,4-triazol-1-yl |
| CF₃ | 1H-1,2,4-triazol-1-yl |
| CF₂CF₃ | 1H-1,2,4-triazol-1-yl |
| CH₂CF₃ | 1H-1,2,4-triazol-1-yl |
| CH(Me)CF₃ | 1H-1,2,4-triazol-1-yl |
| CH₂CH₂F | 1H-1,2,4-triazol-1-yl |
| CH₂CH₂CH₂F | 1H-1,2,4-triazol-1-yl |
| CH₂CF₂CF₃ | 1H-1,2,4-triazol-1-yl |
| CH₂CH₂CF₃ | 1H-1,2,4-triazol-1-yl |
| CH₂CH(Me)CF₃ | 1H-1,2,4-triazol-1-yl |
| (S)-CH₂CH(Me)CF₃ | 1H-1,2,4-triazol-1-yl |
| CH₂CH₂CH₂CH₂F | 1H-1,2,4-triazol-1-yl |
| 2-chloro-2-propenyl | 1H-1,2,4-triazol-1-yl |
| 3,3-dichloro-2-propenyl | 1H-1,2,4-triazol-1-yl |
| CH₂-2-tetrahydrofuranyl | 1H-1,2,4-triazol-1-yl |
| CH₂-2-tetrahydropyranyl | 1H-1,2,4-triazol-1-yl |
| CH₂CN | 1H-1,2,4-triazol-1-yl |
| CH₂NO₂ | 1H-1,2,4-triazol-1-yl |
| CH₂CH₂OH | 1H-1,2,4-triazol-1-yl |
| CH₂CH₂OMe | 1H-1,2,4-triazol-1-yl |
| CH₂CH(Me)OMe | 1H-1,2,4-triazol-1-yl |
| CH(Me)CH₂OMe | 1H-1,2,4-triazol-1-yl |
| CH(Me)CH(OMe)₂ | 1H-1,2,4-triazol-1-yl |
| CH₂-2-dioxolanyl | 1H-1,2,4-triazol-1-yl |
| CH₂CH₂OCF₃ | 1H-1,2,4-triazol-1-yl |
| CH₂CH₂SMe | 1H-1,2,4-triazol-1-yl |
| CH₂CH(Me)SMe | 1H-1,2,4-triazol-1-yl |
| CH₂CH₂S(O)Me | 1H-1,2,4-triazol-1-yl |
| CH₂CH₂S(O)₂Me | 1H-1,2,4-triazol-1-yl |
| CH₂CO₂Me | 1H-1,2,4-triazol-1-yl |
| CH₂CO₂-i-Pr | 1H-1,2,4-triazol-1-yl |
| CH(Me)CO₂Me | 1H-1,2,4-triazol-1-yl |
| CH₂C(O)Me | 1H-1,2,4-triazol-1-yl |
| CH₂CH₂C(O)Me | 1H-1,2,4-triazol-1-yl |
| CH₂SiMe₃ | 1H-1,2,4-triazol-1-yl |
| CH₂CH₂SiMe₃ | 1H-1,2,4-triazol-1-yl |
| CH₂OPh | 1H-1,2,4-triazol-1-yl |
| CH₂Ph | 1H-1,2,4-triazol-1-yl |
| CH₂CH₂Ph | 1H-1,2,4-triazol-1-yl |
| CH(Me)Ph | 1H-1,2,4-triazol-1-yl |
| CH₂-2-Cl-Ph | 1H-1,2,4-triazol-1-yl |
| CH₂-3-Cl-Ph | 1H-1,2,4-triazol-1-yl |
| CH₂-4-Cl-Ph | 1H-1,2,4-triazol-1-yl |
| CH₂-2-thienyl | 1H-1,2,4-triazol-1-yl |
| CH₂-2-pyridinyl | 1H-1,2,4-triazol-1-yl |
| CH₂-3-pyridinyl | 1H-1,2,4-triazol-1-yl |
| CH(Et)₂ | 1H-1,2,4-triazol-1-yl |
| CH₂CH(Et)₂ | 1H-1,2,4-triazol-1-yl |
| CH₂CH(n-Pr)Me | 1H-1,2,4-triazol-1-yl |
| CH(Me)Et | 1H-1,2,4-triazol-1-yl |
| CH(Me)-n-Pr | 1H-1,2,4-triazol-1-yl |
| CH(CF₃)Et | 1H-1,2,4-triazol-1-yl |
| CH(Et)-n-Pr | 1H-1,2,4-triazol-1-yl |
| CH(Me)-n-Bu | 1H-1,2,4-triazol-1-yl |
| 2,2-dimethylpropyl | 1H-1,2,4-triazol-1-yl |
| CH₂CH₂CH(Me)₂ | 1H-1,2,4-triazol-1-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| c-Hex | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| Ph | 2-pyridinyl |
| 2-Cl-phenyl | 2-pyridinyl |
| 3-Cl-phenyl | 2-pyridinyl |
| 4-Cl-phenyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3-dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-Cl-Ph | 2-pyridinyl |
| CH₂-3-Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| CH₂-2-F-Ph | 2-pyridinyl |
| CH₂-3-F-Ph | 2-pyridinyl |
| CH₂-4-F-Ph | 2-pyridinyl |
| CH₂-2-Me-Ph | 2-pyridinyl |
| CH₂-3-Me-Ph | 2-pyridinyl |
| CH₂-4-Me-Ph | 2-pyridinyl |
| CH₂-2-OMe-Ph | 2-pyridinyl |
| CH₂-3-OMe-Ph | 2-pyridinyl |
| CH₂-4-OMe-Ph | 2-pyridinyl |
| cis-2-Me-c-Hex | 2-pyridinyl |
| trans-2-Me-c-Hex | 2-pyridinyl |
| cis-3-Me-c-Hex | 2-pyridinyl |
| trans-3-Me-c-Hex | 2-pyridinyl |
| cis-4-Me-c-Hex | 2-pyridinyl |
| trans-4-Me-c-Hex | 2-pyridinyl |
| Me | CONH₂ |
| Et | CONH₂ |
| i-Pr | CONH₂ |
| n-Pr | CONH₂ |
| i-Bu | CONH₂ |
| n-Bu | CONH₂ |
| s-Bu | CONH₂ |
| 3-Me-Bu | CONH₂ |
| n-pentyl | CONH₂ |
| n-Hex | CONH₂ |
| 2-propenyl | CONH₂ |
| 2-Me-2-propenyl | CONH₂ |
| 3-butenyl | CONH₂ |
| 3-pentenyl | CONH₂ |
| 2-propynyl | CONH₂ |
| 3-butynyl | CONH₂ |
| 4-butynyl | CONH₂ |
| c-Pr | CONH₂ |
| c-pentyl | CONH₂ |
| c-Hex | CONH₂ |
| 2-cyclohexenyl | CONH₂ |
| 3-cyclohexenyl | CONH₂ |
| CH₂-c-Pr | CONH₂ |
| CH₂-c-Hex | CONH₂ |
| CH₂-2-cyclohexenyl | CONH₂ |
| 4-tetrahydropyranyl | CONH₂ |
| 3-tetrahydropyranyl | CONH₂ |
| 3-tetrahydrofuranyl | CONH₂ |
| Ph | CONH₂ |
| 2-Cl-phenyl | CONH₂ |
| 3-Cl-phenyl | CONH₂ |
| 4-Cl-phenyl | CONH₂ |
| 2-pyridinyl | CONH₂ |
| 2-pyrimidyl | CONH₂ |
| 2-pyrazinyl | CONH₂ |
| 2-thiazolyl | CONH₂ |
| 2-oxazolyl | CONH₂ |
| CF₃ | CONH₂ |
| CF₂CF₃ | CONH₂ |
| CH₂CF₃ | CONH₂ |
| CH(Me)CF₃ | CONH₂ |
| CH₂CH₂F | CONH₂ |
| CH₂CH₂CH₂F | CONH₂ |
| CH₂CF₂CF₃ | CONH₂ |
| CH₂CH₂CF₃ | CONH₂ |
| CH₂CH(Me)CF₃ | CONH₂ |
| (S)-CH₂CH(Me)CF₃ | CONH₂ |
| CH₂CH₂CH₂CH₂F | CONH₂ |
| 2-chloro-2-propenyl | CONH₂ |
| 3,3-dichloro-2-propenyl | CONH₂ |
| CH₂-2-tetrahydrofuranyl | CONH₂ |
| CH₂-2-tetrahydropyranyl | CONH₂ |
| CH₂CN | CONH₂ |
| CH₂NO₂ | CONH₂ |
| CH₂CH₂OH | CONH₂ |
| CH₂CH₂OMe | CONH₂ |
| CH₂CH(Me)OMe | CONH₂ |
| CH(Me)CH₂OMe | CONH₂ |
| CH(Me)CH(OMe)₂ | CONH₂ |
| CH₂-2-dioxolanyl | CONH₂ |
| CH₂CH₂OCF₃ | CONH₂ |
| CH₂CH₂SMe | CONH₂ |
| CH₂CH(Me)SMe | CONH₂ |
| CH₂CH₂S(O)Me | CONH₂ |
| CH₂CH₂S(O)₂Me | CONH₂ |
| CH₂CO₂Me | CONH₂ |
| CH₂CO₂-i-Pr | CONH₂ |
| CH(Me)CO₂Me | CONH₂ |
| CH₂C(O)Me | CONH₂ |
| CH₂CH₂C(O)Me | CONH₂ |
| CH₂SiMe₃ | CONH₂ |
| CH₂CH₂SiMe₃ | CONH₂ |
| CH₂OPh | CONH₂ |
| CH₂Ph | CONH₂ |
| CH₂CH₂Ph | CONH₂ |
| CH(Me)Ph | CONH₂ |
| CH₂-2-Cl-Ph | CONH₂ |
| CH₂-3-Cl-Ph | CONH₂ |
| CH₂-4-Cl-Ph | CONH₂ |
| CH₂-2-thienyl | CONH₂ |
| CH₂-2-pyridinyl | CONH₂ |
| CH₂-3-pyridinyl | CONH₂ |
| CH(Et)₂ | CONH₂ |
| CH₂CH(Et)₂ | CONH₂ |
| CH₂CH(n-Pr)Me | CONH₂ |
| CH(Me)Et | CONH₂ |
| CH(Me)-n-Pr | CONH₂ |
| CH(CF₃)Et | CONH₂ |
| CH(Et)-n-Pr | CONH₂ |
| CH(Me)-n-Bu | CONH₂ |
| 2,2-dimethylpropyl | CONH₂ |
| CH₂CH₂CH(Me)₂ | CONH₂ |

TABLE 1e

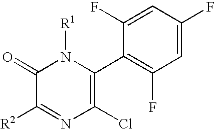

| R¹ | R² |
|---|---|
| 2-F-Ph | 1H-pyrazol-1-yl |
| 3-F-Ph | 1H-pyrazol-1-yl |
| 4-P-Ph | 1H-pyrazol-1-yl |
| 2,3-diF-Ph | 1H-pyrazol-1-yl |
| 2,4-diF-Ph | 1H-pyrazol-1-yl |
| 2,5-diF-Ph | 1H-pyrazol-1-yl |
| 2,6-diF-Ph | 1H-pyrazol-1-yl |
| 3,4-diF-Ph | 1H-pyrazol-1-yl |
| 3,5-diF-Ph | 1H-pyrazol-1-yl |
| 2,3-diCl-Ph | 1H-pyrazol-1-yl |
| 2,4-diCl-Ph | 1H-pyrazol-1-yl |
| 2,5-diCl-Ph | 1H-pyrazol-1-yl |
| 2,6-diCl-Ph | 1H-pyrazol-1-yl |
| 3,4-diCl-Ph | 1H-pyrazol-1-yl |
| 3,5-diCl-Ph | 1H-pyrazol-1-yl |
| 2-OMe-Ph | 1H-pyrazol-1-yl |
| 3-OMe-Ph | 1H-pyrazol-1-yl |
| 4-OMe-Ph | 1H-pyrazol-1-yl |
| 2-Me-Ph | 1H-pyrazol-1-yl |
| 3-Me-Ph | 1H-pyrazol-1-yl |
| 4-Me-Ph | 1H-pyrazol-1-yl |
| 2-CF₃-Ph | 1H-pyrazol-1-yl |
| 3-CF₃-Ph | 1H-pyrazol-1-yl |
| 4-CF₃-Ph | 1H-pyrazol-1-yl |
| 2-CN-Ph | 1H-pyrazol-1-yl |
| 3-CN-Ph | 1H-pyrazol-1-yl |
| 4-CN-Ph | 1H-pyrazol-1-yl |
| 2-NO₂-Ph | 1H-pyrazol-1-yl |
| 3-NO₂-Ph | 1H-pyrazol-1-yl |
| 4-NO₂-Ph | 1H-pyrazol-1-yl |
| 3-(CH═CH₂)-Ph | 1H-pyrazol-1-yl |
| 3-(CCH)-Ph | 1H-pyrazol-1-yl |
| 4-c-Pr-Ph | 1H-pyrazol-1-yl |
| 3-(CH═CCl₂)-Ph | 1H-pyrazol-1-yl |
| 3-(CCCl)-Ph | 1H-pyrazol-1-yl |
| 3-(2,2-diCl-c-Pr)-Ph | 1H-pyrazol-1-yl |
| 2-OCF₃-Ph | 1H-pyrazol-1-yl |
| 3-OCF₃-Ph | 1H-pyrazol-1-yl |
| 4-OCF₃-Ph | 1H-pyrazol-1-yl |
| 3-SMe-Ph | 1H-pyrazol-1-yl |
| 3-S(O)Me-Ph | 1H-pyrazol-1-yl |
| 3-SO₂Me-Ph | 1H-pyrazol-1-yl |
| 3-NHMe-Ph | 1H-pyrazol-1-yl |
| 3-NMe₂-Ph | 1H-pyrazol-1-yl |
| 3-NH-c-Pr-Ph | 1H-pyrazol-1-yl |
| 3-COMe-Ph | 1H-pyrazol-1-yl |
| 3-CO₂Me-Ph | 1H-pyrazol-1-yl |
| 3-CONHMe-Ph | 1H-pyrazol-1-yl |
| 3-CONMe₂-Ph | 1H-pyrazol-1-yl |
| 3-SiMe₃-Ph | 1H-pyrazol-1-yl |
| 2,3-diMe-Ph | 1H-pyrazol-1-yl |
| 2-F-Ph | 1H-1,2,4-triazol-1-yl |
| 3-F-Ph | 1H-1,2,4-triazol-1-yl |
| 4-P-Ph | 1H-1,2,4-triazol-1-yl |
| 2,3-diF-Ph | 1H-1,2,4-triazol-1-yl |
| 2,4-diF-Ph | 1H-1,2,4-triazol-1-yl |
| 2,5-diF-Ph | 1H-1,2,4-triazol-1-yl |
| 2,6-diF-Ph | 1H-1,2,4-triazol-1-yl |
| 3,4-diF-Ph | 1H-1,2,4-triazol-1-yl |
| 3,5-diF-Ph | 1H-1,2,4-triazol-1-yl |
| 2,3-diCl-Ph | 1H-1,2,4-triazol-1-yl |
| 2,4-diCl-Ph | 1H-1,2,4-triazol-1-yl |
| 2,5-diCl-Ph | 1H-1,2,4-triazol-1-yl |
| 2,6-diCl-Ph | 1H-1,2,4-triazol-1-yl |
| 3,4-diCl-Ph | 1H-1,2,4-triazol-1-yl |
| 3,5-diCl-Ph | 1H-1,2,4-triazol-1-yl |
| 2-OMe-Ph | 1H-1,2,4-triazol-1-yl |
| 3-OMe-Ph | 1H-1,2,4-triazol-1-yl |
| 4-OMe-Ph | 1H-1,2,4-triazol-1-yl |
| 2-Me-Ph | 1H-1,2,4-triazol-1-yl |
| 3-Me-Ph | 1H-1,2,4-triazol-1-yl |
| 4-Me-Ph | 1H-1,2,4-triazol-1-yl |
| 2-CF₃-Ph | 1H-1,2,4-triazol-1-yl |
| 3-CF₃-Ph | 1H-1,2,4-triazol-1-yl |
| 4-CF₃-Ph | 1H-1,2,4-triazol-1-yl |
| 2-CN-Ph | 1H-1,2,4-triazol-1-yl |
| 3-CN-Ph | 1H-1,2,4-triazol-1-yl |
| 4-CN-Ph | 1H-1,2,4-triazol-1-yl |
| 2-NO₂-Ph | 1H-1,2,4-triazol-1-yl |
| 3-NO₂-Ph | 1H-1,2,4-triazol-1-yl |
| 4-NO₂-Ph | 1H-1,2,4-triazol-1-yl |
| 3-(CH═CH₂)-Ph | 1H-1,2,4-triazol-1-yl |
| 3-(CCH)-Ph | 1H-1,2,4-triazol-1-yl |
| 4-c-Pr-Ph | 1H-1,2,4-triazol-1-yl |
| 3-(CH═CCl₂)-Ph | 1H-1,2,4-triazol-1-yl |
| 3-(CCCl)-Ph | 1H-1,2,4-triazol-1-yl |
| 3-(2,2-diCl-c-Pr)-Ph | 1H-1,2,4-triazol-1-yl |
| 2-OCF₃-Ph | 1H-1,2,4-triazol-1-yl |
| 3-OCF₃-Ph | 1H-1,2,4-triazol-1-yl |
| 4-OCF₃-Ph | 1H-1,2,4-triazol-1-yl |
| 3-SMe-Ph | 1H-1,2,4-triazol-1-yl |
| 3-S(O)Me-Ph | 1H-1,2,4-triazol-1-yl |
| 3-SO₂Me-Ph | 1H-1,2,4-triazol-1-yl |
| 3-NHMe-Ph | 1H-1,2,4-triazol-1-yl |
| 3-NMe₂-Ph | 1H-1,2,4-triazol-1-yl |
| 3-NH-c-Pr-Ph | 1H-1,2,4-triazol-1-yl |
| 3-COMe-Ph | 1H-1,2,4-triazol-1-yl |
| 3-CO₂Me-Ph | 1H-1,2,4-triazol-1-yl |
| 3-CONHMe-Ph | 1H-1,2,4-triazol-1-yl |
| 3-CONMe₂-Ph | 1H-1,2,4-triazol-1-yl |
| 3-SiMe₃-Ph | 1H-1,2,4-triazol-1-yl |
| 2,3-diMe-Ph | 1H-1,2,4-triazol-1-yl |
| 2-F-Ph | 2-pyridinyl |
| 3-F-Ph | 2-pyridinyl |
| 4-P-Ph | 2-pyridinyl |
| 2,3-diF-Ph | 2-pyridinyl |
| 2,4-diF-Ph | 2-pyridinyl |
| 2,5-diF-Ph | 2-pyridinyl |
| 2,6-diF-Ph | 2-pyridinyl |
| 3,4-diF-Ph | 2-pyridinyl |
| 3,5-diF-Ph | 2-pyridinyl |
| 2,3-diCl-Ph | 2-pyridinyl |
| 2,4-diCl-Ph | 2-pyridinyl |
| 2,5-diCl-Ph | 2-pyridinyl |
| 2,6-diCl-Ph | 2-pyridinyl |
| 3,4-diCl-Ph | 2-pyridinyl |
| 3,5-diCl-Ph | 2-pyridinyl |
| 2-OMe-Ph | 2-pyridinyl |
| 3-OMe-Ph | 2-pyridinyl |
| 4-OMe-Ph | 2-pyridinyl |
| 2-Me-Ph | 2-pyridinyl |
| 3-Me-Ph | 2-pyridinyl |
| 4-Me-Ph | 2-pyridinyl |
| 2-CF₃-Ph | 2-pyridinyl |
| 3-CF₃-Ph | 2-pyridinyl |
| 4-CF₃-Ph | 2-pyridinyl |
| 2-CN-Ph | 2-pyridinyl |
| 3-CN-Ph | 2-pyridinyl |
| 4-CN-Ph | 2-pyridinyl |
| 2-NO₂-Ph | 2-pyridinyl |
| 3-NO₂-Ph | 2-pyridinyl |
| 4-NO₂-Ph | 2-pyridinyl |
| 3-(CH═CH₂)-Ph | 2-pyridinyl |
| 3-(CCH)-Ph | 2-pyridinyl |
| 4-c-Pr-Ph | 2-pyridinyl |
| 3-(CH═CCl₂)-Ph | 2-pyridinyl |
| 3-(CCCl)-Ph | 2-pyridinyl |
| 3-(2,2-diCl-c-Pr)-Ph | 2-pyridinyl |
| 2-OCF₃-Ph | 2-pyridinyl |
| 3-OCF₃-Ph | 2-pyridinyl |
| 4-OCF₃-Ph | 2-pyridinyl |
| 3-SMe-Ph | 2-pyridinyl |
| 3-S(O)Me-Ph | 2-pyridinyl |
| 3-SO₂Me-Ph | 2-pyridinyl |
| 3-NHMe-Ph | 2-pyridinyl |
| 3-NMe₂-Ph | 2-pyridinyl |
| 3-NH-c-Pr-Ph | 2-pyridinyl |
| 3-COMe-Ph | 2-pyridinyl |
| 3-CO₂Me-Ph | 2-pyridinyl |
| 3-CONHMe-Ph | 2-pyridinyl |
| 3-CONMe₂-Ph | 2-pyridinyl |
| 3-SiMe₃-Ph | 2-pyridinyl |
| 2,3-diMe-Ph | 2-pyridinyl |
| 2-F-Ph | CONH₂ |
| 3-F-Ph | CONH₂ |
| 4-P-Ph | CONH₂ |
| 2,3-diF-Ph | CONH₂ |
| 2,4-diF-Ph | CONH₂ |
| 2,5-diF-Ph | CONH₂ |
| 2,6-diF-Ph | CONH₂ |
| 3,4-diF-Ph | CONH₂ |
| 3,5-diF-Ph | CONH₂ |
| 2,3-diCl-Ph | CONH₂ |
| 2,4-diCl-Ph | CONH₂ |
| 2,5-diCl-Ph | CONH₂ |
| 2,6-diCl-Ph | CONH₂ |
| 3,4-diCl-Ph | CONH₂ |
| 3,5-diCl-Ph | CONH₂ |
| 2-OMe-Ph | CONH₂ |
| 3-OMe-Ph | CONH₂ |
| 4-OMe-Ph | CONH₂ |
| 2-Me-Ph | CONH₂ |
| 3-Me-Ph | CONH₂ |
| 4-Me-Ph | CONH₂ |
| 2-CF₃-Ph | CONH₂ |
| 3-CF₃-Ph | CONH₂ |
| 4-CF₃-Ph | CONH₂ |
| 2-CN-Ph | CONH₂ |
| 3-CN-Ph | CONH₂ |
| 4-CN-Ph | CONH₂ |
| 2-NO₂-Ph | CONH₂ |
| 3-NO₂-Ph | CONH₂ |
| 4-NO₂-Ph | CONH₂ |
| 3-(CH═CH₂)-Ph | CONH₂ |
| 3-(CCH)-Ph | CONH₂ |
| 4-c-Pr-Ph | CONH₂ |
| 3-(CH═CCl₂)-Ph | CONH₂ |
| 3-(CCCl)-Ph | CONH₂ |
| 3-(2,2-diCl-c-Pr)-Ph | CONH₂ |
| 2-OCF₃-Ph | CONH₂ |
| 3-OCF₃-Ph | CONH₂ |
| 4-OCF₃-Ph | CONH₂ |
| 3-SMe-Ph | CONH₂ |
| 3-S(O)Me-Ph | CONH₂ |
| 3-SO₂Me-Ph | CONH₂ |
| 3-NHMe-Ph | CONH₂ |
| 3-NMe₂-Ph | CONH₂ |
| 3-NH-c-Pr-Ph | CONH₂ |
| 3-COMe-Ph | CONH₂ |
| 3-CO₂Me-Ph | CONH₂ |
| 3-CONHMe-Ph | CONH₂ |
| 3-CONMe₂-Ph | CONH₂ |
| 3-SiMe₃-Ph | CONH₂ |
| 2,3-diMe-Ph | CONH₂ |

TABLE 1f

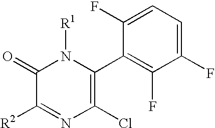

| R¹ | R² |
|---|---|
| 2-F-Ph | 1H-pyrazol-1-yl |
| 3-F-Ph | 1H-pyrazol-1-yl |
| 4-F-Ph | 1H-pyrazol-1-yl |
| 2,3-diF-Ph | 1H-pyrazol-1-yl |
| 2,4-diF-Ph | 1H-pyrazol-1-yl |
| 2,5-diF-Ph | 1H-pyrazol-1-yl |
| 2,6-diF-Ph | 1H-pyrazol-1-yl |
| 3,4-diF-Ph | 1H-pyrazol-1-yl |
| 3,5-diF-Ph | 1H-pyrazol-1-yl |
| 2,3-diCl-Ph | 1H-pyrazol-1-yl |
| 2,4-diCl-Ph | 1H-pyrazol-1-yl |
| 2,5-diCl-Ph | 1H-pyrazol-1-yl |
| 2,6-diCl-Ph | 1H-pyrazol-1-yl |
| 3,4-diCl-Ph | 1H-pyrazol-1-yl |
| 3,5-diCl-Ph | 1H-pyrazol-1-yl |
| 2-OMe-Ph | 1H-pyrazol-1-yl |
| 3-OMe-Ph | 1H-pyrazol-1-yl |
| 4-OMe-Ph | 1H-pyrazol-1-yl |
| 2-Me-Ph | 1H-pyrazol-1-yl |
| 3-Me-Ph | 1H-pyrazol-1-yl |
| 4-Me-Ph | 1H-pyrazol-1-yl |
| 2-CF₃-Ph | 1H-pyrazol-1-yl |
| 3-CF₃-Ph | 1H-pyrazol-1-yl |
| 4-CF₃-Ph | 1H-pyrazol-1-yl |
| 2-CN-Ph | 1H-pyrazol-1-yl |
| 3-CN-Ph | 1H-pyrazol-1-yl |
| 4-CN-Ph | 1H-pyrazol-1-yl |
| 2-NO₂-Ph | 1H-pyrazol-1-yl |
| 3-NO₂-Ph | 1H-pyrazol-1-yl |
| 4-NO₂-Ph | 1H-pyrazol-1-yl |
| 3-(CH═CH₂)-Ph | 1H-pyrazol-1-yl |
| 3-(CCH)-Ph | 1H-pyrazol-1-yl |
| 4-c-Pr-Ph | 1H-pyrazol-1-yl |
| 3-(CH═CCl₂)-Ph | 1H-pyrazol-1-yl |
| 3-(CCCl)-Ph | 1H-pyrazol-1-yl |
| 3-(2,2-diCl-c-Pr)-Ph | 1H-pyrazol-1-yl |
| 2-OCF₃-Ph | 1H-pyrazol-1-yl |
| 3-OCF₃-Ph | 1H-pyrazol-1-yl |
| 4-OCF₃-Ph | 1H-pyrazol-1-yl |
| 3-SMe-Ph | 1H-pyrazol-1-yl |
| 3-S(O)Me-Ph | 1H-pyrazol-1-yl |
| 3-SO₂Me-Ph | 1H-pyrazol-1-yl |
| 3-NHMe-Ph | 1H-pyrazol-1-yl |
| 3-NMe₂-Ph | 1H-pyrazol-1-yl |
| 3-NH-c-Pr-Ph | 1H-pyrazol-1-yl |
| 3-COMe-Ph | 1H-pyrazol-1-yl |
| 3-CO₂Me-Ph | 1H-pyrazol-1-yl |
| 3-CONHMe-Ph | 1H-pyrazol-1-yl |
| 3-CONMe₂-Ph | 1H-pyrazol-1-yl |
| 3-SiMe₃-Ph | 1H-pyrazol-1-yl |
| 2,3-diMe-Ph | 1H-pyrazol-1-yl |
| 2-F-Ph | 1H-1,2,4-triazol-1-yl |
| 3-F-Ph | 1H-1,2,4-triazol-1-yl |
| 4-F-Ph | 1H-1,2,4-triazol-1-yl |
| 2,3-diF-Ph | 1H-1,2,4-triazol-1-yl |
| 2,4-diF-Ph | 1H-1,2,4-triazol-1-yl |
| 2,5-diF-Ph | 1H-1,2,4-triazol-1-yl |
| 2,6-diF-Ph | 1H-1,2,4-triazol-1-yl |
| 3,4-diF-Ph | 1H-1,2,4-triazol-1-yl |
| 3,5-diF-Ph | 1H-1,2,4-triazol-1-yl |
| 2,3-diCl-Ph | 1H-1,2,4-triazol-1-yl |
| 2,4-diCl-Ph | 1H-1,2,4-triazol-1-yl |
| 2,5-diCl-Ph | 1H-1,2,4-triazol-1-yl |
| 2,6-diCl-Ph | 1H-1,2,4-triazol-1-yl |
| 3,4-diCl-Ph | 1H-1,2,4-triazol-1-yl |
| 3,5-diCl-Ph | 1H-1,2,4-triazol-1-yl |
| 2-OMe-Ph | 1H-1,2,4-triazol-1-yl |
| 3-OMe-Ph | 1H-1,2,4-triazol-1-yl |
| 4-OMe-Ph | 1H-1,2,4-triazol-1-yl |
| 2-Me-Ph | 1H-1,2,4-triazol-1-yl |
| 3-Me-Ph | 1H-1,2,4-triazol-1-yl |
| 4-Me-Ph | 1H-1,2,4-triazol-1-yl |
| 2-CF₃-Ph | 1H-1,2,4-triazol-1-yl |
| 3-CF₃-Ph | 1H-1,2,4-triazol-1-yl |
| 4-CF₃-Ph | 1H-1,2,4-triazol-1-yl |
| 2-CN-Ph | 1H-1,2,4-triazol-1-yl |
| 3-CN-Ph | 1H-1,2,4-triazol-1-yl |
| 4-CN-Ph | 1H-1,2,4-triazol-1-yl |
| 2-NO₂-Ph | 1H-1,2,4-triazol-1-yl |
| 3-NO₂-Ph | 1H-1,2,4-triazol-1-yl |
| 4-NO₂-Ph | 1H-1,2,4-triazol-1-yl |
| 3-(CH═CH₂)-Ph | 1H-1,2,4-triazol-1-yl |
| 3-(CCH)-Ph | 1H-1,2,4-triazol-1-yl |
| 4-c-Pr-Ph | 1H-1,2,4-triazol-1-yl |
| 3-(CH═CCl₂)-Ph | 1H-1,2,4-triazol-1-yl |
| 3-(CCCl)-Ph | 1H-1,2,4-triazol-1-yl |
| 3-(2,2-diCl-c-Pr)-Ph | 1H-1,2,4-triazol-1-yl |
| 2-OCF₃-Ph | 1H-1,2,4-triazol-1-yl |
| 3-OCF₃-Ph | 1H-1,2,4-triazol-1-yl |
| 4-OCF₃-Ph | 1H-1,2,4-triazol-1-yl |
| 3-SMe-Ph | 1H-1,2,4-triazol-1-yl |
| 3-S(O)Me-Ph | 1H-1,2,4-triazol-1-yl |
| 3-SO₂Me-Ph | 1H-1,2,4-triazol-1-yl |
| 3-NHMe-Ph | 1H-1,2,4-triazol-1-yl |
| 3-NMe₂-Ph | 1H-1,2,4-triazol-1-yl |
| 3-NH-c-Pr-Ph | 1H-1,2,4-triazol-1-yl |
| 3-COMe-Ph | 1H-1,2,4-triazol-1-yl |
| 3-CO₂Me-Ph | 1H-1,2,4-triazol-1-yl |
| 3-CONHMe-Ph | 1H-1,2,4-triazol-1-yl |
| 3-CONMe₂-Ph | 1H-1,2,4-triazol-1-yl |
| 3-SiMe₃-Ph | 1H-1,2,4-triazol-1-yl |
| 2,3-diMe-Ph | 1H-1,2,4-triazol-1-yl |
| 2-F-Ph | 2-pyridinyl |
| 3-F-Ph | 2-pyridinyl |
| 4-F-Ph | 2-pyridinyl |
| 2,3-diF-Ph | 2-pyridinyl |
| 2,4-diF-Ph | 2-pyridinyl |
| 2,5-diF-Ph | 2-pyridinyl |
| 2,6-diF-Ph | 2-pyridinyl |
| 3,4-diF-Ph | 2-pyridinyl |
| 3,5-diF-Ph | 2-pyridinyl |
| 2,3-diCl-Ph | 2-pyridinyl |
| 2,4-diCl-Ph | 2-pyridinyl |
| 2,5-diCl-Ph | 2-pyridinyl |
| 2,6-diCl-Ph | 2-pyridinyl |
| 3,4-diCl-Ph | 2-pyridinyl |
| 3,5-diCl-Ph | 2-pyridinyl |
| 2-OMe-Ph | 2-pyridinyl |
| 3-OMe-Ph | 2-pyridinyl |
| 4-OMe-Ph | 2-pyridinyl |
| 2-Me-Ph | 2-pyridinyl |
| 3-Me-Ph | 2-pyridinyl |
| 4-Me-Ph | 2-pyridinyl |
| 2-CF₃-Ph | 2-pyridinyl |
| 3-CF₃-Ph | 2-pyridinyl |
| 4-CF₃-Ph | 2-pyridinyl |
| 2-CN-Ph | 2-pyridinyl |
| 3-CN-Ph | 2-pyridinyl |
| 4-CN-Ph | 2-pyridinyl |
| 2-NO₂-Ph | 2-pyridinyl |
| 3-NO₂-Ph | 2-pyridinyl |
| 4-NO₂-Ph | 2-pyridinyl |
| 3-(CH═CH₂)-Ph | 2-pyridinyl |
| 3-(CCH)-Ph | 2-pyridinyl |
| 4-c-Pr-Ph | 2-pyridinyl |
| 3-(CH═CCl₂)-Ph | 2-pyridinyl |
| 3-(CCCl)-Ph | 2-pyridinyl |
| 3-(2,2-diCl-c-Pr)-Ph | 2-pyridinyl |
| 2-OCF₃-Ph | 2-pyridinyl |
| 3-OCF₃-Ph | 2-pyridinyl |
| 4-OCF₃-Ph | 2-pyridinyl |
| 3-SMe-Ph | 2-pyridinyl |
| 3-S(O)Me-Ph | 2-pyridinyl |
| 3-SO₂Me-Ph | 2-pyridinyl |
| 3-NHMe-Ph | 2-pyridinyl |
| 3-NMe₂-Ph | 2-pyridinyl |
| 3-NH-c-Pr-Ph | 2-pyridinyl |
| 3-COMe-Ph | 2-pyridinyl |
| 3-CO₂Me-Ph | 2-pyridinyl |
| 3-CONHMe-Ph | 2-pyridinyl |
| 3-CONMe₂-Ph | 2-pyridinyl |
| 3-SiMe₃-Ph | 2-pyridinyl |
| 2,3-diMe-Ph | 2-pyridinyl |
| 2-F-Ph | CONH₂ |
| 3-F-Ph | CONH₂ |
| 4-F-Ph | CONH₂ |
| 2,3-diF-Ph | CONH₂ |
| 2,4-diF-Ph | CONH₂ |
| 2,5-diF-Ph | CONH₂ |
| 2,6-diF-Ph | CONH₂ |
| 3,4-diF-Ph | CONH₂ |
| 3,5-diF-Ph | CONH₂ |
| 2,3-diCl-Ph | CONH₂ |
| 2,4-diCl-Ph | CONH₂ |
| 2,5-diCl-Ph | CONH₂ |
| 2,6-diCl-Ph | CONH₂ |
| 3,4-diCl-Ph | CONH₂ |
| 3,5-diCl-Ph | CONH₂ |
| 2-OMe-Ph | CONH₂ |
| 3-OMe-Ph | CONH₂ |
| 4-OMe-Ph | CONH₂ |
| 2-Me-Ph | CONH₂ |
| 3-Me-Ph | CONH₂ |
| 4-Me-Ph | CONH₂ |
| 2-CF₃-Ph | CONH₂ |
| 3-CF₃-Ph | CONH₂ |
| 4-CF₃-Ph | CONH₂ |
| 2-CN-Ph | CONH₂ |
| 3-CN-Ph | CONH₂ |
| 4-CN-Ph | CONH₂ |
| 2-NO₂-Ph | CONH₂ |
| 3-NO₂-Ph | CONH₂ |
| 4-NO₂-Ph | CONH₂ |
| 3-(CH═CH₂)-Ph | CONH₂ |
| 3-(CCH)-Ph | CONH₂ |
| 4-c-Pr-Ph | CONH₂ |
| 3-(CH═CCl₂)-Ph | CONH₂ |
| 3-(CCCl)-Ph | CONH₂ |
| 3-(2,2-diCl-c-Pr)-Ph | CONH₂ |
| 2-OCF₃-Ph | CONH₂ |
| 3-OCF₃-Ph | CONH₂ |
| 4-OCF₃-Ph | CONH₂ |
| 3-SMe-Ph | CONH₂ |
| 3-S(O)Me-Ph | CONH₂ |
| 3-SO₂Me-Ph | CONH₂ |
| 3-NHMe-Ph | CONH₂ |
| 3-NMe₂-Ph | CONH₂ |
| 3-NH-c-Pr-Ph | CONH₂ |
| 3-COMe-Ph | CONH₂ |
| 3-CO₂Me-Ph | CONH₂ |
| 3-CONHMe-Ph | CONH₂ |
| 3-CONMe₂-Ph | CONH₂ |
| 3-SiMe₃-Ph | CONH₂ |
| 2,3-diMe-Ph | CONH₂ |

TABLE 2

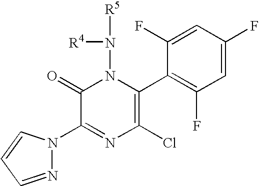

| R⁴ | R⁵ |
|---|---|
| Me | H |
| Et | H |
| i-Pr | H |
| n-Pr | H |
| i-Bu | H |
| n-Bu | H |
| s-Bu | H |
| 3-Me-Bu | H |
| n-pentyl | H |
| n-Hex | H |
| 2-propenyl | H |
| 2-Me-2-propenyl | H |
| 3-butenyl | H |
| 3-pentenyl | H |
| 2-propynyl | H |
| 3-butynyl | H |
| 4-butynyl | H |
| c-Pr | H |
| c-pentyl | H |
| c-Hex | H |
| 2-cyclohexenyl | H |
| 3-cyclohexenyl | H |
| CH₂-c-Pr | H |
| CH₂-c-Hex | H |
| CH₂-2-cyclohexenyl | H |
| 4-tetrahydropyranyl | H |
| 3-tetrahydropyranyl | H |
| 3-tetrahydrofuranyl | H |
| Ph | H |
| 2-Cl-phenyl | H |
| 3-Cl-phenyl | H |
| 4-Cl-phenyl | H |
| 2-pyridinyl | H |
| 2-pyrimidyl | H |
| 2-pyrazinyl | H |
| 2-thiazolyl | H |
| 2-oxazolyl | H |
| CF₃ | H |
| CF₂CF₃ | H |
| CH₂CF₃ | H |
| CH(Me)CF₃ | H |
| CH₂CH₂F | H |
| 2-chloro-2-propenyl | H |
| 3,3-dichloro-2-propenyl | H |
| CH₂-2-tetrahydrofuranyl | H |
| CH₂-2-tetrahydropyranyl | H |
| CH₂CN | H |
| CH₂NO₂ | H |
| CH₂CH₂OH | H |
| CH₂CH₂OMe | H |
| CH₂CH(Me)OMe | H |
| CH(Me)CH₂OMe | H |
| CH(Me)CH(OMe)₂ | H |
| CH₂-2-dioxolanyl | H |
| CH₂CH₂OCF₃ | H |
| CH₂CH₂SMe | H |
| CH₂CH(Me)SMe | H |
| CH₂CH₂S(O)Me | H |
| CH₂CH₂S(O)₂Me | H |
| CH₂CO₂Me | H |
| CH₂CO₂-i-Pr | H |
| CH(Me)CO₂Me | H |
| CH₂C(O)Me | H |
| CH₂CH₂C(O)Me | H |
| CH₂SiMe₃ | H |
| CH₂CH₂SiMe₃ | H |
| CH₂OPh | H |
| CH₂Ph | H |
| CH₂CH₂Ph | H |
| CH(Me)Ph | H |
| CH₂-2-Cl-Ph | H |
| CH₂-3-Cl-Ph | H |
| CH₂-4-Cl-Ph | H |
| CH₂-2-thienyl | H |
| CH₂-2-pyridinyl | H |
| CH₂-3-pyridinyl | H |
| CH(Et)₂ | H |
| CH₂CH(Et)₂ | H |
| CH₂CH₂(n-Pr)Me | H |
| CH(Me)Et | H |
| CH(Me)-n-Pr | H |
| CH(CF₃)Et | H |
| CH(Et)-n-Pr | H |
| CH(Me)-n-Bu | H |
| 2,2-dimethylpropyl | H |
| CH₂CH₂CH(Me)₂ | H |
| Me | Me |
| Et | Me |
| i-Pr | Me |
| n-Pr | Me |
| i-Bu | Me |
| n-Bu | Me |
| s-Bu | Me |
| 3-Me-Bu | Me |
| n-pentyl | Me |
| n-Hex | Me |
| 2-propenyl | Me |
| 2-Me-2-propenyl | Me |
| 3-butenyl | Me |
| 3-pentenyl | Me |
| 2-propynyl | Me |
| 3-butynyl | Me |
| 4-butynyl | Me |
| c-Pr | Me |
| c-pentyl | Me |
| c-Hex | Me |
| 2-cyclohexenyl | Me |
| 3-cyclohexenyl | Me |
| CH₂-c-Pr | Me |
| CH₂-c-Hex | Me |
| CH₂-2-cyclohexenyl | Me |
| 4-tetrahydropyranyl | Me |
| 3-tetrahydropyranyl | Me |
| 3-tetrahydrofuranyl | Me |
| Ph | Me |
| 2-Cl-phenyl | Me |
| 3-Cl-phenyl | Me |
| 4-Cl-phenyl | Me |
| 2-pyridinyl | Me |
| 2-pyrimidyl | Me |
| 2-pyrazinyl | Me |
| 2-thiazolyl | Me |
| 2-oxazolyl | Me |
| CF₃ | Me |
| CF₂CF₃ | Me |
| CH₂CF₃ | Me |
| CH(Me)CF₃ | Me |
| CH₂CH₂F | Me |
| 2-chloro-2-propenyl | Me |
| 3,3-dichloro-2-propenyl | Me |
| CH₂-2-tetrahydrofuranyl | Me |
| CH₂-2-tetrahydropyranyl | Me |
| CH₂CN | Me |
| CH₂NO₂ | Me |
| CH₂CH₂OH | Me |
| CH₂CH₂OMe | Me |
| CH₂CH(Me)OMe | Me |
| CH(Me)CH₂OMe | Me |
| CH(Me)CH(OMe)₂ | Me |
| CH₂-2-dioxolanyl | Me |
| CH₂CH₂OCF₃ | Me |
| CH₂CH₂SMe | Me |
| CH₂CH(Me)SMe | Me |
| CH₂CH₂S(O)Me | Me |
| CH₂CH₂S(O)₂Me | Me |
| CH₂CO₂Me | Me |
| CH₂CO₂-i-Pr | Me |
| CH(Me)CO₂Me | Me |
| CH₂C(O)Me | Me |
| CH₂CH₂C(O)Me | Me |
| CH₂SiMe₃ | Me |
| CH₂CH₂SiMe₃ | Me |
| CH₂OPh | Me |
| CH₂Ph | Me |
| CH₂CH₂Ph | Me |
| CH(Me)Ph | Me |
| CH₂-2-Cl-Ph | Me |
| CH₂-3-Cl-Ph | Me |
| CH₂-4-Cl-Ph | Me |
| CH₂-2-thienyl | Me |
| CH₂-2-pyridinyl | Me |
| CH₂-3-pyridinyl | Me |
| CH(Et)₂ | Me |
| CH₂CH(Et)₂ | Me |
| CH₂CH(n-Pr)Me | Me |
| CH(Me)Et | Me |
| CH(Me)-n-Pr | Me |
| CH(CF₃)Et | Me |
| CH(Et)-n-Pr | Me |
| CH(Me)-n-Bu | Me |
| 2,2-dimethylpropyl | Me |
| CH₂CH₂CH(Me)₂ | Me |

TABLE 3a

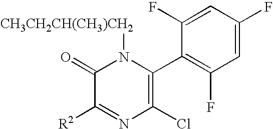

R²
3-Cl-2-pyridinyl
5-Cl-2-pyridinyl
6-Cl-2-pyridinyl
2-pyrimidinyl
5-Cl-2-pyrimidinyl
4-Cl-2-pyrimidinyl
2-thiazolyl
4-thiazolyl
2-oxazolyl
4-oxazolyl
3-Me-2-pyridinyl
5-Me-2-pyridinyl
6-Me-2-pyridinyl
4-Me-2-pyrimidinyl
4-pyrimidinyl
2-Me-4-pyrimidinyl
2,6-di-Me-4-pyrimidinyl
2-pyrazinyl
6-Cl-2-pyrazinyl
3-Cl-2-pyrazinyl
3-pyridazinyl
6-Cl-3-pyridazinyl
6-Me-3-pyridazinyl
4-OMe-2-pyrimidyl
2-Cl-4-pyrimidinyl
3-Me-2-pyrazinyl
1,2,4-triazin-3-yl
1,2,4-triazin-5-yl
4,6-di-Cl-1,3,5-triazin-2-yl
2-benzothiazolyl
2-benzoxazolyl
2-quinolinyl
4-Me-2-quinolinyl
2-quinoxalinyl
1,2,4-benzotriazin-3-yl
N-Me-2-benzimidazolyl
1-isoquinolinyl
3-isoxazolyl
3-isothiazolyl
CONHMe
CONHEt
CONH-n-Pr
NHCHO
NHCO₂Me
2-cinnolinyl
1,8-naphthyridin-2-yl
4-Me-2-quinazolinyl
2-Me-4-quinazolinyl
2-Cl-4-quinazolinyl
6-Cl-2-quinoxalinyl
7-Cl-2-quinoxalinyl
CONH-i-Pr
CONH-c-Pr
CONMe₂
CONEt₂
6,7-di-Cl-2-quinoxalinyl
6-Cl-2-benzothiazolyl
6-NO₂-2-benzothiazolyl
1-Me-1H-imidazol-2-yl
2-Me-1H-1,2,3-triazol-4-yl
1,2,3-oxadiazol-4-yl
1,2,3-thiadiazol-4-yl
1,3,4-thiadiazol-2-yl
3-Cl-1,2,4-thiadiazol-5-yl
3-Me-1,2,4-thiadiazol-5-yl
3-Me-1,2,4-oxadiazol-5-yl
1,3,4-oxadiazol-2-yl
3-Me-1H-pyrazol-1-yl
3-CF₃-1H-pyrazol-1-yl
3-t-Bu-1H-pyrazol-1-yl
3-Br-1H-pyrazol-1-yl
3-Ph-1H-pyrazol-1-yl
3-CN-1H-pyrazol-1-yl
4-CN-1H-pyrazol-1-yl
4-Me-1H-pyrazol-1-yl
4-Ph-1H-pyrazol-1-yl
4-Cl-1H-pyrazol-1-yl
4-Br-1H-pyrazol-1-yl
4-Ph-1H-pyrazol-1-yl
5-Me-1H-pyrazol-1-yl
3,5-di-Me-1H-pyrazol-1-yl
3-CF₃-5-Me-1H-pyrazol-1-yl
3,4,5-tri-Me-1H-pyrazol-1-yl
1H-pyrazolin-2-yl
3-Me-1H-pyrazolin-2-yl
3-Ph-1H-pyrazolin-2-yl
NHCOMe
NHCO₂Et
1H-1,2,4-triazol-1-yl
3-Me-1H-1,2,4-triazol-1-yl
3,5-di-Me-1H-1,2,4-triazol-1-yl
3-SMe-1H-1,2,4-triazol-1-yl
3-Br-1H-1,2,4-triazol-1-yl
3-Cl-1H-1,2,4-triazol-1-yl
1H-1,2,3-triazol-1-yl
4-Me-1H-pyrazolin-2-yl
CONHCH₂CH₂OMe
CONHCH₂CH₂SMe
CONHCH₂CH₂NMe₂
CONHCH₂CCH
CONHCH₂C═CH₂
CONHCH₂CH₂S(O)Me
CONHCH₂CH₂SO₂Me
1H-1,2,3-triazol-2-yl
4,5-di-Br-1H-1,2,3-triazol-1-yl
4,5-di-Br-1H-1,2,3-triazol-1-yl
4,5-di-Me-1H-1,2,3-triazol-1-yl
4,5-di-Me-1H-1,2,3-triazol-1-yl
3-CF₃-1H-1,2,4-triazol-1-yl
NHN═C(Me)₂
NHN═C(CH₂)₄
NHN═C(CH₂)₅
ON═C(Me)₂
ON═C(CH₂)₅
ON═C(CH₂)₄
NHNMe₂
ONMe₂
NHN(CH₂)₅
NHN(CH₂CH₂OCH₂CH₂)
C(S)NH₂
C(Me)═NNHMe
C(Me)═N-1-piperidino
C(Me)═N—OH
C(Me)═N—OMe
C(Me)═NO-i-Pr
CONHCH₂CF₃
CONHCH₂CN
CONH₂CH₂CO₂Me
CONHCH₂SiMe₃
CON(CH₂)₅
NHCOEt
NHCONHMe

TABLE 3b

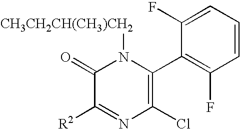

R²
3-Cl-2-pyridinyl
5-Cl-2-pyridinyl
6-Cl-2-pyridinyl
2-pyrimidinyl
5-Cl-2-pyrimidinyl
4-Cl-2-pyrimidinyl
2-thiazolyl
4-thiazolyl
2-oxazolyl
4-oxazolyl
3-Me-2-pyridinyl
5-Me-2-pyridinyl
6-Me-2-pyridinyl
4-Me-2-pyrimidinyl
4-pyrimidinyl
2-Me-4-pyrimidinyl
2,6-di-Me-4-pyrimidinyl
2-pyrazinyl
6-Cl-2-pyrazinyl
3-Cl-2-pyrazinyl
3-pyridazinyl
6-Cl-3-pyridazinyl
6-Me-3-pyridazinyl
4-OMe-2-pyrimidyl
2-Cl-4-pyrimidinyl
3-Me-2-pyrazinyl
1,2,4-triazin-3-yl
1,2,4-triazin-5-yl
4,6-di-Cl-1,3,5-triazin-2-yl
2-benzothiazolyl
2-benzoxazolyl
2-quinolinyl
4-Me-2-quinolinyl
2-quinoxalinyl
1,2,4-benzotriazin-3-yl
N-Me-2-benzimidazolyl
1-isoquinolinyl
3-isoxazolyl
3-isothiazolyl
CONHMe
CONHEt
CONH-n-Pr
NHCHO
NHCO₂Me
2-cinnolinyl
1,8-naphthyridin-2-yl
4-Me-2-quinazolinyl
2-Me-4-quinazolinyl
2-Cl-4-quinazolinyl
6-Cl-2-quinoxalinyl
7-Cl-2-quinoxalinyl
CONH-i-Pr
CONH-c-Pr
CONMe₂
CONEt₂
6,7-di-Cl-2-quinoxalinyl
6-Cl-2-benzothiazolyl
6-NO₂-2-benzothiazolyl
1-Me-1H-imidazol-2-yl
2-Me-1H-1,2,3-triazol-4-yl
1,2,3-oxadiazol-4-yl
1,2,3-thiadiazol-4-yl
1,3,4-thiadiazol-2-yl
3-Cl-1,2,4-thiadiazol-5-yl
3-Me-1,2,4-thiadiazol-5-yl
3-Me-1,2,4-oxadiazol-5-yl
1,3,4-oxadiazol-2-yl
3-Me-1H-pyrazol-1-yl
3-CF₃-1H-pyrazol-1-yl
3-t-Bu-1H-pyrazol-1-yl
3-Br-1H-pyrazol-1-yl
3-Ph-1H-pyrazol-1-yl
3-CN-1H-pyrazol-1-yl
4-CN-1H-pyrazol-1-yl
4-Me-1H-pyrazol-1-yl
4-Ph-1H-pyrazol-1-yl
4-Cl-1H-pyrazol-1-yl
4-Br-1H-pyrazol-1-yl
4-Ph-1H-pyrazol-1-yl
5-Me-1H-pyrazol-1-yl
3,5-di-Me-1H-pyrazol-1-yl
3-CF₃-5-Me-1H-pyrazol-1-yl
3,4,5-tri-Me-1H-pyrazol-1-yl
1H-pyrazolin-2-yl
3-Me-1H-pyrazolin-2-yl
3-Ph-1H-pyrazolin-2-yl
NHCOMe
NHCO₂Et
1H-1,2,4-triazol-1-yl
3-Me-1H-1,2,4-triazol-1-yl
3,5-di-Me-1H-1,2,4-triazol-1-yl
3-SMe-1H-1,2,4-triazol-1-yl
3-Br-1H-1,2,4-triazol-1-yl
3-Cl-1H-1,2,4-triazol-1-yl
1H-1,2,3-triazol-1-yl
4-Me-1H-pyrazolin-2-yl
CONHCH₂CH₂OMe
CONHCH₂CH₂SMe
CONHCH₂CH₂NMe₂
CONHCH₂CCH
CONHCH₂C═CH₂
CONHCH₂CH₂S(O)Me
CONHCH₂CH₂SO₂Me
1H-1,2,3-triazol-2-yl
4,5-di-Br-1H-1,2,3-triazol-1-yl
4,5-di-Br-1H-1,2,3-triazol-1-yl
4,5-di-Me-1H-1,2,3-triazol-1-yl
4,5-di-Me-1H-1,2,3-triazol-1-yl
3-CF₃-1H-1,2,4-triazol-1-yl
NHN═C(Me)₂
NHN═C(CH₂)₄
NHN═C(CH₂)₅
ON═C(Me)₂
ON═C(CH₂)₅
ON═C(CH₂)₄
NHNMe₂
ONMe₂
NHN(CH₂)₅
NHN(CH₂CH₂OCH₂CH₂)
C(S)NH₂
C(Me)═NNHMe
C(Me)═N-1-piperidino
C(Me)═N—OH
C(Me)═N—OMe
C(Me)═NO-i-Pr
CONHCH₂CF₃
CONHCH₂CN
CONHCH₂CO₂Me
CONHCH₂SiMe₃
CON(CH₂)₅
NHCOEt
NHCONHMe

TABLE 3c

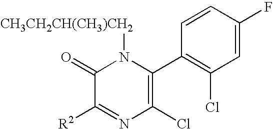

R²
3-Cl-2-pyridinyl
5-Cl-2-pyridinyl
6-Cl-2-pyridinyl
2-pyrimidinyl
5-Cl-2-pyrimidinyl
4-Cl-2-pyrimidinyl
2-thiazolyl
4-thiazolyl
2-oxazolyl
4-oxazolyl
3-Me-2-pyridinyl
5-Me-2-pyridinyl
6-Me-2-pyridinyl
4-Me-2-pyrimidinyl
4-pyrimidinyl
2-Me-4-pyrimidinyl
2,6-di-Me-4-pyrimidinyl
2-pyrazinyl
6-Cl-2-pyrazinyl
3-Cl-2-pyrazinyl
3-pyridazinyl
6-Cl-3-pyridazinyl
6-Me-3-pyridazinyl
4-OMe-2-pyrimidyl
2-Cl-4-pyrimidinyl
3-Me-2-pyrazinyl
1,2,4-triazin-3-yl
1,2,4-triazin-5-yl
4,6-di-Cl-1,3,5-triazin-2-yl
2-benzothiazolyl
2-benzoxazolyl
2-quinolinyl
4-Me-2-quinolinyl
2-quinoxalinyl
1,2,4-benzotriazin-3-yl
N-Me-2-benzimidazolyl
1-isoquinolinyl
3-isoxazolyl
3-isothiazolyl
CONHMe
CONHEt
CONH-n-Pr
NHCHO
NHCO₂Me
2-cinnolinyl
1,8-naphthyridin-2-yl
4-Me-2-quinazolinyl
2-Me-4-quinazolinyl
2-Cl-4-quinazolinyl
6-Cl-2-quinoxalinyl
7-Cl-2-quinoxalinyl
CONH-i-Pr
CONH-c-Pr
CONMe₂
CONEt₂
6,7-di-Cl-2-quinoxalinyl
6-Cl-2-benzothiazolyl
6-NO₂-2-benzothiazolyl
1-Me-1H-imidazol-2-yl
2-Me-1H-1,2,3-triazol-4-yl
1,2,3-oxadiazol-4-yl
1,2,3-thiadiazol-4-yl
1,3,4-thiadiazol-2-yl
3-Cl-1,2,4-thiadiazol-5-yl
3-Me-1,2,4-thiadiazol-5-yl
3-Me-1,2,4-oxadiazol-5-yl
1,3,4-oxadiazol-2-yl
3-Me-1H-pyrazol-1-yl
3-CF₃-1H-pyrazol-1-yl
3-t-Bu-1H-pyrazol-1-yl
3-Br-1H-pyrazol-1-yl
3-Ph-1H-pyrazol-1-yl
3-CN-1H-pyrazol-1-yl
4-CN-1H-pyrazol-1-yl
4-Me-1H-pyrazol-1-yl
4-Ph-1H-pyrazol-1-yl
4-Cl-1H-pyrazol-1-yl
4-Br-1H-pyrazol-1-yl
4-Ph-1H-pyrazol-1-yl
5-Me-1H-pyrazol-1-yl
3,5-di-Me-1H-pyrazol-1-yl
3-CF₃-5-Me-1H-pyrazol-1-yl
3,4,5-tri-Me-1H-pyrazol-1-yl
1H-pyrazolin-2-yl
3-Me-1H-pyrazolin-2-yl
3-Ph-1H-pyrazolin-2-yl
NHCOMe
NHCO₂Et
1H-1,2,4-triazol-1-yl
3-Me-1H-1,2,4-triazol-1-yl
3,5-di-Me-1H-1,2,4-triazol-1-yl
3-SMe-1H-1,2,4-triazol-1-yl
3-Br-1H-1,2,4-triazol-1-yl
3-Cl-1H-1,2,4-triazol-1-yl
1H-1,2,3-triazol-1-yl
4-Me-1H-pyrazolin-2-yl
CONHCH₂CH₂OMe
CONHCH₂CH₂SMe
CONHCH₂CH₂NMe₂
CONHCH₂CCH
CONHCH₂C═CH₂
CONHCH₂CH₂S(O)Me
CONHCH₂CH₂SO₂Me
1H-1,2,3-triazol-2-y1
4,5-di-Br-1H-1,2,3-triazol-1-yl
4,5-di-Br-1H-1,2,3-triazol-1-yl
4,5-di-Me-1H-1,2,3-triazol-1-yl
4,5-di-Me-1H-1,2,3-triazol-1-yl
3-CF₃-1H-1,2,4-triazol-1-yl
NHN═C(Me)₂
NHN═C(CH₂)₄
NHN═C(CH₂)₅
ON═C(Me)₂
ON═C(CH₂)₅
ON═C(CH₂)₄
NHNMe₂
ONMe₂
NHN(CH₂)₅
NHN(CH₂CH₂OCH₂CH₂)
C(S)NH₂
C(Me)═NNHMe
C(Me)═N-1-piperidino
C(Me)═N—OH
C(Me)═N—OMe
C(Me)═NO-i-Pr
CONHCH₂CF₃
CONHCH₂CN
CONHCH₂CO₂Me
CONHCH₂SiMe₃
CON(CH₂)₅
NHCOEt
NHCONHMe

TABLE 4a

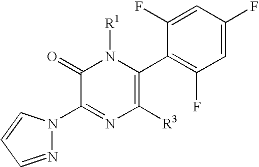

| R¹ | R³ |
|---|---|
| 2-Me-Bu | Cl |
| 2-Me-Bu | F |
| 2-Me-Bu | Br |
| 2-Me-Bu | Me |
| 2-Me-Bu | Et |
| 2-Me-Bu | c-Pr |
| 2-Me-Bu | CF₃ |
| 2-Me-Bu | OMe |
| 2-Me-Bu | SMe |
| 2-Me-Bu | SCF₃ |
| 2-Me-Bu | OCF₂H |
| 2-Me-Bu | CO₂Me |
| 2-Me-Bu | ethenyl |
| 2-Me-Bu | ethynyl |
| 2-Me-Bu | 2,2-di-Cl-c-Pr |
| NH-i-Pr | Cl |
| NH-i-Pr | F |
| NH-i-Pr | Br |
| NH-i-Pr | Me |
| NH-i-Pr | Et |
| NH-i-Pr | c-Pr |
| NH-i-Pr | CF₃ |
| NH-i-Pr | OMe |
| NH-i-Pr | SMe |
| NH-i-Pr | SCF₃ |
| NH-i-Pr | OCF₂H |
| NH-i-Pr | CO₂Me |
| NH-i-Pr | ethenyl |
| NH-i-Pr | ethynyl |
| NH-i-Pr | 2,2-di-Cl-c-Pr |
| i-Bu | Cl |
| i-Bu | F |
| i-Bu | Br |
| i-Bu | Me |
| i-Bu | Et |
| i-Bu | c-Pr |
| i-Bu | CF₃ |
| i-Bu | OMe |
| i-Bu | SMe |
| i-Bu | SCF₃ |
| i-Bu | OCF₂H |
| i-Bu | CO₂Me |
| i-Bu | ethenyl |
| i-Bu | ethynyl |
| i-Bu | 2,2-di-Cl-c-Pr |
| 3-F-Ph | Cl |
| 3-F-Ph | F |
| 3-F-Ph | Br |
| 3-F-Ph | Me |
| 3-F-Ph | Et |
| 3-F-Ph | c-Pr |
| 3-F-Ph | CF₃ |
| 3-F-Ph | OMe |
| 3-F-Ph | SMe |
| 3-F-Ph | SCF₃ |
| 3-F-Ph | OCF₂H |
| 3-F-Ph | CO₂Me |
| 3-F-Ph | ethenyl |
| 3-F-Ph | ethynyl |
| 3-F-Ph | 2,2-di-Cl-c-Pr |

TABLE 4b

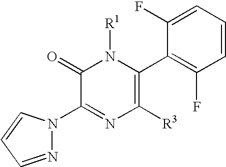

| R¹ | R³ |
|---|---|
| 2-Me-Bu | Cl |
| 2-Me-Bu | F |
| 2-Me-Bu | Br |
| 2-Me-Bu | Me |
| 2-Me-Bu | Et |
| 2-Me-Bu | c-Pr |
| 2-Me-Bu | CF₃ |
| 2-Me-Bu | OMe |
| 2-Me-Bu | SMe |
| 2-Me-Bu | SCF₃ |
| 2-Me-Bu | OCF₂H |
| 2-Me-Bu | CO₂Me |
| 2-Me-Bu | ethenyl |
| 2-Me-Bu | ethynyl |
| 2-Me-Bu | 2,2-di-Cl-c-Pr |
| NH-i-Pr | Cl |
| NH-i-Pr | F |
| NH-i-Pr | Br |
| NH-i-Pr | Me |
| NH-i-Pr | Et |
| NH-i-Pr | c-Pr |
| NH-i-Pr | CF₃ |
| NH-i-Pr | OMe |
| NH-i-Pr | SMe |
| NH-i-Pr | SCF₃ |
| NH-i-Pr | OCF₂H |
| NH-i-Pr | CO₂Me |
| NH-i-Pr | ethenyl |
| NH-i-Pr | ethynyl |
| NH-i-Pr | 2,2-di-Cl-c-Pr |
| i-Bu | Cl |
| i-Bu | F |
| i-Bu | Br |
| i-Bu | Me |
| i-Bu | Et |
| i-Bu | c-Pr |
| i-Bu | CF₃ |
| i-Bu | OMe |
| i-Bu | SMe |
| i-Bu | SCF₃ |
| i-Bu | OCF₂H |
| i-Bu | CO₂Me |
| i-Bu | ethenyl |
| i-Bu | ethynyl |
| i-Bu | 2,2-di-Cl-c-Pr |
| 3-F-Ph | Cl |
| 3-F-Ph | F |
| 3-F-Ph | Br |
| 3-F-Ph | Me |
| 3-F-Ph | Et |
| 3-F-Ph | c-Pr |
| 3-F-Ph | CF₃ |
| 3-F-Ph | OMe |
| 3-F-Ph | SMe |
| 3-F-Ph | SCF₃ |
| 3-F-Ph | OCF₂H |
| 3-F-Ph | CO₂Me |
| 3-F-Ph | ethenyl |
| 3-F-Ph | ethynyl |
| 3-F-Ph | 2,2-di-Cl-c-Pr |

TABLE 5a

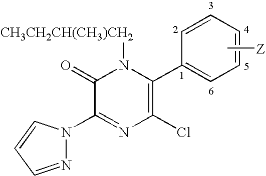

Z
2,3,4,5,6-penta-F
2-F
3-F
4-F
2-Cl
3-Cl
4-Cl
2-OMe
3-OMe
4-OMe
2-Me
3-Me
4-Me
2-CF₃
3-CF₃
4-CF₃
2-Et
2-i-Pr
2-c-Pr
2-Br
2-CN
2-SMe
2-OCF₃
2-SCF₃
2-ethenyl
2-ethynyl
2-OEt
2,4-di-F
2,3-di-F
2,6-di-F
2,5-di-F
2,3,4-tri-F
2,3,5-tri-F
2,3,6-tri-F
2,4,5-tri-F
3,4,5-tri-F
2-F-6-Cl
2-F-4-Cl
2-F-3-Cl
2-F-5-Cl
2-F-6-Me
2-F-4-Me
2-F-4-OMe
2-F-6-OMe
2-F-4-Br
2-F-6-Br
2-F-6-CN
2-F-6-CF₃
2-F-4-CF₃
2,6-di-F-4-Cl
2,6-di-F-4-OMe
2,6-di-F-4-Me
2,6-di-F-4-CF₃
2,6-di-F-4-CN
2,6-di-F-4-SMe
2-Cl-4-F
2-Me-4-F
2-CF₃-4-F
2-CF₃-6-F
2,6-di-Me-4-F
2-OMe-4-F
2-Et-4-F
2,6-di-Me-4-Cl
2,6-di-Me-4-OMe
2,6-di-Me-4-CF₃
2,6-di-Me-4-Br
2,6-di-Me-4-SMe
2-Cl-4-Me
2-CF₃-4-Me
2-OMe-4-Me
2-Br-4-Me
2-Et-4-Me
2-CN-4-Me
2,6-di-Cl-4-F
2,6-di-Cl-4-Me
2,6-di-Cl-4-Br
2,6-di-Cl-4-OMe
2,6-di-Cl-4-SMe
2,4,6-tri-Cl
2,4,6-tri-Me
2,4,5-tri-Me
2,3,6-tri-Me
2,3,4-tri-Me
2,4,5-tri-Cl
2,3,6-tri-Cl
2,3,4-tri-Cl
2,6-di-Et
2,6-di-Et-4-F
2,6-di-Et-4-Cl
2,6-di-F-4-Cl

TABLE 5b

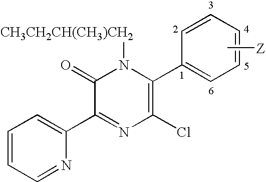

Z
2,3,4,5,6-penta-F
2-F
3-F
4-F
2-Cl
3-Cl
4-Cl
2-OMe
3-OMe
4-OMe
2-Me
3-Me
4-Me
2-CF₃
3-CF₃
4-CF₃
2-Et
2-i-Pr
2-c-Pr
2-Br
2-CN
2-SMe
2-OCF₃
2-SCF₃
2-ethenyl
2-ethynyl
2-OEt
2,4-di-F
2,3-di-F
2,6-di-F
2,5-di-F
2,3,4-tri-F
2,3,5-tri-F
2,3,6-tri-F
2,4,5-tri-F
3,4,5-tri-F
2-F-6-Cl
2-F-4-Cl
2-F-3-Cl
2-F-5-Cl
2-F-6-Me
2-F-4-Me
2-F-4-OMe
2-F-6-OMe
2-F-4-Br
2-F-6-Br
2-F-6-CN
2-F-6-CF₃
2-F-4-CF₃
2,6-di-F-4-Cl
2,6-di-F-4-OMe
2,6-di-F-4-Me
2,6-di-F-4-CF₃
2,6-di-F-4-CN
2,6-di-F-4-SMe
2-Cl-4-F
2-Me-4-F
2-CF₃-4-F
2-CF₃-6-F
2,6-di-Me-4-F
2-OMe-4-F
2-Et-4-F
2,6-di-Me-4-Cl
2,6-di-Me-4-OMe
2,6-di-Me-4-CF₃
2,6-di-Me-4-Br
2,6-di-Me-4-SMe
2-Cl-4-Me
2-CF₃-4-Me
2-OMe-4-Me
2-Br-4-Me
2-Et-4-Me
2-CN-4-Me
2,6-di-Cl-4-F
2,6-di-Cl-4-Me
2,6-di-Cl-4-Br
2,6-di-Cl-4-OMe
2,6-di-Cl-4-SMe
2,4,6-tri-Cl
2,4,6-tri-Me
2,4,5-tri-Me
2,3,6-tri-Me
2,3,4-tri-Me
2,4,5-tri-Cl
2,3,6-tri-Cl
2,3,4-tri-Cl
2,6-di-Et
2,6-di-Et-4-F
2,6-di-Et-4-Cl
2,6-di-F-4-Cl

TABLE 5c

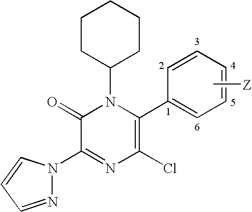

Z
2,3,4,5,6-penta-F
2-F
3-F
4-F
2-Cl
3-Cl
4-Cl
2-OMe
3-OMe
4-OMe
2-Me
3-Me
4-Me
2-CF₃
3-CF₃
4-CF₃
2-Et
2-i-Pr
2-c-Pr
2-Br
2-CN
2-SMe
2-OCF₃
2-SCF₃
2-ethenyl
2-ethynyl
2-OEt
2,4-di-F
2,3-di-F
2,6-di-F
2,5-di-F
2,3,4-tri-F
2,3,5-tri-F
2,3,6-tri-F
2,4,5-tri-F
3,4,5-tri-F
2-F-6-Cl
2-F-4-Cl
2-F-3-Cl
2-F-5-Cl
2-F-6-Me
2-F-4-Me
2-F-4-OMe
2-F-6-OMe
2-F-4-Br
2-F-6-Br
2-F-6-CN
2-F-6-CF₃
2-F-4-CF₃
2,6-di-F-4-Cl
2,6-di-F-4-OMe
2,6-di-F-4-Me
2,6-di-F-4-CF₃
2,6-di-F-4-CN
2,6-di-F-4-SMe
2-Cl-4-F
2-Me-4-F
2-CF₃-4-F
2-CF₃-6-F
2,6-di-Me-4-F
2-OMe-4-F
2-Et-4-F
2,6-di-Me-4-Cl
2,6-di-Me-4-OMe
2,6-di-Me-4-CF₃
2,6-di-Me-4-Br
2,6-di-Me-4-SMe
2-Cl-4-Me
2-CF₃-4-Me
2-OMe-4-Me
2-Br-4-Me
2-Et-4-Me
2-CN-4-Me
2,6-di-Cl-4-F
2,6-di-Cl-4-Me
2,6-di-Cl-4-Br
2,6-di-Cl-4-OMe
2,6-di-Cl-4-SMe
2,4,6-tri-Cl
2,4,6-tri-Me
2,4,5-tri-Me
2,3,6-tri-Me
2,3,4-tri-Me
2,4,5-tri-Cl
2,3,6-tri-Cl
2,3,4-tri-Cl
2,6-di-Et
2,6-di-Et-4-F
2,6-di-Et-4-Cl
2,6-di-F-4-Cl

TABLE 6a

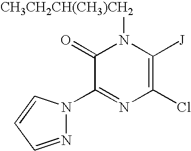

J
3-Cl-2-pyridinyl
3-CF₃-2-pyridinyl
3-Me-2-pyridinyl
3-F-2-pyridinyl
3-Br-2-pyridinyl
3-CN-2-pyridinyl
3-MeO-2-pyridinyl
3,5-di-Me-2-pyridinyl
3,6-di-Me-2-pyridinyl
3,5-di-Cl-2-pyridinyl
2-Cl-3-pyridinyl
2-Me-3-pyridinyl
2-F-3-pyridinyl
2-MeO-3-pyridinyl
2-MeS-3-pyridinyl
4-Cl-3-pyridinyl
4-Me-3-pyridinyl
4-F-3-pyridinyl
4-MeO-3-pyridinyl
4-MeS-3-pyridinyl
2,4-di-Cl-3-pyridinyl
2,4-di-Me-3-pyridinyl
2,4-di-F-3-pyridinyl
2,4,6-tri-Me-3-pyridinyl
2,4,6-tri-F-3-pyridinyl
3,5-di-F-4-pyridinyl
3-Cl-4-pyridinyl
3-Me-4-pyridinyl
3,5-di-Cl-4-pyridinyl
3,5-di-Me-4-pyridinyl
2-Cl-3-thienyl
2-Me-3-thienyl
2-F-3-thienyl
2,4-di-Cl-3-thienyl
2,5-di-Me-3-thienyl
3-Cl-2-thienyl
3-Me-2-thienyl
3-F-2-thienyl
3,5-di-Cl-2-thienyl
3,5-di-Me-2-thienyl
2,4-di-Me-2-thienyl
1-naphthalenyl
2-Me-1-naphthalenyl
2-Cl-1-naphthalenyl
3-Cl-2-quinolinyl
3-Cl-2-quinoxalinyl
2-naphthalenyl
1-Me-2-naphthalenyl
1-Cl-2-naphthalenyl
3,6-di-Cl-2-quinolinyl
3,6-di-Cl-2-quinoxalinyl
3-Me-2-quinolinyl
2-Cl-3-quinolinyl
2-F-3-quinolinyl
2-benzoxazolyl
2-benzothiazolyl
4-quinazolinyl
1-isoquinolinyl
4-quinolinyl
3-Cl-4-quinolinyl
3-Cl-2-pyrazinyl
3-CF₃-2-pyrazinyl
3-Me-2-pyrazinyl
3-F-2-pyrazinyl
3-Br-2-pyrazinyl
3-CN-2-pyrazinyl
3-MeO-2-pyrazinyl
3,5-di-Me-2-pyrazinyl
3,6-di-Me-2-pyrazinyl
3,5-di-Cl-2-pyrazinyl
5-Cl-4-pyrimidinyl
5-Me-4-pyrimidinyl
5-F-4-pyrimidinyl
5-CF₃-4-pyrimidinyl
6-Cl-3-pyridazinyl
2-thiazolyl
2-oxazolyl
2,4-di-Me-5-thiazolyl
2,4-di-Cl-5-thiazolyl
2,5-di-Cl-4-thiazolyl
3,5-di-Me-4-isoxazolyl
3,5-di-Cl-4-isothiazolyl
1,2,3-oxadiazol-4-yl
5-Me-1,2,3-thiadiazol-4-yl
1,3,4-thiadiazol-2-yl
1,3,4-oxadiazol-2-yl
5-Cl-1,2,3-thiadiazol-4-yl
2,5-di-Me-1,2,3-triazol-4-yl
2,5-di-Me-1H-pyrrol-1-yl
2,5-di-Cl-1H-pyrrol-1-yl
2,5-di-Br-1H-pyrrol-1-yl
2-Me-1H-pyrrol-1-yl
2,4-di-Me-1H-pyrrol-1-yl
3,5-di-Me-1H-pyrazol-1-yl
3,5-di-Me-1H-1,2,4-triazol-1-yl
3-CF₃-5-Me-1H-pyrazol-1-yl
1,3,5-tri-Me-1H-pyrazol-4-yl
1,3-di-Me-5-Cl-1H-pyrazol-4-yl
2,5-di-Me-1H-imidazol-1-yl
2-Me-1H-imidazol-1-yl
5-Me-1H-imidazol-1-yl
4-Me-5-thiazolyl
4-Cl-5-thiazolyl
5-Cl-4-thiazolyl
5-Me-4-thiazolyl
3,4,5-tri-Me-1H-pyrazol-1-yl
3,5-di-Me-2-furanyl
2,4-di-Me-3-furanyl
3-CF₃-1,5-di-Me-1H-pyrazol-4-yl
5-Me-1,2,3-oxadiazol-4-yl
5-Cl-1,2,3-oxadiazol-4-yl

TABLE 6b

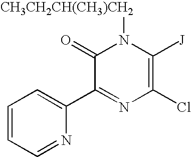

J
3-Cl-2-pyridinyl
3-CF₃-2-pyridinyl
3-Me-2-pyridinyl
3-F-2-pyridinyl
3-Br-2-pyridinyl
3-CN-2-pyridinyl
3-MeO-2-pyridinyl
3,5-di-Me-2-pyridinyl
3,6-di-Me-2-pyridinyl
3,5-di-Cl-2-pyridinyl
2-Cl-3-pyridinyl
2-Me-3-pyridinyl
2-F-3-pyridinyl
2-MeO-3-pyridinyl
2-MeS-3-pyridinyl
4-Cl-3-pyridinyl
4-Me-3-pyridinyl
4-F-3-pyridinyl
4-MeO-3-pyridinyl
4-MeS-3-pyridinyl
2,4-di-Cl-3-pyridinyl
2,4-di-Me-3-pyridinyl
2,4-di-F-3-pyridinyl
2,4,6-tri-Me-3-pyridinyl
2,4,6-tri-F-3-pyridinyl
3,5-di-F-4-pyridinyl
3-Cl-4-pyridinyl
3-Me-4-pyridinyl
3,5-di-Cl-4-pyridinyl
3,5-di-Me-4-pyridinyl
2-Cl-3-thienyl
2-Me-3-thienyl
2-F-3-thienyl
2,4-di-Cl-3-thienyl
2,5-di-Me-3-thienyl
3-Cl-2-thienyl
3-Me-2-thienyl
3-F-2-thienyl
3,5-di-Cl-2-thienyl
3,5-di-Me-2-thienyl
2,4-di-Me-2-thienyl
1-naphthalenyl
2-Me-1-naphthalenyl
2-Cl-1-naphthalenyl
3-Cl-2-quinolinyl
3-Cl-2-quinoxalinyl
2-naphthalenyl
1-Me-2-naphthalenyl
1-Cl-2-naphthalenyl
3,6-di-Cl-2-quinolinyl
3,6-di-Cl-2-quinoxalinyl
3-Me-2-quinolinyl
2-Cl-3-quinolinyl
2-F-3-quinolinyl
2-benzoxazolyl
2-benzothiazolyl
4-quinazolinyl
1-isoquinolinyl
4-quinolinyl
3-Cl-4-quinolinyl
3-Cl-2-pyrazinyl
3-CF₃-2-pyrazinyl
3-Me-2-pyrazinyl
3-F-2-pyrazinyl
3-Br-2-pyrazinyl
3-CN-2-pyrazinyl
3-MeO-2-pyrazinyl
3,5-di-Me-2-pyrazinyl
3,6-di-Me-2-pyrazinyl
3,5-di-Cl-2-pyrazinyl
5-Cl-4-pyrimidinyl
5-Me-4-pyrimidinyl
5-F-4-pyrimidinyl
5-CF₃-4-pyrimidinyl
6-Cl-3-pyridazinyl
2-thiazolyl
2-oxazolyl
2,4-di-Me-5-thiazolyl
2,4-di-Cl-5-thiazolyl
2,5-di-Cl-4-thiazoiyl
3,5-di-Me-4-isoxazolyl
3,5-di-Cl-4-isothiazoiyl
1,2,3-oxadiazol-4-yl
5-Me-1,2,3-thiadiazol-4-yl
1,3,4-thiadiazol-2-yl
1,3,4-oxadiazol-2-yl
5-Cl-1,2,3-thiadiazol-4-yl
2,5-di-Me-1,2,3-triazol-4-yl
2,5-di-Me-1H-pyrrol-1-yl
2,5-di-Cl-1H-pyrrol-1-yl
2,5-di-Br-1H-pyrrol-1-yl
2-Me-1H-pyrrol-1-yl
2,4-di-Me-1H-pyrrol-1-yl
3,5-di-Me-1H-pyrazol-1-yl
3,5-di-Me-1H-1,2,4-triazol-1-yl
3-CF₃-5-Me-1H-pyrazol-1-yl
1,3,5-tri-Me-1H-pyrazol-4-yl
1,3-di-Me-5-Cl-1H-pyrazol-4-yl
2,5-di-Me-1H-imidazol-1-yl
2-Me-1H-imidazol-1-yl
5-Me-1H-imidazol-1-yl
4-Me-5-thiazolyl
4-Cl-5-thiazolyl
5-Cl-4-thiazolyl
5-Me-4-thiazolyl
3,4,5-tri-Me-1H-pyrazol-1-yl
3,5-di-Me-2-furanyl
2,4-di-Me-3-furanyl
3-CF₃-1,5-di-Me-1H-pyrazol-4-yl
5-Me-1,2,3-oxadiazol-4-yl
5-Cl-1,2,3-oxadiazol-4-yl

TABLE 6c

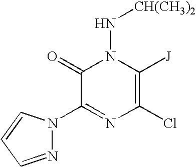

J
3-Cl-2-pyridinyl
3-CF₃-2-pyridinyl
3-Me-2-pyridinyl
3-F-2-pyridinyl
3-Br-2-pyridinyl
3-CN-2-pyridinyl
3-MeO-2-pyridinyl
3,5-di-Me-2-pyridinyl
3,6-di-Me-2-pyridinyl
3,5-di-Cl-2-pyridinyl
2-Cl-3-pyridinyl
2-Me-3-pyridinyl
2-F-3-pyridinyl
2-MeO-3-pyridinyl
2-MeS-3-pyridinyl
4-Cl-3-pyridinyl
4-Me-3-pyridinyl
4-F-3-pyridinyl
4-MeO-3-pyridinyl
4-MeS-3-pyridinyl
2,4-di-Cl-3-pyridinyl
2,4-di-Me-3-pyridinyl
2,4-di-F-3-pyridinyl
2,4,6-tri-Me-3-pyridinyl
2,4,6-tri-F-3-pyridinyl
3,5-di-F-4-pyridinyl
3-Cl-4-pyridinyl
3-Me-4-pyridinyl
3,5-di-Cl-4-pyridinyl
3,5-di-Me-4-pyridinyl
2-Cl-3-thienyl
2-Me-3-thienyl
2-F-3-thienyl
2,4-di-Cl-3-thienyl
2,5-di-Me-3-thienyl
3-Cl-2-thienyl
3-Me-2-thienyl
3-F-2-thienyl
3,5-di-Cl-2-thienyl
3,5-di-Me-2-thienyl
2,4-di-Me-2-thienyl
1-naphthalenyl
2-Me-1-naphthalenyl
2-Cl-1-naphthalenyl
3-Cl-2-quinolinyl
3-Cl-2-quinoxalinyl
2-naphthalenyl
1-Me-2-naphthalenyl
1-Cl-2-naphthalenyl
3,6-di-Cl-2-quinolinyl
3,6-di-Cl-2-quinoxalinyl
3-Me-2-quinolinyl
2-Cl-3-quinolinyl
2-F-3-quinolinyl
2-benzoxazolyl
2-benzothiazolyl
4-quinazolinyl
1-isoquinolinyl
4-quinolinyl
3-Cl-4-quinolinyl
3-Cl-2-pyrazinyl
3-CF₃-2-pyrazinyl
3-Me-2-pyrazinyl
3-F-2-pyrazinyl
3-Br-2-pyrazinyl
3-CN-2-pyrazinyl
3-MeO-2-pyrazinyl
3,5-di-Me-2-pyrazinyl
3,6-di-Me-2-pyrazinyl
3,5-di-Cl-2-pyrazinyl
5-Cl-4-pyrimidinyl
5-Me-4-pyrimidinyl
5-F-4-pyrimidinyl
5-CF₃-4-pyrimidinyl
6-Cl-3-pyrazdazinyl
2-thiazolyl
2-oxazolyl
2,4-di-Me-5-thiazolyl
2,4-di-Cl-5-thiazolyl
2,5-di-Cl-4-thiazolyl
3,5-di-Me-4-isoxazolyl
3,5-di-Cl-4-isothiazolyl
1,2,3-oxadiazol-4-yl
5-Me-1,2,3-thiadiazol-4-yl
1,3,4-thiadiazol-2-yl
1,3,4-oxadiazol-2-yl
5-Cl-1,2,3-thiadiazol-4-yl
2,5-di-Me-1,2,3-triazol-4-yl
2,5-di-Me-1H-pyrrol-1-yl
2,5-di-Cl-1H-pyrrol-1-yl
2,5-di-Br-1H-pyrrol-1-yl
2-Me-1H-pyrrol-1-yl
2,4-di-Me-1H-pyrrol-1-yl
3,5-di-Me-1H-pyrazol-1-yl
3,5-di-Me-1H-1,2,4-triazol-1-yl
3-CF₃-5-Me-1H-pyrazol-1-yl
1,3,5-tri-Me-1H-pyrazol-4-yl
1,3-di-Me-5-Cl-1H-pyrazol-4-yl
2,5-di-Me-1H-imidazol-1-yl
2-Me-1H-imidazol-1-yl
5-Me-1H-imidazol-1-yl
4-Me-5-thiazolyl
4-Cl-5-thiazolyl
5-Cl-4-thiazolyl
5-Me-4-thiazolyl
3,4,5-tri-Me-1H-pyrazol-1-yl
3,5-di-Me-2-furanyl
2,4-di-Me-3-furanyl
3-CF₃-1,5-di-Me-1H-pyrazol-4-yl
5-Me-1,2,3-oxadiazol-4-yl
5-Cl-1,2,3-oxadiazol-4-yl

TABLE 6b

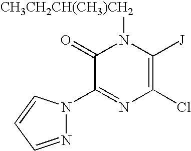

J
Me
Et
i-Pr
n-Pr
i-Bu
n-Bu
s-Bu
3-Me-Bu
n-pentyl
n-Hex
2-propenyl
2-Me-2-propenyl
3-butenyl
3-pentenyl
2-propynyl
3-butynyl
4-butynyl
c-Pr
c-pentyl
c-Hex
2-cyclohexenyl
3-cyclohexenyl
CH₂CH₂SMe
CH₂CH(Me)SMe
CH₂CH₂S(O)Me
CH₂CH₂S(O)₂Me
CH₂CO₂Me
CH₂CO₂-i-Pr
CH(Me)CO₂Me
CH₂C(O)Me
CH₂CH₂C(O)Me
CH₂SiMe₃
CH₂CH₂SiMe₃
2,2-dimethylpropyl
CH₂Ph
CH₂-c-Pr
CH₂CH(n-Pr)Me
CH₂-2-Cl-Ph
CH₂-3-Cl-Ph
CH₂-4-Cl-Ph
CH(Et)₂
CH₂CH(Et)₂
CH₂-c-Hex
CH₂CN
CH₂NO₂
CH₂CH₂OH
CH₂CH₂OMe
CH₂CH(Me)OMe
CH(Me)CH₂OMe
CH(Me)CH(OMe)₂
CH₂-2-dioxolanyl
CH₂CH₂OCF₃
CH₂-2-cyclohexenyl
4-tetrahydropyranyl
3-tetrahydropyranyl
3-tetrahydrofuranyl
CH₂CH₂CH(Me)₂
t-Amyl
CH(Me)Et
CH(Me)-n-Pr
CH(CF₃)Et
CH(Et)-n-Pr
CH(Me)-n-Bu
t-Bu

TABLE 6e

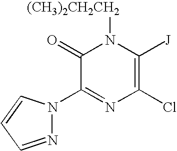

J
Me
Et
i-Pr
n-Pr
i-Bu
n-Bu
s-Bu
3-Me-Bu
n-pentyl
n-Hex
2-propenyl
2-Me-2-propenyl
3-butenyl
3-pentenyl
2-propynyl
3-butynyl
4-butynyl
c-Pr
c-pentyl
c-Hex
2-cyclohexenyl
3-cyclohexenyl
CH₂CH₂SMe
CH₂CH(Me)SMe
CH₂CH₂S(O)Me
CH₂CH₂S(O)₂Me
CH₂CO₂Me
CH₂CO₂-i-Pr
CH(Me)CO₂Me
CH₂C(O)Me
CH₂CH₂C(O)Me
CH₂SiMe₃
CH₂CH₂SiMe₃
2,2-dimethylpropyl
CH₂Ph
CH₂-c-Pr
CH₂CH(n-Pr)Me
CH₂-2-Cl-Ph
CH₂-3-Cl-Ph
CH₂-4-Cl-Ph
CH(Et)₂
CH₂CH(Et)₂
CH₂-c-Hex
CH₂CN
CH₂NO₂
CH₂CH₂OH
CH₂CH₂OMe
CH₂CH(Me)OMe
CH(Me)CH₂OMe
CH(Me)CH(OMe)₂
CH₂-2-dioxolanyl
CH₂CH₂OCF₃
CH₂-2-cyclohexenyl
4-tetrahydropyranyl
3-tetrahydropyranyl
3-tetrahydrofuranyl
CH₂CH₂CH(Me)₂
t-Amyl
CH(Me)Et
CH(Me)-n-Pr
CH(CF₃)Et
CH(Et)-n-Pr
CH(Me)-n-Bu
t-Bu

TABLE 6f

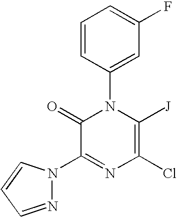

J
Me
Et
i-Pr
n-Pr
i-Bu
n-Bu
s-Bu
3-Me-Bu
n-pentyl
n-Hex
2-propenyl
2-Me-2-propenyl
3-butenyl
3-pentenyl
2-propynyl
3-butynyl
4-butynyl
c-Pr
c-pentyl
c-Hex
2-cyclohexenyl
3-cyclohexenyl
CH₂CH₂SMe
CH₂CH(Me)SMe
CH₂CH₂S(O)Me
CH₂CH₂S(O)₂Me
CH₂CO₂Me
CH₂CO₂-i-Pr
CH(Me)CO₂Me
CH₂C(O)Me
CH₂CH₂C(O)Me
CH₂SiMe₃
CH₂CH₂SiMe₃
2,2-dimethylpropyl
CH₂Ph
CH₂-c-Pr
CH₂CH(n-Pr)Me
CH₂-2-Cl-Ph
CH₂-3-Cl-Ph
CH₂-4-Cl-Ph
CH(Et)₂
CH₂CH(Et)₂
CH₂-c-Hex
CH₂CN
CH₂NO₂
CH₂CH₂OH
CH₂CH₂OMe
CH₂CH(Me)OMe
CH(Me)CH₂OMe
CH(Me)CH(OMe)₂
CH₂-2-dioxolanyl
CH₂CH₂OCF₃
CH₂-2-cyclohexenyl
4-tetrahydropyranyl
3-tetrahydropyranyl
3-tetrahydrofuranyl
CH₂CH₂CH(Me)₂
t-Amyl
CH(Me)Et
CH(Me)-n-Pr
CH(CF₃)Et
CH(Et)-n-Pr
CH(Me)-n-Bu
t-Bu

### Formulation/Utility

Compounds of this invention will generally be used as a formulation or composition with an agriculturally suitable carrier comprising at least one of a liquid diluent, a solid diluent or a surfactant. The formulation or composition ingredients are selected to be consistent with the physical properties of the active ingredient, mode of application and environmental factors such as soil type, moisture and temperature. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films (including seed treatment), and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders. | | | |
| Suspensions, Emulsions, | 1-50 | 40-99 | 0-50 |
| Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Typical solid diluents are described in Watkins, et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Detergents and Emulsifiers Annual, Allured Publ. Corp., Ridgewood, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, glycerol esters, poly-oxyethylene/polyoxypropylene block copolymers, and alkylpolyglycosides where the number of glucose units, referred to as degree of polymerization (D.P.), can range from 1 to 3 and the alkyl units can range from C₆ to C₁₄ (see Pure and Applied Chemistry 72, 1255-1264). Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, propylene carbonate, dibasic esters, paraffins, alkylbenzenes, alkylnaphthalenes, glycerine, triacetine, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, acetates such as hexyl acetate, heptyl acetate and octyl acetate, and alcohols such as methanol, cyclohexanol, decanol, benzyl and tetrahydrofurfuryl alcohol.

Useful formulations of this invention may also contain materials well known to those skilled in the art as formulation aids such as antifoams, film formers and dyes. Antifoams can include water dispersible liquids comprising polyorganosiloxanes like Rhodorsil® 416. The film formers can include polyvinyl acetates, polyvinyl acetate copolymers, polyvinylpyrrolidone-vinyl acetate copolymer, polyvinyl alcohols, polyvinyl alcohol copolymers and waxes. Dyes can include water dispersible liquid colorant compositions like Pro-lzed® Colorant Red. One skilled in the art will appreciate that this is a non-exhaustive list of formulation aids. Suitable examples of formulation aids include those listed herein and those listed in McCutcheon's 2001, Volume 2: Functional Materials published by MC Publishing Company and PCT Publication WO 03/024222.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modem Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Table A.

### EXAMPLE A

High Strength Concentrate

| | |
|---|---|
| Compound 1 | 98.5% |
| silica aerogel | 0.5% |
| synthetic amorphous fine silica | 1.0%. |

### EXAMPLE B

Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0%. |

### EXAMPLE C

Granule

| | |
|---|---|
| Compound 1 | 10.0% |
| attapulgite granules (low volatile matter, | 90.0%. |
| 0.71/0.30 mm; U.S.S. No. 25-50 sieves) | |

### EXAMPLE D

Aqueous Suspension

| | |
|---|---|
| Compound 1 | 25.0% |
| hydrated attapulgite | 3.0% |
| crude calcium ligninsulfonate | 10.0% |
| sodium dihydrogen phosphate | 0.5% |
| water | 61.5%. |

### EXAMPLE E

Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

### EXAMPLE F

Microemulsion

| | |
|---|---|
| Compound 1 | 1.0% |
| triacetine | 30.0% |
| C₈-C₁₀ alkylpolyglycoside | 30.0% |
| glyceryl monooleate | 19.0% |
| water | 20.0%. |

The compounds of this invention are useful as plant disease control agents. The present invention therefore further comprises a method for controlling plant diseases caused by plant pathogens comprising applying to the plant or portion thereof to be protected, or to the plant seed or seedling to be protected, an effective amount of a compound of the invention or a fungicidal composition containing said compound. The compounds and compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include:
Oomycetes, including Phytophthora diseases such as Phytophthora infestans, Phytophthora megasperma, Phytophthora parasitica, Phytophthora cinnamoni, Phytophthora capsici; Pythium diseases such as Pythium aphanidermatum; and diseases in the Peronosporaceae family, such as Plasmopara viticola, Peronospora spp. (including Peronospora tabacina and Peronospora parasitica), Pseudoperonospora spp. (including Pseudoperonospora cubensis), and Bremia lactucae.
Ascomycetes, including Alternaria diseases such as Alternaria solani and Alternaria brassicae; Guignardia diseases such as Guignardia bidwell; Venturia diseases such as Venturia inaequalis; Septoria diseases such as Septoria nodorum and Septoria tritici; powdery mildew diseases such as Erysiphe spp. (including Erysiphe graminis and Erysiphe polygoni), Uncinula necatur, Sphaerotheca fuligena, and Podosphaera leucotricha; Pseudocercosporella herpotrichoides; Botrytis diseases such as Botytis cinerea; Monilinia fructicola; Sclerotinia diseases such as Sclerotinia sclerotiorum; Magnaporthe grisea; Phomopsis viticola; Helminthosporium diseases such as Helminthosporium tritici repentis; Pyrenophora teres; anthracnose diseases such as Glomerella or Colletotrichum spp. (such as Colletotrichum graminicola and Colletotrichum orbiculare); Gaeumannomyces graminis
Basidiomycetes, including rust diseases caused by Puccinia spp. (such as Puccinia recondita, Puccinia striiformis, Puccinia hordei, Puccinia graminis, and Puccinia arachidis); Hemileia vastatrix; Uromyces appendiculatus; Phakopsora pachyrhizi; and bunt and smut diseases such as Ustilago spp. and Telletia caries.
Other pathogens including Rhizoctonia spp (such as Rhizoctonia solani); Fusarium diseases such as Fusarium roseum, Fusarium graminearum, Fusarium oxysporum; Verticillium dahliae; Sclerotium rolfsii; Rynchosporium secalis; Cercosporidium personatum, Cercospora arachidicola and Cercospora beticola.
And other genera and species closely related to these pathogens.
In addition to their fungicidal activity, the compositions or combinations can also have activity against bacteria such as Erwinia amylovora; Xanthomonas campestris; Pseudomonas syringae; and other related species.

Plant disease control, preventatively and curatively, is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to the seed to protect the seed and seedling.

Rates of application for these compounds can be influenced by many factors of the environment and should be determined under actual use conditions. Foliage can normally be protected when treated at a rate of from less than 1 g/ha to 5,000 g/ha of active ingredient. Seed and seedlings can normally be protected when seed is treated at a rate of from 0.1 to 10 g per kilogram of seed.

Compounds of this invention can also be mixed with one or more other insecticides, fungicides, nematocides, bactericides, acaricides, growth regulators, chemosterilants, semiochemicals, repellents, attractants, pheromones, feeding stimulants or other biologically active compounds to form a multi-component pesticide giving an even broader spectrum of agricultural protection. Examples of such agricultural protectants with which compounds of this invention can be formulated are: insecticides such as abamectin, acephate, azinphos-methyl, bifenthrin, buprofezin, carbofuran, chlorfenapyr, chlorpyrifos, chlorpyrifos-methyl, cyflumetofen, cyfluthrin, beta-cyfluthrin, cyhalothrin, lambda-cyhalothrin, deltamethrin, diafenthiuron, diazinon, diflubenzuron, dimefluthrin, dimethoate, dinotefuran, esfenvalerate, fenoxycarb, fenpropathrin, fenvalerate, fipronil, flonicamid, flubendiamide, flucythrinate, tau-fluvalinate, fonophos, imidacloprid, indoxacarb, isofenphos, malathion, metaflumizone, metaldehyde, methamidophos, methidathion, methomyl, methoprene, methoxychlor, metofluthrin, monocrotophos, noviflumuron, oxamyl, parathion, parathion-methyl, permethrin, phorate, phosalone, phosmet, phosphamidon, pirimicarb, profenofos, profluthrin, pyrafluprole, pyridalyl, pyriprole, rotenone, spirodiclofen, spiromesifen, spirotetramat, sulprofos, tebufenozide, tefluthrin, terbufos, tetrachlorvinphos, thiamethoxam, thiodicarb, tralomethrin, trichlorfon and triflumuron; fungicides such as acibenzolar, amisulbrom, azaconazole, azoxystrobin, benalaxyl, benomyl, benthiavalicarb, binomial, bitertanol, blasticidin-S, Bordeaux mixture (Tribasic copper sulfate), boscalid/nicobifen, bromuconazole, buthiobate, carboxin, carpropamid (KTU 3616), captafol, captan, carbendazim, 5-chloro-7-(4-methyl-piperidin-1-yl)-6-(2,4,6-trifluorophenyl)-[1,2,4]triazolo[1,5-a]pyrimidine, chloroneb, chlorothalonil, clotrimazole, copper oxychloride, copper salts such as copper sulfate and copper hydroxide, cyazofamid, cyflunamid, cymoxanil, cyproconazole, cyprodinil (CGA 219417), diclocymet (S-2900), diclomezine, dicloran, difenoconazole, dimethomorph, dimoxystrobin, diniconazole, diniconazole-M, discostrobin, dithianon, dodemorph, dodine, econazole, etaconazole, edifenphos, epoxiconazole (BAS 480F), ethaboxam, famoxadone, fenamidone (RP 407213), fenarimol, fenbuconazole, fencaramid (SZX0722), fenfuram, fenhexamide, fenoxanil, fenpiclonil, fenpropidin, fenpropimorph, fentin acetate, fentin hydroxide, ferbam, ferimzone, fluazinam, fludioxonil, flumetover (RPA 403397), fluopicolide, fluoxastrobin, fluquinconazole, flusilazole, flutolanil, flutriafol, folpet, fosetyl-aluminum, furalaxyl, furametapyr (S-82658), hexaconazole, guazatine, imazalil, iminoctadine, ipconazole, iprobenfos, iprodione, iprovalicarb, isoconazole, isoprothiolane, kasugamycin, kresoxim-methyl, mancozeb, mandipropamid, maneb, mefenoxam, mepronil, metalaxyl, metconazole, metiram-zink, metominostrobin/fenominostrobin, mepanipyrim, metrafenone, miconazole, myclobutanil, neo-asozin (ferric methanearsonate), nuarimol, orysastrobin, oxadixyl, penconazole, pencycuron, penthiopyrad, phosphonic acid, picobenzamid, picoxystrobin, probenazole, prochloraz, procymidone, propamocarb, propiconazole, propineb, proquinazid, prothioconazole, pyraclostrobin, pryazophos, pyrifenox, pyrimethanil, pyrifenox, pyroquilon, quinconazole, quinoxyfen, silthiofam, simeconazole, spiroxamine, sulfur, tebuconazole, tetraconazole, thiabendazole, thifluzamide, thiophanate-methyl, thiram, tiadinil, triadimefon, triadimenol, triarimol, tridemorph, trimoprhamide tricyclazole, trifloxystrobin, triforine, triticonazole, uniconazole, validamycin, vinclozolin, zineb, ziram, and zoxamide (RH 7281); nematocides such as aldoxycarb and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents such as Bacillus thuringiensis, Bacillus thuringiensis delta endotoxin, baculovirus, and entomopathogenic bacteria, virus and fungi. The weight ratios of these various mixing partners to compounds of this invention typically are between 100:1 and 1:100, preferably between 30:1 and 1:30, more preferably between 10:1 and 1:10, and most preferably between 4:1 and 1:4.

Of note are combinations of compounds of Formula 1 (e.g. Compound 15) with azoxystrobin, kesoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, tebuconazole, triticonazole, famoxadone, prochloraz, penthiopyrad and boscalid/nicobifen.

Preferred for better control of plant diseases caused by fungal plant pathogens (e.g., lower use rate or broader spectrum of plant pathogens controlled) or resistance management are mixtures of a compound of this invention with a fungicide selected from the group: azoxystrobin, kesoxim-methyl, trifloxystrobin, pyraclostrobin, picoxystrobin, dimoxystrobin, metominostrobin/fenominostrobin, quinoxyfen, metrafenone, cyflufenamid, fenpropidine, fenpropimorph, cyproconazole, epoxiconazole, flusilazole, metconazole, propiconazole, proquinazid, tebuconazole, triticonazole, famoxadone and penthiopyrad.

Specifically preferred mixtures (compound numbers refer to compounds in Index Table A) are selected from the group: combinations of Compound 1, Compounds 155, Compounds 161, Compound 170, Compound 298, Compound 303, Compound 324 or Compound 357 with azoxystrobin, combinations of combinations of Compound 1, Compounds 155, Compounds 161, Compound 170, Compound 298, Compound 303, Compound 324 or Compound 357 with kesoxim-methyl, combinations of combinations of Compound 1, Compounds 155, Compounds 161, Compound 170, Compound 298, Compound 303, Compound 324 or Compound 357 with trifloxystrobin, combinations of combinations of Compound 1, Compounds 155, Compounds 161, Compound 170, Compound 298, Compound 303, Compound 324 or Compound 357 with pyraclostrobin, combinations of combinations of Compound 1, Compounds 155, Compounds 161, Compound 170, Compound 298, Compound 303, Compound 324 or Compound 357 with picoxystrobin, combinations of combinations of Compound 1, Compounds 155, Compounds 161, Compound 170, Compound 298, Compound 303, Compound 324 or Compound 357 with dimoxystrobin, combinations of combinations of Compound 1, Compounds 155, Compounds 161, Compound 170, Compound 298, Compound 303, Compound 324 or Compound 357 with metominostrobin/fenominostrobin, combinations of combinations of Compound 1, Compounds 155, Compounds 161, Compound 170, Compound 298, Compound 303, Compound 324 or Compound 357 with quinoxyfen, combinations of combinations of Compound 1, Compounds 155, Compounds 161, Compound 170, Compound 298, Compound 303, Compound 324 or Compound 357 with metrafenone, combinations of combinations of Compound 1, Compounds 155, Compounds 161, Compound 170, Compound 298, Compound 303, Compound 324 or Compound 357 with cyflufenamid, combinations of combinations of Compound 1, Compounds 155, Compounds 161, Compound 170, Compound 298, Compound 303, Compound 324 or Compound 357 with fenpropidine, combinations of combinations of Compound 1, Compounds 155, Compounds 161, Compound 170, Compound 298, Compound 303, Compound 324 or Compound 357 with fenpropimorph, combinations of combinations of Compound 1, Compounds 155, Compounds 161, Compound 170, Compound 298, Compound 303, Compound 324 or Compound 357 with cyproconazole, combinations of combinations of Compound 1, Compounds 155, Compounds 161, Compound 170, Compound 298, Compound 303, Compound 324 or Compound 357 with epoxiconazole, combinations of combinations of Compound 1, Compounds 155, Compounds 161, Compound 170, Compound 298, Compound 303, Compound 324 or Compound 357 with flusilazole, combinations of combinations of Compound 1, Compounds 155, Compounds 161, Compound 170, Compound 298, Compound 303, Compound 324 or Compound 357 with metconazole, combinations of combinations of Compound 1, Compounds 155, Compounds 161, Compound 170, Compound 298, Compound 303, Compound 324 or Compound 357 with propiconazole, combinations of combinations of Compound 1, Compounds 155, Compounds 161, Compound 170, Compound 298, Compound 303, Compound 324 or Compound 357 with proquinazid, combinations of combinations of Compound 1, Compounds 155, Compounds 161, Compound 170, Compound 298, Compound 303, Compound 324 or Compound 357 with tebuconazole, combinations of combinations of Compound 1, Compounds 155, Compounds 161, Compound 170, Compound 298, Compound 303, Compound 324 or Compound 357 with triticonazole, combinations of combinations of Compound 1, Compounds 155, Compounds 161, Compound 170, Compound 298, Compound 303, Compound 324 or Compound 357 with famoxadone, and combinations of combinations of Compound 1, Compounds 155, Compounds 161, Compound 170, Compound 298, Compound 303, Compound 324 or Compound 357 with penthiopyrad.

Plant disease control, preventatively and curatively, is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to the seed to protect the seed and seedling.

Rates of application for these compounds can be influenced by many factors of the environment and should be determined under actual use conditions. Foliage can normally be protected when treated at a rate of from less than 1 g/ha to 5,000 g/ha of active ingredient. Seed and seedlings can normally be protected when seed is treated at a rate of from 0.1 to 10 g per kilogram of seed.

The weight ratios of these various mixing partners to compounds of this invention typically are between 100:1 and 1:100, preferably between 30:1 and 1:30, more preferably between 10:1 and 1:10, and most preferably between 4:1 and 1:4.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to the seed to protect the seed and seedling.

Rates of application for these compounds can be influenced by many factors of the environment and should be determined under actual use conditions. Foliage can normally be protected when treated at a rate of from less than 1 g/ha to 5,000 g/ha of active ingredient. Seed and seedlings can normally be protected when seed is treated at a rate of from 0.1 to 10 g per kilogram of seed.

The following TESTS demonstrate the control efficacy of compounds of this invention on specific pathogens. The pathogen control protection afforded by the compounds is not limited, however, to these species. See Index Tables A and B for compound descriptions. The following abbreviations are used in the Index Tables which follow: t means tertiary, s means secondary, n means normal, i means iso, c means cyclo, Me means methyl, Et means ethyl, Pr means propyl, i-Pr means isopropyl, Bu means butyl, i-Bu means isobutyl, Hex means hexyl, c-Hex means cyclohexyl, Ph means phenyl, OMe means methoxy, SMe means methylthio, CN means cyano, NO₂ means nitro, 2-Cl-4-F means 2-chloro-4-fluoro, TMS means trimethylsilyl, and other substituent abbreviations are defined analogously. The abbreviation “Ex.” stands for “Example” and is followed by a number indicating in which example the compound is prepared.

INDEX TABLE A

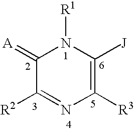

| Cmpd | | | | | M.P. |
|---|---|---|---|---|---|
| No. | R¹ | R² | R³ | J | (° C.) |
| 1 | i-Bu | 1H-pyrazol-1-yl | Cl | 2,6-di-F-Ph | * |
| (Ex. 1) | | | | | |
| 2 | i-Bu | 2-pyridinyl | Cl | 2,6-di-F-Ph | * |
| (Ex. 2) | | | | | |
| 3 | i-Bu | 1H-1,2,4-triazol-1-yl | Cl | 2,6-di-F-Ph | * |
| 4 | i-Bu | 4,5-dihydro-1H-pyrazol- | Cl | 2,6-di-F-Ph | * |
| | | 1-yl | | | |
| 5 | i-Bu | CN | Cl | 2,6-di-F-Ph | 100-102 |
| (Ex. 10) | | | | | |
| 6 | i-Bu | 1H-pyrazol-1-yl | Cl | 2,4-di-F-Ph | 147-149 |
| 7 | i-Bu | 1H-pyrazol-1-yl | Cl | 2,4,6-tri-F-Ph | 111-113 |
| 8 | i-Bu | 1H-1,2,4-thiazol-1-yl | Cl | 2,4,6-tri-F-Ph | 128-129 |
| 9 | i-Bu | 1H-pyrazol-1-y1 | Cl | 2-Cl-6-F-Ph | * |
| 10 | i-Bu | 1H-1,2,4-triazol-1-yl | Cl | 2-Cl-6-F-Ph | * |
| 11 | 2-Me-Bu | 1H-pyrazol-1-yl | Cl | 2,6-di-F-Ph | 108-109 |
| 12 | 2-Me-Bu | 1H-1,2,4-triazol-1-yl | Cl | 2,6-di-F-Ph | 68-70 |
| 13 | i-Bu | 1H-pyrazol-1-yl | OMe | 2-Cl-6-F-Ph | * |
| 14 | Me | 1H-pyrazol-1-yl | Cl | 2-F-Ph | * |
| 15 | Me | 1H-1,2,4-triazol-1-yl | Cl | 2-F-Ph | * |
| 16 | CH₂-c-Pr | 4,5-dihydro-1H-pyrazol- | Cl | 2,6-di-F-Ph | * |
| | | 1-yl | | | |
| 17 | CH₂-c-Pr | 1H-1,2,4-triazol-1-yl | Cl | 2,6-di-F-Ph | * |
| 18 | CH₂-c-Pr | 1H-pyrazol-1-yl | Cl | 2,6-di-F-Ph | * |
| 19 | 2-Me-Bu | 1H-pyrazol-1-yl | Cl | 2,4,6-tri-F-Ph | * |
| 20 | 2-Me-Bu | 1H-1,2,4-triazol-1-yl | Cl | 2,4,6-tri-F-Ph | * |
| 21 | 2-Me-Bu | CN | Cl | 2,6-di-F-Ph | * |
| 22 | i-Bu | 1H-pyrazol-1-yl | Cl | 4-Cl-Ph | * |
| 23 | i-Bu | 1H-1,2,4-triazol-1-yl | Cl | 4-Cl-Ph | * |
| 24 | i-Bu | 3,-5-diMe-1H-1,2,4- | Cl | 4-Cl-Ph | * |
| | | triazol-1-yl | | | |
| 25 | 1-Me-Bu | 1H-pyrazol-1-yl | Cl | 2,6-di-F-Ph | * |
| 26 | i-Pr | 1H-pyrazol-1-yl | Cl | 2,6-di-F-Ph | * |
| 27 | n-Pr | 1H-pyrazol-1-yl | Cl | 2,6-di-F-Ph | * |
| 28 | n-Pentyl | 1H-pyrazol-1-yl | Cl | 2,6-di-F-Ph | * |
| 29 | 1,2-di-Me-Pr | 1H-pyrazol-1-yl | Cl | 2,6-di-F-Ph | * |
| 30 | 2-Me-2-Propenyl | 1H-pyrazol-1-yl | Cl | 2,6-di-F-Ph | * |
| 31 | CH₂-c-Hex | 1H-pyrazol-1-yl | Cl | 2,6-di-F-Ph | * |
| 32 | s-Bu | 1H-1,2,4-triazol-1-yl | Cl | 2,6-di-F-Ph | * |
| 33 | i-Pr | 1H-1,2,4-triazol-1-yl | Cl | 2,6-di-F-Ph | * |
| 34 | n-Pr | 1H-1,2,4-triazol-1-yl | Cl | 2,6-di-F-Ph | * |
| 35 | 3-Me-Bu | 1H-pyrazol-1-yl | Cl | 2,6-di-F-Ph | * |
| 36 | i-Bu | 1H-pyrazol-1-yl | Cl | 4-CF₃-Ph | * |
| 37 | i-Bu | 1H-pyrazol-1-yl | Cl | 4-OMe-Ph | * |
| 38 | i-Bu | 1H-pyrazol-1-yl | Cl | 4-OCF₃-Ph | * |
| 39 | i-Bu | 1H-1,2,4-triazol-1-yl | Cl | 2,3-di-Cl-Ph | * |
| 40 | i-Bu | 1H-1,2,4-triazol-1-yl | Cl | 2,6-di-Cl-Ph | 176-181 |
| 41 | i-Bu | 1H-pyrazol-1-yl | Cl | 2,3-di-Cl-Ph | 104-109 |
| 42 | i-Bu | 1H-pyrazol-1-yl | Cl | 3,5-di-Cl-Ph | 182-187 |
| 43 | i-Bu | 1H-pyrazol-1-yl | Cl | 2,6-di-Cl-Ph | 145-148 |
| 44 | i-Bu | 1H-1,2,4-triazol-1-yl | Cl | 2-F-4-Cl-Ph | * |
| 45 | i-Bu | 1H-pyrazol-1-yl | Cl | 2-F-4-Cl-Ph | * |
| 46 | i-Bu | 1H-pyrazol-1-yl | Cl | 2-F-4-CF₃-Ph | * |
| 47 | c-Hex | 1H-pyrazol-1-yl | Cl | 2,6-di-F-Ph | * |
| 48 | i-Bu | NHN═C(Me)₂ | Cl | 2,6-di-F-Ph | * |
| 49 | c-Hex | 1H-pyrazol-1-yl | OMe | 2,6-di-F-Ph | 174-176 |
| 50 | i-Bu | 1H-pyrazol-1-yl | Cl | 2,3-di-F-Ph | * |
| 51 | i-Bu | 1H-1,2,4-triazol-1-yl | Cl | 2-Cl-4-F-Ph | 147-148 |
| 52 | i-Bu | 1H-1,2,4-triazol-1-yl | Cl | 2,3-di-F-Ph | 144-145 |
| 53 | i-Bu | 1H-pyrazol-1-yl | Cl | 2-Cl-4-F-Ph | 137-139 |
| (Ex. 6) | | | | | |
| 54 | i-Bu | 1H-1,2,4-triazol-1-yl | Cl | 2,5-di-F-Ph | 152-154 |
| 55 | i-Bu | 1H-1,2,4-triazol-1-yl | Cl | 2,3,6-tri-F-Ph | * |
| 56 | i-Bu | 1H-pyrazol-1-yl | Cl | 2,5-di-F-Ph | * |
| 57 | i-Bu | 1H-pyrazol-1-yl | Cl | 2,3,6-tri-F-Ph | * |
| 58 | CH₂CH═CH₂ | 1H-pyrazol-1-yl | Cl | 2,6-di-F-Ph | * |
| 59 | 2,2-di-Me-Pr | 1H-pyrazol-1-yl | Cl | 2,6-di-F-Ph | * |
| 60 | 2-Me-Bu | 1H-1,2,3-triazol-1-yl | Cl | 2,6-di-F-Ph | * |
| 61 | 2-Me-Bu | 1H-imidazol-1-yl | Cl | 2,6-di-F-Ph | * |
| 62 | 2-Me-Bu | 3,5-di-Me-1H-pyrazol-1- | Cl | 2,6-di-F-Ph | * |
| | | yl | | | |
| 63 | 2-Me-Bu | 3-CF₃-1H-pyrazol-1-yl | Cl | 2,6-di-F-Ph | * |
| 64 | 2-Me-Bu | 3-Me-5-CF₃-1H-pyrazol- | Cl | 2,6-di-F-Ph | * |
| | | 1-yl | | | |
| 65 | 2-Me-Bu | 3-Br-1H-pyrazol-1-yl | Cl | 2,6-di-F-Ph | * |
| 66 | 2-Me-Bu | 3-Me-1H-pyrazol-1-yl | Cl | 2,6-di-F-Ph | * |
| 67 | 2-Me-Bu | 4,5-di-CN-1H-imidazol- | Cl | 2,6-di-F-Ph | * |
| | | 1-yl | | | |
| 68 | 2-Me-Bu | 4,5-di-Cl-1H-imidazol-1- | Cl | 2,6-di-F-Ph | * |
| | | yl | | | |
| 69 | 2-Me-Bu | 2-Me-1H-imidazol-1-yl | Cl | 2,6-di-F-Ph | * |
| 70 | 2-Me-Bu | 2-Et-1H-imidazol-1-yl | Cl | 2,6-di-F-Ph | * |
| 71 | 2-Me-Bu | 2-n-Pr-1H-imidazol-1-yl | Cl | 2,6-di-F-Ph | * |
| 72 | 2-Me-Bu | 2-i-Pr-1H-imidazol-1-yl | Cl | 2,6-di-F-Ph | * |
| 73 | i-Bu | NHN═C(CH₂)₄ | Cl | 2,6-di-F-Ph | * |
| 74 | i-Bu | 1H-pyrazol-1-yl | Cl | 4-t-Bu-Ph | * |
| 75 | i-Bu | 1H-1,2,4-triazol-1-yl | Cl | 4-t-Bu-Ph | * |
| 76 | i-Bu | 1H-1,2,4-triazol-1-yl | Cl | 4-CF₃-Ph | * |
| 77 | i-Bu | 1H-1,2,4-triazol-1-yl | Cl | 4-OCF₃-Ph | * |
| 78 | i-Bu | NHN═C(Me)(CO₂Me) | Cl | 2,6-di-F-Ph | * |
| 79 | i-Bu | 1H-1,2,4-triazol-1-yl | Cl | 2,4-di-Me-Ph | * |
| 80 | i-Bu | 1H-pyrazol-1-yl | Cl | 2,4-di-Me-Ph | * |
| 81 | 2-Me-Bu | 3-CN-1H-pyrazol-1-yl | Cl | 2,6-di-F-Ph | * |
| 82 | 2-Me-Bu | 1H-pyrrol-1-yl | Cl | 2,6-di-F-Ph | * |
| 83 | i-Bu | 1-Me-1H-imidazol-5-yl | Cl | 2,6-di-F-Ph | * |
| 84 | i-Bu | 4-pyridinyl | Cl | 2,6-di-F.-Ph | * |
| 85 | i-Bu | 1-Me-1H-imidazol-4-yl | Cl | 2,6-di-F-Ph | 191-193 |
| (Ex. 11) | | | | | |
| 86 | i-Bu | 1H-pyrazol-1-yl | Cl | 2,4,6-tri-OMe-Ph | 166-171 |
| 87 | i-Bu | 1H-pyrazol-1-yl | Cl | 2-F-6-CF₃-Ph | * |
| 88 | i-Bu | 1H-pyrazol-1-yl | Cl | 3,4-methylenedioxy- | 178-182 |
| | | | | Ph | |
| 89 | i-Bu | 1H-pyrazol-1-yl | Cl | 5-Br-3,4- | 221-223 |
| | | | | methylenedioxy-Ph | |
| 90 | i-Bu | 1H-pyrazol-1-yl | Cl | 2-Naphthalenyl | * |
| 91 | CH₂-tetrahydrofuran- | 1H-pyrazol-1-yl | Cl | 2,6-di-F-Ph | * |
| | 2-yl | | | | |
| 92 | CH₂-tetrahydrofuran- | 1H-1,2,4-triazol-1-yl | Cl | 2,6-di-F-Ph | * |
| | 2-yl | | | | |
| 93 | 1-Me-2-OMe-Et | 1H-pyrazol-1-yl | Cl | 2,6-di-F-Ph | 165-168 |
| 94 | 1-Me-2-OMe-Et | 1H-1,2,4-triazol-1-yl | Cl | 2,6-di-F-Ph | 153-156 |
| 95 | 2-OMe-Et | 1H-pyrazol-1-yl | Cl | 2,6-di-F-Ph | 166-168 |
| 96 | 2-OMe-Et | 1H-1,2,4-triazol-1-yl | Cl | 2,6-di-F-Ph | 131-134 |
| 97 | 2-SMe-Et | 1H-pyrazol-1-yl | Cl | 2,6-di-F-Ph | 99-100 |
| 98 | 2-SMe-Et | 1H-1,2,4-triazol-1-yl | Cl | 2,6-di-F-Ph | 143-145 |
| 99 | i-Bu | 1H-pyrazol-1-yl | Br | 2,6-di-F-Ph | * |
| (Ex. 9) | | | | | |
| 100 | i-Bu | 1-Me-1H-pyrazol-4-yl | Cl | 2,6-di-F-Ph | * |
| 101 | i-Bu | 2-thiazolyl | Cl | 2,6-di-F-Ph | 120-124 |
| 102 | i-Bu | 1-Me-1H-imidazol-2-yl | Cl | 2,6-di-F-Ph | 220-224 |
| 103 | i-Bu | 1-Me-1H-pyrazol-5-yl | Cl | 2,6-di-F-Ph | * |
| 104 | Benzyl | 1H-pyrazol-1-yl | Cl | 2,6-di-F-Ph | 155-156 |
| 105 | Benzyl | 1H-1,2,4-triazol-1-yl | Cl | 2,6-di-F-Ph | 73-76 |
| 106 | Benzyl | CN | Cl | 2,6-di-F-Ph | 125-130 |
| 107 | CH(Me)Ph | 1H-pyrazol-1-yl | Cl | 2,6-di-F-Ph | 170-172 |
| 108 | CH(Me)Ph | 1H-1,2,4-triazol-1-yl | Cl | 2,6-di-F-Ph | 197-200 |
| 109 | CH(Me)Ph | CN | Cl | 2,6-di-F-Ph | 193-197 |
| 110 | 2-Me-Bu | SCN | Cl | 2,6-di-F-Ph | * |
| 111 | i-Bu | 1H-pyrazol-1-yl | Cl | 2,4-di-Cl-Ph | * |
| 112 | i-Bu | 1H-1,2,4-triazol-1-yl | Cl | 2,4,6-tri-OMe-Ph | * |
| 113 | i-Bu | 1H-1,2,4-triazol-1-yl | Cl | 2-F-6-CF₃-Ph | * |
| 114 | i-Bu | 1H-1,2,4-triazol-1-yl | Cl | 3,4-methylenedioxy- | 188-194 |
| | | | | Ph | |
| 115 | i-Bu | 1H-1,2,4-triazol-1-yl | Cl | 5-Br-3,4- | 198-202 |
| | | | | methylenedioxy-Ph | |
| 116 | i-Bu | 1H-1,2,4-triazol-1-yl | Cl | 3,5-di-Cl-Ph | * |
| 117 | i-Bu | 1H-1,2,4-triazol-1-yl | Cl | 2-F-4-CF₃-Ph | 159-163 |
| 118 | i-Bu | 1H-1,2,4-triazol-1-yl | Cl | 2,4-di-Cl-Ph | 147-149 |
| 119 | i-Bu | 1H-1,2,4-triazol-1-yl | Cl | 2-Naphthanenyl | * |
| 120 | i-Bu | 1H-1,2,4-triazol-1-yl | Cl | 2,3,6-tri-Cl-Ph | 81-84 |
| 121 | i-Bu | 1H-pyrazol-1-yl | Cl | 2,3,6-tri-Cl-Ph | 73-78 |
| 122 | CH₂CO₂Me | 1H-pyrazol-1-yl | Cl | 2,6-di-F-Ph | 158-160 |
| 123 | CH₂CO₂Me | 1H-1,2,4-triazol-1-yl | Cl | 2,6-di-F-Ph 72-76 | |
| 124 | CH₂CH₂Cl | 1H-pyrazol-1-yl | Cl | 2,6-di-F-Ph | 196-197 |
| 125 | CH₂CH₂Cl | 1H-1,2,4-thiazol-1-yl | Cl | 2,6-di-F-Ph212-215 | |
| 126 | CH₂CH₂SO₂Me | 1H-pyrazol-1-yl | Cl | 2,6-di-F-Ph207-212 | |
| 127 | CH₂CH₂SO₂Me | 1H-1,2,4-thiazol-1-yl | Cl | 2,6-di-F-Ph | 186-190 |
| 128 | i-Bu | 1H-1,2,4-thiazol-1-yl | Cl | CH₂Ph | * |
| 129 | i-Bu | 1H-1,2,4-triazol-1-yl | Cl | 1-Naphthanenyl | * |
| 130 | i-Bu | 1H-pyrazol-1-yl | Cl | CH₂Ph | * |
| 131 | i-Bu | 1H-pyrazol-1-yl | Cl | 1-Naphthanenyl | * |
| 132 | i-Bu | 1H-pyrazol-1-yl | Cl | CH(Me)Ph | * |
| 133 | i-Bu | 1H-1,2,4-triazol-1-yl | Cl | CH(Me)Ph | * |
| 134 | 2-Me-Bu | NHNMe₂ | Cl | 2,6-di-F-Ph | * |
| 135 | 2-Me-Bu | NH-N-Morpholinyl | Cl | 2,6-di-F-Ph | * |
| 136 | i-Bu | 1H-1,2,4-triazol-1-yl | Cl | 3,5-di-Me-Ph | * |
| 137 | i-Bu | 1H-pyrazol-1-yl | Cl | 3,5-di-Me-Ph | 129-131 |
| 138 | i-Bu | 1H-1,2,4-triazol-1-yl | Cl | 2,3,5-tri-F-Ph | * |
| 139 | i-Bu | 1H-pyrazol-1-yl | Cl | 2,3,5-tri-F-Ph | * |
| 140 | i-Bu | 1H-1,2,4-triazol-1-yl | Cl | 2-CF₃-Ph | * |
| 141 | i-Bu | 1H-pyrazol-1-yl | Cl | 2-CF₃-Ph | * |
| 142 | i-Bu | 1H-1,2,4-triazol-1-yl | Cl | 4-F-Ph | * |
| 143 | i-Bu | 1H-pyrazol-1-yl | Cl | 4-F-Ph | * |
| 144 | i-Bu | 1H-1,2,4-triazol-1-yl | Cl | 4-CN-Ph | * |
| 145 | i-Bu | 1H-pyrazol-1-yl | Cl | 4-CN-Ph | * |
| 146 | i-Bu | 1H-1,2,4-triazol-1-yl | Cl | 4-CO₂Me-Ph | * |
| 147 | i-Bu | 1H-pyrazol-1-yl | Cl | 4-CO₂Me-Ph | * |
| 148 | n-Bu | 1H-pyrazol-1-yl | Cl | 2,6-di-F-Ph | 127-128 |
| 149 | i-Bu | 1H-pyrazol-1-yl | Me | 2,6-di-F-Ph | 105-106 |
| (Ex. 9) | | | | | |
| 150 | i-Bu | 1H-pyrazol-1-yl | Cl | 3,5-bis-CF₃-Ph | * |
| 151 | i-Bu | 1H-pyrazol-1-yl | Cl | 5-Cl-2-furyl | 148-152 |
| 152 | i-Bu | 1H-pyrazol-1-yl | Cl | 5-Cl-2-thienyl | * |
| 153 | i-Bu | 1H-pyrazol-1-yl | Cl | 3-furyl | 123-126 |
| 154 | i-Bu | 1H-pyrazol-1-yl | Cl | 3-thienyl | 117-119 |
| 155 | (2S)-Me-Bu | 1H-pyrazol-1-yl | Cl | 2,4,6-tri-F-Ph | 112-113 |
| 156 | i-Bu | 1H-1,2,4-triazol-1-yl | Cl | 5-Cl-2-furyl | 145-146 |
| 157 | i-Bu | 1H-1,2,4-triazol-1-yl | Cl | 5-Cl-2-thienyl | 133-135 |
| 158 | i-Bu | 1H-1,2,4-triazol-1-yl | Cl | 3-furyl | 134-138 |
| 159 | i-Bu | 1H-1,2,4-triazol-1-yl | Cl | 3-thienyl | 144-148 |
| 160 | i-Bu | 1H-1,2,4-triazol-1-yl | Cl | 3-thienyl | 156-158 |
| 161 | Ph | 1H-pyrazol-1-yl | Cl | 2,6-di-F-Ph | * |
| 162 | Ph | 1H-1,2,4-triazol-1-yl | Cl | 2,6-di-F-Ph | 220-225 |
| 163 | Ph | CN | Cl | 2,6-di-F-Ph | 193-198 |
| 164 | 2-Me-Bu | 1H-pyrazol-1-yl | Cl | 2-Cl-4-F-Ph | * |
| 165 | 2-Me-Bu | 1H-1,2,4-triazol-1-yl | Cl | 2-Cl-4-F-Ph | * |
| 166 | 2-Me-Bu | 4-Cl-1H-pyrazol-1-yl | Cl | 2-Cl-4-F-Ph | * |
| 167 | 2-Me-Bu | 3-CF₃-1H-pyrazol-1-yl | Cl | 2-Cl-4-F-Ph | 116-119 |
| 168 | 2-Me-Bu | 3,5-di-Me-1H-pyrazol-1- | Cl | 2-Cl-4-F-Ph | 139-142 |
| | | yl | | | |
| 169 | 2-Me-Bu | 4-Me-1H-pyrazol-1-yl | Cl | 2-Cl-4-F-Ph | * |
| 170 | 2-Me-Bu | 3-Me-1H-pyrazol-1-yl | Cl | 2-Cl-4-F-Ph | * |
| 171 | i-Bu | 1H-pyrazol-1-yl | Cl | 2,3,4,5,6-penta-F-Ph | 136-138 |
| 172 | i-Bu | 1H-pyrazol-1-yl | Cl | Ph | * |
| 173 | i-Bu | 1H-1,2,4-triazol-1-yl | Cl | Ph | * |
| 174 | i-Bu | 1H-pyrazol-1-yl | Cl | 2-F-Ph | * |
| 175 | i-Bu | 1H-1,2,4-triazol-1-yl | Cl | 2-F-Ph | * |
| 176 | i-Bu | 1H-pyrazol-1-yl | Cl | 2-OMe-Ph | * |
| 177 | i-Bu | 1H-1,2,4-triazol-1-yl | Cl | 2-OMe-Ph | * |
| 178 | i-Bu | 3-pyridinyl | Cl | 2,6-di-F-Ph | 175-178 |
| 179 | 2-OMe-Et | 3-CF₃-1H-imidazol-1-yl | Cl | 2,6-di-F-Ph | * |
| 180 | 1-Me-Bu | 1H-pyrazol-1-yl | Cl | 2,6-di-F-Ph | * |
| 181 | 1-Et-Pr | 1H-pyrazol-1-yl | Cl | 2,6-di-F-Ph | * |
| 182 | 3,3-di-Me-Bu | 1H-pyrazol-1-yl | Cl | 2,6-di-F-Ph | * |
| 183 | 2-Et-Bu | 1H-pyrazol-1-yl | Cl | 2,6-di-F-Ph | * |
| 184 | Et | 1H-pyrazol-1-yl | Cl | 2,6-di-F-Ph | * |
| 185 | Me | 1H-pyrazol-1-yl | Cl | 2,6-di-F-Ph | * |
| 186 | 2-propynyl | 1H-pyrazol-1-yl | Cl | 2,6-di-F-Ph | * |
| 187 | CH₂-2-Furyl | 1H-pyrazol-1-yl | Cl | 2,6-di-F-Ph | 200-204 |
| 188 | CH₂-2-Furyl | 1H-1,2,4-triazol-1-yl | Cl | 2,6-di-F-Ph | 188-190 |
| 189 | CH₂-2-Furyl | CN | Cl | 2,6-di-F-Ph | 138-140 |
| 190 | i-Bu | Imidazo[1,2-a]pyridin-3- | Cl | 2,6-di-F-Ph | 194-197 |
| | | yl | | | |
| 191 | 2-Me-Bu | 1H-1,2,3-triazol-2-yl | Cl | 2,6-di-F-Ph | |
| 192 | i-Bu | 1H-1,2,4-triazol-1-yl | Cl | 2-Cl-Ph | * |
| 193 | i-Bu | 1H-pyrazol-1-yl | Cl | 2-Cl-Ph | * |
| 194 | i-Bu | 1H-1,2,4-triazol-1-yl | Cl | 2-Br-Ph | * |
| 195 | i-Bu | 1H-pyrazol-1-yl | Cl | 2-Br-Ph | * |
| 196 | i-Bu | 1H-1,2,4-triazol-1-yl | Cl | 2-Et-Ph | * |
| 197 | i-Bu | 1H-pyrazol-1-yl | Cl | 2-Et-Ph | * |
| 198 | 1-Me-2-SMe-Et | 1H-pyrazol-1-yl | Cl | 2,6-di-F-Ph | 126-127 |
| 199 | 1-Me-2-SMe-Et | 1H-1,2,4-triazol-1-yl | Cl | 2,6-di-F-Ph | 192-194 |
| 200 | 1-Me-2-SO₂Me-Et | 1H-pyrazol-1-yl | Cl | 2,6-di-F-Ph | 232-236 |
| 201 | 1-Me-2-SO₂Me-Et | 1H-1,2,4-triazol-1-yl | Cl | 2,6-di-F-Ph | 209-211 |
| 202 | i-Bu | 1H-pyrazol-1-yl | Cl | 2,6-di-Me-Ph | * |
| 203 | i-Bu | 1H-pyrazol-1-yl | Cl | t-Bu | 119-122 |
| 204 | i-Bu | 1H-pyrazol-1-yl | Cl | i-Pr | 87-89 |
| 205 | i-Bu | 1H-1,2,4-triazol-1-yl | Cl | 2,6-di-Me-Ph | 148-151 |
| 206 | i-Bu | 1H-1,2,4-triazol-1-yl | Cl | t-Bu | * |
| 207 | i-Bu | 1H-1,2,4-triazol-1-yl | Cl | i-Pr | 102-105 |
| 208 | i-Bu | 4-Me-2-pyridinyl | Cl | 2,6-di-F-Ph | * |
| 209 | i-Bu | 5-Me-2-pyridinyl | Cl | 2,6-di-F-Ph | * |
| (Ex. 12) | | | | | |
| 210 | i-Bu | 3-Me-2-pyridinyl | Cl | 2,6-di-F-Ph | * |
| 211 | 1-Me-2-OMe-ethenyl | 1H-pyrazol-1-yl | Cl | 2,6-di-F-Ph | 210-215 |
| 212 | 1-Me-2-OMe-ethenyl | 1H-1,2,4-triazol-1-yl | Cl | 2,6-di-F-Ph | 152-156 |
| 213 | 1-Me-2-OH-Et | 1H-pyrazol-1-yl | Cl | 2,6-di-F-Ph | 229-234 |
| 214 | 1-Me-2-Cl-Et | 1H-1,2,4-triazol-1-yl | Cl | 2,6-di-F-Ph | 202-205 |
| 215 | i-Bu | 1H-1,2,3-triazol-1-yl | Cl | 2,6-di-F-Ph | * |
| 216 | 2-Me-Bu | 4,5-dihydro-1H-pyrazol- | Cl | 2-Cl-4-F-Ph | * |
| | | 1-yl | | | |
| 217 | 2-Me-Bu | 4,5-diliydro-1H-pyrazol- | Cl | 2-Cl-4-F-Ph | 154-156 |
| | | 3-one-1-yl | | | |
| 218 | (2S)-Me-Bu | 4,5-dihydro-1H-pyrazol- | Cl | 2,4,6-tri-F-Ph | * |
| | | 1-yl | | | |
| 219 | (2S)-Me-Bu | 3-Me-1H-pyrazol-1-yl | Cl | 2,4,6-tri-F-Ph | * |
| 220 | 4-Cl-benzyl | CN | Cl | 2,6-di-F-Ph | 140-145 |
| 221 | 4-Cl-benzyl | 1H-pyrazol-1-yl | Cl | 2,6-di-F-Ph | 205 |
| 222 | 4-Cl-benzyl | 1H-1,2,4-triazol-1-yl | Cl | 2,6-di-F-Ph | 160-165 |
| 223 | 2-Me-c-Hex | CN | Cl | 2,6-di-F-Ph | 135-140 |
| 224 | 2-Me-c-Hex | 1H-pyrazol-1-yl | Cl | 2,6-di-F-Ph | 204-208 |
| 225 | 2-Me-c-Hex | 1H-1,2,4-triazol-1-yl | Cl | 2,6-di-F-Ph | 155-160 |
| 226 | n-Bu | 4,5-dihydro-1H-pyrazol- | Cl | 2,4,6-tri-F-Ph | * |
| | | 1-yl | | | |
| 227 | n-Bu | 1H-pyrazol-1-yl | Cl | 2,4,6-tri-F-Ph | 128-129 |
| 228 | n-Bu | 3-Me-1H-pyrazol-1-yl | Cl | 2,4,6-tri-F-Ph | 123-124 |
| 229 | n-Bu | 1H-1,2,4-triazol-1-yl | Cl | 2,4,6-tri-F-Ph | 134-135 |
| 230 | i-Bu | 1H-1,2,4-triazol-1-yl | Cl | 3-F-Ph | * |
| 231 | i-Bu | 1H-pyrazol-1-yl | Cl | 3-F-Ph | * |
| 232 | i-Bu | 1H-1,2,4-triazol-1-yl | Cl | 3-CF₃-Ph | * |
| 233 | i-Bu | 1H-pyrazol-1-yl | Cl | 3-CF₃-Ph | * |
| 234 | i-Bu | 1H-1,2,4-triazol-1-yl | Cl | 3-Br-Ph | * |
| 235 | i-Bu | 1H-1,2,4-triazol-1-yl | Cl | 3-pyridinyl | * |
| 236 | i-Bu | 1H-1,2,4-triazol-1-yl | Cl | 2-Cl-3-pyridinyl | * |
| 237 | i-Bu | 1H-pyrazol-1-yl | Cl | 3-pyridinyl | * |
| 238 | 1-Me-2-F-Et | 1H-pyrazol-1-yl | Cl | 2,6-di-F-Ph | 214-216 |
| 239 | n-Bu | 5-Me-1H-pyrazol-1-yl | Cl | 2,4,6-tri-F-Ph | * |
| 240 | n-Bu | 2-pyridinyl | Cl | 2,4,6-tri-F-Ph | 134-135 |
| 241 | i-Bu | 3-Me-1H-pyrazol-1-yl | Cl | 2,6-di-F-Ph | 135-136 |
| 242 | i-Bu | 3-Me-1H-pyrazol-1-yl | Br | 2,6-di-F-Ph | 131-132 |
| 243 | i-Bu | 3-Me-1H-pyrazol-1-yl | Me | 2,6-di-F-Ph | 113-114 |
| 244 | i-Bu | 1H-pyrazol-1-yl | Cl | 2-Cl-3-pyridinyl | * |
| 245 | i-Bu | 1H-1,2,4-triazol-1-yl | Cl | 3-OMe-Ph | * |
| 246 | i-Bu | 1H-pyrazol-1-yl | Cl | 3-OMe-Ph | * |
| 247 | i-Bu | 1H-1,2,4-triazol-1-yl | Cl | 3-Me-Ph | * |
| 248 | i-Bu | 1H-pyrazol-1-yl | Cl | 3-Me-Ph | * |
| 249 | i-Bu | 1H-1,2,4-triazol-1-yl | Cl | 3-CN-Ph | * |
| 250 | i-Bu | 1H-pyrazol-1-yl | Cl | 3-CN-Ph | * |
| 251 | i-Bu | 1H-pyrazol-1-yl | Cl | 2,3-methylenedioxy- | * |
| | | | | Ph | |
| 252 | i-Bu | 1H-pyrazol-1-yl | Cl | 2,2-difluoro- | 151-154 |
| | | | | benzodioxol-6-yl | |
| 253 | i-Bu | 1H-pyrazol-1-yl | Cl | 2-Cl-4,5- | 207-210 |
| | | | | methylenedioxy-Ph | |
| 254 | i-Bu | 1H-pyrazol-1-yl | Cl | 2,4,6-tri-Me-Ph | 144-146 |
| 255 | i-Bu | 3-Me-1H-pyrazol-1-yl | Cl | 2,3-methylenedioxy- | 145-146 |
| | | | | Ph | |
| 256 | i-Bu | 3-Me-1H-pyrazol-1-yl | Cl | 2,2-difluoro- | 164-165 |
| | | | | benzodioxol-6-yl | |
| 257 | i-Bu | 3-Me-1H-pyrazol-1-yl | Cl | 2-Cl-4,5- | 201-204 |
| | | | | methylenedioxy-Ph | |
| 258 | i-Bu | 3-Me-1H-pyrazol-1-yl | Cl | 2,4,6-tri-Me-Ph | * |
| 259 | 2-Me-Bu | 3-Br-1H-pyrazol-1-yl | Cl | 2-Cl-4-F-Ph | * |
| 260 | 2-Me-Bu | 3-Me-4-Br-1H-pyrazol- | Cl | 2-Cl-4-F-Ph | * |
| | | 1-yl | | | |
| 261 | 2-Me-Bu | 3-(MeO)₂CH-1H- | Cl | 2-Cl-4-F-Ph | 128-132 |
| | | pyrazol-1-yl | | | |
| 262 | (2S)-Me-Bu | 3-Me-1H-pyrazol-1-yl | Cl | 2-Cl-4-F-Ph | * |
| 263 | (2S)-Me-Bu | 1H-pyrazol-1-yl | Cl | 2-Cl-4-F-Ph | * |
| 264 | (2S)-Me-Bu | 4,5-dihydro-1H-pyrazol- | Cl | 2-Cl-4-F-Ph | * |
| | | 1-yl | | | |
| 265 | n-Bu | 3-Me-1H-pyrazol-1-yl | Cl | 2-Cl-4-F-Ph | * |
| 266 | n-Bu | 1H-pyrazol-1-yl | Cl | 2-Cl-4-F-Ph | 134-137 |
| 267 | n-Bu | 4,5-dihydro-1H-pyrazol- | Cl | 2-Cl-4-F-Ph | * |
| | | 1-yl | | | |
| 268 | 2-OH-Et | 1H-pyrazol-1-yl | Cl | 2,6-di-F-Ph | 229-232 |
| 269 | 2-F-Et | 1H-pyrazol-1-yl | Cl | 2,6-di-F-Ph | 194-196 |
| 270 | 2-Br-Et | 1H-pyrazol-1-yl | Cl | 2,6-di-F-Ph | 211-212 |
| 271 | 4-OMe-benzyl | 1H-pyrazol-1-yl | Cl | 2,6-di-F-Ph | 235-236 |
| (Ex. 4) | | | | | |
| 272 | 4-F-benzyl | 1H-pyrazol-1-yl | Cl | 2,6-di-F-Ph | 151-154 |
| 273 | 4-F-benzyl | 1H-1,2,4-triazol-1-yl | Cl | 2,6-di-F-Ph | 92-97 |
| 274 | 4-F-benzyl | CN | Cl | 2,6-di-F-Ph | * |
| 275 | 3-Cl-benzyl | 1H-pyrazol-1-yl | Cl | 2,6-di-F-Ph | 174-175 |
| 276 | 3-Cl-benzyl | 1H-1,2,4-triazol-1-yl | Cl | 2,6-di-F-Ph | 143-145 |
| 277 | 3-Cl-benzyl | CN | Cl | 2,6-di-F-Ph | 134-136 |
| 278 | Ph | 3-Me-1H-pyrazol-1-yl | Cl | 2,6-di-F-Ph | 184-185 |
| 279 | benzyl | 3-Me-1H-pyrazol-1-yl | Cl | 2,6-di-F-Ph | * |
| 280 | 2-F-benzyl | 3-Me-1H-pyrazol-1-yl | Cl | 2,6-di-F-Ph | * |
| 281 | (2S)-Me-Bu | 1H-pyrazol-1-yl | Br | 2-Cl-4-F-Ph | * |
| 282 | (2S)-Me-Bu | 3-Me-1H-pyrazol-1-yl | Br | 2-Cl-4-F-Ph | * |
| 283 | (2S)-Me-Bu | 4,5-dihydro-1H-pyrazol- | Br | 2-Cl-4-F-Ph | * |
| | | 1-yl | | | |
| 284 | n-Bu | 1H-pyrazol-1-yl | Br | 2-Cl-4-F-Ph | 130-132 |
| 285 | n-Bu | 3-Me-1H-pyrazol-1-yl | Br | 2-Cl-4-F-Ph | * |
| 286 | n-Bu | 4,5-dihydro-1H-pyrazol- | Br | 2-Cl-4-F-Ph | * |
| | | 1-yl | | | |
| 287 | Ph | 1H-pyrazol-1-yl | Cl | 2,4,6-tri-F-Ph | 180-182 |
| 288 | Benzyl | 1H-pyrazol-1-yl | Cl | 2-Cl-Ph | 123-126 |
| 289 | i-Bu | 3-Me-1H-pyrazol-1-yl | Cl | i-Pr | 147-149 |
| 290 | i-Bu | 1H-pyrazol-1-yl | Cl | c-Pentyl | * |
| 291 | i-Bu | 1H-pyrazol-1-yl | Cl | c-Hex | 112-114 |
| 292 | i-Bu | 1H-pyrazol-1-yl | Cl | i-Bu | * |
| 293 | i-Bu | 3-Me-1H-pyrazol-1-yl | Cl | c-Pentyl | 154-159 |
| 294 | i-Bu | 3-Me-1H-pyrazol-1-yl | Cl | c-Hex | 155-159 |
| 295 | i-Bu | 3-Me-1H-pyrazol-1-yl | Cl | i-Bu | 113-114 |
| 296 | 2-Me-Bu | CONH-i-Pr | Cl | 2,6-di-F-Ph | * |
| 297 | 2-Me-Bu | CONHMe | Cl | 2,6-di-F-Ph | * |
| 298 | 2-Me-Bu | CONH₂ | Cl | 2,6-di-F-Ph | * |
| 299 | 2-Me-Bu | CONH-n-Pr | Cl | 2,6-di-F-Ph | * |
| 300 | 2-Me-Bu | CONHCH₂-c-Pr | Cl | 2,6-di-F-Ph | * |
| 301 | 2-Me-Bu | CONH-2-Me-Bu | Cl | 2,6-di-F-Ph | * |
| 302 | i-Bu | 1H-pyrazol-1-yl | Cl | 2-Cl-4-F-Ph^{a,c} | * |
| (Ex. 7) | | | | | |
| 303 | i-Bu | 1H-pyrazol-1-yl | Cl | 2-Cl-4-F-Ph^{b,c} | * |
| (Ex. 7) | | | | | |
| 304 | benzyl | 1H-pyrazol-1-yl | Cl | i-Pr | 117-119 |
| 305 | benzyl | 3-Me-1H-pyrazol-1-yl | Cl | i-Pr | 95-99 |
| 306 | Ph | 1H-pyrazol-1-yl | Cl | i-Pr | 190-193 |
| 307 | Ph | 3-Me-1H-pyrazol-1-yl | Cl | i-Pr | 148-151 |
| 308 | CH₂CO₂Me | 3-Me-1H-pyrazol-1-yl | Cl | 2,6-di-F-Ph | 147-149 |
| 309 | 1-Me-2-SMe-Et | 3-Me-1H-pyrazol-1-yl | Cl | 2,6-di-F-Ph | 127-129 |
| 310 | 2-OMe-Et | 3-Me-1H-pyrazol-1-yl | Cl | 2,6-di-F-Ph | 135-138 |
| 311 | 2-SMe-Et | 3-Me-1H-pyrazol-1-yl | Cl | 2,6-di-F-Ph | 145-146 |
| 312 | 1-Me-2-OMe-Et | 2-CN-1H-pyrrol-1-yl | Cl | 2,6-di-F-Ph | 139-142 |
| 313 | CH₂CO₂Me | 3-Me-4-1H-pyrazol-1-yl | Cl | 2,6-di-F-Ph | 69-72 |
| 314 | i-Bu | 1-pyrrolidinyl | Cl | 2,6-di-F-Ph | * |
| 315 | i-Bu | 1H-pyrazol-1-yl | Cl | c-Pr | * |
| 316 | i-Bu | 1H-pyrazol-1-yl | Cl | Et | * |
| 317 | i-Bu | 1H-pyrazol-1-yl | Cl | n-Pentyl | * |
| 318 | i-Bu | 1H-pyrazol-1-yl | Cl | 2,4,6-tri-Cl-Ph | * |
| 319 | i-Bu | 4,5-dihydro-1H-pyrazol- | Cl | 2,4,6-tri-Cl-Ph | * |
| | | 3-one-1-yl | | | |
| 320 | 2-F-Ph | 1H-pyrazol-1-yl | Cl | 2,4,6-tri-F-Ph | * |
| 321 | Ph | 3-Me-1H-pyrazol-1-yl | Cl | 2,4,6-tri-F-Ph | 200-202 |
| 322 | Ph | 1H-pyrazol-1-yl | Cl | 2-Cl-4-F-Ph | 123-125 |
| 323 | Ph | 3-Me-1H-pyrazol-1-yl | Cl | 2-Cl-4-F-Ph | 128-129 |
| 324 | 3-F-Ph | 1H-pyrazol-1-yl | Cl | 2,4,6-tri-F-Ph | * |
| 325 | i-Bu | 2-CN-1H-pyrrol-1-yl | Cl | 2,6-di-F-Ph | 156-157 |
| 326 | 3-Me-Ph | 1H-pyrazol-1-yl | Cl | 2,6-di-F-Ph | 127-129 |
| 327 | 3-Me-Ph | 3-Me-1H-pyrazol-1-yl | Cl | 2,6-di-F-Ph | 75-79 |
| 328 | 2-Me-Ph | 1H-pyrazol-1-yl | Cl | 2,6-di-F-Ph | 193-197 |
| 329 | 2-Me-Ph | 3-Me-1H-pyrazol-1-yl | Cl | 2,6-di-F-Ph | 175-176 |
| 330 | 2-F-benzyl | 1H-pyrazol-1-yl | Cl | 2,6-di-F-Ph | 143-145 |
| 331 | 4-F-benzyl | 3-Me-1H-pyrazol-1-yl | Cl | 2,6-di-F-Ph | 153-155 |
| 332 | 4-Me-Ph | 3-Me-1H-pyrazol-1-yl | Cl | 2,6-di-F-Ph | 234-236 |
| 333 | 4-Me-Ph | 1H-pyrazol-1-yl | Cl | 2,6-di-F-Ph | 219-222 |
| 334 | 2,4-di-Me-Ph | 1H-pyrazol-1-yl | Cl | 2,6-di-F-Ph | 203-206 |
| 335 | 2,4-di-Me-Ph | 3-Me-1H-pyrazol-1-yl | Cl | 2,6-di-F-Ph | 218-221 |
| 336 | 2-F-benzyl | 1H-pyrazol-1-yl | Cl | 2,6-di-F-Ph | 163-165 |
| 337 | 3-F-Ph | 1H-pyrazol-1-yl | Cl | 2,6-di-F-Ph | 235-240 |
| 338 | 3-F-Ph | 3-Me-1H-pyrazol-1-yl | Cl | 2,6-di-F-Ph | 194-196 |
| 339 | 2-F-Ph | 1H-pyrazol-1-yl | Cl | 2,6-di-F-Ph | 200-220 |
| 340 | 2-F-Ph | 3-Me-1H-pyrazol-1-yl | Cl | 2,6-di-F-Ph | 207-208 |
| 341 | 4-F-benzyl | 3-Me-1H-pyrazol-1-yl | Cl | 2,6-di-F-Ph | * |
| 342 | i-Bu | 1H-pyrazol-1-yl | H | 2,6-di-F-Ph | 91-92 |
| (Ex. 3) | | | | | |
| 343 | i-Bu | 2-pyridinyl | Cl | 2,6-di-F-Ph | HCl Salt |
| 344 | 3-Br-benzyl | 1H-pyrazol-1-yl | Cl | 2,4,6-tri-F-Ph | * |
| 345 | 2-Br-benzyl | 1H-pyrazol-1-yl | Cl | 2,4,6-tri-F-Ph | * |
| 346 | 4-CF₃-benzyl | 1H-pyrazol-1-yl | Cl | 2,4,6-tri-F-Ph | * |
| 347 | 3-CF₃-benzyl | 1H-pyrazol-1-yl | Cl | 2,4,6-tri-F-Ph | * |
| 348 | 2-CF₃-benzyl | 1H-pyrazol-1-yl | Cl | 2,4,6-tri-F-Ph | * |
| 349 | 2-Me-Bu | CONHCH₂CH₂OH | Cl | 2,6-di-F-Ph | * |
| 350 | i-Bu | 2-pyrazinyl | Cl | 2,6-di-F-Ph | 102-102 |
| 351 | 3-Br-Ph | 1H-pyrazol-1-yl | Cl | 2,4,6-tri-F-Ph | * |
| 352 | 4-Br-Ph | 1H-pyrazol-1-yl | Cl | 2,4,6-tri-F-Ph | * |
| 353 | 3-CF₃-Ph | 1H-pyrazol-1-yl | Cl | 2,4,6-tri-F-Ph | * |
| 354 | 4-CF₃-Ph | 1H-pyrazol-1-yl | Cl | 2,4,6-tri-F-Ph | * |
| 355 | 2-F-Ph | 3-Me-1H-pyrazol-1-yl | Cl | 2,4,6-tri-F-Ph | 192-194 |
| 356 | 3-F-Ph | 3-Me-1H-pyrazol-1-yl | Cl | 2,4,6-tri-F-Ph | 183-185 |
| 357 | Ph | 1H-pyrazol-1-yl | Cl | 2,3,6-tri-F-Ph | 250-260 |
| 358 | Ph | 3-Me-1H-pyrazol-1-yl | Cl | 2,3,6-tri-F-Ph | 193-194 |
| 359 | Ph | 2-pyridinyl | Me | 2,6-di-F-Ph | * |
| 360 | 2-Br-Ph | 1H-pyrazol-1-yl | Cl | 2,6-di-F-Ph | * |
| 361 | c-Pr | 1H-pyrazol-1-yl | Cl | 2,6-di-F-Ph | * |
| 362 | 2-Ph-c-Pr | 1H-pyrazol-1-yl | Cl | 2,6-di-F-Ph | * |
| 363 | c-Bu | 1H-pyrazol-1-yl | Cl | 2,6-di-F-Ph | * |
| 364 | i-Bu | 2-pyrazinyl | Cl | 2,6-di-F-Ph | HCl Salt |
| 365 | 3,4-di-F-Ph | 1H-pyrazol-1-yl | Cl | 2,4,6-tri-F-Ph | 188-190 |
| 366 | 3,4-di-F-Ph | 3-Me-1H-pyrazol-1-yl | Cl | 2,4,6-tri-F-Ph | 198-200 |
| 367 | 3,5-di-F-Ph | 1H-pyrazol-1-yl | Cl | 2,4,6-tri-F-Ph | 220-221 |
| 368 | 3,5-di-F-Ph | 3-Me-1H-pyrazol-1-yl | Cl | 2,4,6-tri-F-Ph | 221-223 |
| 369 | 2,3-di-F-Ph | 1H-pyrazol-1-yl | Cl | 2,4,6-tri-F-Ph | 174-176 |
| 370 | 2,3-di-F-Ph | 3-Me-1H-pyrazol-1-yl | Cl | 2,4,6-tri-F-Ph | 210-211 |
| 371 | 4-Cl-Ph | 1H-pyrazol-1-yl | Cl | 2,4,6-tri-F-Ph | 205-206 |
| 372 | 4-Cl-Ph | 3-Me-1H-pyrazol-1-yl | Cl | 2,4,6-tri-F-Ph | 188-191 |
| 373 | 3-Cl-Ph | 1H-pyrazol-1-yl | Cl | 2,4,6-tri-F-Ph | 170-173 |
| 374 | 3-Cl-Ph | 3-Me-1H-pyrazol-1-yl | Cl | 2,4,6-tri-F-Ph | 178-179 |
| 375 | 2-Cl-Ph | 1H-pyrazol-1-yl | Cl | 2,4,6-tri-F-Ph | 188-190 |
| 376 | 2-Cl-Ph | 3-Me-1H-pyrazol-1-yl | Cl | 2,4,6-tri-F-Ph | 215-217 |
| 377 | 4-Br-benzyl | 1H-pyrazol-1-yl | Cl | 2,4,6-tri-F-Ph | * |
| 378 | 2,3-di-Me-Ph | 1H-pyrazol-1-yl | Cl | 2,6-di-F-Ph | 177-179 |
| 379 | 2,3-di-Me-Ph | 3-Me-1H-pyrazol-1-yl | Cl | 2,6-di-F-Ph | 214-215 |
| 380 | 2,3-di-F-Ph | 1H-pyrazol-1-yl | Cl | 2,6-di-F-Ph | 218-219 |
| 381 | 2,3-di-F-Ph | 3-Me-1H-pyrazol-1-yl | Cl | 2,6-di-F-Ph | 193-194 |
| 382 | 4-F-Ph | 1H-pyrazol-1-yl | Cl | 2,6-di-F-Ph | 195-196 |
| 383 | 4-F-Ph | 3-Me-1H-pyrazol-1-yl | Cl | 2,6-di-F-Ph | 198-199 |
| 384 | 3,5-di-F-Ph | 1H-pyrazol-1-yl | Cl | 2,6-di-F-Ph | 233-235 |
| 385 | 3,5-di-F-Ph | 3-Me-1H-pyrazol-1-yl | Cl | 2,6-di-F-Ph | 210-211 |
| 386 | 2,4-di-F-Ph | 1H-pyrazol-1-yl | Cl | 2,6-di-F-Ph | 111-113 |
| 387 | 2,4-di-F-Ph | 3-Me-1H-pyrazol-1-yl | Cl | 2,6-di-F-Ph | 178-179 |
| 388 | 3,4-di-F-Ph | 1H-pyrazol-1-yl | Cl | 2,6-di-F-Ph | 233-235 |
| 389 | 3,4-di-F-Ph | 3-Me-1H-pyrazol-1-yl | Cl | 2,6-di-F-Ph | 205-206 |
| 390 | i-Bu | 3-Me-1H-pyrazol-1-yl | Cl | s-Bu | * |
| 391 | i-Bu | 1H-pyrazol-1-yl | Cl | 2,6-di-Cl-4-CF₃-Ph | * |
| 392 | N═C(Me)₂ | 1H-pyrazol-1-yl | Cl | 2,6-di-F-Ph | * |
| (Ex. 4) | | | | | |
| 393 | 2-OMe-Ph | 1H-pyrazol-1-yl | Cl | 2,6-di-F-Ph | * |
| 394 | 3-OMe-Ph | 1H-pyrazol-1-yl | Cl | 2,6-di-F-Ph | * |
| 395 | 4-OMe-Ph | 1H-pyrazol-1-yl | Cl | 2,6-di-F-Ph | * |
| 396 | 2-OCF₃-Ph | 1H-pyrazol-1-yl | Cl | 2,6-di-F-Ph | * |
| 397 | 2-OCF₃-Ph | 1H-pyrazol-1-yl | Cl | 2,6-di-F-Ph | * |
| 398 | 4-OCF₃-Ph | 1H-pyrazol-1-yl | Cl | 2,6-di-F-Ph | * |
| 399 | 2-CF₃-Ph | 1H-pyrazol-1-yl | Cl | 2,6-di-F-Ph | * |
| 400 | NH₂ | 1H-pyrazol-1-yl | Cl | 2,6-di-F-Ph | * |
| (Ex. 4) | | | | | |
| 401 | i-Bu | 2-pyridinyl | I | 2,6-di-F-Ph | * |
| 402 | 3,4-di-Cl-Ph | 1H-pyrazol-1-yl | Cl | 2,4,6-tri-F-Ph | 219-220 |
| 403 | 3,4-di-Cl-Ph | 3-Me-1H-pyrazol-1-yl | Cl | 2,4,6-tri-F-Ph | 218-220 |
| 404 | 3,5-di-Cl-Ph | 1H-pyrazol-1-yl | Cl | 2,4,6-tri-F-Ph | 210-211 |
| 405 | 3,5-di-Cl-Ph | 3-Me-1H-pyrazol-1-yl | Cl | 2,4,6-tri-F-Ph | 208-210 |
| 406 | 3-Cl-4-F-Ph | 1H-pyrazol-1-yl | Cl | 2,4,6-tri-F-Ph | 198-200 |
| 407 | 3-Cl-4-F-Ph | 3-Me-1H-pyrazol-1-yl | Cl | 2,4,6-tri-F-Ph | 118-121 |
| 408 | 4-F-Ph | 1H-pyrazol-1-yl | Cl | 2,4,6-tri-F-Ph | 150-152 |
| 409 | 4-F-Ph | 3-Me-1H-pyrazol-1-yl | Cl | 2,4,6-tri-F-Ph | 188-192 |
| 410 | 3,4,5-tri-F-Ph | 1H-pyrazol-1-yl | Cl | 2,4,6-tri-F-Ph | 238-240 |
| 411 | 3,4,5-tri-F-Ph | 3-Me-1H-pyrazol-1-yl | Cl | 2,4,6-tri-F-Ph | 190-193 |
| 412 | Et | CONH₂ | Cl | 2-Cl-4-F-Ph | * |
| 413 | n-Pr | CONH₂ | Cl | 2,4,6-tri-F-Ph | * |
| 414 | 3-F-Ph | CONH₂ | Cl | 2,4,6-tri-F-Ph | * |
| (Ex. 8) | | | | | |
| 415 | 2-Me-Bu | CONH₂ | Cl | 2,4,6-tri-F-Ph | * |
| 416 | 2-Me-Bu | CONH-Propargyl | Cl | 2,4,6-tri-F-Ph | * |
| 417 | 2-Me-Bu | CONHOMe | Cl | 2,4,6-tri-F-Ph | * |
| 418 | c-Hex | 1H-pyrazol-1-yl | Cl | 2,4,6-tri-F-Ph | * |
| 419 | Ph | CONH₂ | Cl | 2,3,6-tri-F-Ph | * |
| 420 | Ph | CONH₂ | Cl | 2,4,6-tri-F-Ph | * |
| 421 | 2-Me-Bu | 1H-pyrazol-1-yl | Cl | 2-F-4-OMe-Ph | * |
| 422 | i-Bu | NHC(═O)H | Cl | 2,6-di-F-Ph | * |
| (Ex. 13) | | | | | |
| 423 | i-Bu | NH(C═O)CH₃ | Cl | 2,6-di-F-Ph | * |
| 424 | i-Bu | 1H-pyrazol-1-yl | Cl | s-Bu | * |
| (Ex. 5) | | | | | |

*See Index Table C for ¹H NMR data.
^{a}Compound 302 has a retention time of 22.6 minutes; see Example 7.
^{b}Compound 303 has a retention time of 18.9 minutes; see Example 7.
^{c}Compounds 302 and 303 are atropisomers of each other.

INDEX TABLE B

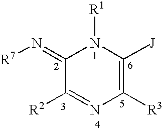

| Cmpd | | | | | | M.P. |
|---|---|---|---|---|---|---|
| No. | R¹ | R² | R7 | R³ | J | (° C.) |
| 425 | i-Bu | CN | H | Me | 2,4-di-F-Ph | 95-97 |
| 426 | 2-Me-Bu | CN | H | Me | 2,4-di-F-Ph | 73-77 |
| (Ex. 14) | | | | | | |
| 427 | i-Bu | CONH₂ | H | Me | 2,4-di-F-Ph | 172-177 |
| 428 | 2-Me-Bu | CONH₂ | H | Me | 2,4-di-F-Ph | 148-156 |
| 429 | i-Bu | CONH₂ | H | F | 2,4-di-F-Ph | 152-158 |
| 430 | 2-Me-Bu | CN | Ac | Me | 2,4-di-F-Ph | * |
| (Ex. 14) | | | | | | |
| 431 | i-Bu | CN | Ac | Me | Ph | * |

*See Index Table C for ¹H NMR data.

INDEX TABLE C

| Compd. No. | ¹H NMR Data (CDCl₃ solution unless indicated otherwise)^{a} |
|---|---|
| 1 | δ 9.1 (m, 1H), 7.9 (m, 1H), 7.5 (m, 1H), 7.1 (m, 2H), 6.5 (m, 1H), 3.8 (d, 2H), |
| | 2.0 (m, 1H), 0.8 (d, 6H). |
| 2 | δ 8.86 (m, 1H), 8.43 (m, 1H), 7.83 (m, 1H), 7.59 (m, 1H), 7.38 (m, 1H), 7.12 (m, |
| | 2H), 3.79 (d, 2H), 2.00 (m, 1H), 0.79 (d, 6H). |
| 3 | δ 9.81 (s, 1H), 8.21 (s, 1H), 7.62 (m, 1H), 7.14 (m, 2H), 3.84 (d, 2H), 2.00 (m, 1H), |
| | 0.80 (d, 6H). |
| 4 | δ 7.46 (m, 1H), 7.21 (d, 1H), 7.04 (m, 2H), 4.33 (m, 2H), 3.61 (d, 2H), 2.97 (m, 2H), |
| | 1.94 (m, 1H), 0.75 (d, 6H). |
| 9 | δ 9.12 (m, 1H), 7.89 (m, 1H), 7.53 (m, 1H), 7.42 (m, 1H), 7.21 (m, 1H), 6.51 (m, |
| | 1H), 3.77 (m, 2H), 1.95 (m, 1H), 0.81 (m, 6H). |
| 10 | δ 9.81 (s, 1H), 8.21 (s, 1H), 7.57 (m, 1H), 7.45 (m, 1H), 7.24 (m, 1H), 3.84 (m, 2H), |
| | 1.96 (m, 1H), 0.82 (m, 6H). |
| 13 | δ 9.2 (m, 1H), 7.91 (m, 1H), 7.45 (m, 1H), 7.35 (m, 1H), 6.52 (m, 1H), 3.94 (m, 1H), |
| | 3.92 (s, 3H), 3.65 (m, 1H), 2.02 (m, 1H), 0.81 (d, 6H). |
| 14 | δ 9.11 (m, 1H), 7.88 (m, 1H), 7.55 (m, 1H), 7.36 (m, 2H), 7.29 (m, 1H), 6.44 (m, |
| | 1H), 3.45 (s, 3H). |
| 15 | δ 9.81 (s, 1H), 8.21 (m, 1H), 7.62 (m, 1H), 7.41 (m, 2H), 7.31 (m, 1H), 3.45 (s, 3H). |
| 16 | δ 7.5 (m, 1H), 7.20 (s, 1H), 7.0 (t, 2H), 4.3 (t, 2H), 3.6 (d, 2H), 2.9 (t, 2H), 0.9 (m, |
| | 1H), 0.4 (m, 2H), 0.9 (m, 2H). |
| 17 | δ 9.8 (s, 1H), 8.2 (s, 1H), 7.6 (m, 1H), 7.1 (t, 2H) 3.9 (d, 2H), 1.0 (m, 1H), 0.5 (m, |
| | 2H), 0.2 (m, 2H). |
| 18 | δ 9.1 (d, 1H), 7.8 (d, 1H), 7.6 (m, 1H), 7.1 (t, 2H), 6.5 (t, 1H), 3.8 (d, 2H), 1.0 (m, |
| | 1H), 1.4 (m, 2H), 0.2 (m, 2H). |
| 19 | δ 9.1 (d, 1H), 7.9 (d, 1H), 6.9 (t, 2H), 6.5 (m, 1H), 3.8 (d, 2H), 1.2 (m, 1H), 1.0 (m, |
| | 1H), 0.77-0.75 (m, 6H). |
| 20 | δ 9.7 (s, 1H), 8.2 (s, 1H), 6.9 (t, 2H), 3.8 (d, 2H), 1.7 (m, 1H), 1.2 (m, 1H), 1.0 (m, |
| | 1H), 0.79-0.73 (m, 6H). |
| 21 | δ 7.65 (m, 1H), 7.15 (t, 2H), 3.8 (d, 2H), 1.7 (m, 1H), 1.2 (m, 1H), 1.0 (m, 1H), |
| | 0.75-0.68 (m, 6H). |
| 22 | δ 9.07 (d, 1H), 7.88 (s, 1H), 7.59-7.50 (m, 2H), 7.29 (d, 2H), 6.55-6.49 (m, 1H), |
| | 3.82 (d, 2H), 2.07-1.96 (m, 1H), 0.76 (d, 6H). |
| 23 | δ 9.77 (s, 1H), 8.20 (s, 1H), 7.57 (d, 2H), 7.30 (d, 2H), 3.86 (d, 2H), 2.03 (d, 1H), |
| | 0.78 (d, 6H). |
| 24 | δ 7.57 (d, 2H), 7.30 (d, 2H), 3.81 (d, 2H), 2.57 (s, 3H), 2.44 (s, 3H), 2.07-1.96 (m, |
| | 1H), 0.77 (d, 6H). |
| 25 | δ 9.05 (d, 1H), 7.90 (d, 1H), 7.55 (m, 1H), 7.11 (t, 2H), 6.50 (t, 1H), 2.35 (m, 1H), |
| | 1.85 (m, 1H), 1.57 (m, 4H), 0.75 (t, 3H). |
| 26 | δ 9.04 (d, 1H), 7.89 (d, 1H), 7.58 (m, 1H), 7.12 (t, 2H), 6.50 (t, 1H), 4.15 (m, 1H), |
| | 1.58 (m, 6H). |
| 27 | δ 9.15 (d, 1H), 7.90 (d, 1H), 7.60 (m, 1H), 7.13 (t, 2H), 6.50 (t, 1H), 3.85 (t, 2H), |
| | 1.62 (m, 2H), 0.80 (t, 3H). |
| 28 | δ 9.11 (d, 1H), 7.88 (d, 1H), 7.60 (m, 1H), 7.13 (t, 2H), 6.51 (t, 1H), 3.85 (t, 2H), |
| | 1.58 (m, 2H), 1.16 (m, 4H), 0.79 (t, 3H). |
| 29 | δ 9.05 (d, 1H), 7.90 (d, 1H), 7.58 (m, 1H), 7.12 (t, 2H), 6.52 (t, 1H), 3.40 (m, 1H), |
| | 2.90 (m, 1H), 1.60 (d, 3H), 0.82 (d, 3H), 0.73 (d, 3H). |
| 30 | δ 9.13 (d, 1H), 7.91 (d, 1H), 7.58 (m, 1H), 7.06 (t, 2H), 6.52 (t, 1H), 4.78 (s, 1H), |
| | 4.56 (s, 2H), 4.37 (s, 1H), 1.58 (s, 3H). |
| 31 | δ 9.10 (d, 1H), 7.90 (d, 1H), 7.58 (m, 1H), 7.12 (t, 2H), 6.50 (t, 1H), 3.80 (d, 2H), |
| | 1.60 (m, 3H), 1.50 (m, 2H), 1.10 (m, 2H), 0.90 (m, 2H), 0.78 (m, 2H). |
| 32 | δ 9.76 (s, 1H), 8.20 (s, 1H), 7.60 (m, 1H), 7.15 (t, 2H), 3.85 (m, 1H), 2.35 (m, 1H), |
| | 1.88 (m, 1H), 1.60 (d, 3H), 0.78 (t, 3H). |
| 33 | δ 9.77 (s, 1H), 8.20 (s, 2H), 7.61 (m, 1H), 7.13 (t, 2H), 4.20 (m, 1H), 1.60 (m, 6H). |
| 34 | δ 9.81 (s, 1H), 8.21 (s, 1H), 7.62 (m, 1H), 7.16 (t, 2H), 3.88 (t, 2H), 1.62 (m, 2H), |
| | 0.82 (t, 3H). |
| 35 | δ 9.10 (d, 1H), 7.88 (d, 1H), 7.57 (m, 1H), 7.13 (t, 2H), 6.50 (t, 1H), 3.89 (t, 2H), |
| | 1.45 (m, 3H), 0.73 (m, 6H). |
| 36 | δ 9.08 (d, 1H), 7.89 (d, 1H), 7.83 (d, 2H), 7.51 (d, 2H), 6.52 (d, 1H), 3.80 (d, 2H), |
| | 2.04 (s, 1H), 0.76 (d, 6H). |
| 37 | δ 9.06 (d, 1H), 7.87 (s, 1H), 7.32-7.16 (m, 2H), 7.05 (d, 2H), 6.57-6.42 (m, 1H), |
| | 3.97-3.76 (m, 5H), 2.11-1.97 (m, 1H), 0.75 (d, 6H). |
| 38 | δ 9.07 (d, 1H), 7.88 (s, 1H), 7.41 (s, 4H), 6.52-6.49 (m, 1H), 3.82 (d, 2H), 1.99 (s, |
| | 1H), 0.76 (d, 6H). |
| 39 | δ 9.10 (d, 1H), 7.92-7.87 (m, 1H), 7.72-7.64 (m, 1H), 7.42 (t, 1H), 7.35-7.30 (m, 1H), |
| | 6.52 (m, 1H), 4.15 (m, 1H), 3.41-3.31 (m, 1H), 0.85 (d, 3H), 0.74 (d, 3H). |
| 44 | δ 9.79 (s, 1H), 8.21 (s, 1H), 8.13-8.00 (m, 1H), 7.48-7.28 (m, 2H), 3.39-3.24 (m, 2H), |
| | 2.01-1.82 (m, 1H), 0.85-0.66 (m, 6H). |
| 45 | δ 9.09 (d, 1H), 7.93-7.88 (m, 1H), 7.37-7.28 (m, 3H), 6.53-6.49 (m, 1H), |
| | 4.01-3.93 (m, 1H), 3.67-3.58 (m, 1H), 2.04-1.95 (m, 1H), 0.80 (d, 3H), 0.76 (d, 3H). |
| 46 | δ 9.10 (d, 1H), 7.92-7.88 (m, 1H), 7.67-7.62 (m, 1H), 7.58-7.52 (m, 2H), 6.52 (m, |
| | 1H), 4.00-3.91 (m, 1H), 3.65-3.55 (m, 1H), 2.03-1.96 (m, 1H), 0.80 (d, 3H), 0.76 (d, |
| | 3H). |
| 47 | δ 9.0 (d, 1H), 7.8 (s, 1H), 7.5 (m, 1H), 7.1 (m, 2H), 6.5 (d, 1H), 3.6 (br s, 1H), 2.6 (br |
| | s, 2H), 1.8 (d, 2H), 1.7 (d, 2H), 1.6 (d, 1H), 1.2 (m, 1H), 1.0 (m, 2H). |
| 48 | δ 9.01 (m, 1H), 7.44 (m, 1H), 7.08 (m, 2H), 3.62 (d, 2H), 2.19 (s, 3H), 2.03 (s, 3H), |
| | 1.85 (m, 1H), 0.75 (d, 6H). |
| 50 | δ 9.10 (d, 1H), 7.92-7.87 (m, 1H), 7.45-7.37 (m, 1H), 7.35-7.28 (m, 1H), |
| | 7.16-7.11 (m, 1H), 6.51 (m, 1H), 3.99-3.89 (m, 1H), 3.73-3.63 (m, 1H), 2.01 (m, 1H), |
| | 0.81-0.75 (m, 6H). |
| 55 | δ 9.81 (s, 1H), 8.22 (s, 1H), 7.51-7.43 (m, 1H), 7.14-7.09 (m, 1H), 3.90-3.80 (m, 2H), |
| | 2.04-1.97 (m, 1H), 0.84-0.80 (m, 6H). |
| 56 | δ 9.09 (d, 1H), 7.89 (d, 1H), 7.29-7.22 (m, 2H), 7.12-7.06 (m, 1H), 6.51 (m, 1H), |
| | 4.04-3.94 (m, 1H), 3.70-3.61 (m, 1H), 2.04-1.98 (m, 1H), 0.82-0.76 (m, 6H). |
| 57 | δ 9.11 (d, 1H), 7.91 (d, 1H), 7.48-7.39 (m, 1H), 7.12-7.05 (m, 1H), 6.51 (m, 1H), |
| | 3.84-3.74 (m, 2H), 2.03-1.94 (m, 1H), 0.83-0.78 (m, 6H). |
| 58 | δ 9.11 (d, 1H), 7.89 (d, 1H), 7.58 (m, 1H), 7.09 (t, 2H), 6.51 (t, 1H), 5.77 (m, 1H), |
| | 5.16 (d, 1H), 4.89 (d, 1H), 4.55 (d, 2H). |
| 59 | δ 9.08 (d, 1H), 7.89 (d, 1H), 7.57 (m, 1H), 7.09 (t, 2H), 6.50 (t, 1H), 3.97 (s, 2H), |
| | 0.82 (s, 9H). |
| 60 | δ 8.98 (d, 1H), 7.85 (d, 1H), 7.63 (m, 1H), 7.15 (t, 2H), 3.87 (d, 2H), 1.72 (m, 1H), |
| | 1.26 (m, 1H), 1.06 (m, 1H), 0.42 (m, 6H). |
| 61 | δ 9.08 (d, 1H), 8.08 (d, 1H), 7.60 (m, 1H), 7.18 (s, 1H), 7.12 (t, 2H), 3.83 (d, 2H), |
| | 1.68 (m, 1H), 1.22 (m, 1H), 1.03 (m, 1H), 0.75 (d, 3H), 0.71 (t, 3H). |
| 62 | δ 7.60 (m, 1H), 7.15 (t, 2H), 6.04 (s, 1H), 3.80 (d, 2H), 2.42 (s, 3H), 2.32 (s, 3H), |
| | 1.75 (m, 1H), 1.23 (m, 1H), 1.03 (m, 1H), 0.74 (d, 3H), 0.69 (t, 3H). |
| 63 | δ 9.08 (d, 1H), 7.60 (m, 1H), 7.13 (t, 2H), 6.74 (d, 1H), 3.86 (d, 2H), 1.70 (m, 1H), |
| | 1.22 (m, 1H), 1.04 (m, 1H), 0.74 (d, 3H), 0.70 (t, 3H). |
| 64 | δ 7.62 (m, 1H), 7.15 (t, 2H), 6.47 (s, 1H), 3.83 (d, 2H), 2.46 (s, 3H), 1.70 (m, 1H), |
| | 1.23 (m, 1H), 1.02 (m, 1H), 0.75 (d, 3H), 0.70 (t, 3H). |
| 65 | δ 9.01 (d, 1H), 7.59 (m, 1H), 7.12 (t, 2H), 6.51 (d, 1H), 3.83 (d, 2H), 1.69 (m, 1H), |
| | 1.22 (m, 1H), 1.01 (m, 1H), 0.74 (d, 3H), 0.69 (t, 3H). |
| 66 | δ 9.03 (d, 1H), 7.57 (m, 1H), 7.10 (t, 2H), 6.31 (d, 1H), 3.82 (d, 2H), 2.44 (s, 3H), |
| | 1.70 (m, 1H), 1.23 (m, 1H), 1.01 (m, 1H), 0.75 (d, 3H), 0.70 (t, 3H). |
| 67 | δ 9.21 (s, 1H), 7.66 (m, 1H), 7.17 (t, 2H), 3.90 (d, 2H), 1.72 (m, 1H), 1.23 (m, 1H), |
| | 1.03 (m, 1H), 0.77 (d, 3H), 0.72 (t, 3H). |
| 68 | δ 8.31 (s, 1H), 7.63 (m, 1H), 7.15 (t, 2H), 3.84 (d, 2H), 1.70 (m, 1H), 1.22 (m, 1H), |
| | 1.05 (m, 1H), 0.76 (d, 3H), 0.71 (t, 3H). |
| 69 | δ 7.89 (d, 1H), 7.60 (m, 1H), 7.13 (t, 2H), 6.98 (d, 1H), 3.81 (d, 2H), 2.69 (s, 3H), |
| | 1.70 (m, 1H), 1.22 (m, 1H), 1.02 (m, 1H), 0.75 (d, 3H), 0.70 (t, 3H). |
| 70 | δ 8.85 (d, 1H), 7.60 (m, 1H), 7.15 (t, 2H), 7.02 (d, 1H), 3.82 (d, 2H), 3.03 (q, 2H), |
| | 1.71 (m, 1H), 1.39 (t, 3H), 1.24 (m, 1H), 1.03 (m, 1H), 0.75 (d, 3H), 0.73 (t, 3H). |
| 71 | δ 8.04 (d, 1H), 7.60 (m, 1H), 7.15 (t, 2H), 7.01 (d, 1H), 3.81 (d, 2H), 3.0 (t, 2H), |
| | 1.82 (q, 2H), 1.70 (m, 1H), 1.23 (m, 1H), 1.02 (t, 4H), 0.75 (d, 3H), 0.72 (t, 3H). |
| 72 | δ 7.71 (d, 1H), 7.60 (m, 1H), 7.14 (t, 2H), 7.04 (d, 1H), 3.81 (d, 2H), 3.50 (m, 1H), |
| | 1.7 (m, 1H), 1.39 (d, 6H), 1.21 (m, 1H), 1.04 (m, 1H), 0.75 (d, 3H), 0.71 (t, 3H). |
| 73 | δ 8.85 (m, 1H), 7.50 (m, 1H), 7.04 (m, 2H), 3.65 (d, 2H), 2.63 (m, 2H), 2.43 (m, 2H), |
| | 1.95 (m, 1H), 1.81 (m, 3H), 0.75 (d, 6H). |
| 74 | δ 9.06 (d, 1H), 7.87 (s, 1H), 7.54 (d, 2H), 7.31-7.20 (m, 2H), 6.49 (d, 1H), 3.85 (d, |
| | 2H), 2.06-1.96 (m, 1H), 1.38 (s, 9H), 0.74 (d, 6H). |
| 75 | δ 9.77 (s, 1H), 8.19 (s, 1H), 7.57 (d, 2H), 7.26 (d, 2H), 3.89 (d, 2H), 2.05-1.97 (m, |
| | 1H), 1.39 (s, 9H), 0.75 (d, 6H). |
| 76 | δ 9.78 (s, 1H), 8.21 (s, 1H), 7.86 (d, 2H), 7.52 (d, 2H), 3.84 (d, 2H), 2.02 (dt, 1H), |
| | 0.78 (d, 6H). |
| 77 | δ 9.77 (s, 1H), 8.20 (s, 1H), 7.42 (s, 4H), 3.86 (d, 2H), 2.01 (s, 1H), 0.78 (d, 6H). |
| 78 | δ 9.38 (m, 1H), 7.52 (m, 1H), 7.10 (m, 2H), 3.90 (s, 3H), 3.63 (m, 2H), 2.33 (d, 3H), |
| | 1.85 (m, 1H), 0.75 (d, 6H). |
| 79 | δ 9.77 (s, 1H), 8.20 (s, 1H), 7.21-7.17 (m, 2H), 7.12 (m, 1H), 4.10-4.03 (m, 1H), |
| | 3.57-3.49 (m, 1H), 2.43 (s, 3H), 2.14 (s, 3H), 2.03-1.96 (m, 1H), 0.82 (d, 3H), 0.75 (d, |
| | 3H). |
| 80 | δ 9.07 (d, 1H), 7.87 (m, 1H), 7.21-7.09 (m, 3H), 6.50 (m, 1H), 4.04 (m, 1H), |
| | 3.52-3.42 (m, 1H), 2.41 (s, 3H), 2.13 (s, 3H), 2.04-1.96 (m, 1H), 0.80 (d, 3H), 0.74 (d, |
| | 3H). |
| 81 | δ 9.12 (d, 1H), 7.61 (m, 1H), 7.14 (t, 2H), 6.86 (d, 1H), 3.87 (d, 2H), 1.72 (m, 1H), |
| | 1.22 (m, 1H), 1.03 (m, 1H), 0.75 (d, 3H), 0.72 (t, 3H). |
| 82 | δ 8.10 (t, 2H), 7.58 (m, 1H), 7.10 (t, 2H), 6.34 (t, 2H), 3.79 (d, 2H), 1.68 (m, 1H), |
| | 1.22 (m, 1H), 1.02 (m, 1H), 0.74 (d, 3H), 0.70 (t, 3H). |
| 83 | δ 8.57 (s, 1H), 7.67-7.60 (m, 2H), 7.10 (t, 2H), 4.06 (s, 3H), 3.75 (d, 2H), |
| | 2.05-1.93 (m, 1H), 0.77 (d, 6H). |
| 84 | δ 8.75 (d, 2H), 8.34 (d, 2H), 7.65-7.57 (m, 1H), 7.14 (t, 2H), 3.79 (d, 2H), |
| | 2.04-1.95 (m, 1H), 0.79 (d, 6H). |
| 87 | δ 9.12 (d, 1H), 7.90 (d, 1H), 7.79-7.70 (m, 2H), 7.54 (m, 1H), 6.51 (m, 1H), |
| | 4.24-4.17 (m, 1H), 3.15 (m, 1H), 2.03-1.95 (m, 1H), 0.87 (d, 3H), 0.74 (d, 3H). |
| 90 | δ 9.10 (d, 1H), 8.02 (d, 1H), 7.97-7.89 (m, 3H), 7.85 (s, 1H), 7.63 (m, 2H), 7.38 (m, |
| | 1H), 6.51 (m, 1H), 3.93-3.83 (m, 2H), 2.12-2.01 (m, 1H), 0.72 (m, 6H). |
| 91 | δ 9.07 (s, 1H), 7.97-7.85 (m, 1H), 7.63-7.49 (m, 1H), 7.15-7.02 (m, 2H), |
| | 6.59-6.45 (m, 1H), 4.33-4.16 (m, 2H), 3.72-3.56 (m, 2H), 3.41-3.31 (m, 1H), 2.03-1.94 (m, |
| | 1H), 1.81-1.72 (m, 1H), 1.69-1.59 (m, 1H), 1.49-1.38 (m, 1H). |
| 92 | δ 9.77 (s, 1H), 8.29-8.19 (m, 1H), 7.65-7.55 (m, 1H), 7.19-7.02 (m, 2H), |
| | 4.40-4.27 (m, 1H), 4.28-4.19 (m, 1H), 3.77-3.65 (m, 1H), 3.67-3.51 (m, 2H), 2.02 (m, 1H), |
| | 1.83-1.73 (m, 1H), 1.72-1.57 (m, 1H), 1.51-1.36 (m, 1H). |
| 99 | δ 9.0 (d, 1H), 7.8 (s, 1H), 7.6 (m, 1H), 7.1 (m, 2H), 6.5 (d, 1H), 3.8 (d, 2H), 1.9 (m, |
| | 1H), 0.7 (d, 6H). |
| 100 | δ 8.57 (s, 1H), 8.35 (s, 1H), 7.55 (m, 1H), 7.09 (t, 2H), 3.97 (s, 3H), 3.73 (d, 2H), |
| | 1.95 (m, 1H), 0.77 (d, 6H). |
| 103 | δ 7.62-7.55 (m, 3H), 7.12 (t, 2H), 4.29 (s, 3H), 3.76 (d, 2H), 2.03-1.94 (m, 1H), |
| | 0.78 (d, 6H). |
| 110 | δ 7.6 (m, 1H), 7.1 (t, 2H), 3.7 (d, 2H), 1.6 (m, 1H), 1.2 (m, 1H), 1.0 (m, 1H), 0.70 (m, |
| | 6H). |
| 111 | δ 9.04 (d, 1H), 7.83 (d, 1H), 7.55 (d, 1H), 7.41 (m, 1H), 7.30 (d, 1H), 6.45 (m, 1H), |
| | 4.02 (m, 1H), 3.40-3.31 (m, 1H), 1.95 (m, 1H), 0.79 (d, 3H), 0.69 (d, 3H). |
| 112 | δ 9.73 (s, 1H), 8.13 (s, 1H), 6.18 (s, 2H), 3.87 (s, 3H), 3.76 (m, 8H), 1.99-1.88 (m, |
| | 1H), 0.74 (d, 6H). |
| 113 | δ 9.81 (s, 1H), 8.22 (s, 1H), 7.83-7.72 (m, 2H), 7.55 (t, 1H), 4.24 (m, 1H), 3.22 (m, |
| | 1H), 2.00 (m, 1H), 0.88 (d, 3H), 0.76 (d, 3H). |
| 116 | δ 9.77 (s, 1H), 8.20 (s, 1H), 7.60 (m, 1H), 7.27 (m, 2H), 3.85 (d, 2H), 2.05 (m, 1H), |
| | 0.82 (d, 6H). |
| 119 | δ 9.80 (s, 1H), 8.21 (s, 1H), 8.04 (d, 1H), 7.95 (m, 2H), 7.87 (s, 1H), 7.65 (m, 2H), |
| | 7.39 (m, 1H), 4.00-3.88 (m, 2H), 2.07 (m, 1H), 0.74 (m, 6H). |
| 128 | δ 9.71 (s, 1H), 8.18 (s, 1H), 7.39-7.28 (m, 3H), 7.12 (d, 2H), 4.35 (s, 2H), 3.95 (d, |
| | 2H), 2.24 (m, 1H), 0.98 (d, 6H). |
| 129 | δ 9.83 (s, 1H), 8.23 (s, 1H), 8.08 (d, 1H), 8.00 (d, 1H), 7.68-7.52 (m, 4H), 7.41 (d, |
| | 1H), 4.06 (dd, 1H), 3.38 (m, 1H), 1.92 (m, 1H), 0.69 (m, 6H). |
| 130 | δ 9.01 (d, 1H), 7.86 (d, 1H), 7.37-7.26 (m, 3H), 7.12 (d, 2H), 6.47 (m, 1H), 4.31 (s, |
| | 2H), 3.89 (d, 2H), 2.22 (m, 1H), 0.96 (d, 6H). |
| 131 | δ 9.13 (d, 1H), 8.05 (d, 1H), 7.98 (m, 1H), 7.91 (d, 1H), 7.67-7.50 (m, 4H), 7.44 (d, |
| | 1H), 6.53 (m, 1H), 4.02 (m, 1H), 3.31 (m, 1H), 1.92 (m, 1H), 0.66 (m, 6H). |
| 132 | δ 8.99 (d, 1H), 7.84 (d, 1H), 7.35 (t, 2H), 7.27 (m, 1H), 7.21 (d, 2H), 6.46 (m, 1H), |
| | 4.76 (s, 1H), 4.09 (br s, 2H), 2.13 (m, 1H), 1.86 (d, 3H), 0.93 (d, 3H), 0.83 (br s, 3H). |
| 133 | δ 9.69 (s, 1H), 8.16 (s, 1H), 7.36 (m, 2H), 7.29 (m, 1H), 7.20 (d, 2H), 4.79 (br s, 1H), |
| | 4.14 (br s, 2H), 2.16 (m, 1H), 1.88 (d, 3H), 0.96 (d, 3H), 0.88 (br s, 3H). |
| 134 | δ 7.50 (m, 1H), 7.14 (m, 1H), 7.03 (m, 2H), 3.65 (m, 2H), 2.76 (m, 6H), 1.61 (m, |
| | 1H), 0.72 (d, 6H). |
| 135 | δ 7.48 (m, 1H), 7.24 (s, 1H), 7.02 (m, 2H), 3.89 (d, 3H), 3.62 (m, 2H), 3.03 (m, 3H), |
| | 1.25 (m, 2H), 0.99 (m, 2H), 0.71 (m, 3H), 0.66 (m, 3H). |
| 136 | δ 9.76 (s, 1H), 8.19 (s, 1H), 7.19 (s, 1H), 6.93 (s, 2H), 3.87 (d, 2H), 2.41 (s, 6H), |
| | 2.09 (m, 1H), 0.78 (d, 6H). |
| 138 | δ 9.79 (s, 1H), 8.21 (s, 1H), 7.22 (m, 1H), 6.90 (m, 1H), 3.98 (m, 1H), 3.72 (m, 1H), |
| | 2.02 (m, 1H), 0.82 (m, 6H). |
| 139 | δ 9.09 (d, 1H), 7.89 (d, 1H), 7.20 (m, 1H), 6.92 (m, 1H), 6.51 (m, 1H), 3.92 (m, 1H), |
| | 3.68 (m, 1H), 2.02 (m, 1H), 0.80 (m, 6H). |
| 140 | δ 9.80 (s, 1H), 8.21 (s, 1H), 7.92 (m, 1H), 7.83-7.73 (m, 2H), 7.49 (d, 1H), 4.24 (dd, |
| | 1H), 3.11 (dd, 1H), 2.14 (dd, 1H), 0.87 (d, 3H), 0.73 (d, 3H). |
| 141 | δ 9.10 (d, 1H), 7.88 (m, 2H), 7.75 (m, 2H), 7.47 (d, 1H), 6.51 (dd, 1H), 4.22 (dd, |
| | 1H), 3.05 (dd, 1H), 2.12 (m, 1H), 0.85 (d, 3H), 0.71 (d, 3H). |
| 142 | δ 9.77 (s, 1H), 8.20 (s, 1H), 7.36 (m, 2H), 7.29 (d, 2H), 3.87 (d, 2H), 2.02 (s, 1H), |
| | 0.77 (d, 6H). |
| 143 | δ 9.07 (d, 1H), 7.88 (d, 1H), 7.34 (m, 2H), 7.26 (m, 2H), 6.50 (dd, 1H), 3.83 (d, 2H), |
| | 2.00 (m, 1H), 0.76 (d, 6H). |
| 144 | δ 9.78 (s, 1H), 8.21 (s, 1H), 7.90 (m, 2H), 7.52 (d, 2H), 3.82 (d, 2H), 2.00 (td, 1H), |
| | 0.78 (d, 6H). |
| 145 | δ 9.08 (d, 1H), 7.87 (m, 3H), 7.51 (d, 2H), 6.52 (dd, 1H), 3.78 (d, 2H), 1.98 (dt, 1H), |
| | 0.76 (d, 6H). |
| 146 | δ 9.78 (s, 1H), 8.27-8.19 (m, 3H), 7.47-7.43 (m, 2H), 4.00 (s, 3H), 3.85 (d, 2H), |
| | 2.05 (s, 1H), 0.76 (d, 6H). |
| 147 | δ 9.08 (d, 1H), 8.23 (d, 2H), 7.89 (d, 1H), 7.45 (d, 2H), 6.51 (m, 1H), 3.99 (s, 3H), |
| | 3.81 (d, 2H), 1.98 (m, 1H), 0.74 (d, 6H). |
| 150 | δ 9.09 (d, 1H), 8.09 (s, 1H), 7.90 (m, 1H), 7.86 (s, 2H), 6.54 (m, 1H), 3.75 (d, 2H), |
| | 2.00 (m, 1H), 0.78 (d, 6H). |
| 152 | δ 9.09 (d, 1H), 7.88 (d, 1H), 7.02 (d, 1H), 6.98 (d, 1H), 6.50 (m, 1H), 3.93 (d, 2H), |
| | 2.16 (m, 1H), 0.85 (d, 6H). |
| 161 | δ 9.10 (s, 1H), 7.90 (s, 1H), 7.30 (m, 6H), 6.85-6.80 (t, 2H), 6.50 (s, 1H). |
| 164 | δ 9.08 (m, 1H), 7.89 (d, 1H), 7.40 (m, 1H), 7.34 (m, 1H), 7.20 (m, 1H), 6.50 (m, 1H), |
| | 4.10 (m, 1H), 3.48 (m, 1H), 1.75 (m, 1H), 1.22-0.95 (m, 2H), 0.72 (m, 6H). |
| 165 | δ 9.80 (d, 1H), 8.20 (s, 1H), 7.40 (m, 2H), 7.24 (m, 1H), 4.12 (m, 1H), 3.54 (m, 1H), |
| | 1.89-1.62 (m, 1H), 1.29-0.92 (m, 2H), 0.72 (m, 6H). |
| 166 | δ 9.11 (s, 1H), 7.81 (s, 1H), 7.38 (m, 2H), 7.20 (m, 1H), 4.12 (m, 1H), 3.48 (m, 1H), |
| | 1.70 (m, 1H), 1.32-0.89 (m, 2H), 0.74 (m, 6H). |
| 169 | δ 8.85 (s, 1H), 7.71 (s, 1H), 7.40 (m, 1H), 7.32 (m, 1H), 7.18 (m, 1H), 4.10 (m, 1H), |
| | 3.46 (m, 1H), 2.16 (s, 3H), 1.72 (m, 1H), 1.10 (m, 2H), 0.72 (m, 6H). |
| 170 | δ 9.02 (d, 1H), 7.40 (m, 1H), 7.32 (m, 1H), 7.18 (m, 1H), 6.31 (d, 1H), 4.09 (m, 1H), |
| | 3.45 (m, 1H), 2.44 (s, 3H), 1.72 (m, 1H), 1.02 (m, 2H), 0.72 (m, 6H). |
| 172 | δ 9.07 (d, 1H), 7.88 (d, 1H), 7.54 (m, 3H), 7.32 (m, 2H), 6.50 (dd, 1H), 3.84 (d, 2H), |
| | 2.02 (m, 1H), 0.75 (d, 6H). |
| 173 | δ 9.78 (s, 1H), 8.20 (s, 1H), 7.58 (m, 3H), 7.35 (td, 2H), 3.88 (d, 2H), 2.02 (m, 1H), |
| | 0.76 (d, 6H). |
| 174 | δ 9.09 (d, 1H), 7.88 (d, 1H), 7.58 (m, 1H), 7.36 (m, 2H), 7.26 (m, 1H), 6.50 (dd, 1H), |
| | 3.98 (dd, 1H), 3.66 (dd, 1H), 1.98 (m, 1H), 0.78 (d, 3H), 0.74 (d, 3H). |
| 175 | δ 9.79 (s, 1H), 8.20 (m, 1H), 7.62 (m, 1H), 7.38 (m, 2H), 7.29 (t, 1H), 4.02 (dd, 1H), |
| | 3.71 (dd, 1H), 1.99 (m, 1H), 0.79 (d, 3H), 0.75 (d, 3H). |
| 176 | δ 9.07 (d, 1H), 7.87 (d, 1H), 7.52 (d, 1H), 7.26 (dd, 1H), 7.10 (m, 1H), 7.04 (d, 1H), |
| | 6.49 (dd, 1H), 4.07 (dd, 1H), 3.82 (s, 3H), 3.46 (dd, 1H), 2.00 (m, 1H), 0.79 (d, 3H), |
| | 0.70 (d, 3H). |
| 177 | δ 9.79 (s, 1H), 8.20 (s, 1H), 7.56 (s, 1H), 7.26 (dd, 1H), 7.16 (m, 1H), 7.06 (d, 1H), |
| | 4.10 (m, 1H), 3.83 (s, 3H), 3.52 (dd, 1H), 2.00 (q, 1H), 0.80 (d, 3H), 0.71 (d, 3H). |
| 179 | δ 9.05 (s, 1H), 8.48 (m, 1H), 7.58 (m, 1H), 7.10 (m, 2H), 4.18 (m, 2H), 3.52 (m, 2H), |
| | 3.17 (s, 3H). |
| 180 | δ 9.03 (d, 1H), 7.88 (d, 1H), 7.57 (m, 1H), 7.11 (t, 2H), 6.50 (t, 1H), 3.90 (m, 1H), |
| | 2.23 (m, 1H), 1.86 (m, 1H), 1.58 (d, 3H), 1.17 (m, 2H), 0.80 (t, 3H). |
| 181 | δ 9.02 (d, 1H), 7.90 (d, 1H), 7.59 (m, 1H), 7.13 (t, 2H), 6.50 (t, 1H), 3.51 (m, 1H), |
| | 2.22 (m, 2H), 2.01 (m, 2H), 0.79 (m, 6H). |
| 182 | δ 9.11 (d, 1H), 8.90 (d, 1H), 7.58 (m, 1H), 7.13 (t, 2H), 6.54 (t, 1H), 3.93 (m, 2H), |
| | 1.50 (m, 2H), 0.74 (s, 9H). |
| 183 | δ 9.11 (d, 1H), 7.90 (d, 1H), 7.59 (m, 1H), 7.12 (t, 2H), 6.51 (t, 1H), 3.92 (d, 2H), |
| | 1.46 (m, 1H), 1.18 (m, 4H), 0.65 (t, 6H). |
| 184 | δ 9.12 (d, 1H), 8.89 (d, 1H), 7.60 (m, 1H), 7.13 (t, 2H), 6.51 (t, 1H), 3.97 (q, 2H), |
| | 1.21 (t, 3H). |
| 185 | δ 9.13 (d, 1H), 7.90 (d, 1H), 7.60 (m, 1H), 7.13 (t, 2H), 6.52 (t, 1H), 3.48 (s, 3H). |
| 186 | δ 9.15 (d, 1H), 7.90 (d, 1H), 7.60 (m, 1H), 7.13 (t, 2H), 6.50 (t, 1H), 4.71 (s, 2H), |
| | 2.23 (s, 1H). |
| 191 | δ 8.00 (d, 2H), 7.60 (m, 1H), 7.15 (t, 2H), 3.85 (m, 2H), 1.80 (m, 1H), 1.25 (m, 1H), |
| | 1.1-1.0 (m, 1H), 0.74 (m, 6H). |
| 192 | δ 9.79 (s, 1H), 8.21 (s, 1H), 7.60 (m, 1H), 7.52 (m, 2H), 7.40 (dd, 1H), 4.13 (dq, 1H), |
| | 3.48 (dd, 1H), 2.02 (m, 1H), 0.84 (d, 3H), 0.75 (d, 3H). |
| 193 | δ 9.10 (d, 1H), 7.89 (d, 1H), 7.58 (m, 1H), 7.48 (m, 2H), 7.40 (m, 1H), 6.50 (m, 1H), |
| | 4.10 (m, 1H), 3.43 (dd, 1H), 2.02 (dt, 1H), 0.83 (d, 3H), 0.73 (d, 3H). |
| 194 | δ 9.80 (s, 1H), 8.21 (s, 1H), 7.78 (dd, 1H), 7.55 (td, 1H), 7.47 (td, 1H), 7.40 (dd, 1H), |
| | 4.15 (td, 1H), 3.44 (dd, 1H), 2.02 (m, 1H), 0.85 (d, 3H), 0.75 (d, 3H). |
| 195 | δ 9.11 (s, 1H), 7.89 (s, 1H), 7.75 (m, 1H), 7.52 (td, 1H), 7.42 (m, 2H), 6.51 (s, 1H), |
| | 4.12 (m, 1H), 3.39 (dd, 1H), 2.03 (dd, 1H), 0.84 (d, 3H), 0.73 (d, 3H). |
| 196 | δ 9.78 (s, 1H), 8.21 (s, 1H), 7.53 (dd, 1H), 7.45 (m, 1H), 7.39 (s, 1H), 7.24 (dd, 1H), |
| | 4.10 (m, 1H), 3.39 (s, 1H), 2.40 (m, 2H), 2.02 (m, 1H), 1.26 (t, 3H), 0.82 (d, 3H), |
| | 0.71 (d, 3H). |
| 197 | δ 9.08 (d, 1H), 7.88 (d, 1H), 7.50 (m, 1H), 7.44 (m, 1H), 7.36 (m, 1H), 7.24 (dd, 1H), |
| | 6.50 (dd, 1H), 4.11 (dd, 1H), 3.34 (dd, 1H), 2.42 (m, 2H), 2.00 (m, 1H), 1.20 (t, 3H), |
| | 0.81 (d, 3H), 0.70 (d, 3H). |
| 202 | δ 9.08 (d, 1H), 7.89 (d, 1H), 7.34 (m, 1H), 7.19 (d, 2H), 6.50 (m, 1H), 3.72 (d, 2H), |
| | 2.17 (s, 6H), 1.76 (m, 1H), 0.78 (d, 6H). |
| 206 | δ 9.68 (s, 1H), 8.17 (s, 1H), 4.33 (br s, 2H), 2.30 (m, 1H), 1.66 (s, 9H), 0.84 (d, 6H). |
| 208 | δ 8.70 (s, 1H), 8.26 (s, 1H), 7.58 (m, 1H), 7.21 (d, 1H), 7.12 (t, 2H), 3.78 (d, 2H), |
| | 2.45 (s, 3H), 2.02 (m, 1H), 0.78 (d, 6H). |
| 209 | δ 8.68 (s, 1H), 8.40 (s, 1H), 7.60 (m, 2H), 7.12 (t, 2H), 3.79 (d, 2H), 2.44 (s, 3H), |
| | 2.02 (m, 1H), 0.78 (d, 6H). |
| 210 | δ 8.69 (s, 1H), 7.64 (m, 1H), 7.50 (m, 1H), 7.45 (m, 1H), 7.16 (t, 2H), 3.87 (d, 2H), |
| | 2.42 (s, 3H), 1.22 (m, 1H), 0.80 (d, 6H). |
| 215 | δ 9.81 (s, 1H), 8.21 (m, 1H), 7.52 (m, 1H), 7.45 (m, 1H), 7.21 (m, 1H), 4.01 (m, 1H) |
| | 3.65 (m, 1H), 2.00 (m, 1H), 0.81 (d, 6H). |
| 216 | δ 7.38 (m, 1H), 7.27 (m, 1H), 7.20 (s, 1H), 7.12 (m, 1H), 4.30 (m, 2H), 3.98 (m, 1H), |
| | 3.26 (m, 1H), 2.97 (t, 2H), 1.82-1.56 (m, 1H), 1.23-0.86 (m, 2H), 0.71 (m, 6H). |
| 218 | δ 7.2 (s, 1H), 6.8 (m, 2H), 4.3 (d, 2H), 3.6 (d, 2H), 2.9 (d, 2H) 1.6 (m, 1H), 1.2 (m, |
| | 1H), 0.9 (m, 1H), 0.7 (m, 6H). |
| 219 | δ 9.0 (d, 1H), 6.8 (m, 2H), 6.3 (d, 1H), 3.8 (d, 2H), 2.4 (s, 3H), 1.6 (m, 1H), 1.2 (m, |
| | 1H), 1.0 (m, 1H), 0.7 (m, 6H). |
| 226 | δ 7.21 (s, 1H), 6.82 (m, 2H), 4.33 (m, 2H), 3.67 (m, 2H), 2.99 (m, 2H), 1.45 (m, 2H), |
| | 1.19 (m, 2H), 0.75 (m, 3H). |
| 230 | δ 9.77 (s, 1H), 8.20 (s, 1H), 7.58 (d, 1H), 7.33-7.26 (m, 1H), 7.17-7.07 (m, 2H), |
| | 3.87 (d, 2H), 2.04 (s, 1H), 0.79 (d, 6H). |
| 231 | δ 9.07 (s, 1H), 7.89 (s, 1H), 7.54 (s, 1H), 7.30-7.20 (m, 1H), 7.16-7.06 (m, 2H), |
| | 6.51 (s, 1H), 3.83 (d, 2H), 2.08-1.98 (m, 1H), 0.77 (d, 6H). |
| 232 | δ 9.79 (s, 1H), 8.21 (s, 1H), 7.87 (d, 1H), 7.75 (t, 1H), 7.65 (s, 1H), 7.58 (d, 1H), |
| | 3.83 (d, 2H), 2.02 (m, 1H), 0.78 (dd, 6H). |
| 233 | δ 9.08 (d, 1H), 7.89 (d, 1H), 7.83 (d, 1H), 7.72 (t, 1H), 7.63 (s, 1H), 7.56 (d, 1H), |
| | 6.51 (m, 1H), 3.79 (d, 2H), 2.00 (dt, 1H), 0.76 (d, 6H). |
| 234 | δ 9.78 (s, 1H), 8.20 (s, 1H), 7.73 (d, 1H), 7.52 (t, 1H), 7.47 (t, 1H), 7.30 (d, 1H), |
| | 3.86 (dd, 2H), 2.04 (m, 1H), 0.79 (s, 6H). |
| 235 | δ 9.78 (s, 1H), 8.84 (dd, 1H), 8.65 (d, 1H), 8.21 (s, 1H), 7.73 (dt, 1H), 7.55 (dd, 1H), |
| | 3.87 (d, 2H), 1.98 (m, 1H), 0.78 (s, 6H). |
| 236 | δ 9.81 (s, 1H), 8.66 (dd, 1H) 8.22 (s, 1H), 7.80 (dd, 1H), 7.51 (dd, 1H), 4.19 (dd, |
| | 1H), 3.42 (dd, 1H), 2.01 (dd, 1H), 0.87 (d, 3H), 0.76 (d, 3H). |
| 237 | δ 9.10 (s, 1H), 8.82 (d, 1H), 8.63 (s, 1H), 7.91 (s, 1H), 7.72 (dd, 1H), 7.55 (dd, 1H), |
| | 6.50 (s, 1H), 3.82 (d, 2H), 1.98 (m, 1H), 0.75 (dd, 6H). |
| 239 | δ 7.67 (s, 1H), 6.92 (m, 2H), 6.23 (m, 1H), 3.83 (m, 2H), 2.49 (s, 3H), 1.55 (m, 2H), |
| | 1.19 (m, 2H), 0.79 (m, 3H). |
| 244 | δ 9.11 (d, 1H), 8.63 (dd, 1H), 7.91 (d, 1H), 7.79 (dd, 1H), 7.48 (dd, 1H), 6.52 (m, |
| | 1H), 4.15 (d, 1H), 3.36 (dd, 1H), 1.98 (m, 1H), 0.85 (d, 3H), 0.74 (d, 3H). |
| 245 | δ 9.79 (s, 1H), 8.20 (s, 1H), 7.45 (t, 1H), 7.13 (d, 1H), 6.90 (d, 1H), 6.85 (s, 1H), |
| | 3.88 (s, 3H), 3.90 (d, 2H), 2.08 (m, 1H), 0.77 (d, 6H). |
| 246 | δ 9.05 (s, 1H), 7.88 (s, 1H), 7.44 (t, 1H), 7.10 (d, 1H), 6.90 (d, 1H), 6.85 (s, 1H), |
| | 3.87 (s, 3H), 3.86 (d, 2H), 2.10 (m, 1H), 0.78 (d, 6H). |
| 247 | δ 9.73 (s, 1H), 8.19 (s, 1H), 7.50-7.35 (m, 2H), 7.16 (s, 1H), 3.88 (m, 1H), 2.46 (s, |
| | 3H), 2.10 (m, 1H), 0.76 (m, 6H). |
| 248 | δ 9.07 (s, 1H), 7.85 (s, 1H), 7.50-7.35 (m, 2H), 7.16 (s, 1H), 6.50 (s, 1H), 3.88 (m, |
| | 1H), 2.46 (s, 3H), 2.10 (m, 1H), 0.76 (m, 6H). |
| 249 | δ 9.78 (s, 1H), 8.21 (s, 1H), 7.90 (d, 1H), 7.75 (t, 1H), 7.70 (s, 1H), 7.61 (d, 1H), |
| | 3.82 (d, 2H), 1.98 (m, 1H), 0.79 (d, 6H). |
| 250 | δ 9.05 (s, 1H), 7.92 (s, 1H), 7.90 (d, 1H), 7.75 (t, 1H), 7.70 (s, 1H), 7.61 (d, 1H), |
| | 6.52 (s, 1H), 3.82 (d, 2H), 1.98 (m, 1H), 0.79 (d, 6H). |
| 251 | δ 9.08 (d, 1H), 7.87 (d, 1H), 7.00 (m, 2H), 6.78 (m, 1H), 6.49 (m, 1H), 6.02 (m, 2H), |
| | 4.00 (m, 1H), 3.82-3.72 (m, 1H), 2.02 (m, 1H), 0.76 (m, 6H). |
| 258 | δ 9.00 (d, 1H), 6.99 (s, 2H), 6.30 (d, 1H), 3.70 (d, 2H), 2.44 (s. 3H), 2.35 (s, 3H), |
| | 2.11 (s, 6H), 1.80 (m, 1H), 0.78 (d, 6H). |
| 259 | δ 9.00 (d, 1H), 7.45-7.30 (m, 2H), 7.26-7.15 (m, 1H), 6.52 (d, 1H), 4.10 (m, 1H), |
| | 3.52 (m, 1H), 1.87-1.58 (m, 1H), 1.24-0.96 (m, 2H), 0.72 (m, 6H). |
| 260 | δ 9.12 (s, 1H), 7.40 (m, 2H), 7.18 (m, 1H), 4.06 (m, 1H), 3.46 (m, 1H), 2.41 (s, 3H), |
| | 1.64 (m, 1H), 1.23-0.98 (m, 2H), 0.72 (m, 6H). |
| 262 | δ 9.02 (d, 1H), 7.40 (m, 1H), 7.33 (m, 1H), 7.18 (m, 1H), 6.31 (d, 1H), 4.10 (m, 1H), |
| | 3.44 (m, 1H), 2.44 (s, 3H), 1.70 (m, 1H), 1.23-0.89 (m, 2H), 0.72 (m, 6H). |
| 263 | δ 9.15-9.02 (m, 1H), 7.89 (d, 1H), 7.46-7.30 (m, 2H), 7.20 (m, 1H), 6.50 (m, 1H), |
| | 4.10 (m, 1H), 3.57-3.40 (m, 1H), 1.89-1.60 (m, 1H), 1.32-0.91 (m, 2H), 0.72 (m, 6H). |
| 264 | δ 7.34 (m, 1H), 7.28 (m, 1H), 7.20 (t, 1H), 7.12 (m, 1H), 4.28 (m, 2H), 3.96 (m, 1H), |
| | 3.26 (m, 1H), 2.97 (t, 2H), 1.83-1.54 (m, 1H), 1.25-0.85 (m, 2H), 0.72 (m, 6H). |
| 265 | δ 9.03 (d, 1H), 7.38 (m, 2H), 7.18 (m, 1H), 6.31 (d, 1H), 4.10 (m, 1H), 3.51 (m, 1H), |
| | 2.44 (s, 3H), 1.76-1.59 (m, 1H), 1.55-1.38 (m, 1H), 1.25-1.13 (m, 2H), 0.77 (t, 3H). |
| 267 | δ 7.38 (m, 1H), 7.28 (m, 1H), 7.18 (m, 1H), 7.12 (m, 1H), 4.28 (m, 2H), 3.90 (m, |
| | 1H), 3.30 (m, 1H), 2.98 (m, 2H), 1.69-1.53 (m, 1H), 1.47-1.32 (m, 1H), 1.22-1.09 (m, |
| | 2H), 0.75 (t, 3H). |
| 274 | δ 7.7-7.6 (m, 1H), 7.1-7.05 (t, 2H), 6.9-6.8 (m, 4H), 5.12 (s, 2H). |
| 279 | δ 9.05 (s, 1H), 7.5-7.45 (m, 1H), 7.2-7.15 (m, 3H), 7.0-6.95 (t, 2H), 6.85 (s, 2H), |
| | 6.25 (s, 1H), 5.18 (s, 2H), 2.45 (s, 3H). |
| 280 | δ 9.05 (s, 1H), 7.5-7.4 (m, 1H), 7.2 (m, 1H), 7.0-6.8 (m, 5H), 6.3 (s, 1H), 5.27 (s, |
| | 2H), 2.45 (s, 3H). |
| 281 | δ 9.07 (m, 1H), 7.89 (d, 1H), 7.40 (m, 1H), 7.34 (m, 1H), 7.18 (m, 1H), 6.50 (m, 1H), |
| | 4.18 (m, 1H), 3.46 (m, 1H), 1.90-1.59 (m, 1H), 1.33-0.90 (m, 2H), 0.72 (m, 6H). |
| 282 | δ 8.99 (d, 1H), 7.38 (m, 1H), 7.30 (m, 1H), 7.18 (m, 1H), 6.30 (d, 1H), 4.10 (m, 1H), |
| | 3.44 (m, 1H), 2.43 (s, 3H), 1.66 (m, 1H), 1.31-1.03 (m, 2H), 0.72 (m, 6H). |
| 283 | δ 7.36 (m, 1H), 7.26 (m, 1H), 7.20 (m, 1H), 7.12 (m, 1H), 4.36-4.22 (m, 2H), |
| | 4.00 (m, 1H), 3.26 (m, 1H), 2.96 (m, 2H), 1.62 (m, 1H), 0.85-1.22 (m, 2H), 0.72 (m, 6H). |
| 285 | δ 8.99 (d, 1H), 7.36 (m, 2H), 7.20 (m, 1H), 6.30 (d, 1H), 4.06 (m, 1H), 3.50 (m, 1H), |
| | 2.43 (s, 3H), 1.73-1.39 (m, 2H), 1.25-1.14 (m, 2H), 0.77 (t, 3H). |
| 286 | δ 7.35 (m, 1H), 7.28 (m, 1H), 7.20 (m, 1H), 7.14 (m, 1H), 4.28 (m, 2H), 3.96 (m, |
| | 1H), 3.32 (m, 1H), 2.96 (m, 2H), 1.58 (m, 1H), 1.36 (m, 1H), 1.14 (m, 2H), 0.74 (m, |
| | 3H). |
| 290 | δ 8.95 (s, 1H), 7.82 (s, 1H), 6.45 (s, 1H), 4.16 (m, 1H), 3.26 (m, 1H), 2.30 (m, 2H), |
| | 2.12 (m, 1H), 1.96 (m, 5H), 1.70 (m, 2H), 1.01 (d, 6H). |
| 292 | δ 8.98 (s, 1H), 7.84 (s, 1H), 6.47 (s, 1H), 4.03 (d, 2H), 2.78 (d, 2H), 2.20 (m, 1H) |
| | 2.05 (m, 1H), 1.06-0.96 (m, 12H). |
| 296 | δ 9.38 (d, 1H), 7.60 (m, 1H), 7.12 (t, 2H), 4.37 (m, 1H), 3.80 (d, 2H), 1.60 (s, 1H), |
| | 1.29 (d, 6H), 1.21 (m, 1H), 1.02 (m, 1H), 0.72 (m, 6H). |
| 297 | δ 9.48 (s, 1H), 7.60 (m, 1H), 7.13 (t, 2H), 3.83 (d, 2H), 3.07 (d, 3H), 1.68 (m, 1H), |
| | 1.21 (m, 1H), 1.03 (m, 1H), 0.72 (m, 6H). |
| 298 | δ 9.20 (s, 1H), 7.60 (m, 1H), 7.14 (t, 2H), 6.25 (s, 1H), 3.85 (d, 2H), 1.72 (m, 1H), |
| | 1.21 (t, 1H), 1.02 (m, 1H), 0.73 (m, 6H). |
| 299 | δ 9.50 (s, 1H), 7.60 (m, 1H), 7.13 (t, 2H), 3.85 (d, 2H), 3.48 (q, 2H), 1.65 (m, 2H), |
| | 1.64 (m, 1H), 1.22 (m, 1H), 1.01 (t, 3H), 0.72 (m, 6H). |
| 300 | δ 9.53 (s, 1H), 7.60 (m, 1H), 7.13 (t, 2H), 3.83 (d, 2H), 3.39 (m, 2H), 1.71 (m, 1H), |
| | 1.22 (m, 2H), 1.01 (m, 1H), 0.73 (m, 6H), 0.58 (d, 2H), 0.31 (d, 2H). |
| 301 | δ 9.46 (s, 1H), 7.62 (m, 1H), 7.17 (t, 2H), 3.82 (d, 2H), 3.50 (m, 2H), 1.67 (m, 2H), |
| | 1.57 (s, 3H), 1.38 (s, 2H), 1.22 (m, 1H), 1.03 (m, 1H), 0.92 (m, 3H), 0.73 (m, 6H). |
| 302 | δ 9.10 (br s, 1H), 7.89 (br s, 1H), 7.40 (m, 1H), 7.34 (m, 1H), 7.20 (m, 1H), 6.51 (br |
| | s, 1H), 4.10 (m, 1H), 3.40 (m, 1H), 2.00 (m, 1H), 0.85 (d, 3H), 0.75 (d, 3H). |
| 303 | 9.09 (br s, 1H), 7.89 (br s, 1H), 7.39 (m, 1H), 7.34 (m, 1H), 7.20 (m, 1H), 6.52 (br s, |
| | 1H), 4.10 (m, 1H), 3.38 (m, 1H), 2.00 (m, 1H), 0.84 (d, 3H), 0.75 (d, 3H). |
| 314 | δ 7.56 (m, 1H), 7.09 (t, 2H), 4.02 (t, 2H), 3.70 (d, 2H), 2.62 (t, 2H), 2.22 (m, 2H), |
| | 1.91 (s, 1H), 0.76 (d, 6H). |
| 315 | δ 8.99 (d, 1H), 7.84 (d, 1H), 6.46 (m, 1H), 4.34 (d, 2H), 2.28 (m, 1H), 1.78 (m, 1H), |
| | 1.35 (m, 2H), 0.96 (m, 8H). |
| 316 | δ 8.97 (s, 1H), 7.83 (s, 1H) 6.46 (s, 1H) 4.02 (d, 2H), 2.90 (m, 2H), 2.22 (m, 1H), |
| | 1.26 (m, 3H), 1.00 (d, 6H). |
| 317 | δ 8.96 (d, 1H), 7.83 (s, 1H), 6.46 (s, 1H), 4.01 (d, 2H), 2.83 (m, 2H), 2.22 (m, 1H), |
| | 1.60 (m, 2H), 1.42 (m, 4H), 1.00 (d, 6H), 0.94 (t, 3H). |
| 318 | δ 9.12 (d, 1H), 7.90 (s, 1H), 7.55 (s, 2H), 6.52 (m, 1H), 3.76 (d, 2H), 1.88 (m, 1H), |
| | 0.85 (d, 6H). |
| 319 | δ 7.49 (s, 2H), 4.38 (t, 2H), 3.55 (d, 2H), 2.80 (m, 2H), 1.76 (m, 1H), 0.80 (d, 6H). |
| 320 | δ 9.1 (s, 1H), 7.93 (s, 1H), 7.4-7.3 (m, 1H), 7.25-7.15 (m, 3H), 6.7-6.6 (m, 2H), |
| | 6.5 (s, 1H). |
| 341 | δ 9.05 (s, 1H), 7.35-7.25 (m, 1H), 7.2-7.15 (m, 1H), 7.0-6.85 (m, 3H), 6.6 (d, 1H), |
| | 6.55 (d, 1H), 6.3 (s, 1H), 5.16 (m, 2H), 2.45 (s, 3H). |
| 343 | δ 9.53 (s, 1H), 9.25 (s, 1H), 8.50 (s, 1H), 8.01 (s, 1H), 7.66 (s, 1H), 7.18 (m, 2H), |
| | 3.90 (s, 2H), 2.06 (m, 1H), 0.81 (d, 6H). |
| 344 | δ 9.12 (d, 1H), 7.93 (s, 1H), 7.40 (d, 1H), 7.12 (t, 1H), 6.90 (t, 2H), 6.80 (t, 2H), |
| | 6.57 (d, 1H), 5.15 (s, 2H). |
| 345 | δ 9.17 (d, 1H), 7.93 (d, 1H), 7.42 (d, 1H), 7.22 (t, 1H), 7.12 (t, 1H), 6.88 (d, 1H), |
| | 6.70 (t, 2H), 6.58 (d, 1H), 5.35 (s, 2H). |
| 346 | δ 9.10 (d, 1H), 7.92 (s, 1H), 7.50 (d, 2H), 7.06 (d, 2H), 6.77 (t, 2H), 6.57 (d, 1H), |
| | 5.23 (s, 2H). |
| 347 | δ 9.12 (d, 1H), 7.92 (d, 1H), 7.56 (d, 1H), 7.40 (t, 1H), 7.26 (d, 1H), 6.99 (s, 1H), |
| | 6.76 (t, 2H), 6.58 (t, 1H), 5.24 (s, 2H). |
| 348 | δ 9.16 (d, 1H), 7.97 (s, 1H), 7.59 (d, 1H), 7.49 (t, 1H), 7.38 (t, 1H), 6.97 (d, 1H), |
| | 6.67 (t, 2H), 6.56 (t, 1H), 5.41 (s, 2H). |
| 349 | δ 9.56 (s, 1H), 7.60 (m, 1H), 7.13 (t, 2H), 3.84 (d, 2H), 3.39 (t, 2H), 1.72 (m, 1H), |
| | 1.60 (s, 1H), 1.2 (m, 1H), 1.1 (m, 1H), 1.0 (m, 1H), 0.73 (m, 6H), 0.58 (q, 2H), |
| | 0.32 (q, 2H). |
| 351 | δ 9.08 (t, 1H), 7.92 (d, 1H), 7.54 (d, 1H), 7.39 (s, 1H), 7.25 (m, 1H), 7.18 (t, 1H), |
| | 6.64 (m, 2H), 6.52 (d, 1H). |
| 352 | δ 9.04 (t, 1H), 7.92 (d, 1H), 7.52 (d, 2H), 7.05 (d, 2H), 6.62 (t, 2H), 6.50 (d, 1H). |
| 353 | δ 9.05 (t, 1H), 7.93 (d, 1H), 7.64 (d, 1H), 7.55 (t, 1H), 7.48 (t, 1H), 7.42 (s, 1H), |
| | 6.63 (m, 2H), 6.51 (d, 1H). |
| 354 | δ 9.05 (t, 1H), 7.96 (d, 1H), 7.66 (d, 2H), 7.37 (d, 2H), 6.63 (t, 2H), 6.56 (d, 1H). |
| 359 | δ 8.84 (d, 1H), 8.42 (d, 1H), 7.80 (t, 1H), 7.55 (m, 1H), 7.35 (m, 1H), 7.11 (t, 2H), |
| | 3.75 (d, 2H), 2.21 (s, 3H), 1.98 (m, 1H), 0.75 (d, 6H). |
| 360 | δ 9.12 (t, 1H), 7.93 (d, 1H), 7.62 (d, 1H), 7.34 (m, 2H), 7.26 (t, 1H), 6.63 (t, 1H), |
| | 6.60 (t, 1H), 6.58 (d, 1H). |
| 361 | δ 9.08 (t, 1H), 7.88 (d, 1H), 7.58 (t, 1H), 7.10 (t, 2H), 6.50 (d, 1H), 3.02 (m, 1H), |
| | 0.91 (q, 2H), 0.77 (q, 2H). |
| 362 | δ 9.12 (t, 1H), 7.90 (d, 1H), 7.30 (t, 1H), 7.28 (d, 1H), 7.18 (m, 2H), 7.00 (t, 1H), |
| | 6.89 (d, 2H), 6.81 (t, 1H), 6.52 (d, 1H), 3.22 (m, 1H), 2.20 (m, 1H), 1.41 (t, 2H). |
| 363 | δ 9.03 (t, 1H), 7.88 (d, 1H), 7.57 (m, 1H), 7.09 (t, 2H), 6.50 (d, 1H), 4.44 (m, 1H), |
| | 2.78 (m, 2H), 2.08 (m, 2H), 1.30 (m, 1H), 0.90 (m, 1H). |
| 364 | δ 9.91 (s, 1H), 9.13 (s, 1H), 8.74 (m, 1H), 7.64 (s, 1H), 7.16 (t, 2H), 3.85 (d, 2H), |
| | 1.21 (t, 1H), 0.81 (d, 6H). |
| 377 | δ 9.01 (t, 1H), 7.92 (d, 1H), 7.39 (d, 2H), 6.80 (m, 4H), 6.55 (d, 1H), 5.13 (s, 2H). |
| 390 | δ 8.88 (d, 1H), 6.25 (d, 1H), 4.22 (m, 1H), 4.06 (m, 1H), 3.00 (m, 1H), 2.41 (s, 3H), |
| | 2.08 (m, 2H), 1.92 (m, 1H), 1.47 (d, 3H), 1.02 (m, 6H), 0.92 (m, 3H). |
| 391 | δ 9.13 (s, 1H), 7.92 (s, 1H), 7.78 (s, 2H), 6.53 (s, 1H), 3.76 (d, 2H), 1.88 (m, 1H), |
| | 0.85 (d, 6H). |
| 392 | δ 9.10 (s, 1H), 7.90 (s, 1H), 7.48 (m, 1H), 7.08 (m, 1H), 6.98 (m, 1H), 6.51 (s, 1H), |
| | 2.10 (s, 3H), 1.94 (s, 3H). |
| 393 | δ 9.11 (t, 1H), 7.91 (d, 1H), 7.26 (m, 3H), 6.96 (t, 1H), 6.85 (m, 2H), 6.75 (t, 1H), |
| | 6.48 (d, 1H), 3.69 (s, 3H). |
| 394 | δ 9.10 (t, 1H), 7.92 (d, 1H), 7.35 (t, 1H), 7.25 (t, 1H), 6.82 (m, 4H), 6.77 (s, 1H), |
| | 6.50 (d, 1H), 3.74 (s, 3H). |
| 395 | δ 9.10 (t, 1H), 7.91 (d, 1H), 7.33 (t, 1H), 7.11 (d, 2H), 6.83 (m, 4H), 6.50 (d, 1H), |
| | 3.76 (s, 3H). |
| 396 | δ 9.06 (t, 1H), 7.93 (d, 1H), 7.38 (d, 2H), 7.26 (m, 3H), 7.88 (t, 1H), 7.78 (t, 1H), |
| | 6.50 (d, 1H). |
| 397 | δ 9.06 (t, 1H), 7.92 (d, 1H), 7.41 (t, 1H), 7.32 (t, 1H), 7.20 (d, 2H), 7.13 (s, 1H), |
| | 6.83 (m, 2H), 6.52 (d, 1H). |
| 398 | δ 9.06 (t, 1H), 7.92 (d, 1H), 7.35 (m, 1H), 7.25 (d, 2H), 7.20 (d, 2H), 6.84 (t, 2H), |
| | 6.50 (d, 1H). |
| 399 | δ 9.06 (t, 1H), 7.92 (d, 1H), 7.68 (d, 1H), 7.55 (m, 2H), 7.35 (m, 2H), 6.84 (t, 2H), |
| | 6.50 (d, 1H). |
| 400 | δ 9.08 (m, 1H), 7.91 (s, 1H), 7.56 (m, 1H), 7.10 (m, 2H), 6.54 (s, 1H), 5.43 (s, 2H). |
| 401 | δ 8.94 (d, 1H), 8.69 (d, 1H), 8.15 (s, 1H), 7.69 (s, 1H), 7.63 (s, 1H), 7.15 (t, 2H), |
| | 3.85 (d, 2H); 2.07 (s, 1H) 0.81 (d, 6H). |
| 412 | δ 9.20 (s, 1H), 7.38 (m, 2H), 7.26 (m, 1H), 6.27 (s, 1H), 3.67 (q, 2H), 1.20 (t, 3H). |
| 413 | δ 9.18 (s, 1H), 6.94 (t, 2H), 6.23 (s, 1H), 3.86 (t, 2H), 1.61 (m, 2H), 0.84 (t, 3H). |
| 414 | δ 8.98 (s, 2H), 7.40 (m, 1H), 7.12 (m, 2H), 7.63 (m, 2H), 6.24 (s, 1H). |
| 415 | δ 9.18 (s, 1H), 6.92 (t, 2H), 6.13 (s, 1H), 3.82 (d, 2H), 1.75 (m, 1H), 1.25 (m, 1H), |
| | 1.08 (m, 1H), 0.75 (m, 6H). |
| 416 | δ 9.68 (s, 1H), 6.92 (t, 2H), 4.33 (t, 2H), 3.82 (t, 2H), 2.25 (t, 1H), 1.75 (m, 1H), |
| | 1.25 (m, 2H), 1.05 (m, 1H), 0.75 (m, 6H). |
| 417 | δ 7.91 (s, 1H), 6.92 (t, 2H), 3.93 (s, 3H), 3.81 (m, 2H), 1.75 (m, 1H), 1.25 (m, 1H), |
| | 1.05 (m, 1H), 0.75 (m, 6H). |
| 418 | δ 9.0 (d, 1H), 7.8 (s, 1H), 6.9 (m, 2H), 6.4 (d, 1H), 3.6 (br s, 1H), 2.6 (br s, 2H), |
| | 1.8 (d, 2H), 1.7 (d, 2H), 1.6 (d, 1H), 1.2 (m, 1H), 1.0 (m, 2H). |
| 419 | δ 9.02 (m, 1H), 7.42 (m, 3H), 7.18 (m, 3H), 6.80 (t, 1H), 6.18 (m, 1H). |
| 420 | δ 9.04 (m, 1H), 7.40 (m, 3H), 7.16 (m, 2H), 6.62 (t, 2H), 6.15 (m, 1H). |
| 421 | δ 9.08 (d, 1H) 7.88 (d, 1H), 7.24 (m, 1H), 6.88 (m, 1H), 6.76 (m, 1H), 6.48 (m, 1H), |
| | 4.04 (m, 1H), 3.89 (s, 3H), 3.70 (m, 1H), 1.74 (m, 1H), 1.22 (m, 1H), 0.95 (m, 1H), |
| | 0.72 (m, 6H). |
| 422 | δ 9.41 (d, 1H), 9.15-9.08 (m, 1H), 7.62-7.53 (m, 1H), 7.14-7.07 (m, 2H), 3.71 (d, |
| | 2H), 1.94-1.84 (m, 1H), 0.76 (d, 6H). |
| 423 | δ 8.97-8.89 (br s, 1H), 7.61-7.51 (m, 1H), 7.13-7.06 (m, 2H), 3.69 (d, 2H), 2.57 (s, |
| | 3H), 1.92-1.80 (m, 1H), 0.75 (d, 6H). |
| 424 | δ 8.96 (d, 1H), 7.83 (d, 1H), 6.48-6.43 (m, 1H), 4.31-4.19 (m, 1H), 4.13-4.01 (m, |
| | 1H), 3.07-2.96 (m, 1H), 2.13-2.02 (m, 2H), 2.01-1.89 (m, 1H), 1.49 (d, 3H), |
| | 1.04-0.92 (m, 9H). |
| 430 | δ 7.25 (m, 1H), 7.14 (dd, 1H), 7.07 (dd, 1H), 3.96 (br s, 1H), 3.84 (br s, 1H), 2.31 (s, |
| | 3H), 2.09 (s, 3H), 1.82 (m, 1H), 1.17 (m, 1H), 1.01 (m, 1H), 0.72 (m, 6H). |
| 431 | δ 7.59 (m, 3H), 7.24 (m, 2H), 3.82 (br s, 2H), 2.30 (s, 3H), 2.12 (m, 1H), 2.06 (s, |
| | 3H), 1.22 (m, 1H), 0.90 (m, 1H), 0.72 (d, 6H). |

^{a}¹H NMR data are in ppm downfield from tetramethylsilane. Couplings are designated by (s)-singlet, (d)-doublet, (t)-triplet, (q)-quartet, (m)-multiplet, (dd)-doublet of doublets, (dt)-doublet of triplets, (dq)-doublet of quartets, (br s)-broad singlet and (td)-triplet of doublets.

### BIOLOGICAL EXAMPLES OF THE INVENTION

### General Protocol for Preparing Test Suspensions for Tests A-L

The test compounds were first dissolved in acetone in an amount equal to 3% of the final volume and then suspended at the desired concentration (in ppm) in acetone and purified water (50/50 mix) containing 250 ppm of the surfactant Trem® 014 (polyhydric alcohol esters). The resulting test suspensions were then used in tests A-L. Spraying a 200 ppm test suspension (a “&” next to the rating value indicates a 150 ppm test suspension, a “#” next to the rating value indicates a 100 ppm test suspension, a “*” next to the rating value indicates a 40 ppm test suspension) to the point of run-off on the test plants was the equivalent of a rate of 500 g/ha.

### Test A

The test suspension was sprayed to the point of run-off on bluegrass seedlings. The following day the seedlings were inoculated with a spore suspension of Pythium aphanidermatum (the causal agent of bluegrass pythium blight) and incubated in a covered saturated atmosphere at 27° C. for 48 h, and then the covers are removed and the plants left at 27° C. for 3 additional days, after which disease ratings were made.

### Test B

The test suspension was sprayed to the point of run-off on cucumber seedlings. The following day the seedlings were inoculated with a spore suspension of Colletotrichum orbiculare (the causal agent of cucumber Colletotrichum anthracnose) and incubated in saturated atmosphere at 20° C. for 24 h, and moved to a growth chamber at 24° C. for 5 additional days, after which disease ratings were made.

### Test C

The test suspension was sprayed to the point of run-off on cucumber seedlings. The following day the seedlings were inoculated with a spore suspension of Sclerotinia sclerotiorum. (the causal agent of cucumber white mold) and incubated in saturated atmosphere at 24° C. for 24 h, and then moved to a growth chamber at 24° C. for 6 additional days, after which disease ratings were made.

### Test D

Grape seedlings were inoculated with a spore suspension of Plasmopara viticola (the causal agent of grape downy mildew) and incubated in a saturated atmosphere at 20° C. for 24 h. After a short drying period, the test suspension was sprayed to the point of run-off on the grape seedlings and then moved to a growth chamber at 20° C. for 5 days, after which the test units were placed back into a saturated atmosphere at 20° C. for 24 h. Upon removal, disease ratings were made.

### Test E

The test suspension was sprayed to the point of run-off on tomato seedlings. The following day the seedlings were inoculated with a spore suspension of Botrytis cinerea (the causal agent of tomato botrytis) and incubated in saturated atmosphere at 20° C. for 48 h, and then moved to a growth chamber at 24° C. for 1 additional day, after which disease ratings were made.

### Test F

The test suspension was sprayed to the point of run-off on tomato seedlings. The following day the seedlings were inoculated with a spore suspension of Phytophthora infestans (the causal agent of tomato late blight) and incubated in a saturated atmosphere at 20° C. for 24 h, and then moved to a growth chamber at 20° C. for 5 days, after which disease ratings were made.

### Test G

The test suspension was sprayed to the point of run-off on creeping bent grass seedlings. The following -day the seedlings were inoculated with a spore suspension of Rhizoctonia oryzae. (the causal agent of turf brown patch) and incubated in a saturated atmosphere at 27° C. for 24 h, and then moved to a growth chamber at 27° C. for 5 days, after which disease ratings were made.

### Test H

The test suspension was sprayed to the point of run-off on wheat seedlings. The following day the seedlings were inoculated with a spore suspension of Septoria nodorum. (the causal agent of wheat glum blotch) and incubated in a saturated atmosphere at 20° C. for 48 h, and then moved to a growth chamber at 22° C. for 5 days, after which disease ratings were made.

### Test I

The test suspension was sprayed to the point of run-off on wheat seedlings. The following day the seedlings were inoculated with a spore suspension of fusarium graminearium. (the causal agent of wheat head scab) and incubated in a saturated atmosphere at 20° C. for 72 h, and then moved to a growth chamber at 22° C. for 5 days, after which disease ratings were made.

### Test J

The test suspension was sprayed to the point of run-off on wheat seedlings. The following day the seedlings were inoculated with a spore suspension of Septoria tritici. (the causal agent of wheat leaf blotch) and incubated in a saturated atmosphere at 20° C. for 48 h, and then moved to a growth chamber at 20° C. for 20 days, after which disease ratings were made.

### Test K

The test suspension was sprayed to the point of run-off on wheat seedlings. The following day the seedlings were inoculated with a spore suspension of Puccinia recondita f. sp. tritici. (the causal agent of wheat leaf rust) and incubated in a saturated atmosphere at 20° C. for 24 h, and then moved to a growth chamber at 20° C. for 7 days, after which disease ratings were made.

### Test L

The test suspension was sprayed to the point of run-off on wheat seedlings. The following day the seedlings were inoculated with a spore dust of Erysiphe graminis f. sp. tritici, (the causal agent of wheat powdery mildew) and incubated in a growth chamber at 20° C. for 7 days, after which disease ratings were made.

Results for Tests A-L are given in Table A. In the table, a rating of 100 indicates 100% disease control and a rating of 0 indicates no disease control (relative to the controls). A dash (-) indicates no test results. All results are for 200 ppm except where followed by a “&” which indicates 150 ppm, or followed by a “#” which indicates 100 ppm, or followed by a “*” which indicates 40 ppm.

TABLE A

| Cmpd No | Test A | Test B | Test C | Test D | Test E | Test F | Test G | Test H | Test I | Test J | Test K | Test L |
|---|---|---|---|---|---|---|---|---|---|---|---|---|
| 1 | 0 | 0 | 0 | 0 | 100 | 0 | 78 | 99 | 0 | — | 99 | 93 |
| 2 | 38 | 100 | 97 | 77 | 100 | 0 | 100 | 100 | 97 | 99 | 100 | 100 |
| 3 | 0 & | 0 & | — | 12 & | 88 & | 0 & | 100 & | 98 & | 0 & | — | 99 & | 94 & |
| 4 | 0 | 0 | — | 12 | 96 | 0 | 99 | 99 | 0 | — | 98 | 96 |
| 5 | 71 | 33 | 0 | 24 | 71 | — | 26 | 0 | 20 | — | 99 | 97 |
| 6 | 0 | 100 | 100 | 0 | 100 | — | 31 | 100 | 20 | — | 100 | 97 |
| 7 | 51 | 100 | 95 | 88 | 100 | 0 | 95 | 100 | 99 | 100 | 100 | 100 |
| 8 | 0 | 0 | 98 | 30 | 100 | — | 54 | 99 | 0 | — | 100 | 94 |
| 9 | 62 | 100 | 98 | 49 | 100 | 88 | 82 | 100 | 80 | — | 100 | 99 |
| 10 | 0 | 0 | 98 | 55 | 100 | 61 | 97 | 100 | 93 | — | 99 | 0 |
| 11 | 57 | 100 | 99 | 52 | 100 | 90 | 99 | 100 | 87 | — | 100 | 99 |
| 12 | 62 | 0 | 99 | 65 | 100 | 61 | 100 | 100 | 66 | — | 98 | 93 |
| 13 | 51 | 0 | 0 | 0 | 88 | 95 | 37 | 0 | 77 | — | 74 | 0 |
| 14 | 0 | 0 | 0 | 0 | 79 | 27 | 0 | 0 | 0 | — | 0 | 21 |
| 15 | 0 | 0 | 0 | 0 | 0 | 0 | 0 | 0 | 0 | — | 0 | 0 |
| 16 | 0 | 0 | 0 | 8 | 99 | 84 | 0 | 0 | 0 | — | 68 | 26 |
| 17 | 0 | 0 | — | 54 | 100 | 0 | 99 | 100 | 94 | 87 | 100 | 99 |
| 18 | 0 | 100 | 99 | 98 | 100 | 0 | 100 | 100 | 0 | 100 | 100 | 100 |
| 19 | 0 | 100 | 98 | 46 | 100 | 27 | 100 | 100 | 90 | 100 | 100 | 100 |
| 20 | 0 | 0 | 0 | 0 | 100 | 30 | 26 | 98 | 0 | — | 100 | 98 |
| 21 | 0 | 0 | 0 | 15 | 99 | 0 | 31 | 0 | 0 | — | 96 | 96 |
| 22 | 0 | 0 | — | 0 | 100 | 0 | 0 | 0 | 0 | 98 | 79 | 0 |
| 23 | 0 | 21 | 0 | 0 | 99 | 0 | 33 | 98 | 68 | 98 | 68 | 0 |
| 24 | 0 | 0 | 0 | 5 | 0 | 0 | 0 | 0 | 0 | 80 | 0 | 0 |
| 25 | 0 | 100 | — | 0 | 99 | 0 | 100 | 100 | 0 | 100 | 100 | 100 |
| 26 | 0 | 99 | — | 16 | 100 | 0 | 92 | 100 | 0 | 96 | 100 | 100 |
| 27 | 0 | 99 | — | 47 | 100 | 0 | 67 | 100 | 0 | 100 | 100 | 100 |
| 28 | 0 | 98 | — | 0 | 92 | 0 | 31 | 99 | 0 | 100 | 97 | 97 |
| 29 | 0 | 99 | — | 0 | 80 | 0 | 95 | 99 | 0 | 100 | 100 | 98 |
| 30 | 0 | 99 | — | 0 | 99 | 0 | 98 | 100 | 0 | 100 | 100 | 100 |
| 31 | 0 | 0 | — | 0 | 97 | 0 | 0 | 73 | 0 | 100 | 88 | 69 |
| 32 | 0 | 0 | 95 | 15 | 100 | 0 | 92 | 100 | — | 32 | 100 | 0 |
| 33 | 0 | 0 | 16 | 34 | 100 | 0 | 100 | 100 | — | 100 | 100 | 0 |
| 34 | 0 | 0 | 16 | 10 | 100 | 26 | 100 | 100 | — | 97 | 99 | 89 |
| 35 | 0 | 0 | 0 | 5 | 100 | 0 | 90 | 100 | — | 100 | 88 | 97 |
| 36 | — | — | — | 0 | 0 | 0 | — | 0 | — | — | 0 | 0 |
| 37 | — | — | — | 0 | 100 | 0 | — | 100 | — | — | 89 | 0 |
| 38 | — | — | — | 0 | 0 | 0 | — | 0 | — | — | 0 | 0 |
| 39 | 0 | 0 | 0 | 0 | 99 | 0 | 0 | 89 | 0 | — | 67 | 0 |
| 40 | 0 | 0 | 0 | 0 | 100 | 0 | 65 | 99 | 0 | — | 99 | 21 |
| 41 | 0 | 68 | 98 | 8 | 100 | 0 | 47 | 100 | 0 | — | 83 | 0 |
| 42 | 0 | 0 | 0 | 0 | 0 | 0 | 0 | 0 | 60 | — | 0 | 0 |
| 43 | 0 | 99 | 94 | 0 | 100 | 0 | 92 | 100 | 0 | 100* | 100 | 96 |
| 44 | 0* | 0* | 0* | 0* | 95* | 0* | 16* | 0* | 0* | — | 0* | 0* |
| 45 | 0 | 100 | 85 | 0 | 100 | 0 | 26 | 100 | 82 | 100* | 100 | 85 |
| 46 | 0 | 0 | 0 | 0 | 0 | 0 | 0 | 0 | 78 | — | 0 | 0 |
| 47 | 74 | 54 | 0 | 0 | 100 | 0 | 92 | 99 | 78 | 100 | 95 | 99 |
| 48 | 0 | 0 | 0 | 0 | 83 | 0 | 16 | 0 | 0 | — | 80 | 0 |
| 49 | 0 | 0 | 0 | 8 | 47 | 0 | 16 | 0 | 0 | — | 0 | 21 |
| 50 | 0 | 0 | 98 | 0 | 100 | 0 | 95 | 100 | 0 | 100* | 100 | 94 |
| 51 | 0 | 98 | 99 | 0 | 100 | 0 | 31 | 100 | 94 | — | 100 | 63 |
| 52 | 0 | 0 | 90 | 0 | 98 | 0 | 16 | 98 | 0 | — | 74 | 21 |
| 53 | 0 | 100 | 99 | 0 | 100 | 0 | 82 | 100 | 90 | 100* | 100 | 99 |
| 54 | 0 | 0 | 92 | 0 | 100 | 0 | 84 | 90 | 0 | — | 98 | 0 |
| 55 | 0 | 0 | 85 | 0 | 100 | 0 | 96 | 95 | 78 | — | 99 | 0 |
| 56 | 0 | 99 | 99 | 29 | 100 | 0 | 92 | 100 | 60 | 100* | 99 | 99 |
| 57 | 0 | 100 | 98 | 32 | 100 | 0 | 98 | 100 | 0 | 99* | 100 | 100 |
| 58 | 0 | 99 | 0 | 47 | 100 | 0 | 92 | 100 | | 98 | 100 | 99 |
| 59 | 0 | | — | 0 | 100* | 0 | 49* | 73* | 0 | 100* | 94* | 98* |
| 60 | 0 | 0* | — | 0 | 95* | 0 | 0* | 0* | 0 | 40* | 68* | 0* |
| 61 | 0 | 0 | — | 0 | 0 | 0 | 0 | 0 | 0 | 40 | 0 | 0 |
| 62 | 0 | 0 | — | 0 | 31 | 0 | 0 | 0 | 0 | 15 | 83 | 0 |
| 63 | 0 | 0 | — | 0 | 0 | 0 | 72 | 0 | 0 | 90 | 89 | 0 |
| 64 | 0 | 0 | — | 16 | 54 | 0 | 24 | 0 | 0 | 71 | 97 | 0 |
| 65 | 0 | 33 | — | 0 | 99 | 0 | 83 | 0 | 0 | 100 | 98 | 0 |
| 66 | 0 | 62* | 0* | 0 | 100* | 0 | 95* | 89* | 0 | 100* | 97* | 99* |
| 67 | 0 | 0* | 0 | 0 | 0* | 0 | 0* | 0* | 0 | 24 | 0* | 0* |
| 68 | 0 | 0* | 0 | 0 | 0* | 0 | 0* | 0* | 0 | 26* | 0* | 0* |
| 69 | 0 | — | 0 | 0 | 0* | 0 | 0* | 0* | 0 | 62* | 61* | 0* |
| 70 | 0 | 0* | 0 | 0 | 0* | 0 | 0* | 0* | 0 | 83* | 55* | 0* |
| 71 | 0 | 0* | 0 | 0 | 0* | 0 | 30* | 0* | 0 | 64* | 68* | 0* |
| 72 | 0 | 0* | 0 | 0 | 0* | 0 | 58* | 0* | 0 | 74* | 61* | 53* |
| 73 | 0 | 0 | | 0 | 97 | 0 | 0 | 0 | 0 | 59 | 80 | 0 |
| 74 | 0 | 0* | 0 | 0 | 0* | 0 | 0* | 0* | 0 | 26* | 80* | 75* |
| 75 | 0 | 0* | 0 | 0 | 0* | 0 | 0* | 0* | 0 | 7* | 0* | 0* |
| 76 | 0 | 0* | 0 | 0 | 0* | 21 | 0* | 0* | 0 | 0* | 0* | 0* |
| 77 | 0 | 0* | 0 | 0 | 0* | 0 | 0* | 0* | 0 | 0* | 0* | 0* |
| 78 | 0 | 0 | 0 | 0 | 0 | 0 | 0 | 0 | 0 | 91 | 0 | 0 |
| 79 | 0 | 83 | | 0 | 100 | 0 | 46 | 100 | 0 | 100 | 97 | 92 |
| 80 | 0 | 99 | 0* | 0 | 100 | 0 | 47 | 100 | 0 | 100 | 100 | 94 |
| 81 | 0 | 0* | 0 | 0 | 93* | 0 | 31* | 0* | 0 | 52* | 68* | 0* |
| 82 | 0 | 0* | 0 | 0 | 0* | 0 | 0* | 73* | 0 | 5* | 68* | 0* |
| 83 | 0 | 9 | 64 | 0 | 0 | 47 | | 0 | 0 | 18 | 79 | 0 |
| 84 | 0* | 0* | 0* | 5* | 0* | 0* | 0* | 0* | | 20* | 0* | 0* |
| 85 | 0 | 80 | 95 | 0 | 100 | 0 | | 97 | 55 | 100 | 99 | 0 |
| 86 | 0 | 0 | 4 | 0 | 0 | 0 | 0 | 0 | 23 | 0 | 0 | 0 |
| 87 | 0 | 100 | 95 | 0 | 100 | 0 | 99 | 100 | 18 | 74 | 100 | 100 |
| 88 | 0 | 52 | 99 | 0 | 100 | 24 | 16 | 99 | 0 | 100 | 97 | 0 |
| 89 | 0 | 0 | | 0 | 0 | 0 | 0 | 0 | 0 | 0 | 0 | 0 |
| 90 | 0 | 0 | 63 | 16 | 0 | 0 | | 0 | 0 | 0 | 0 | 0 |
| 91 | 0 | 0* | 0 | 0 | 92* | 0 | 0* | 33* | 0 | 93* | 0* | 0* |
| 92 | 0 | 0* | 0 | 0 | 0* | 0 | 0* | 0* | 0 | 7* | 0* | 0* |
| 93 | 0 | 0* | 0 | 0 | 79* | 60 | 70* | 96* | 0 | 83* | 98* | 97* |
| 94 | 0 | 0* | 0 | 0 | 0* | 0 | 0* | 0* | 0 | 14* | 0* | 0* |
| 95 | 0 | 0 | 0 | 92 | 99 | 0 | 68 | 100 | | 95 | 94 | 0 |
| 96 | 0 | 0* | 0 | 0 | 0* | 0 | 0* | 0* | 0 | 0* | 0* | 0* |
| 97 | 0 | 0* | 0 | 64 | 100* | 0 | 0* | 0* | 0 | 24* | 0* | 0* |
| 98 | 0 | 0* | 0 | 0 | 0* | 0 | 0* | 0* | 0 | 26* | 0* | 0* |
| 99 | 0 | 99 | — | 0 | 100 | 0 | 82 | 100 | 0 | 100 | 100 | 100 |
| 100 | 0 | 0 | — | 0 | 0 | 0 | 0 | 0 | 0 | 3 | 26 | 13 |
| 101 | 0 | 0* | 0 | 15 | 0* | 0 | 0* | 0* | | 53* | 0* | 0* |
| 102 | 0 | 0 | — | 0 | 0 | 0 | 0 | 0 | 0 | 0 | 0 | 0 |
| 103 | 0 | 0 | — | 0 | 0 | 0 | 0 | 0 | 0 | 3 | 0 | 0 |
| 104 | 0 | 100 | 0 | 9 | 100 | 0 | 95 | 100 | 0 | 100 | 100 | 100 |
| 105 | 0 | 0 | — | 0 | 100 | 0 | 24 | 64 | 0 | 98 | 100 | 0 |
| 106 | 0 | 0 | — | 0 | 0 | 0 | 0 | 0 | 0 | 12 | 91 | 94 |
| 107 | 0 | 0 | — | 0 | 0 | 0 | 0 | 0 | 69 | 93 | 98 | 0 |
| 108 | 0 | 0 | — | 0 | 56 | 0 | 0 | 0 | 0 | 11 | 0 | 0 |
| 109 | 0 | 0 | — | 0 | 0 | 0 | 0 | 0 | 0 | 0 | 0 | 0 |
| 110 | — | — | — | — | — | — | — | — | — | — | — | — |
| 111 | — | — | — | — | — | — | — | — | — | — | — | — |
| 112 | 0 | 0* | 0 | 0 | 0* | 0 | 0* | 0* | 57 | 0* | 0* | 0* |
| 113 | 0 | 0* | 93 | 0 | 0* | 0 | 26* | 0* | 98 | 2* | 0* | 0* |
| 114 | 0 | 0* | 0 | 0 | 97* | 0 | 0* | 99* | 0 | 89* | 0* | 0* |
| 115 | 0 | 0* | 0 | 0 | 0* | 0 | 0* | 0* | 0 | 0* | 0* | 0* |
| 116 | 0 | 0* | 0 | 0 | 0* | 53 | 0* | 0* | 0 | 5* | 0* | 0* |
| 117 | 0 | 0* | 0 | 0 | 0* | 0 | 0* | 0* | 83 | 2* | 0* | 0* |
| 118 | 0 | 96 | 98 | 0 | 99 | 16 | 71 | 92 | 81 | 100 | 91 | 0 |
| 119 | 0 | 0* | 0 | 0 | 0* | 0 | 0* | 0* | 76 | 68* | 0* | 0* |
| 120 | 0 | 0* | 93 | 0 | 99* | 0 | 0* | 64* | 86 | 91* | 55* | 0* |
| 121 | 0 | 91* | | 0 | 100* | 0 | 44* | 99* | 0 | 100* | 99* | 0* |
| 122 | 0* | 0* | 0* | 58* | 49* | 0* | 16* | 0* | 0* | 7* | 28* | 0* |
| 123 | 0* | 0* | 0* | 0* | 0* | 0* | 0* | 0* | 0* | 19* | 0* | 0* |
| 124 | 0* | 0* | 0* | 0* | 99* | 0* | 21* | 100* | 0* | 100* | 100* | 0* |
| 125 | 0* | 0* | 0* | 0* | 0* | 0* | 0* | 0* | 0* | 71* | 55* | 0* |
| 126 | 0* | 0* | 0* | 0* | 0* | 0* | 0* | 0* | 0* | 19* | 0* | 0* |
| 127 | 0* | 0* | 0* | 0* | 0* | 0* | 0* | 0* | 0* | 16 | 0* | 0* |
| 128 | 0* | 0* | 0* | 0* | 0* | 0* | 0* | 0* | 0* | 0* | 0* | 0* |
| 129 | 0* | 0* | 0* | 0* | 0* | 0* | 0* | 0* | 0* | 19* | 0* | 0* |
| 130 | 0* | 0* | 0* | 0* | 0* | 0* | 0* | 0* | 0* | 16* | 0* | 0* |
| 131 | 0* | 0* | 0* | 0* | 0* | 0* | 0* | 0* | 0* | 52* | 0* | 0* |
| 132 | 0* | 0* | 0* | 0* | 0* | 0* | 0* | 0* | 0* | 9* | 0* | 0* |
| 133 | 0* | 0* | 0* | 0* | 0* | 0* | 0* | 0* | 0* | 23* | 0* | 0* |
| 134 | 0 | 0* | — | 0 | 0* | 0 | 0* | 0* | 89 | 59* | 28* | 0* |
| 135 | 0 | 0* | — | 0 | 0* | 0 | 0* | 0* | 0 | 0* | 0* | 0* |
| 136 | 0 | 0* | — | 40 | 0* | 58 | 0* | 0* | 0 | 28* | 0* | 0* |
| 137 | 0 | 0* | — | 0 | 0* | 0 | 0* | 0* | 0 | 30 | 0* | 0* |
| 138 | 0# | 0# | 24# | 12# | 83# | 0# | 55# | 0# | — | 46# | 0# | 0# |
| 139 | 0* | 0* | 95* | 0* | 100* | 0* | 91* | 97* | 0* | 100* | 99* | 99* |
| 140 | 0 | 0 | 83 | 12 | 99 | 0 | 34 | 0 | — | 41 | 0 | 0 |
| 141 | 0 | 96 | 63 | 12 | 92 | 0 | 96 | 73 | — | 99 | 60 | 79 |
| 142 | 64 | 16 | 0 | 4 | 100 | 0 | 0 | 100 | — | 100 | 27 | 81 |
| 143 | 0 | 0 | 0 | 12 | 99 | 0 | 47 | 0 | — | 100 | 68 | 0 |
| 144 | 0 | 0 | 0 | 12 | 0 | 0 | 0 | 0 | — | 89 | 0 | 0 |
| 145 | 0 | 0 | 49 | 20 | 99 | 0 | 40 | 0 | — | 100 | 68 | 0 |
| 146 | 0* | 0* | 0* | 0* | 0* | 0* | 0* | 0* | 0* | 5* | 0* | 0* |
| 147 | 0 | 0 | 0 | 92 | 97 | 93 | 0 | 0 | 0 | 96 | 80 | 0 |
| 148 | 0 | 47* | — | 73 | 99* | 0 | 81* | 100* | 0 | 100* | 97* | 98* |
| 149 | 0 | 0* | — | 0 | 100* | 0 | 99* | 98* | 0 | 100* | 100* | 100* |
| 150 | 0* | 0* | — | 0* | 0* | 0* | 0* | 0* | 0* | 0* | 0* | 0* |
| 151 | 0 | 0* | — | 31 | 98* | 33 | 0* | 0* | 0 | 12* | 19* | 0* |
| 152 | 0 | 0* | — | 16 | 100* | 0 | 0* | 0* | 0 | 91* | 55* | 0* |
| 153 | 0 | 0* | — | 31 | 79* | 0 | 0* | 0* | 0 | 20* | 0* | 0* |
| 154 | 0 | 0* | — | 50 | 99* | 0 | 0* | 0* | 0 | 45* | 0* | 0* |
| 155 | 0* | 100* | 99* | 47* | 100* | 0* | 100* | 100* | 0* | 100* | 100* | 100* |
| 156 | 0 | 0* | — | 0 | 0* | 0 | 0* | 0* | 0 | 14* | 0* | 0* |
| 157 | 0 | 0* | — | 0 | 0* | 0 | 0* | 0* | 49 | 47* | 0* | 0* |
| 158 | 0 | 0* | — | 0 | 0* | 0 | 0* | 0* | 0 | 15* | 0* | 0* |
| 159 | 0 | 0* | — | 0 | 0* | 47 | 0* | 0* | 0 | 69* | 0* | 0* |
| 160 | — | — | — | — | — | — | — | — | — | — | — | — |
| 161 | 0* | 77* | — | 0* | 100* | 0* | 68* | 100* | 69* | 97* | 100* | 90* |
| 162 | 0 | 9 | 0 | 8 | 85 | 0 | 47 | 0 | — | 100 | 27 | 0 |
| 163 | 0* | 0* | — | 0* | 0* | 0* | 0* | 0* | 0* | 3* | 0* | 0* |
| 164 | 0 | 100 | 91 | 44 | 100 | 0 | 74 | 100 | | 100 | 100 | 100 |
| 165 | 0* | 52* | — | 31* | 100* | 0* | 16* | 100* | 0* | 100* | 100* | 100* |
| 166 | 0* | 17* | — | 0* | 100* | 0* | 26* | 0* | 0* | 100* | 86* | 0* |
| 167 | 0* | 0* | — | 0* | 0* | 0* | 0* | 0* | 0* | 93* | 80* | 0* |
| 168 | 0* | 0* | — | 0* | 0* | 0* | 0* | 0* | 0* | 36* | 0* | 0* |
| 169 | 0* | 98* | — | 0* | 99* | 0* | 0* | 97* | 0* | 100* | 92* | 75* |
| 170 | 0* | 100* | — | 0* | 100* | 0* | 26* | 100* | 0* | 100* | 100* | 99* |
| 171 | 0* | 0 | — | 0* | 100* | 0* | 95* | 82* | 0* | 52* | 96* | 99* |
| 172 | 0* | 0* | 0* | 0* | 07* | 57* | 41* | 0* | 0* | 64* | 0* | 0* |
| 173 | 0* | 0* | 0* | 0* | 0* | 0* | 0* | 0* | 0* | 45* | 0* | 0* |
| 174 | 0* | 0* | 0* | 15* | 100* | 0* | 62* | 99* | — | 100* | 99* | 98* |
| 175 | 0 | 0 | 0 | 12 | 99 | 0 | 91 | 99 | — | 98 | 60 | 92 |
| 176 | 0 | 0 | 0 | 4 | 99 | 0 | 56 | 73 | — | 94 | 0 | 0 |
| 177 | 0* | 0* | 0* | 0* | 0* | 0* | 0* | 0* | 0* | 7* | 0* | 0* |
| 178 | 0* | 0* | — | 0* | 0* | 0* | 0* | 0* | 0* | 5* | 0* | 0* |
| 179 | 0* | 0* | — | 0* | 0* | 0* | 0* | 0* | 0* | 0* | 0* | 0* |
| 180 | 0* | 66* | — | 0* | 99* | 0* | 94* | 99* | 0* | 98* | 100* | 95* |
| 181 | 0* | 51* | — | 0* | 99* | 0* | 100* | 99* | 0* | 100* | 100* | 98* |
| 182 | 0 | 0 | 0 | 0 | 91 | 0 | 31 | 0 | | 100 | 85 | 85 |
| 183 | 0* | 0* | — | 0* | 100* | 0* | 81* | 89* | 0* | 100* | 99* | 92* |
| 184 | 0* | 0* | — | 67* | 100* | 0* | 88* | 100* | 0* | 96* | 98* | 98* |
| 185 | 0* | 0* | 0* | 8* | 81* | 0* | 0* | 0* | | 8* | 0* | 90* |
| 186 | 0* | 16* | — | 0* | 99* | 0* | 67* | 100* | 0* | 92* | 100* | 90* |
| 187 | 0* | 0* | — | 0* | 96* | 0* | 0* | 0* | 0* | 77* | 91* | 0* |
| 188 | 0* | 0* | — | 0* | 56* | 0* | 0* | 0* | 0* | 23* | 0* | 0* |
| 189 | 0* | 0* | — | 0* | 0* | 0* | 0* | 0* | 0* | 15* | 0* | 0* |
| 190 | 0 | 0 | — | 0 | 0 | 0 | 0 | 0 | 0 | 0 | 0 | 0 |
| 191 | 0 | 0 | — | 0 | 97 | 0 | 0 | 0 | 0 | 36 | 86 | 0 |
| 192 | 0* | 0* | — | 0* | 99* | 0* | 31* | 0* | 0* | 98* | 89* | 0* |
| 193 | 0* | 29* | — | 17* | 100* | 0* | 33* | 89* | 0* | 99* | 100* | 68* |
| 194 | 0* | 0* | — | 0* | 95* | 0* | 0* | 0* | 0* | 88* | 92* | 0* |
| 195 | 0* | 26* | — | 0* | 99* | 0* | 47* | 87* | 0* | 100* | 100* | 0* |
| 196 | 0* | 0* | — | 0* | 0* | 0* | 0* | 0* | 44* | 0* | 0* | 0* |
| 197 | 0* | 0* | — | 0* | 41* | 0* | 16* | 0* | 0* | 23* | 0* | 0* |
| 198 | 0* | 0* | 0* | 8* | 0* | 0* | 0* | 0* | — | 0* | 0* | 0* |
| 199 | 0* | 0* | 0* | 4* | 0* | 0* | 0* | 0* | — | 1* | 0* | 0* |
| 200 | 0* | 0* | 0* | 8* | 0* | 0* | 0* | 0* | — | 9* | 0* | 0* |
| 201 | 0* | 0* | 0* | 0* | 0* | 0* | 0* | 0* | — | 0* | 0* | 0* |
| 202 | 45 | 93 | 0 | 12 | 100 | 0 | 99 | 99 | — | 100 | 100 | 99 |
| 203 | 74 | 0 | 0 | 12 | 0 | 0 | 0 | 0 | — | 0 | 0 | 0 |
| 204 | 0 | 0 | 0 | 12 | 98 | 0 | 95 | 0 | — | 90 | 98 | 99 |
| 205 | 0 | 0 | 0 | 12 | 100 | 0 | 85 | 0 | — | 100 | 97 | 81 |
| 206 | 0 | 0 | 0 | 8 | 0 | 0 | 0 | 0 | — | 3 | 0 | 56 |
| 207 | 0 | 0 | 0 | 8 | 50 | 0 | 47 | 0 | — | 70 | 0 | 95 |
| 208 | 0 | 46 | 0 | 12 | 92 | 0 | 0 | 69 | — | 100 | 88 | 0 |
| 209 | 56 | 54 | 0 | 8 | 99 | 0 | 34 | 0 | — | 96 | 85 | 0 |
| 210 | 0 | 0 | 0 | 16 | 0 | 0 | 0 | 0 | — | 1 | 0 | 0 |
| 211 | 0* | 0* | 0* | 4* | 0* | 0* | 0* | 0* | — | 25* | 27* | 0* |
| 212 | 0* | 0* | 0* | 8* | 0* | 0* | 0* | 0* | — | 0* | 0* | 0* |
| 213 | 0* | 0* | 0* | 32* | 41* | 0* | 0* | 0* | — | 0* | 92* | 0* |
| 214 | 0* | 0* | 0* | 12* | 0* | 0* | 0* | 0* | — | 9* | 69* | 0* |
| 215 | 0* | 0* | 0* | 29* | 99* | 0* | 0* | 0* | — | 0* | 55* | 0* |
| 216 | 0* | 53* | 0* | 0* | 100* | 0* | 0* | 89* | — | 97* | 94* | 94* |
| 217 | 0* | 0* | 90* | 0* | 100* | 0* | 0* | 86* | — | 96* | 86* | 62* |
| 218 | 0* | 99* | 99* | 29* | 100* | 0* | 43* | 100* | — | 100* | 100* | 100* |
| 219 | 0* | 100* | 70* | 36* | 100* | 0* | 99* | 100* | — | 100* | 100* | 100* |
| 220 | 0* | 0* | 0* | 0* | 0* | 0* | 0* | 0* | — | 25* | 0* | 0* |
| 221 | 0* | 0* | 0* | 0* | 99* | 0* | 16* | 98* | — | 100* | 89* | 0* |
| 222 | 0* | 0* | 0* | 0* | 92* | 0* | 0* | 60* | — | 86* | 79* | 0* |
| 223 | 0* | 0* | 0* | 0* | 0* | 0* | 0* | 0* | — | 17* | 0* | 51* |
| 224 | 0* | 66* | 0* | 0* | 100* | 0* | 92* | 99* | — | 100* | 94* | 83* |
| 225 | 0* | 0* | 0* | 0* | 100* | 0* | 0* | 73* | — | 100* | 73* | 0* |
| 226 | 0* | 0* | 0* | 0* | 0* | 0* | 0* | 95* | — | 100* | 82* | 46* |
| 227 | 0* | 96* | 0* | 96* | 100* | 0* | 100* | 100* | — | 100* | 100* | 99* |
| 228 | 0* | 98* | 0* | 77* | 100* | 0* | 99* | 100* | — | 99* | 99* | 99* |
| 229 | 0* | 0* | 0* | 0* | 95* | 0* | 16* | 82* | — | 57* | 17* | 88* |
| 230 | 0* | 0* | 0* | 0* | 82* | 0* | 0* | 0* | 0* | 24* | 0* | 0* |
| 231 | 0* | 0* | 0* | 0* | 99* | 0* | 0* | 0* | 0* | 89* | 28* | 0* |
| 232 | 0* | 0* | 0* | 0* | 0* | 0* | 0* | 0* | 0* | 0* | 0* | 0* |
| 233 | 0* | 0* | 0* | 0* | 0* | 0* | 0* | 0* | 0* | 0* | 0* | 0* |
| 234 | 0* | 0* | 0* | 0* | 0* | 0* | 0* | 0* | 0* | 0* | 0* | 0* |
| 235 | 45* | 0* | 0* | 0* | 0* | 0* | 0* | 0* | 0* | 0* | 0* | 0* |
| 236 | 0* | 0* | 0* | 0* | 0* | 0* | 0* | 0* | 0* | 0* | 0* | 0* |
| 237 | 0* | 0* | 0* | 0* | 0* | 0* | 0* | 0* | 0* | 24* | 0* | 0* |
| 238 | 0* | 63* | 0* | 0* | 99* | 0* | 93* | 98* | 0* | 96* | 100* | 74* |
| 239 | 0* | 0* | 0* | 0* | 0* | 0* | 0* | 0* | — | 19* | 0* | 0* |
| 240 | 0* | 9* | 0* | 0* | 100* | 0* | 0* | 78* | — | 93* | 67* | 98* |
| 241 | 0* | 92* | 0* | 33* | 100* | 0* | 85* | 95* | — | 100* | 100* | 93* |
| 242 | 0* | 73* | 0* | 0* | 100* | 0* | 82* | 94* | 0* | 100* | 92* | 99* |
| 243 | 0* | 0* | 0* | 0* | 100* | 0* | 68* | 0* | — | 86* | 45* | 98* |
| 244 | 0* | 0* | 0* | 31* | 42* | 0* | 0* | 0* | — | 9* | 8* | 0* |
| 245 | 0* | 0* | 0* | 0* | 0* | 0* | 0* | 0* | — | 46* | 0* | 0* |
| 246 | 0* | 0* | 0* | 9 | 99* | 0* | 0* | 0* | — | 26* | 0* | 78* |
| 247 | 0* | 0* | 0* | 0* | 0* | 0* | 0* | 0* | — | 35* | 0* | 0* |
| 248 | 0* | 0* | 0* | 0* | 66* | 0* | 0* | 0* | — | 44* | 0* | 0* |
| 249 | 0* | 0* | 0* | 0* | 0* | 0* | 0* | 0* | — | 26* | 0* | 0* |
| 250 | 0* | 0* | 0* | 0* | 0* | 0* | 0* | 0* | — | 30* | 0* | 0* |
| 251 | 0 | 0 | 0 | 0 | 99 | 0 | 21 | 82 | — | 97 | 17 | 0 |
| 252 | 0 | 0 | 0 | 0 | 0 | 0 | 0 | 0 | — | 53 | 0 | 0 |
| 253 | 0 | 0 | 0 | 0 | 0 | 0 | 0 | 0 | — | 66 | 0 | 0 |
| 254 | 0* | 0* | 0* | 0* | 94* | 0* | 41* | 0* | — | 99* | 59* | 54* |
| 255 | 0 | 0 | 0 | 0 | 99 | 0 | 0 | 0 | 0 | 71 | 61 | 0 |
| 256 | 0 | 0 | 0 | 0 | 0 | 0 | 0 | 0 | 0 | 38 | 0 | 0 |
| 257 | 81 | 0 | 0 | 0 | 99 | 0 | 0 | 0 | 0 | 71 | 74 | 0 |
| 258 | 0* | 0* | 49* | 0* | 59* | 0* | 0* | 0* | 0* | 82* | 41* | 0* |
| 259 | 82 | 94 | 0 | 0 | 100 | 0 | 16 | 0 | 0 | 100 | 89 | 59 |
| 260 | 0 | 9 | 0 | 0 | 99 | 0 | 0 | 0 | 0 | 97 | 80 | 50 |
| 261 | 0 | 100 | 83 | 40 | 100 | 0 | 0 | 99 | 0 | 100 | 100 | 98 |
| 262 | 0* | 100* | — | 17* | 100* | 39* | 76* | 100* | 0* | 100* | 100* | 99* |
| 263 | 0* | 100* | — | 0* | 100* | 68* | 84* | 100* | 0* | 100* | 100* | 98* |
| 264 | 0* | 81* | — | 0* | 99* | 80* | 0* | 0* | 0* | 100* | 91* | 83* |
| 265 | 0* | 90* | 0* | 0* | 100* | 0* | 50* | 95* | 0* | 100* | 94* | 91* |
| 266 | 0* | 99* | 0* | 0* | 100* | 0* | 90* | 99* | 0* | 100* | 99* | 99* |
| 267 | 0* | 0* | 0* | 0* | 100* | 0* | 0* | 0* | 0* | 60* | 68* | 0* |
| 268 | 0* | 0* | 0* | 0* | 0* | 0* | 0* | 0* | 0* | 7* | 82* | 0* |
| 269 | 0* | 0* | 0* | 0* | 0* | 0* | 0* | 100* | 0* | 99* | 91* | 0* |
| 270 | 0* | 0* | 0* | 24* | 85* | 0* | 0* | 0* | 0* | 95* | 94* | 0* |
| 271 | 0* | 0* | 0* | 0* | 0* | 0* | 0* | 0* | 0* | 0* | 32* | 0* |
| 272 | 0* | 95* | 44* | 57* | 100* | 0* | 59* | 99* | 0* | 100* | 100* | 100* |
| 273 | 0* | 0* | 0* | 0* | 99* | 0* | 0* | 0* | 0* | 92* | 86* | 0* |
| 274 | 0* | 0* | 0* | 0* | 0* | 0* | 0* | 0* | 0* | 45* | 91* | 71* |
| 275 | 0* | 47* | 0* | 0* | 100* | 0* | 0* | 99* | 0* | 100* | 97* | 61* |
| 276 | 0* | 0* | 0* | 0* | 98* | 0* | 0* | 78* | 0* | 100* | 94* | 0* |
| 277 | 0* | 0* | 0* | 0* | 0* | 0* | 0* | 0* | 0* | 63* | 41* | 0* |
| 278 | 0* | 88* | 0* | 31* | 100* | 0* | 92* | 100* | 0* | 100* | 100* | 99* |
| 279 | 0* | 78* | 0* | 0* | 99* | 0* | 41* | 98* | 0* | 100* | 100* | 99* |
| 280 | 0* | 57* | 0* | 0* | 99* | 0* | 0* | 96* | 0* | 100* | 98* | 69* |
| 281 | 0* | 100* | 0* | 0* | 100* | 0* | 47* | 100* | 0* | 100* | 100* | 100* |
| 282 | 0* | 100* | 0* | 0* | 100* | 0* | 0* | 99* | 0* | 100* | 100* | 100* |
| 283 | 0* | 94* | 0* | 0* | 98* | 47* | 0* | 0* | 0* | 100* | 96* | 97* |
| 284 | 0* | 0* | 0* | 0* | 94* | 16* | 0* | 99* | 0* | 100* | 95* | 96* |
| 285 | 0* | 0* | 0* | 0* | 99* | 0* | 0* | 85* | 0* | 100* | 89* | 92* |
| 286 | 0* | 0* | — | 0* | 97* | 41* | 0* | 0* | 0* | 37* | 0* | 0* |
| 287 | 56* | 99* | — | 47* | 100* | 46* | 93* | 99* | 97* | 100* | 99* | 99* |
| 288 | 0 | 91 | 0 | 0 | 100 | 0 | 0 | 100 | 0 | 100* | 91 | 91 |
| 289 | 0 | 0 | 0 | 0 | 100 | 57 | 95 | 0 | 0 | 96 | 100 | 79 |
| 290 | 0 | 0 | 0 | 0 | 100 | 0 | 77 | 0 | 0 | 100 | 100 | 99 |
| 291 | 0 | 0 | 0 | 33 | 97 | 0 | 0 | 0 | 0 | 100 | 88 | 71 |
| 292 | 0 | 0 | 0 | 0 | 0 | 0 | 0 | 0 | 0 | 38 | 27 | 74 |
| 293 | 0 | 0 | 0 | 0 | 95 | 0 | 0 | 0 | 0 | 97 | 88 | 0 |
| 294 | 0 | 0 | 0 | 0 | 31 | 0 | 0 | 0 | 0 | 95 | 54 | 0 |
| 295 | 0 | 0 | 0 | 0 | 0 | 0 | 0 | 0 | 0 | 53 | 27 | 0 |
| 296 | 0 | 93 | 85 | 53 | 100 | 0 | 0 | 100 | 0 | 100 | 100 | 93 |
| 297 | 0 | 73 | — | 88 | 99 | 92 | 78 | 100 | 75 | 99 | 98 | 97 |
| 298 | 88 | 66 | — | 85 | 99 | 100 | 78 | 99 | 96 | 100 | 100 | 89 |
| 299 | 69 | 40 | — | 40 | 99 | 90 | 31 | 99 | 100 | 100 | 100 | 95 |
| 300 | 0 | 93 | 0 | 40 | 100 | 0 | 16 | 99 | 0 | 100 | 100 | 97 |
| 301 | 0 | 0 | 0 | 0 | 100 | 0 | 0 | 87 | 0 | 98 | 99 | 68 |
| 302 | 0 | 98* | 60 | 0 | 100* | 0 | 94* | 99* | 0 | 100* | 100* | 98* |
| 303 | 0 | 100* | 94 | 58 | 100* | 0 | 100* | 100* | 0 | 100* | 100* | 100* |
| 304 | 0 | 0 | — | 0 | 99 | 49 | 31 | 0 | 0 | 100 | 100 | 46 |
| 305 | 0 | 0 | — | 0 | 99 | 50 | 16 | 0 | 0 | 93 | 97 | 52 |
| 306 | 0 | 0 | — | 0 | 0 | 30 | 0 | 0 | 0 | 44 | 51 | 0 |
| 307 | 0 | 0 | — | 0 | 88 | 84 | 0 | 0 | 49 | 20 | 51 | 0 |
| 308 | 0* | 0* | — | 0* | 0* | 0* | 0* | 0* | 0* | 29* | 9* | 0* |
| 309 | 0* | 0* | — | 0* | 99* | 0* | 0* | 0* | 0* | 5* | 0* | 0* |
| 310 | 0* | 0* | — | 0* | 0* | 0* | 0* | 64* | 0* | 42* | 74* | 0* |
| 311 | 0* | 0* | — | 0* | 94* | 0* | 0* | 0* | 0* | 74* | 74* | 0* |
| 312 | 0 | 0 | — | 40 | 0 | 9 | 0 | 0 | 0 | 13 | 0 | 0 |
| 313 | 0* | 0* | — | 0* | 0* | 0* | 0* | 0* | 0* | 12* | 0* | 0* |
| 314 | 88 | 0 | — | 0 | 0 | 0 | 0 | 0 | 18 | 0 | 0 | 0 |
| 315 | 69 | 0 | — | 0 | 0 | 0 | 0 | 0 | 0 | 7 | 0 | 0 |
| 316 | 54 | 0 | — | 0 | 0 | 0 | 0 | 0 | 0 | 0 | 0 | 0 |
| 317 | 0 | 0 | — | 0 | 0 | 0 | 0 | 0 | 0 | 28 | 41 | 0 |
| 318 | 0* | 82* | — | 0* | 99* | 0* | 78* | 97* | 0* | 100* | 97* | 67* |
| 319 | 0* | 33* | — | 0* | 99* | 0* | 16* | 92* | 0* | 98* | 91* | 76* |
| 320 | 74* | 88* | — | 0* | 99* | 0* | 80* | 100* | 0* | 100* | 99* | 96* |
| 321 | 0* | 94* | — | 17* | 99* | 0* | 70* | 100* | 82* | 100* | 99* | 0* |
| 322 | 0* | 63* | — | 0* | 99* | 0* | 16* | 98* | 0* | 100* | 92* | 0* |
| 323 | 0* | 78* | — | 0* | 99* | 0* | 16* | 97* | 0* | 100* | 96* | 0* |
| 324 | 0* | 99* | — | 0* | 99* | 0* | 97* | 100* | 49* | 100* | 100* | 100* |
| 325 | 0* | 0* | — | 0* | 0* | 0* | 0* | 0 | 0* | 7* | 0 | 0* |
| 326 | 0* | 0* | — | 0* | 99* | 0* | 16* | 100* | 0* | 97* | 97* | 94* |
| 327 | 93* | 37* | — | 0* | 99* | 0* | 0* | 98* | 0* | 97* | 95* | 78* |
| 328 | 0* | 0* | — | 0* | 99* | 0* | 87* | 100* | 31* | 100* | 98* | 88* |
| 329 | 83* | 31* | — | 0* | 99* | 0* | 26* | 100* | 0* | 100* | 96* | 76* |
| 330 | 0* | 0* | — | 0* | — | 0* | 41* | 99* | 0* | 100* | 100* | 71* |
| 331 | 0* | 90* | — | 47* | — | 0* | 16* | 100* | 0* | 100* | 100* | 76* |
| 332 | 0* | 0* | — | 24* | — | 0* | 0* | 94* | 0* | 98* | 95* | 71* |
| 333 | 0* | 37* | — | 47* | — | 0* | 17* | 99* | 0* | 98* | 99* | 0* |
| 334 | 0* | 0* | — | 0* | — | 0* | 0* | 0 | 0* | 96* | 91* | 0* |
| 335 | 0* | 0* | — | 0* | — | 0* | 0* | 98* | 0* | 99* | 91* | 71* |
| 336 | 0* | 0* | — | 0* | 99* | 0* | 41* | 99* | | 100* | 100* | 98* |
| 337 | 0* | 76* | — | 0* | — | 0* | 29* | 99* | 0* | 100* | 95* | 0* |
| 338 | 0* | 80* | — | 0* | — | 0* | 16* | 100* | 0* | 98* | 99* | 0* |
| 339 | 0* | 0* | — | 0* | — | 0* | 0* | 99* | 0* | 100* | 89* | 0* |
| 340 | 0* | 0* | — | 0* | — | 0* | 0* | 99* | 0* | 95* | 95* | 0* |
| 341 | 0* | 52* | — | 79* | — | 0* | 0* | 99* | 0* | 98* | 100* | 63* |
| 342 | 0 | 0 | — | 0 | — | 0 | 95 | 0 | 0 | 82 | 98 | 96 |
| 343 | — | — | — | — | — | — | — | — | — | — | — | — |
| 344 | 0 | 0* | — | 0 | 92* | 0 | 16* | 99* | 93 | 100* | 99* | 0* |
| 345 | 0 | 88* | — | 0 | 98* | 0 | 31* | 99* | 91 | 100* | 100* | 88* |
| 346 | 0 | 0* | — | 0 | 0* | 0 | 0* | 0* | 0 | 13* | 74* | 0* |
| 347 | 0 | 0* | — | 0 | 0* | 0 | 0* | 0* | 0 | 83* | 93* | 25* |
| 348 | 0 | 0* | — | 0 | 90* | 0 | 16* | 64* | 0 | 99* | 97* | 44* |
| 349 | 0 | 0* | — | 0 | 0* | 0 | 0* | 0* | 0 | 25* | 96* | 0* |
| 350 | 80 | 0 | — | 0 | 98 | 0 | 99 | 98 | 80 | 85 | 99 | 86 |
| 351 | 0 | 99* | — | 0 | 99* | 0 | 0* | 99* | 78 | 100* | 100* | 100* |
| 352 | 0 | 99* | — | 0 | 99* | 0 | 31* | 97* | 63 | 100* | 99* | 81* |
| 353 | 0 | 16* | — | 0 | 98* | 0 | 0* | 98* | 0 | 100* | 91* | 79* |
| 354 | 0 | 9* | — | 0 | 95* | 0 | 47* | 91* | 0 | 100* | 97* | 48* |
| 355 | 0* | 99* | — | 0* | 99* | 0* | 66* | 100* | — | 100* | 99* | 57* |
| 356 | 0* | 99* | — | 0* | 100* | 0* | 93* | 100* | — | 100* | 100* | 100* |
| 357 | 0* | 83* | — | 0* | 99* | — | 84* | 98* | 91* | 91* | 99* | 20* |
| 358 | 0* | 0* | — | 0* | 99* | — | 92* | 100* | — | 100* | 100* | 91* |
| 359 | 81* | 0* | — | 8* | 0* | 0* | 0* | 0* | 65* | 85* | 0* | 0* |
| 360 | 0 | 0* | — | 0 | 99* | 0 | 65* | 100* | 86 | 100* | 100* | 99* |
| 361 | 0 | 0* | — | 0 | 92* | 0 | 31* | 98* | 0 | 3* | 83* | 0* |
| 362 | 0 | 0* | — | 0 | 0* | 0 | 0* | 0* | 0 | 0* | 0* | 0* |
| 363 | 0 | 0* | — | 0 | 99* | 0 | 93* | 100* | 0 | 59* | 100* | 99* |
| 364 | 0 | 0* | — | 0 | 99* | 0 | 16* | 0* | 82 | 0* | 92* | 88* |
| 365 | 0* | 99* | — | 16* | 98* | 0* | 99* | 100* | — | 100* | 100* | 96* |
| 366 | 0* | 99* | — | 0* | 99* | 0* | 89* | 100* | — | 96* | 100* | 93* |
| 367 | 0* | 100* | — | 0* | 99* | 0* | 99* | 100* | — | 99* | 100* | 99* |
| 368 | 0* | 99* | — | 0* | 99* | 0* | 99* | 100* | — | 100* | 100* | 99* |
| 369 | 0* | 100* | — | 0* | 99* | 0* | 99* | 100* | — | 100* | 100* | 98* |
| 370 | 0* | 100* | — | 0* | 99* | 0* | 92* | 100* | — | 100* | 100* | 96* |
| 371 | 0* | 99* | — | 0* | 99* | 0* | 99* | 100* | — | 100* | 100* | 98* |
| 372 | 0* | 100* | — | 0* | 99* | 0* | 82 | 100* | — | 100* | 100* | 98 |
| 373 | 0* | 99* | — | 0* | 100* | 0* | 93* | 99* | 78* | 96* | 100* | 100* |
| 374 | 0* | 100* | — | 0* | 99* | 0* | 78* | 100* | 0* | 100* | 100* | 100* |
| 375 | 0* | 100* | — | 0* | 99* | 0* | 95* | 100* | 80* | 100* | 100* | 100* |
| 376 | 0* | 100* | — | 40 | 99* | 0* | 98* | 100* | 0* | 100* | 100* | 100* |
| 377 | 0* | 31* | — | 0* | 99* | 0 | 0* | 0* | 0 | 100* | 98* | 94* |
| 378 | 0* | 0* | — | 0* | 98* | 0* | 51* | 98* | 0* | 100* | 98* | 91* |
| 379 | 0* | 0* | — | 0* | 98* | 0* | 0* | 90* | 0* | 100* | 95* | 40* |
| 380 | 0* | 96* | — | 0* | 99* | 0* | 60 | 99* | 60* | 99* | 100* | 99* |
| 381 | 0* | 78* | — | 0* | 100* | 0* | 16* | 99* | 0* | 100* | 100* | 76* |
| 382 | 0* | 63* | — | 0* | 99* | 0* | 16 | 99* | 0* | 100* | 100* | 50* |
| 383 | 0* | 63* | — | 0* | 99* | 0* | 49* | 100* | 0* | 100* | 100* | 99* |
| 384 | 0* | 90* | — | 15* | 99* | 0* | 47* | 99* | 0* | 98* | 100* | 59* |
| 385 | 0* | 99* | — | 0* | 99* | 0* | 78* | 99* | 0* | 100* | 100* | 89* |
| 386 | 0* | 87* | — | 57* | 99* | 0* | 99* | 100* | 0* | 96* | 100* | 68* |
| 387 | 0* | 0* | — | 0* | 99* | 0* | 60* | 89* | 0* | 99* | 100* | 0* |
| 388 | 0* | 67* | — | 0* | 99* | 0* | 0* | 97* | 0* | 100* | — | 0* |
| 389 | 0* | 98* | — | 0* | 99* | 0* | 31* | 94* | 0* | 100* | — | 0* |
| 390 | 0 | 0 | — | 8 | 98 | 0 | 99 | 0 | 0 | — | 100 | 100 |
| 391 | 0* | 0* | — | 0* | 0* | 0* | 0* | 0* | 0* | 33* | 88* | 0* |
| 392 | 0 | 0 | — | 0 | 99 | 0 | 69 | 78 | 0 | — | 99 | 59 |
| 393 | 0 | 41* | — | — | 29* | 0 | 0* | 73* | 0 | 33* | 89* | 0* |
| 394 | 0 | 99* | — | — | 99* | 0 | 47* | 100* | 78 | 100* | 100* | 99* |
| 395 | 0 | 57* | — | — | 80* | 0 | 0* | 95* | 0 | 99* | 99* | 0* |
| 396 | 0 | 13* | — | — | 70* | 0 | 0* | 89* | 0 | 85* | 94* | 91* |
| 397 | 0 | 37* | — | — | 99* | 0 | 0* | 94* | 0 | 94* | 80* | 0* |
| 398 | 0 | 56* | — | — | 98* | 0 | 24* | 75* | 0 | 97* | 97* | 78* |
| 399 | 0 | 88* | — | — | 97* | 0 | 24* | 90* | 0 | 100* | 99* | 93* |
| 400 | 0* | 0* | — | 0* | 0* | 0* | 0* | 0* | 0* | 0* | — | v |
| 401 | — | 0 | — | 0* | 96 | 0 | 85 | 95 | 0 | 32* | — | 98 |
| 402 | — | 98* | — | 0* | 99* | 0* | 0* | 100* | 0* | 99* | — | 57* |
| 403 | — | 99* | — | 0* | 99* | 0* | 0 | 100* | 0* | 98* | — | 99* |
| 404 | — | 0* | — | 0* | 98* | 0* | 9* | 100* | 0* | 97* | — | 0* |
| 405 | — | 36* | — | 0* | 99* | 0* | 17* | 98* | 0* | 99* | — | 0* |
| 406 | — | 84* | — | 0* | 99* | 0* | 50* | 99* | 0* | 100* | — | 0* |
| 407 | — | 32* | — | 0* | 99* | 0* | 9* | 99* | 0* | 99* | — | 0* |
| 408 | — | 98* | — | 0* | 99* | 0* | 91* | 100* | 0* | 99* | — | 99* |
| 409 | — | 98* | — | 0* | 99* | 0* | 24* | 99* | 0* | 99* | — | 60* |
| 410 | — | 97* | — | 0* | 99* | 0* | 17* | 99* | 0* | 98* | — | 0* |
| 411 | — | 98* | — | 0* | 99* | 0* | 16* | 99* | 0* | 98* | — | 87* |
| 412 | — | 0* | — | 16 | 29* | 9 | 0* | 0* | 0 | 0* | — | 0* |
| 413 | 68 | 0* | — | 64 | 99* | 95 | 0* | 99* | 0 | 93* | — | 98* |
| 414 | 95 | 0* | — | 40 | 99* | 85 | 0* | 0* | 35 | 97* | — | 98* |
| 415 | 86 | 91* | — | 95 | 99* | 99 | 0* | 100* | 0 | 95* | — | 99* |
| 416 | | 99 | — | — | 99 | — | — | 100 | 0 | — | 100 | 99 |
| 417 | 0 | 71* | — | 86 | 99* | 93 | 0* | 60* | 0 | 0 | 100* | 76 |
| 418 | 0 | 99 | — | 47 | 99 | 0 | 99 | 100 | 0 | — | 99 | 100 |
| 419 | 100 | — | — | 32 | 97* | 95 | 0* | 94* | 73 | — | 100* | 0* |
| 420 | 98 | — | — | 31 | 94* | 100 | 0* | 96* | 60 | — | 100* | 41* |
| 421 | 0 | 99 | — | 3 | 99 | 0 | 34 | 100 | 0 | — | 100 | 94 |
| 422 | — | — | — | — | — | — | — | — | — | — | — | — |
| 423 | 0 | 0 | — | 0 | 100 | 0 | 95 | 0 | 60 | 46* | 97 | 97 |
| 424 | 0 | 0 | — | 0 | 96 | 0 | 99 | 0 | 0 | 37* | 100 | 100 |
| 425 | 0 | 0 | — | 0 | 0 | 16 | 0 | 0 | 0 | 3 | 0 | 0 |
| 426 | 82 | 0 | — | 0 | 0 | 0 | 0 | 0 | 0 | 0 | 0 | 80 |
| 427 | 0 | 0 | — | 0 | 0 | 0 | 0 | 0 | 0 | 0 | 0 | 0 |
| 428 | 0 | 0 | — | 0 | 49 | 0 | 0 | 0 | 0 | 87 | 95 | 0 |
| 429 | 88 | 0 | — | 0 | 0 | 0 | 0 | 0 | 62 | 0 | 0 | 0 |
| 430 | 0 | 0 | — | 0 | 96 | 0 | 0 | 0 | 0 | 86* | | 93 |
| 431 | 88 | 0 | — | 46 | 37 | 0 | 16 | 0 | 28 | 0 | 32 | 0 |
| 432 | 0 | 0 | — | 0 | 48 | 0 | 16 | 0 | — | 0 | 0 | 0 |

What is claimed is:

1. A compound selected from Formula 1, an N-oxide or an agriculturally suitable salt thereof, 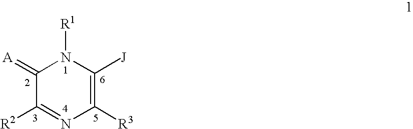
wherein
R¹ is NR⁴R⁵, N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G²;
A is O, S or NR⁷;
R⁷ is H, C₁-C₄ alkyl, C₁-C₄ haloalkyl, C₂-C₆ alkylcarbonyl or C₂-C₆ alkoxycarbonyl;
R² is cyano, NR⁸—N═CR⁹R¹⁰, O—N═CR⁹R¹⁰, NR⁸—NR¹¹R¹², O—NR¹¹R¹², CR¹³═NOR¹⁴, CR¹³═NNR¹¹R¹², C(W)NR²²R²³, NC(═O)R³⁰, NC(═O)NR³¹ or NC(═O)OR³²; or
R² is a 5- or 6-membered heteroaromatic ring or a 8-, 9- or 10-membered heteroaromatic bicyclic ring system, each ring or ring system optionally substituted with up to 5 substituents selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents selected from R²⁴;
W is O, S or ═NR²⁵;
R³ is H, halogen, cyano, C₁-C₆ alkyl, C₁-C₄ haloalkyl, C₃-C₆ cycloalkyl, C₃-C₆ halocycloalkyl, C₂-C₆ alkenyl, C₃-C₆ alkynyl, C₁-C₄ alkoxy, C₁-C₄ haloalkoxy, C₁-C₄ alkylthio, C₁-C₄ haloalkylthio, C₂-C₅ alkoxycarbonyl, hydroxycarbonyl, —SCN or —CHO;
each R⁴ and R⁵ are independently H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R⁴ and R⁵ are taken together as —(CH₂)₃—, —(CH₂)₄—, —(CH₂)₅—, —(CH₂)₆—, —CH₂CH₂OCH₂CH₂— or CH₂CH(CH₃)OCH(CH₃)CH₂—;
R⁶ is H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl;
R⁸ is H, C₁-C₄ alkyl or C₁-C₄ haloalkyl;
R⁹ is C₁-C₄ alkyl or C₁-C₄ haloalkyl;
R¹⁰ is H, C₁-C₄ alkyl or C₁-C₄ haloalkyl; or
R⁹ and R¹⁰ are taken together as —(CH₂)₃—, —(CH₂)₄—, —(CH₂)₅— or —(CH₂)₆—;
R¹¹ is H, C₁-C₄ alkyl or C₁-C₄ haloalkyl;
R¹² is H, C₁-C₄ alkyl, C₁-C₄ haloalkyl, C₂-C₃ alkylcarbonyl or C₂-C₃ alkoxycarbonyl; or
R¹¹ and R¹² are taken together as —(CH₂)₄—, —(CH₂)₅, —CH₂CH₂OCH₂CH₂— or —CH₂CH(CH₃)OCH(CH₃)CH₂—;
R¹³ is H, NH₂, C₁-C₄ alkyl or C₁-C₄ haloalkyl;
R¹⁴ is H, C₁-C₄ alkyl or C₁-C₄ haloalkyl;
J is C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents selected from the group consisting of halogen, cyano, nitro, hydroxy, C₁-C₄ alkoxy, C₁-C₄ haloalkoxy, C₁-C₄ alkylthio, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl and C₁-C₄ alkylamino; or
J is a phenyl, benzyl, naphthalene, 5- or 6-membered heteroaromatic ring or 8-, 9- or 10-membered heteroaromatic bicyclic ring system, each ring or ring system optionally substituted with up to 5 substituents selected from halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl and C₃-C₆ trialkylsilyl;
G¹ is a 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents selected from R¹⁷;
G² is a phenyl ring, 5- or 6-membered heteroaromatic ring, each ring or ring system optionally substituted with from 1 to 4 substituents independently selected from R¹⁸;
each R¹⁷ is independently C₁-C₂ alkyl, C₁-C₂ haloalkyl, halogen, cyano, nitro or C₁-C₂ alkoxy;
each R¹⁸ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, (C₁-C₄ alkyl)(C₃-C₆ cycloalkyl)amino, C₂₋C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R¹⁹ and R²¹ are independently H, C₁-C₄ alkyl, C₁-C₄ haloalkyl or C₃-C₈ cycloalkyl; or
R¹⁹ and R²¹ are taken together as —(CH₂)₄—, —(CH₂)₅, —CH₂CH₂OCH₂CH₂— or —CH₂CH(CH₃)OCH(CH₃)CH₂—;
each R²² and R²³ are independently H, C₁-C₄ alkyl, C₃-C₈ cycloalkyl, C₄-C₈ cycloalkylalkyl each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R²² and R²³ are taken together as —(CH₂)₄—, —(CH₂)₅, —CH₂CH₂OCH₂CH₂— or —CH₂CH(CH₃)OCH(CH₃)CH₂—;
each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ alkoxyalkyl, C₃-C₆ dialkoxyalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl; and
R²⁵ is H, C₁-C₄ alkyl or C₁-C₄ haloalkyl;
R³⁰ is H, C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, 5- or 6-membered heteroaromatic ring, each ring or ring system optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy and C₁-C₄ haloalkoxy; and
each R³¹ and R³² are independently C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, C₂-C₄ halogen, cyano, nitro, C₁-C₄ alkoxy and C₁-C₄ haloalkoxy.
provided that when R² is NR⁸—N═CR⁹R¹⁰, O—N═CR⁹R¹⁰, NR⁸—NR¹¹R¹², O—NR¹¹R¹², CR¹³═NOR¹⁴ or CR¹³═NNR¹¹R¹², then J is phenyl substituted with at least one substituent selected from halogen and methyl.

2. The compound of claim 1 wherein
A is O or S;
R¹ is C₂-C₆ alkyl, C₂-C₆ haloalkyl, C₃-C₈ cycloalkyl, C₄-C₈ alkylcycloalkyl, NR⁴R⁵, G¹ or G²;
R² is cyano or C(W)NR²²R²³; or a 5- or 6-membered heteroaromatic ring; or a 5- or 6-membered saturated or partially saturated heterocyclic ring, optionally including 1-3 C(═O) group as ring members;
R³ is halogen or C₁-C₆ alkyl;
R⁴ and R⁵ are independently H, C₃-C₆ alkyl or C₃-C₆ haloalkyl; and
J is phenyl optionally substituted at the 2, 3, 4 and 6 positions with substituents selected from methyl, methoxy, chloro and fluoro.

3. The compound of claim 2 wherein
A is O;
R¹ is C₂-C₆ alkyl, C₂-C₆ haloalkyl, C₃-C₈ cycloalkyl, C₄-C₈ alkylcycloalkyl, G¹ or G²;
R² is 5- or 6-membered heteroaromatic ring, cyano or CONH₂;
R³ is halogen or methyl; and
J is phenyl optionally substituted at the 2, 3, 4 and 6 positions with substituents selected from methyl, chloro and fluoro.

4. The compound of claim 3 wherein
R¹ is C₂-C₆ alkyl, C₂-C₆ haloalkyl or phenyl, optionally substituted with from 1 to 4 substituents independently selected from R¹⁸;
R² is 5- or 6-membered heteroaromatic ring or CONH₂; and
R³ is bromo, chloro, fluoro or methyl.

5. The compound of claim 4 wherein
R¹ is C₄-C₆ alkyl, C₄-C₆ haloalkyl or phenyl, optionally substituted with from 1 to 4 substituents independently selected from R¹⁸;
R² is 1H-pyrazol-1-yl, 1H-1,2,4-triazol-1-yl or 2-pyridinyl, each optionally substituted with halogen, cyano, C₁-C₆ alkyl or C₁-C₄ haloalkyl; or CONH₂; and
J is 2,4-difluorophenyl, 2,6-difluorophenyl, 2,4,6-trifluorophenyl, 2,3,6-trifluorophenyl, 2-chloro-4-fluorophenyl or 2-chloro-6-fluorophenyl.

6. A compound of claim 1 selected from the group consisting of:
5-Chloro-6-(2,6-difluorophenyl)-1-(2-methylpropyl)-3-(1H-pyrazol-1-yl)-2(1H)-pyrazinone,
5-Chloro-1-[(2S)-2-methylbutyl)-3-(1H-pyrazol-1-yl)-6-(2,4,6-trifluorophenyl)-2(1H)-pyrazinone,
5-Chloro-6-(2,6-difluorophenyl)-1-phenyl-3-(1H-pyrazol-1-yl)-2(1H)-pyrazinone,
5-Chloro-6-(2-chloro-4-fluorophenyl)-1-(2-methylbutyl)-3-(3-methyl-1H-pyrazol-1-yl)-2(1H)-pyrazinone,
6-Chloro-5-(2,6-difluorophenyl)-3,4-dihydro-4-(2-methylbutyl)-3-oxopyrazinecarboxamide,
5-Chloro-6-(2-chloro-4-fluorophenyl)-1-(2-methylpropyl)-3-(1H-pyrazol-1-yl)-2(1H)-pyrazinone,
5-Chloro-1-(3-fluorophenyl)-3-(1H-pyrazol-1-yl)-6-(2,4,6-trifluorophenyl)-2(1H)-pyrazinone,
5-Chloro-1-phenyl-3-(1H-pyrazol-1-yl)-6-(2,3,6-trifluorophenyl)-2(1H)-pyrazinone.

7. A fungicidal composition comprising (a) a fungicidally effective amount of a compound selected from the compounds of claim 1, and (b) at least one additional component selected from the group consisting of surfactants, solid diluents and liquid diluents.

8. A fungicidal composition comprising (a) a fungicidally effective amount of a compound selected from the compounds of claim 1, their N-oxides and agriculturally suitable salts, and (b) at least one other fungicide.

9. A method for controlling plant diseases caused by fungal plant pathogens comprising applying to the plant or portion thereof, or to the plant seed, a fungicidally effective amount of a compound of claim 1.
